SEQUENCE LISTING (178 sequences)

SEQ ID NO: 1 - protein, 119 aa, synthetic construct [sequence omitted]

SEQ ID NO: 2 - protein, 107 aa, synthetic construct [sequence omitted]

SEQ ID NO: 3 - protein, 242 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSSCGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIK

SEQ ID NO: 4 - protein, 120 aa, synthetic construct [sequence omitted]

SEQ ID NO: 5 - protein, 107 aa, synthetic construct [sequence omitted]

SEQ ID NO: 6 - protein, 247 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 7 - protein, 116 aa, synthetic construct
EVKLVESGGGLVKPGGSLRLSCATSGFSFSSYYMYWVRQAPGKRLEWVAYISSGSEIYYSDSVKGRFTISRDSAKNTLYLQMNSLRAEDTAVYYCARLGDDGMDWWGQGTTWTVSS

SEQ ID NO: 8 - protein, 107 aa, synthetic construct
DIVMTQSPSSLSASVGDRVTITCRASQNVRTAVAWFQQKPGKAPKALIYLASNRHTGVPDRFTGSGSGTEFTLTISNLQPEDFATYFCLQHNSYPLTFGGGTKVEIK

SEQ ID NO: 9 - protein, 237 aa, synthetic construct
EVKLVESGGGLVKPGGSLRLSCATSGFSFSSYYMYWVRQAPGKRLEWVAYISSGSEIYYSDSVKGRFTISRDSAKNTLYLQMNSLRAEDTAVYYCARLGDDGMDWWGQGTTWTVSSGGGSGGGGSGGGGSDIVMTQSPSSLSASVGDRVTITCRASQNVRTAVAWFQQKPGKAPKALIYLASNRHTGVPDRFTGSGSGTEFTLTISNLQPEDFATYFCLQHNSYPLTFGGGTKVEIK

SEQ ID NO: 10 - protein, 108 aa, synthetic construct
ADIVLTQSPDSLAVSLGERVTINCKSSQPLEYSNNQWNYLAWYQQKPGQSPKLLISWASTRKSGVPDRFSGSGSGTDFTLTISSVQAEDVAVYYCGQYSDYPNTFGAG

SEQ ID NO: 11 - protein, 119 aa, synthetic construct
QVQLVQSGAEVVKPGASVKISCKASGYPFTQYFIHWVKQNPGQRLEWIGQISSSYATVDYNQKFKGKATLTVDTSASIAYMELSSLRSEDTAVYYCVRSGNYEEYAMDYWGQGTLVTVS

SEQ ID NO: 12 - protein, 254 aa, synthetic construct
ADIVLTQSPDSLAVSLGERVTINCKSSQPLEYSNNQWNYLAWYQQKPGQSPKLLISWASTRKSGVPDRFSGSGSGTDFTLTISSVQAEDVAVYYCGQYSDYPNTFGAGTKLEIKRGGGGSGGGGSGGGGSGGGGSQVQLVQSGAEVVKPGASVKISCKASGYPFTQYFIHWVKQNPGQRLEWIGQISSSYATVDYNQKFKGKATLTVDTSASIAYMELSSLRSEDTAVYYCVRSGNYEEYAMDYWGQGTLVTVS

SEQ ID NO: 13 - protein, 112 aa, synthetic construct
AAQPADIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGG

SEQ ID NO: 14 - protein, 120 aa, synthetic construct
EVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSS

SEQ ID NO: 15 - protein, 259 aa, synthetic construct
AAQPADIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSS

SEQ ID NO: 16 - protein, 121 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 17 - protein, 110 aa, synthetic construct
GGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 18 - protein, 242 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLEL

SEQ ID NO: 19 - protein, 490 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKSCGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 20 - protein, 495 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 21 - protein, 486 aa, synthetic construct
DIVMTQSPSSLSASVGDRVTITCRASQNVRTAVAWFQQKPGKAPKALIYLASNRHTGVPDRFTGSGSGTEFTLTISNLQPEDFATYFCLQHNSYPLTFGGGTKVEIKGGGGSGGGGSGGGGSEVKLVESGGGLVKPGGSLRLSCATSGFSFSSYYMYWVRQAPGKRLEWVAYISSGSEIYYSDSVKGRFTISRDSAKNTLYLQMNSLRAEDTAVYYCARLGDDGMDWWGQGTTWTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 22 - protein, 502 aa, synthetic construct
ADIVLTQSPDSLAVSLGERVTINCKSSQPLEYSNNQWNYLAWYQQKPGQSPKLLISWASTRKSGVPDRFSGSGSGTDFTLTISSVQAEDVAVYYCGQYSDYPNTFGAGTKLEIKRGGGGSGGGGSGGGGSGGGGSQVQLVQSGAEVVKPGASVKISCKASGYPFTQYFIHWVKQNPGQRLEWIGQISSSYATVDYNQKFKGKATLTVDTSASIAYMELSSLRSEDTAVYYCVRSGNYEEYAMDYWGQGTLVTVSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 23 - protein, 507 aa, synthetic construct
AAQPADIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 24 - protein, 16 aa, synthetic construct
SCGGGSGGGGSGGGGS

SEQ ID NO: 25 - protein, 19 aa, synthetic construct
GGGGSGGGGSGGGGSGGGG

SEQ ID NO: 26 - protein, 14 aa, synthetic construct
GGGSGGGGSGGGGS

SEQ ID NO: 27 - protein, 20 aa, synthetic construct
GGGGSGGGGSGGGGSGGGGS

SEQ ID NO: 28 - protein, 12 aa, synthetic construct
GGSGGSGGSGGS

SEQ ID NO: 29 - protein, 5 aa, synthetic construct
GGGGS

SEQ ID NO: 30 - protein, 6 aa, synthetic construct
TKVEIK

SEQ ID NO: 31 - protein, 7 aa, synthetic construct
TKLEIKR

SEQ ID NO: 32 - protein, 6 aa, synthetic construct
TKLELK

SEQ ID NO: 33 - skipped

SEQ ID NO: 34 - protein, 22 aa, synthetic construct
MWWRLWWLLLLLLLLWPMVWAA

SEQ ID NO: 35 - DNA, 324 bp, synthetic construct
  source 1..324: mol_type = other DNA
gctgacatagtgctgacccagagtcctgattccctggctgtgtctctgggagagagagtgaccatcaattgcaagtccagccagcccctggaatacagcaataatcagtggaactacctggcttggtaccagcagaaacctggacagtcccctaagctgctgattagctgggccagcaccagaaagtctggagtcccagacagattttctggttctggctctggaactgacttcaccctcacaatcagctctgtgcaggctgaagatgtggctgtgtattactgtggccagtactctgattatcccaacacctttggggctggc

SEQ ID NO: 36 - DNA, 599 bp, synthetic construct
  source 1..599: mol_type = other DNA
caggtgcagctggtgcagtctggagctgaggtggtgaaaccaggggcttctgtgaagatttcctgtaaggcctctggctaccctttcactcagtacttcattcattgggtcaagcagaatcctgggcagagactggaatggattggacaaatctcctccagctatgccacagtggactacaatcagaagttcaagggcaaggcaaccctgactgtggacacatctgccagcattgcctatatggaactgagcagcctgagatctgaagatacagctgtgtattactgtgtgagatctggcaattatgaggagtatgccatggactactggggacagggcaccctggtgactgtgtctggaggaggaggttctgacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcag

SEQ ID NO: 37 - DNA, 363 bp, synthetic construct
  source 1..363: mol_type = other DNA
gacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggag

SEQ ID NO: 38 - DNA, 330 bp, synthetic construct
  source 1..330: mol_type = other DNA
ggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagctgaag

SEQ ID NO: 39 - DNA, 303 bp, synthetic construct
  source 1..303: mol_type = other DNA
gacattcagatgactcagtctcccagctccctgtctgcctctgtgggagacagagtgacaatcacatgtaaggcctcccaggatgtgtctattggagtggcctggtaccagcagaaacctggaaaggccccaaaactgctgatctactctgcctcctacaggtatacaggagtgccttccagattctctggctctggctctggcacagatttcactcttaccatcagttccctccagcctgaggattttgctacctactattgccagcagtactacatctatccttacacatttggccaggga

SEQ ID NO: 40 - DNA, 357 bp, synthetic construct
  source 1..357: mol_type = other DNA
gaggtgcagctggtggagtctgggggaggcctggtgcagccagggggctcactgagactgagctgtgctgcctctgggtttacatttacagactataccatggactgggtgagacaggccccagggaagggcctggagtgggtggcagatgtgaacccaaattctggagggtccatctacaatcagaggttcaaggggagatttaccctgtctgtggatagaagcaagaacacactgtaccttcagatgaactccctgagagctgaggacacagctgtgtactactgtgccagaaacctgggcccatccttttactttgactactggggacagggcacactggtgacagtctcctct

SEQ ID NO: 41 - DNA, 363 bp, synthetic construct
  source 1..363: mol_type = other DNA
gatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggag

SEQ ID NO: 42 - DNA, 329 bp, synthetic construct
  source 1..329: mol_type = other DNA
gggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgtcagcagtggagttccaatccactcacttttggggcaggcacaaaactggagctgaa

SEQ ID NO: 43 - DNA, 303 bp, synthetic construct
  source 1..303: mol_type = other DNA
gacattcagatgacacagtccccatcttccctgtctgcctctgtgggagacagggtgacaatcacttgtagggccagccaggatgtgaacacagctgtggcctggtatcagcagaaacctggaaaggccccaaagctgctgatctactcagcttccttcctgtattctggagtgccatctaggttttctggctccaggtctggcacagactttaccctgaccatctcttctctgcagcctgaggactttgccacttactactgtcagcagcattacacaacccccccaacctttggccagggc

SEQ ID NO: 44 - DNA, 357 bp, synthetic construct
  source 1..357: mol_type = other DNA
gaggtgcagctggtggagtctgggggaggcctggtgcagcctggaggctccctgagactctcctgtgctgcctctggattcaatatcaaagatacttacatccactgggtgagacaggcccctggcaaaggcctggaatgggtggccagaatctatcctactaatggctacaccagatatgctgactctgtgaagggcagatttacaatctctgctgacacctctaaaaatacagcctacctgcagatgaattccctgagagctgaagacacagcagtgtactactgcagcaggtggggtggagatggcttctatgccatggactattggggccagggcaccctggtgacagtctca

SEQ ID NO: 45 - DNA, 363 bp, synthetic construct
  source 1..363: mol_type = other DNA
gacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaa

SEQ ID NO: 46 - DNA, 330 bp, synthetic construct
  source 1..330: mol_type = other DNA
ggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaag

SEQ ID NO: 47 - DNA, 321 bp, synthetic construct
  source 1..321: mol_type = other DNA
gacattgtgatgacacagtccccctctagcctgtctgcttctgtgggagacagggtgacaatcacatgcagagcctctcagaatgtgagaacagctgtggcatggttccagcagaaaccaggaaaggcccccaaggccctgatctacctggcctctaacaggcacacaggggtgcctgataggttcacaggctctggatctgggacagagtttaccctgaccatcagcaacctgcagcctgaggactttgctacatacttctgcctgcagcacaacagctatcctctgacttttggaggaggcaccaaagtggagatcaag

SEQ ID NO: 48 - DNA, 348 bp, synthetic construct
  source 1..348: mol_type = other DNA
gaggtaaagctggtggagtctggaggaggcctggtgaaaccagggggctctctgagactgagttgtgccacatctggcttttcctttagcagctattatatgtactgggtgaggcaggccccaggcaaaaggctggagtgggtggcctacatcagctctggctctgaaatttattactctgactctgtcaagggcagattcaccatcagcagagattctgccaagaacaccctgtatctccagatgaactctctgagagctgaggacacagctgtgtattactgtgctagactgggagatgatggaatggactggtggggccagggaaccacatggacagtgtcttct

SEQ ID NO: 49 - DNA, 363 bp, synthetic construct
  source 1..363: mol_type = other DNA
gacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaa

SEQ ID NO: 50 - DNA, 330 bp, synthetic construct
  source 1..330: mol_type = other DNA
ggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaa

SEQ ID NO: 51 - DNA, 357 bp, synthetic construct
  source 1..357: mol_type = other DNA
gcggcgcagccggcggatattgtgctgacccagaccccgagcagcctgccggtgagcgtgggcgaaaaagtgaccatgacctgcaaaagcagccagaccctgctgtatagcaacaaccagaaaaactatctggcgtggtatcagcagaaaccgggccagagcccgaaactgctgattagctgggcgtttacccgcaaaagcggcgtgccggatcgctttaccggcagcggcagcggcaccgattttaccctgaccattggcagcgtgaaagcggaagatctggcggtgtattattgccagcagtatagcaactatccgtggacctttggcggcggcacccgcctggaaattaaacgc

SEQ ID NO: 52 - DNA, 360 bp, synthetic construct
  source 1..360: mol_type = other DNA
gaagtgcagctgcagcagagcggcccggaagtggtgaaaaccggcgcgagcgtgaaaattagctgcaaagcgagcggctatagctttaccggctattttattaactgggtgaaaaaaaacagcggcaaaagcccggaatggattggccatattagcagcagctatgcgaccagcacctataaccagaaatttaaaaacaaagcggcgtttaccgtggataccagcagcagcaccgcgtttatgcagctgaacagcctgaccagcgaagatagcgcggtgtattattgcgtgcgcagcggcaactatgaagaatatgcgatggattattggggccagggcaccagcgtgaccgtgagcagc

SEQ ID NO: 53 - DNA, 762 bp, synthetic construct
  source 1..762: mol_type = other DNA
gctgacatagtgctgacccagagtcctgattccctggctgtgtctctgggagagagagtgaccatcaattgcaagtccagccagcccctggaatacagcaataatcagtggaactacctggcttggtaccagcagaaacctggacagtcccctaagctgctgattagctgggccagcaccagaaagtctggagtcccagacagattttctggttctggctctggaactgacttcaccctcacaatcagctctgtgcaggctgaagatgtggctgtgtattactgtggccagtactctgattatcccaacacctttggggctggcacaaagctggaaatcaagagaggaggtggaggttctggaggagggggatctggtggagggggctctggaggaggagggtctcaggtgcagctggtgcagtctggagctgaggtggtgaaaccaggggcttctgtgaagatttcctgtaaggcctctggctaccctttcactcagtacttcattcattgggtcaagcagaatcctgggcagagactggaatggattggacaaatctcctccagctatgccacagtggactacaatcagaagttcaagggcaaggcaaccctgactgtggacacatctgccagcattgcctatatggaactgagcagcctgagatctgaagatacagctgtgtattactgtgtgagatctggcaattatgaggagtatgccatggactactggggacagggcaccctggtgactgtgtct

SEQ ID NO: 54 - DNA, 726 bp, synthetic construct
  source 1..726: mol_type = other DNA
gacattcagatgactcagtctcccagctccctgtctgcctctgtgggagacagagtgacaatcacatgtaaggcctcccaggatgtgtctattggagtggcctggtaccagcagaaacctggaaaggccccaaaactgctgatctactctgcctcctacaggtatacaggagtgccttccagattctctggctctggctctggcacagatttcactcttaccatcagttccctccagcctgaggattttgctacctactattgccagcagtactacatctatccttacacatttggccagggaacaaaagtggagatcaagtcctgtggagggggatctgggggaggggggtcaggagggggaggctctgaggtgcagctggtggagtctgggggaggcctggtgcagccagggggctcactgagactgagctgtgctgcctctgggtttacatttacagactataccatggactgggtgagacaggccccagggaagggcctggagtgggtggcagatgtgaacccaaattctggagggtccatctacaatcagaggttcaaggggagatttaccctgtctgtggatagaagcaagaacacactgtaccttcagatgaactccctgagagctgaggacacagctgtgtactactgtgccagaaacctgggcccatccttttactttgactactggggacagggcacactggtgacagtctcctct

SEQ ID NO: 55 - DNA, 741 bp, synthetic construct
  source 1..741: mol_type = other DNA
gacattcagatgacacagtccccatcttccctgtctgcctctgtgggagacagggtgacaatcacttgtagggccagccaggatgtgaacacagctgtggcctggtatcagcagaaacctggaaaggccccaaagctgctgatctactcagcttccttcctgtattctggagtgccatctaggttttctggctccaggtctggcacagactttaccctgaccatctcttctctgcagcctgaggactttgccacttactactgtcagcagcattacacaacccccccaacctttggccagggcaccaaggtggagatcaagggaggagggggaagtgggggaggaggatctggaggagggggatcaggagggggaggctcagaggtgcagctggtggagtctgggggaggcctggtgcagcctggaggctccctgagactctcctgtgctgcctctggattcaatatcaaagatacttacatccactgggtgagacaggcccctggcaaaggcctggaatgggtggccagaatctatcctactaatggctacaccagatatgctgactctgtgaagggcagatttacaatctctgctgacacctctaaaaatacagcctacctgcagatgaattccctgagagctgaagacacagcagtgtactactgcagcaggtggggtggagatggcttctatgccatggactattggggccagggcaccctggtgacagtctcatct

SEQ ID NO: 56 - DNA, 714 bp, synthetic construct
  source 1..714: mol_type = other DNA
gacattgtgatgacacagtccccctctagcctgtctgcttctgtgggagacagggtgacaatcacatgcagagcctctcagaatgtgagaacagctgtggcatggttccagcagaaaccaggaaaggcccccaaggccctgatctacctggcctctaacaggcacacaggggtgcctgataggttcacaggctctggatctgggacagagtttaccctgaccatcagcaacctgcagcctgaggactttgctacatacttctgcctgcagcacaacagctatcctctgacttttggaggaggcaccaaagtggagatcaagggaggaggaggttctgggggaggaggatctggaggaggaggatctgaggtaaagctggtggagtctggaggaggcctggtgaaaccagggggctctctgagactgagttgtgccacatctggcttttcctttagcagctattatatgtactgggtgaggcaggccccaggcaaaaggctggagtgggtggcctacatcagctctggctctgaaatttattactctgactctgtcaagggcagattcaccatcagcagagattctgccaagaacaccctgtatctccagatgaactctctgagagctgaggacacagctgtgtattactgtgctagactgggagatgatggaatggactggtggggccagggaaccacatggacagtgtcttct

SEQ ID NO: 57 - DNA, 777 bp, synthetic construct
  source 1..777: mol_type = other DNA
gcggcgcagccggcggatattgtgctgacccagaccccgagcagcctgccggtgagcgtgggcgaaaaagtgaccatgacctgcaaaagcagccagaccctgctgtatagcaacaaccagaaaaactatctggcgtggtatcagcagaaaccgggccagagcccgaaactgctgattagctgggcgtttacccgcaaaagcggcgtgccggatcgctttaccggcagcggcagcggcaccgattttaccctgaccattggcagcgtgaaagcggaagatctggcggtgtattattgccagcagtatagcaactatccgtggacctttggcggcggcacccgcctggaaattaaacgcggcggcggcggcagcggcggcggcggcagcggcggcggcggcagcggcggcggcggcagcgaagtgcagctgcagcagagcggcccggaagtggtgaaaaccggcgcgagcgtgaaaattagctgcaaagcgagcggctatagctttaccggctattttattaactgggtgaaaaaaaacagcggcaaaagcccggaatggattggccatattagcagcagctatgcgaccagcacctataaccagaaatttaaaaacaaagcggcgtttaccgtggataccagcagcagcaccgcgtttatgcagctgaacagcctgaccagcgaagatagcgcggtgtattattgcgtgcgcagcggcaactatgaagaatatgcgatggattattggggccagggcaccagcgtgaccgtgagcagc

SEQ ID NO: 58 - DNA, 1502 bp, synthetic construct
  source 1..1502: mol_type = other DNA
gctgacatagtgctgacccagagtcctgattccctggctgtgtctctgggagagagagtgaccatcaattgcaagtccagccagcccctggaatacagcaataatcagtggaactacctggcttggtaccagcagaaacctggacagtcccctaagctgctgattagctgggccagcaccagaaagtctggagtcccagacagattttctggttctggctctggaactgacttcaccctcacaatcagctctgtgcaggctgaagatgtggctgtgtattactgtggccagtactctgattatcccaacacctttggggctggcacaaagctggaaatcaagagaggaggtggaggttctggaggagggggatctggtggagggggctctggaggaggagggtctcaggtgcagctggtgcagtctggagctgaggtggtgaaaccaggggcttctgtgaagatttcctgtaaggcctctggctaccctttcactcagtacttcattcattgggtcaagcagaatcctgggcagagactggaatggattggacaaatctcctccagctatgccacagtggactacaatcagaagttcaagggcaaggcaaccctgactgtggacacatctgccagcattgcctatatggaactgagcagcctgagatctgaagatacagctgtgtattactgtgtgagatctggcaattatgaggagtatgccatggactactggggacagggcaccctggtgactgtgtctggaggaggaggttctgacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggagggtggctcaggtgggtctgggggctctggaggctctggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagct

SEQ ID NO: 59 - DNA, 1413 bp, synthetic construct
  source 1..1413: mol_type = other DNA
gacattcagatgactcagtctcccagctccctgtctgcctctgtgggagacagagtgacaatcacatgtaaggcctcccaggatgtgtctattggagtggcctggtaccagcagaaacctggaaaggccccaaaactgctgatctactctgcctcctacaggtatacaggagtgccttccagattctctggctctggctctggcacagatttcactcttaccatcagttccctccagcctgaggattttgctacctactattgccagcagtactacatctatccttacacatttggccagggaacaaaagtggagatcaagtcctgtggagggggatctgggggaggggggtcaggagggggaggctctgaggtgcagctggtggagtctgggggaggcctggtgcagccagggggctcactgagactgagctgtgctgcctctgggtttacatttacagactataccatggactgggtgagacaggccccagggaagggcctggagtgggtggcagatgtgaacccaaattctggagggtccatctacaatcagaggttcaaggggagatttaccctgtctgtggatagaagcaagaacacactgtaccttcagatgaactccctgagagctgaggacacagctgtgtactactgtgccagaaacctgggcccatccttttactttgactactggggacagggcacactggtgacagtctcctctgggggaggaggctctgatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggagggggggtctgggggctctggaggctctgggggcagtgggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgt

SEQ ID NO: 60 - DNA, 1485 bp, synthetic construct
  source 1..1485: mol_type = other DNA
gacattcagatgacacagtccccatcttccctgtctgcctctgtgggagacagggtgacaatcacttgtagggccagccaggatgtgaacacagctgtggcctggtatcagcagaaacctggaaaggccccaaagctgctgatctactcagcttccttcctgtattctggagtgccatctaggttttctggctccaggtctggcacagactttaccctgaccatctcttctctgcagcctgaggactttgccacttactactgtcagcagcattacacaacccccccaacctttggccagggcaccaaggtggagatcaagggaggagggggaagtgggggaggaggatctggaggagggggatcaggagggggaggctcagaggtgcagctggtggagtctgggggaggcctggtgcagcctggaggctccctgagactctcctgtgctgcctctggattcaatatcaaagatacttacatccactgggtgagacaggcccctggcaaaggcctggaatgggtggccagaatctatcctactaatggctacaccagatatgctgactctgtgaagggcagatttacaatctctgctgacacctctaaaaatacagcctacctgcagatgaattccctgagagctgaagacacagcagtgtactactgcagcaggtggggtggagatggcttctatgccatggactattggggccagggcaccctggtgacagtctcatctgggggagggggcagtgacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaaggaggttctggaggctctggaggctctgggggctctggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaag

SEQ ID NO: 61 - DNA, 1458 bp, synthetic construct
  source 1..1458: mol_type = other DNA
gacattgtgatgacacagtccccctctagcctgtctgcttctgtgggagacagggtgacaatcacatgcagagcctctcagaatgtgagaacagctgtggcatggttccagcagaaaccaggaaaggcccccaaggccctgatctacctggcctctaacaggcacacaggggtgcctgataggttcacaggctctggatctgggacagagtttaccctgaccatcagcaacctgcagcctgaggactttgctacatacttctgcctgcagcacaacagctatcctctgacttttggaggaggcaccaaagtggagatcaagggaggaggaggttctgggggaggaggatctggaggaggaggatctgaggtaaagctggtggagtctggaggaggcctggtgaaaccagggggctctctgagactgagttgtgccacatctggcttttcctttagcagctattatatgtactgggtgaggcaggccccaggcaaaaggctggagtgggtggcctacatcagctctggctctgaaatttattactctgactctgtcaagggcagattcaccatcagcagagattctgccaagaacaccctgtatctccagatgaactctctgagagctgaggacacagctgtgtattactgtgctagactgggagatgatggaatggactggtggggccagggaaccacatggacagtgtcttctggaggaggaggatctgacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaaggaggctctggaggctctggaggctctggaggcagtggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaa

SEQ ID NO: 62 - DNA, 1521 bp, synthetic construct
  source 1..1521: mol_type = other DNA
gcggcgcagccggcggatattgtgctgacccagaccccgagcagcctgccggtgagcgtgggcgaaaaagtgaccatgacctgcaaaagcagccagaccctgctgtatagcaacaaccagaaaaactatctggcgtggtatcagcagaaaccgggccagagcccgaaactgctgattagctgggcgtttacccgcaaaagcggcgtgccggatcgctttaccggcagcggcagcggcaccgattttaccctgaccattggcagcgtgaaagcggaagatctggcggtgtattattgccagcagtatagcaactatccgtggacctttggcggcggcacccgcctggaaattaaacgcggcggcggcggcagcggcggcggcggcagcggcggcggcggcagcggcggcggcggcagcgaagtgcagctgcagcagagcggcccggaagtggtgaaaaccggcgcgagcgtgaaaattagctgcaaagcgagcggctatagctttaccggctattttattaactgggtgaaaaaaaacagcggcaaaagcccggaatggattggccatattagcagcagctatgcgaccagcacctataaccagaaatttaaaaacaaagcggcgtttaccgtggataccagcagcagcaccgcgtttatgcagctgaacagcctgaccagcgaagatagcgcggtgtattattgcgtgcgcagcggcaactatgaagaatatgcgatggattattggggccagggcaccagcgtgaccgtgagcagcggcggcggcggcagcgatattaaactgcagcagagcggcgcggaactggcgcgcccgggcgcgagcgtgaaaatgagctgcaaaaccagcggctatacctttacccgctataccatgcattgggtgaaacagcgcccgggccagggcctggaatggattggctatattaacccgagccgcggctataccaactataaccagaaatttaaagataaagcgaccctgaccaccgataaaagcagcagcaccgcgtatatgcagctgagcagcctgaccagcgaagatagcgcggtgtattattgcgcgcgctattatgatgatcattattgcctggattattggggccagggcaccaccctgaccgtgagcagcgtggaaggcggcagcggcggcagcggcggcagcggcggcagcggcggcgtggatgatattcagctgacccagagcccggcgattatgagcgcgagcccgggcgaaaaagtgaccatgacctgccgcgcgagcagcagcgtgagctatatgaactggtatcagcagaaaagcggcaccagcccgaaacgctggatttatgataccagcaaagtggcgagcggcgtgccgtatcgctttagcggcagcggcagcggcaccagctatagcctgaccattagcagcatggaagcggaagatgcggcgacctattattgccagcagtggagcagcaacccgctgacctttggcgcgggcaccaaactggaactgaaa

SEQ ID NO: 63-74 - skipped (12 entries)

SEQ ID NO: 75 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 76-79 - skipped (4 entries)

SEQ ID NO: 80 - DNA, 601 bp, synthetic construct
  source 1..601: mol_type = other DNA
taacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 81 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 82 - DNA, 7061 bp, synthetic construct
  source 1..7061: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctgctgctgctgctcctgtggcctatggtgtgggctgctgctgacatagtgctgacccagagtcctgattccctggctgtgtctctgggagagagagtgaccatcaattgcaagtccagccagcccctggaatacagcaataatcagtggaactacctggcttggtaccagcagaaacctggacagtcccctaagctgctgattagctgggccagcaccagaaagtctggagtcccagacagattttctggttctggctctggaactgacttcaccctcacaatcagctctgtgcaggctgaagatgtggctgtgtattactgtggccagtactctgattatcccaacacctttggggctggcacaaagctggaaatcaagagaggaggtggaggttctggaggagggggatctggtggagggggctctggaggaggagggtctcaggtgcagctggtgcagtctggagctgaggtggtgaaaccaggggcttctgtgaagatttcctgtaaggcctctggctaccctttcactcagtacttcattcattgggtcaagcagaatcctgggcagagactggaatggattggacaaatctcctccagctatgccacagtggactacaatcagaagttcaagggcaaggcaaccctgactgtggacacatctgccagcattgcctatatggaactgagcagcctgagatctgaagatacagctgtgtattactgtgtgagatctggcaattatgaggagtatgccatggactactggggacagggcaccctggtgactgtgtctggaggaggaggttctgacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggagggtggctcaggtgggtctgggggctctggaggctctggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagctgaagtgacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 83 - DNA, 7025 bp, synthetic construct
  source 1..7025: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctcctgctgctcctgctgtggccaatggtgtgggctgctgacattcagatgactcagtctcccagctccctgtctgcctctgtgggagacagagtgacaatcacatgtaaggcctcccaggatgtgtctattggagtggcctggtaccagcagaaacctggaaaggccccaaaactgctgatctactctgcctcctacaggtatacaggagtgccttccagattctctggctctggctctggcacagatttcactcttaccatcagttccctccagcctgaggattttgctacctactattgccagcagtactacatctatccttacacatttggccagggaacaaaagtggagatcaagtcctgtggagggggatctgggggaggggggtcaggagggggaggctctgaggtgcagctggtggagtctgggggaggcctggtgcagccagggggctcactgagactgagctgtgctgcctctgggtttacatttacagactataccatggactgggtgagacaggccccagggaagggcctggagtgggtggcagatgtgaacccaaattctggagggtccatctacaatcagaggttcaaggggagatttaccctgtctgtggatagaagcaagaacacactgtaccttcagatgaactccctgagagctgaggacacagctgtgtactactgtgccagaaacctgggcccatccttttactttgactactggggacagggcacactggtgacagtctcctctgggggaggaggctctgatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggagggggggtctgggggctctggaggctctgggggcagtgggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgtcagcagtggagttccaatccactcacttttggggcaggcacaaaactggagctgaagtaacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 84 - DNA, 7040 bp, synthetic construct
  source 1..7040: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggaggctctggtggctcctcctgctgctgctgctgctgtggccaatggtgtgggctgctgacattcagatgacacagtccccatcttccctgtctgcctctgtgggagacagggtgacaatcacttgtagggccagccaggatgtgaacacagctgtggcctggtatcagcagaaacctggaaaggccccaaagctgctgatctactcagcttccttcctgtattctggagtgccatctaggttttctggctccaggtctggcacagactttaccctgaccatctcttctctgcagcctgaggactttgccacttactactgtcagcagcattacacaacccccccaacctttggccagggcaccaaggtggagatcaagggaggagggggaagtgggggaggaggatctggaggagggggatcaggagggggaggctcagaggtgcagctggtggagtctgggggaggcctggtgcagcctggaggctccctgagactctcctgtgctgcctctggattcaatatcaaagatacttacatccactgggtgagacaggcccctggcaaaggcctggaatgggtggccagaatctatcctactaatggctacaccagatatgctgactctgtgaagggcagatttacaatctctgctgacacctctaaaaatacagcctacctgcagatgaattccctgagagctgaagacacagcagtgtactactgcagcaggtggggtggagatggcttctatgccatggactattggggccagggcaccctggtgacagtctcatctgggggagggggcagtgacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaaggaggttctggaggctctggaggctctgggggctctggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaagtgacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 85 - DNA, 7013 bp, synthetic construct
  source 1..7013: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctgttgctgctgctgctctggcccatggtgtgggctgctgacattgtgatgacacagtccccctctagcctgtctgcttctgtgggagacagggtgacaatcacatgcagagcctctcagaatgtgagaacagctgtggcatggttccagcagaaaccaggaaaggcccccaaggccctgatctacctggcctctaacaggcacacaggggtgcctgataggttcacaggctctggatctgggacagagtttaccctgaccatcagcaacctgcagcctgaggactttgctacatacttctgcctgcagcacaacagctatcctctgacttttggaggaggcaccaaagtggagatcaagggaggaggaggttctgggggaggaggatctggaggaggaggatctgaggtaaagctggtggagtctggaggaggcctggtgaaaccagggggctctctgagactgagttgtgccacatctggcttttcctttagcagctattatatgtactgggtgaggcaggccccaggcaaaaggctggagtgggtggcctacatcagctctggctctgaaatttattactctgactctgtcaagggcagattcaccatcagcagagattctgccaagaacaccctgtatctccagatgaactctctgagagctgaggacacagctgtgtattactgtgctagactgggagatgatggaatggactggtggggccagggaaccacatggacagtgtcttctggaggaggaggatctgacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaaggaggctctggaggctctggaggctctggaggcagtggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaataacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 7010 bp, synthetic construct
  source 1..7010: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccgcggcgcagccggcggatattgtgctgacccagaccccgagcagcctgccggtgagcgtgggcgaaaaagtgaccatgacctgcaaaagcagccagaccctgctgtatagcaacaaccagaaaaactatctggcgtggtatcagcagaaaccgggccagagcccgaaactgctgattagctgggcgtttacccgcaaaagcggcgtgccggatcgctttaccggcagcggcagcggcaccgattttaccctgaccattggcagcgtgaaagcggaagatctggcggtgtattattgccagcagtatagcaactatccgtggacctttggcggcggcacccgcctggaaattaaacgcggcggcggcggcagcggcggcggcggcagcggcggcggcggcagcggcggcggcggcagcgaagtgcagctgcagcagagcggcccggaagtggtgaaaaccggcgcgagcgtgaaaattagctgcaaagcgagcggctatagctttaccggctattttattaactgggtgaaaaaaaacagcggcaaaagcccggaatggattggccatattagcagcagctatgcgaccagcacctataaccagaaatttaaaaacaaagcggcgtttaccgtggataccagcagcagcaccgcgtttatgcagctgaacagcctgaccagcgaagatagcgcggtgtattattgcgtgcgcagcggcaactatgaagaatatgcgatggattattggggccagggcaccagcgtgaccgtgagcagcggcggcggcggcagcgatattaaactgcagcagagcggcgcggaactggcgcgcccgggcgcgagcgtgaaaatgagctgcaaaaccagcggctatacctttacccgctataccatgcattgggtgaaacagcgcccgggccagggcctggaatggattggctatattaacccgagccgcggctataccaactataaccagaaatttaaagataaagcgaccctgaccaccgataaaagcagcagcaccgcgtatatgcagctgagcagcctgaccagcgaagatagcgcggtgtattattgcgcgcgctattatgatgatcattattgcctggattattggggccagggcaccaccctgaccgtgagcagcgtggaaggcggcagcggcggcagcggcggcagcggcggcagcggcggcgtggatgatattcagctgacccagagcccggcgattatgagcgcgagcccgggcgaaaaagtgaccatgacctgccgcgcgagcagcagcgtgagctatatgaactggtatcagcagaaaagcggcaccagcccgaaacgctggatttatgataccagcaaagtggcgagcggcgtgccgtatcgctttagcggcagcggcagcggcaccagctatagcctgaccattagcagcatggaagcggaagatgcggcgacctattattgccagcagtggagcagcaacccgctgacctttggcgcgggcaccaaactggaactgaaataacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 87 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 88 - protein, 242 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKSCGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 89 - protein, 247 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 90 - protein, 238 aa, synthetic construct
DIVMTQSPSSLSASVGDRVTITCRASQNVRTAVAWFQQKPGKAPKALIYLASNRHTGVPDRFTGSGSGTEFTLTISNLQPEDFATYFCLQHNSYPLTFGGGTKVEIKGGGGSGGGGSGGGGSEVKLVESGGGLVKPGGSLRLSCATSGFSFSSYYMYWVRQAPGKRLEWVAYISSGSEIYYSDSVKGRFTISRDSAKNTLYLQMNSLRAEDTAVYYCARLGDDGMDWWGQGTTWTVSS

SEQ ID NO: 91 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 92 - DNA, 98 bp, synthetic construct
  source 1..98: mol_type = other DNA
gtaagtttagtcttttttgtcttttatttcaggtcccggatccggtggtggtgcaaatcaaagaactgctcctcagtcgatgttgcctttacttctag

SEQ ID NO: 93 - protein, 1255 aa, synthetic construct [sequence omitted]

SEQ ID NO: 94 - protein, 241 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 95 - protein, 119 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSS

SEQ ID NO: 96 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 97 - protein, 10 aa, synthetic construct
GFTFTDYTMD

SEQ ID NO: 98 - protein, 17 aa, synthetic construct
DVNPNSGGSIYNQRFKG

SEQ ID NO: 99 - protein, 10 aa, synthetic construct
NLGPSFYFDY

SEQ ID NO: 100 - protein, 9 aa, synthetic construct
KASQDVSIG

SEQ ID NO: 101 - protein, 6 aa, synthetic construct
SASYRY

SEQ ID NO: 102 - protein, 8 aa, synthetic construct
QQYYIYPY

SEQ ID NO: 103 - protein, 8 aa, synthetic construct
GFNIKDTY

SEQ ID NO: 104 - protein, 8 aa, synthetic construct
IYPTNGYT

SEQ ID NO: 105 - protein, 13 aa, synthetic construct
SRWGGDGFYAMDY

SEQ ID NO: 106 - protein, 6 aa, synthetic construct
QDVNTA

SEQ ID NO: 107 - skipped

SEQ ID NO: 108 - protein, 9 aa, synthetic construct
QQHYTTPPT

SEQ ID NO: 109 - protein, 5 aa, synthetic construct
SYYMY

SEQ ID NO: 110 - protein, 16 aa, synthetic construct
ISSGSEIYYSDSVKGR

SEQ ID NO: 111 - protein, 8 aa, synthetic construct
LGDDGMDW

SEQ ID NO: 112 - protein, 11 aa, synthetic construct
RASQNVRTAVA

SEQ ID NO: 113 - protein, 7 aa, synthetic construct
LASNRHT

SEQ ID NO: 114 - protein, 9 aa, synthetic construct
LQHNSYPLT

SEQ ID NO: 115 - protein, 5 aa, synthetic construct
QYFIH

SEQ ID NO: 116 - protein, 17 aa, synthetic construct
QISSSYATVDYNQKFKG

SEQ ID NO: 117 - protein, 11 aa, synthetic construct
SGNYEEYAMDY

SEQ ID NO: 118 - protein, 17 aa, synthetic construct
KSSQPLEYSNNQWNYLA

SEQ ID NO: 119 - protein, 7 aa, synthetic construct
WASTRKS

SEQ ID NO: 120 - protein, 9 aa, synthetic construct
GQYSDYPNT

SEQ ID NO: 121 - protein, 5 aa, synthetic construct
GYFIN

SEQ ID NO: 122 - protein, 17 aa, synthetic construct
HISSSYATSTYNQKFKN

SEQ ID NO: 123 - protein, 11 aa, synthetic construct
SGNYEEYAMDY

SEQ ID NO: 124 - protein, 17 aa, synthetic construct
KSSQTLLYSNNQKNYLA

SEQ ID NO: 125 - protein, 7 aa, synthetic construct
WAFTRKS

SEQ ID NO: 126 - protein, 9 aa, synthetic construct
QQYSNYPWT

SEQ ID NO: 127 - protein, 5 aa, synthetic construct
RYTMH

SEQ ID NO: 128 - protein, 17 aa, synthetic construct
YINPSRGYTNYNQKFKD

SEQ ID NO: 129 - protein, 10 aa, synthetic construct
YYDDHYCLDY

SEQ ID NO: 130 - protein, 10 aa, synthetic construct
RASSSVSYMN

SEQ ID NO: 131 - protein, 7 aa, synthetic construct
DTSKVAS

SEQ ID NO: 132 - protein, 9 aa, synthetic construct
QQWSSNPLT

SEQ ID NO: 133 - protein, 5 aa, synthetic construct
RYTMH

SEQ ID NO: 134 - protein, 17 aa, synthetic construct
YINPSRGYTNYNQKFKD

SEQ ID NO: 135 - protein, 10 aa, synthetic construct
YYDDHYCLDY

SEQ ID NO: 136 - protein, 10 aa, synthetic construct
SASSSVSYMN

SEQ ID NO: 137 - protein, 7 aa, synthetic construct
DTSKLAS

SEQ ID NO: 138 - protein, 9 aa, synthetic construct
QQWSSNPFT

SEQ ID NO: 139 - DNA, 82 bp, synthetic construct
  source 1..82: mol_type = other DNA
aacttgtttattgcagcttataatggttacaaataaagcaatagcatcacaaatttcacaaataaagcatttttttcactgc

SEQ ID NO: 140 - protein, 254 aa, synthetic construct
QVQLVQSGAEVVKPGASVKISCKASGYPFTQYFIHWVKQNPGQRLEWIGQISSSYATVDYNQKFKGKATLTVDTSASIAYMELSSLRSEDTAVYYCVRSGNYEEYAMDYWGQGTLVTVSTKLEIKRGGGGSGGGGSGGGGSGGGGSADIVLTQSPDSLAVSLGERVTINCKSSQPLEYSNNQWNYLAWYQQKPGQSPKLLISWASTRKSGVPDRFSGSGSGTDFTLTISSVQAEDVAVYYCGQYSDYPNTFGAG

SEQ ID NO: 141 - protein, 259 aa, synthetic construct
EVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSSTRLEIKRGGGGSGGGGSGGGGSGGGGSAAQPADIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGG

SEQ ID NO: 142 - protein, 189 aa, synthetic construct
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 143 - DNA, 725 bp, synthetic construct
  source 1..725: mol_type = other DNA
gacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggagggtggctcaggtgggtctgggggctctggaggctctggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagct

SEQ ID NO: 144 - DNA, 672 bp, synthetic construct
  source 1..672: mol_type = other DNA
gatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggagggggggtctgggggctctggaggctctgggggcagtgggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgt

SEQ ID NO: 145 - DNA, 729 bp, synthetic construct
  source 1..729: mol_type = other DNA
gacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaaggaggttctggaggctctggaggctctgggggctctggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaag

SEQ ID NO: 146 - DNA, 729 bp, synthetic construct
  source 1..729: mol_type = other DNA
gacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaaggaggctctggaggctctggaggctctggaggcagtggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaa

SEQ ID NO: 147 - DNA, 357 bp, synthetic construct
  source 1..357: mol_type = other DNA
caggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagt

SEQ ID NO: 148 - DNA, 321 bp, Adeno-associated virus
  source 1..321: mol_type = genomic DNA
gatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 149 - DNA, 723 bp, synthetic construct
  source 1..723: mol_type = other DNA
caggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 150 - DNA, 795 bp, synthetic construct
  source 1..795: mol_type = other DNA
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 151 - RNA, 11 nt, synthetic construct
  source 1..11: mol_type = other RNA
cattgtatgtc

SEQ ID NO: 152 - RNA, 11 nt, synthetic construct
  source 1..11: mol_type = other RNA
tcgtttatgga

SEQ ID NO: 153 - RNA, 11 nt, synthetic construct
  source 1..11: mol_type = other RNA
cagtttatggt

SEQ ID NO: 154 - RNA, 11 nt, synthetic construct
  source 1..11: mol_type = other RNA
cattgtatggt

SEQ ID NO: 155 - RNA, 11 nt, synthetic construct
  source 1..11: mol_type = other RNA
tagtgtatgct

SEQ ID NO: 156 - RNA, 11 nt, synthetic construct
  source 1..11: mol_type = other RNA
tcttttatgtc

SEQ ID NO: 157 - RNA, 11 nt, synthetic construct
  source 1..11: mol_type = other RNA
tgttttatgtc

SEQ ID NO: 158 - RNA, 11 nt, synthetic construct
  source 1..11: mol_type = other RNA
tagtttatgtc

SEQ ID NO: 159 - RNA, 11 nt, synthetic construct
  source 1..11: mol_type = other RNA
tagtgtatgtc

SEQ ID NO: 160 - RNA, 11 nt, synthetic construct
  source 1..11: mol_type = other RNA
tagcgcatggc

SEQ ID NO: 161 - RNA, 11 nt, synthetic construct
  source 1..11: mol_type = other RNA
tggtatatggc

SEQ ID NO: 162 - RNA, 11 nt, synthetic construct
  source 1..11: mol_type = other RNA
tagtttatggc

SEQ ID NO: 163 - RNA, 11 nt, synthetic construct
  source 1..11: mol_type = other RNA
cagtgtatggc

SEQ ID NO: 164 - RNA, 11 nt, synthetic construct
  source 1..11: mol_type = other RNA
cattgtatggc

SEQ ID NO: 165 - RNA, 11 nt, synthetic construct
  source 1..11: mol_type = other RNA
ccgtttatggg

SEQ ID NO: 166 - RNA, 11 nt, synthetic construct
  source 1..11: mol_type = other RNA
acttgtatggg

SEQ ID NO: 167 - RNA, 11 nt, synthetic construct
  source 1..11: mol_type = other RNA
cattttatggg

SEQ ID NO: 168 - RNA, 11 nt, synthetic construct
  source 1..11: mol_type = other RNA
tagtgtatggg

SEQ ID NO: 169 - RNA, 11 nt, synthetic construct
  source 1..11: mol_type = other RNA
tagtttatggg

SEQ ID NO: 170 - RNA, 11 nt, synthetic construct
  source 1..11: mol_type = other RNA
tgttttatggg

SEQ ID NO: 171 - RNA, 11 nt, synthetic construct
  source 1..11: mol_type = other RNA
tcttttatggg

SEQ ID NO: 172 - RNA, 11 nt, synthetic construct
  source 1..11: mol_type = other RNA
tagtgtatggc

SEQ ID NO: 173 - RNA, 11 nt, synthetic construct
  source 1..11: mol_type = other RNA
tgttttatggc

SEQ ID NO: 174 - RNA, 11 nt, synthetic construct
  source 1..11: mol_type = other RNA
tcttttatggc

SEQ ID NO: 175 - DNA, 598 bp, synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 176 - DNA, 581 bp, synthetic construct
  source 1..581: mol_type = other DNA
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 177 - DNA, 816 bp, synthetic construct
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 178 - DNA, 10726 bp, synthetic construct
  source 1..10726: mol_type = other DNA
cgggccccccctcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtgagtaattgacaaagccaaacaccaccatttgccgagcactttagagtttacaggtttgtttctcttgaccctcaaaacaaacctgtgaggcatagggagtattgctatcccttaagaattcacccccagtgtgcccatcaaaacctcccaggctgagtctgcacagttgaaggaggaaggataggaatgggagggtcgatgggtgaaagcatgattctcttaaccagtccagattatcaggtaatcccttcaacaaccaccacccactccctgggcaatccagctggagtttacagacagacttagctggctatagcaccaccgtgctactctctgttcttcctggttgctcaaatgccctagaaaagtggaacaggtgagcatcaactcacagggctctatgctggctgctgctgcgagggatgttatgctatagtaccaggggccaccattccataggcacttcctgtgtttaataccctatatgctttacttcatctcatcttcctccatatcctgagaggtggttctattcttctccccattttacggatgaaaaaaccgagacacagaaaggtgaaatagcttaagataaatggtgccttgcagccttagactctggtggcctctagttaatgtgggaaattaagggtgaggggattggcagctgatggagggtgcagggtgccagacagaggcgtttagctctgatcccttagcaatagagagtccttgtaggcacttggtcaggcgagtgatgcgatgaaagctgtgtttaagaaagattatgctttctgctgatttcatacccccaacacccaagctctgaggcccctcctcacaggtccttgcagggctggccaaaataaagcagcttcactccgttgtgctgctttccagctaatgtgtctgtttggcagaagtttccctcaaaggcagatcagtgaaataagcagaagcctcgacccccctttgtcagccagagctgctgaagtgccttgccccagggtcactttgtgtgaggggattagagagcactggggctgccaagaaacactgccgtttctacagattagcaggacgctggcttgtggccttctagcgaggctcagagctgcggtggccctagtctgcatgggctaaagacaagctccatctcctgtccttgttccctccttcctgggcacagccgccctgcttcttggttctctctgttggttcctgtccgcacggtagttaggctggcagcgtgtgtaggatttggcttagaagattgacaacattgcctttgagcccttctttgctactcctccctctcccctcccatcagactcctctctggagtctgctctgcgaggcctctgctctgtggtatcccagcagccttctcagccttgacttccagaagggggctgtgcagtgtccggggtgtgcaggccccagacacggggtaggctcatggagatccaagtgctgatctagtgtcaaggctggcctggagactgggctgggttggtgtctgcctgctgtggtcatgtgccctcccttgggcctgtatcctctctccagacttgctgcagggagaggtggcagatgtcagcctagttctggcctctcagagcagcatggcagctccctttcactcaggcccaggctgggccctcctgctggctgacccctggggagagggtgctccagagctccccaaggaacagcttcccgaagcagccaggccagcccagaggggctgtggccaatcctgaagctttatgttcctgctgacattttttctaagttttctcttgctttcctcttaaatgccaatctggagagtctccgttaggagaaatggaccccagccaggaagaagagttgagttgtatttaaaacacgagctccccctaaagcatccttctttagcttctaaggagaggcagagactgacaggcaggactcagcaggaaaaggtacccccctgacctgctcagtcaggccctaggcccagctccacccagcctgtggcccccagagtttcggtaaagagttccctgggccttaaggaaccttgagagagcatttgaggggtgccaccacaaacttggcagaaaaaaccctccccctccaagtccagtcctagagaaggagctggcaaccttgccttgctttgtaagcaaaagcctcttagggcttgagctcagatgtagtgtttgagctgtggctggtgccctgccccatcagggagccaatggtagacatcctatgggcatctttgttttccgtaagagcaggctgtctggggatgggccagaggaagaggcgacctggagtcaaccaagaggaggccttaaccaagccttaaccacagaggttaaccaagccttgaaagcgcttccccctgagcaggcaggaagcactgagtccacatggttgcctcgctgtttcatttccttacactcaattctctcagtctttaaatgatcacttggccttgaagttacggatatttggggtctgaactgaagttgaagaaaagaggaaatgatttaagctttgtttaagattaggggccaggtgcggtggctcacgcctgtaatcccagcaccttgggagcctgaggcgggtggatcacctgaggtcaggagttccagaccagcctggccaacatagcaaaacccagtctctactaaaaataacaataaaaaaattagccaggtgtggtgacacatgcctgtaatcccagttactcaggaggctgaggcagaattgcttgaacttgagaggtggaggttgtagtgagccaagaccgcaccactgcactccagcctggcgacagagccagactccgtctcaaaaacaacaacaaaaaagattagaagaagcccattactgccttctggccacccactcgcacagacaccaaaactgcagcccacacctcgccatcctcgtgctctgccctgggacaccccaggcacagtgtgtccttcgttttctgtaagggtgggctgggagcagggacggacagggcctgtgggcacctctcatggtcacttccttcttgctcacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaattacatgttaatcaataaaccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaacaaacagcttttttggggtgaacatattgactgaattggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtac

(19) United States
(12) Patent Application Publication
(10) Pub. No.: US 2026/0022397 A1
(43) Pub. Date: Jan. 22, 2026

(54) ADENO-ASSOCIATED VIRUS VECTORS AND METHODS OF THEIR USE FOR REDUCING THE RISK OF, TREATING, AND PREVENTING METASTASIS

(71) Applicants: Vironexis Biotherapeutics Inc., La Jolla, CA (US); RESEARCH INSTITUTE AT NATIONWIDE CHILDREN'S HOSPITAL, Columbus, OH (US)

(72) Inventors: Timothy P. Cripe, La Jolla, CA (US); Landon Gyulay, La Jolla, CA (US); Brian Kaspar, La Jolla, CA (US); Allan Kaspar, La Jolla, CA (US); Samit Varma, La Jolla, CA (US); Jacob Gardinier Scott, La Jolla, CA (US)

(21) Appl. No.: 18/998,414

(22) PCT Filed: Jul. 25, 2023
(86) PCT No.: PCT/US2023/070960

§ 371 Date: Jan. 24, 2025

Related U.S. Application Data
(60) Provisional application No. 63/391,962, filed on Jul. 25, 2022.

(51) Int. Cl. C12N 15/86; A61K 39/00; A61K 39/395; A61K 48/00; A61P 35/04; C07K 16/28; C07K 16/32

(52) U.S. Cl. CPC C12N 15/86; A61K 39/39558; A61K 48/005; A61P 35/04; C07K 16/2809; C07K 16/32; A61K 2039/505; C07K 2317/31; C07K 2317/622; C07K 2317/73; C07K 2317/92; C07K 2319/00; C12N 2750/14143

(57) ABSTRACT

Provided herein are recombinant adeno-associated viral (rAAV) vectors expressing a bispecific fusion protein that binds HER2 and CD3, and methods of using the same for reducing the risk of, preventing, or treating metastasis. The disclosure provides for rAAV vectors for delivering a sequence encoding the bispecific fusion protein, and methods of using the same for reducing the risk of, preventing, or treating metastasis.

### CROSS-REFERENCE

This application claims the benefit of and priority to U.S. Provisional Patent Application No. 63/391,962, filed Jul. 25, 2022, which is incorporated by reference in its entirety herein.

### FIELD

The disclosure generally relates to adeno-associated virus (AAV) vectors for delivering a transgene sequence, encoding a bispecific fusion protein including a HER2 binding site and a CD3 binding site. The present disclosure further relates to methods of killing circulating tumor cells thereby reducing the risk, delaying the onset, and preventing cancer and metastatic disease.

### BACKGROUND

Cancer remains a significant worldwide health problem and is the second leading cause of death in the United States. Breast cancer persists as a leading cause of death in women. Current treatment options for breast cancer are not effective for all patients and can often be associated with significant adverse side effects.

Cancer immunotherapies are a promising modality for treatment as they exhibit greater specificity than conventional chemotherapeutics and can facilitate destruction of tumor cells by eliciting a patient's own immune system. Bispecific T cell engager proteins are recombinant fusion proteins that have been described in the prior art that bind to both tumor cells and T cells thereby stimulating destruction of the tumor cells.

Adeno-associated viruses (AAV) have been used as gene therapy vectors to achieve long-term, consistent bloodstream levels of cancer immunotherapies. For example, AAVs encoding a bispecific αCD19-αCD3 protein achieved persistence in the bloodstream for greater than one year and anti-tumor efficacy in a CD19+ lymphoma model (Cripe et al., Science Advances).

Given that metastases are thought to arise from circulating tumor cells in what could be thought of as a “leukemia compartment” of solid tumors, long-term, persistent immunologic pressure targeting cancer may be effectively used to prevent development of metastases. Since circulating tumor cells exist outside of the immunosuppressive solid tumor microenvironment, they may be more vulnerable to immunotherapy.

HER2 (ErbB2) is a transmembrane glycoprotein, which belongs to the epidermal growth factor receptor family. It is a receptor tyrosine kinase and regulates cell survival, proliferation, and growth and therefore plays a prominent role in many human malignancies. The ERBB2 gene is amplified or overexpressed in approximately 30% of human breast cancers. Patients with HER2-overexpressing breast cancer have substantially lower overall survival rates and shorter disease-free intervals than patients whose cancers do not overexpress HER2. Moreover, overexpression of HER2 leads to increased breast cancer metastasis. Over-expression of HER2 is also known to occur in many other cancer types, including ovarian, esophageal, bladder and gastric cancer, salivary duct carcinoma, adenocarcinoma of the lung and aggressive forms of uterine cancer, such as uterine serous endometrial carcinoma.

### BRIEF SUMMARY

The present disclosure is directed to compositions and methods of using adeno-associated virus vectors for expressing bi-specific fusion proteins for reducing the risk of, preventing, and treating cancer and metastasis.

In some aspects, the present disclosure provides a recombinant adeno-associated viral (rAAV) vector, comprising from 5′ to 3′: a) a 5′ AAV inverted terminal repeat (ITR); b) a promoter; c) a transgene encoding a bispecific fusion protein comprising: (i) a HER2 binding site comprising a light chain variable region (VL) and a heavy chain variable region (VH) of an anti-HER2 antibody, (ii) a linker peptide, and (iii) a CD3 binding site comprising a VH and a VL of an anti-CD3 antibody; d) a modified RNA stability regulatory element (MRE) and e) a 3′ AAV ITR. In some embodiments, the promoter is selected from the group consisting of a chicken β-actin promoter, an elongation factor 1α (EF1α) promoter, a simian virus 40 (SV40) promoter, or a CAG promoter. In some embodiments, the promoter is a CAG promoter. In some embodiments, the promoter comprises a sequence at least 95% identical to SEQ ID NO: 87. In some embodiments, the anti-HER2 antibody VL comprises a CDR1, CDR2, and CDR3 sequence of SEQ ID NO: 106, SEQ ID NO: 107, and SEQ ID NO: 108, respectively, and the anti-HER2 antibody VH comprises a CDR1, CDR2, and CDR3 sequence of SEQ ID NO: 103, SEQ ID NO: 104, and SEQ ID NO: 105, respectively. In some embodiments, the anti-HER2 antibody VL and VH comprise sequences at least 95% identical to SEQ ID NO: 5 and SEQ ID NO: 4, respectively. In some embodiments, the HER2 binding site is a single chain variable fragment (scFv). In some embodiments, anti-HER2 antibody VL is fused to the anti-HER2 antibody VH using an scFv linker peptide comprising of SEQ ID NO: 25. In some embodiments, the HER2 binding site comprises a sequence at least 95% identical to SEQ ID NO: 89. In some embodiments, the anti-HER2 antibody VL comprises a complementarity determining region 1 (CDR1), complementarity determining region 2 (CDR2), and complementarity determining region 3 (CDR3) sequence of SEQ ID NO: 100, SEQ ID NO: 101, and SEQ ID NO: 102, respectively, and the anti-HER2 antibody VH comprises a CDR1, CDR2, and CDR3 sequence of SEQ ID NO: 97, SEQ ID NO: 98, and SEQ ID NO: 99, respectively. In some embodiments, the anti-HER2 antibody VL and VH comprise sequences at least 95% identical to SEQ ID NO: 2 and SEQ ID NO: 1, respectively. In some embodiments, the HER2 binding site is a single chain variable fragment (scFv). In some embodiments, the anti-HER2 antibody VL is fused to the anti-HER2 antibody VH using an scFv linker peptide comprising a sequence of SEQ ID NO: 24. In some embodiments, the scFv comprises a sequence at least 95% identical to SEQ ID NO: 88. In some embodiments, the anti-HER2 antibody VL comprises a CDR1, CDR2, and CDR3 sequence of SEQ ID NO: 112, SEQ ID NO: 113, and SEQ ID NO: 114, respectively, and the anti-HER2 antibody VH comprises a CDR1, CDR2, and CDR3 sequence of SEQ ID NO: 109, SEQ ID NO: 110, and SEQ ID NO: 111, respectively. In some embodiments, the anti-HER2 antibody VL and VH comprise sequences at least 95% identical to SEQ ID NO: 8 and SEQ ID NO: 7, respectively. In some embodiments, the HER2 binding site is a single chain variable fragment (scFv). In some embodiments, anti-HER2 antibody VL is fused to the anti-HER2 antibody VH using an scFv linker peptide comprising of SEQ ID NO: 26. In some embodiments, the HER2 binding site comprises a sequence at least 95% identical to SEQ ID NO: 90. In some embodiments, the anti-HER2 antibody VL comprises a CDR1, CDR2, and CDR3 sequence of SEQ ID NO: 118, SEQ ID NO: 119, and SEQ ID NO: 120, respectively, and the anti-HER2 antibody VH comprises a CDR1, CDR2, and CDR3 sequence of SEQ ID NO: 115, SEQ ID NO: 116, and SEQ ID NO: 117, respectively. In some embodiments, the anti-HER2 antibody VL and VH comprise sequences at least 95% identical to SEQ ID NO: 10 and SEQ ID NO: 11, respectively. In some embodiments, the HER2 binding site is a single chain variable fragment (scFv). In some embodiments, the anti-HER2 antibody VL is fused to the anti-HER2 antibody VH by an scFv linker peptide comprising an amino acid sequence of SEQ ID NO: 27. In some embodiments, the HER2 binding site comprises a sequence at least 95% identical to SEQ ID NO: 12. In some embodiments, the anti-HER2 antibody VL comprises a CDR1, CDR2, and CDR3 sequence of SEQ ID NO: 124, SEQ ID NO: 125, and SEQ ID NO: 126, respectively, and the anti-HER2 antibody VH comprises a CDR1, CDR2, and CDR3 sequence of SEQ ID NO: 121, SEQ ID NO: 122, and SEQ ID NO: 123, respectively. In some embodiments, the anti-HER2 antibody VL and VH comprise sequences at least 95% identical to SEQ ID NO: 13 and SEQ ID NO: 14, respectively. In some embodiments, the HER2 binding site is a single chain variable fragment (scFv). In some embodiments, anti-HER2 antibody VL is fused to the anti-HER2 antibody VH by an scFv linker peptide comprising an amino acid sequence of SEQ ID NO: 27. In some embodiments, the HER2 binding site comprises a sequence at least 95% identical to SEQ ID NO: 15. In some embodiments, the linker peptide comprises a sequence identical to SEQ ID NO: 29. In some embodiments, the anti-CD3 antibody VH comprises a CDR1, CDR2, and CDR3 sequence of SEQ ID NO: 127, SEQ ID NO: 128, and SEQ ID NO: 129, respectively, and the anti-CD3 antibody VL comprises a CDR1, CDR2, and CDR3 sequence of SEQ ID NO: 130, SEQ ID NO: 131, and SEQ ID NO: 132, respectively. In some embodiments, the anti-CD3 antibody VH and VL comprise sequences at least 95% identical to SEQ ID NO: 16 and SEQ ID NO: 17, respectively. In some embodiments, the CD3 binding site is a single chain variable fragment (scFv). In some embodiments, the anti-CD3 antibody VH is fused to the anti-CD3 antibody VL using an scFv linker peptide comprising a sequence identical to SEQ ID NO: 28. In some embodiments, the CD3 binding site comprises a sequence at least 95% identical to SEQ ID NO: 18. In some embodiments, the rAAV vector further comprises a Kozak sequence. In some embodiments, the rAAV vector further comprises a polyadenylation sequence 3′ of the transgene sequence and 5′ of the 3′ AAV ITR. In some embodiments, the polyadenylation sequence is a bovine growth hormone (BGH) polyadenylation sequence at least 95% identical to SEQ ID NO: 81. In some embodiments, the vector further comprises an antibiotic resistance gene sequence. In some embodiments, the antibiotic resistance gene is a kanamycin resistance gene. In some embodiments, the AAV is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the bispecific fusion protein comprises an amino acid sequence at least 90% identical to SEQ ID NO: 19, SEQ ID NO: 20, SEQ ID NO: 21, SEQ ID NO: 22, or SEQ ID NO: 23. In some embodiments, the transgene comprises a nucleotide sequence at least 95% identical to SEQ ID NO: 59, SEQ ID NO: 60, SEQ ID NO: 61, SEQ ID NO: 58, or SEQ ID NO: 62. In some embodiments, the transgene comprises reduced CpG dinucleotides and/or increased methylation of CpG dinucleotides as compared to a parental equivalent. In some embodiments, the rAAV vector comprises a sequence at least 90% identical to SEQ ID NO: 83, SEQ ID NO: 84, SEQ ID NO: 85, SEQ ID NO: 82, or SEQ ID NO: 86.

In some aspects, the present disclosure provides a recombinant adeno-associated viral (rAAV) vector, comprising from 5′ to 3′: a) a 5′ AAV inverted terminal repeat (ITR) comprising a sequence at least 90% identical to SEQ ID NO: 75 or SEQ ID NO: 91; b) a promoter; c) a transgene comprising a sequence encoding a bispecific fusion protein comprising, (i) a HER2 binding site comprising a light chain variable region (VL) comprising a CDR1, CDR2, and CDR3 sequence of SEQ ID NO: 106, SEQ ID NO: 107, and SEQ ID NO: 108, respectively, and a heavy chain variable region (VH) comprising a CDR1, CDR2, and CDR3 sequence of SEQ ID NO: 103, SEQ ID NO: 104, and SEQ ID NO: 105, respectively of an anti-HER2 antibody; (ii) a linker peptide comprising a sequence according to SEQ ID NO: 29, and (iii) a CD3 binding site comprising a VH comprising a CDR1, CDR2, and CDR3 sequence of SEQ ID NO: 127, SEQ ID NO: 128, and SEQ ID NO: 129, respectively, and a VL comprising a CDR1, CDR2, and CDR3 sequence of SEQ ID NO: 130, SEQ ID NO: 131, and SEQ ID NO: 132, respectively of an anti-CD3 antibody; d) a modified RNA stability regulatory element (MRE) and e) a 3′ AAV ITR comprising a sequence at least 90% identical to SEQ ID NO: 75 or SEQ ID NO: 91.

In some aspects, the present disclosure provides a recombinant adeno-associated viral (rAAV) vector, comprising from 5′ to 3′: a) a 5′ AAV inverted terminal repeat (ITR) comprising a sequence at least 90% identical SEQ ID NO: 75 or SEQ ID NO: 91; b) a promoter; c) a transgene encoding a bispecific fusion protein comprising a sequence at least 90% identical to SEQ ID NO: 19, SEQ ID NO: 20, SEQ ID NO: 21, SEQ ID NO: 22, or SEQ ID NO: 23; d) modified RNA stability regulatory element (MRE) and e) a 3′ AAV ITR comprising a sequence at least 90% identical to SEQ ID NO: 75 or SEQ ID NO: 91.

In some aspects, the present disclosure provides a recombinant adeno-associated viral (rAAV) vector comprising a sequence at least 90% identical to SEQ ID NO: 20.

In some aspects, the present disclosure provides a method of reducing the risk of metastatic disease in a patient comprising administering to the patient an effective amount of a recombinant adeno-associated viral (rAAV) vector described herein or pharmaceutical formulation thereof.

In some aspects, the present disclosure provides a method of delaying the onset of metastatic disease in a patient comprising administering to the patient an effective amount of a recombinant adeno-associated viral (rAAV) vector described herein or pharmaceutical formulation thereof.

In some aspects, the present disclosure provides a method of preventing metastatic disease in a patient comprising administering to the patient an effective amount of a recombinant adeno-associated viral (rAAV) vector described herein or pharmaceutical formulation thereof.

In some aspects, the present disclosure provides a method of promoting T cell-mediated killing of circulating tumor cells in a patient comprising administering to the patient an effective amount of a recombinant adeno-associated viral (rAAV) vector described herein or pharmaceutical formulation thereof. In some embodiments, the rAAV or pharmaceutical formulation thereof is administered concurrently with treatment of a primary tumor. In some embodiments, the primary tumor is a breast tumor. In some embodiments, treatment of the primary tumor includes surgical resection, radiation therapy, chemotherapy, or immunotherapy.

In some aspects, the present disclosure provides a method of preventing cancer in a patient predisposed to developing HER2+ tumors comprising administering to the patient an effective amount of a recombinant adeno-associated viral (rAAV) vector described herein or pharmaceutical formulation thereof.

In some aspects, the present disclosure provides a method of preventing cancer relapse in a patient in remission for a HER2+ cancer comprising administering to the patient an effective amount of a recombinant adeno-associated viral (rAAV) vector described herein or pharmaceutical formulation thereof. In some embodiments, the rAAV or pharmaceutical formulation thereof is administered with a checkpoint inhibitor selected from the group consisting of: a CTLA-4 inhibitor, a PD-1 inhibitor, and a PD-L1 inhibitor. In some embodiments, the checkpoint inhibitor is selected from the group consisting of: pembrolizumab, ipilimumab, nivolumab, and atezolizumab.

In some aspects, the present disclosure provides a pharmaceutical formulation comprising a recombinant adeno-associated viral (rAAV) vector described herein, and a pharmaceutically acceptable carrier.

In some aspects, the present disclosure provides a method of reducing the risk of metastatic disease in a patient comprising administering to the patient an effective amount of a recombinant adeno-associated viral (rAAV) vector or pharmaceutical formulation thereof. In some aspects, the rAAV vector comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the present disclosure provides a method of delaying the onset of metastatic disease in a patient comprising administering to the patient an effective amount of an adeno-associated virus (AAV) or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the present disclosure provides a method of preventing metastatic disease in a patient comprising administering to the patient an effective amount of a recombinant adeno-associated viral (rAAV) vector or pharmaceutical formulation thereof. In some aspects, the rAAV vector comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the present disclosure provides a method of promoting T cell-mediated killing of circulating tumor cells in a patient comprising administering to the patient an effective amount of an adeno-associated virus (AAV) or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the rAAV or pharmaceutical formulation thereof is administered concurrently with treatment of a primary tumor. In some aspects, the primary tumor is a breast tumor. In some aspects, treatment of the primary tumor comprises surgical resection, radiation therapy, chemotherapy, or immunotherapy.

In some aspects, the present disclosure provides a method of preventing cancer in a patient predisposed to developing HER2+ tumors comprising administering to the patient an effective amount of an adeno-associated virus (AAV) or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the present disclosure provides a method of preventing cancer relapse in a patient in remission for a HER2+ cancer comprising administering to the patient an effective amount of an adeno-associated virus (AAV) or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises a HER2 binding site comprising a heavy chain variable region (VH) and a light chain variable region (VL) of an anti-HER2 antibody; a linker peptide; and a CD3 binding site comprising a VH and a VL of an anti-CD3 antibody.

In some aspects, the present disclosure provides a recombinant adeno-associated virus vector comprising from 5′ to 3′:a 5′ AAV inverted terminal repeat (ITR); a promoter; a transgene comprising a sequence encoding a bispecific fusion protein comprising a sequence at least 95% identical to SEQ ID NO: 19. SEQ ID NO: 20, SEQ ID NO: 21, SEQ ID NO: 22, or SEQ ID NO: 23; and a 3′ AAV ITR.

In some aspects, the rAAV or pharmaceutical formulation thereof is administered with a checkpoint inhibitor selected from the group comprising: a CTLA-4 inhibitor, a PD-1 inhibitor, and a PD-L1 inhibitor. In some aspects, the checkpoint inhibitor is selected from the group consisting of: pembrolizumab, ipilimumab, nivolumab, and atezolizumab.

In some aspects, the 5′ AAV ITR comprises a sequence at least 95% identical to SEQ ID NO: 75. In some aspects, the 3′ AAV ITR comprises a sequence at least 95% identical to SEQ ID NO: 91.

In some aspects, the promoter is selected from a chicken β-actin promoter, an elongation factor 1α (EF1α) promoter, a simian virus 40 (SV40) promoter, and a CAG promoter. In some aspects, the promoter is a CAG promoter. In some aspects, the promoter comprises a sequence at least 95% identical to SEQ ID NO: 87.

In some aspects, the anti-HER2 antibody VL of the HER2 binding site has a complementarity determining region 1 (CDR1), a complementarity determining region 2 (CDR2), and complementarity determining region 3 (CDR3) sequence of SEQ ID NO: 100, SEQ ID NO: 101, and SEQ ID NO: 102, respectively, and the anti-HER2 antibody VH of the HER2 binding site has a CDR1, CDR2, and CDR3 sequence of SEQ ID NO: 97, SEQ ID NO: 98 and SEQ ID NO: 99, respectively. In some aspects, the anti-HER2 antibody VL and VH have sequences at least 95% identical to SEQ ID NO: 2 and SEQ ID NO: 1, respectively. In some aspects, the HER2 binding site is a single chain variable fragment (scFv). In some aspects, the scFv comprises the VL of the HER2 binding site is fused to the anti-HER2 antibody VH of the HER2 binding site by an scFv linker peptide comprising a sequence of SEQ ID NO: 24. In some aspects, the scFv comprises a sequence at least 95% identical to SEQ ID NO: 88.

In some aspects, the anti-HER2 antibody VL of the HER2 binding site has a CDR1, a CDR2, and CDR3 sequence of SEQ ID NO: 106, SEQ ID NO: 107, and SEQ ID NO: 108, respectively, and the anti-HER2 antibody VH of the HER2 binding site has a CDR1, CDR2, and CDR3 sequence of SEQ ID NO: 103, SEQ ID NO: 104, and SEQ ID NO: 105, respectively. In some aspects, the anti-HER2 antibody VL and VH of the HER2 binding site have sequences at least 95% identical to SEQ ID NO: 5 and SEQ ID NO: 4, respectively. In some aspects, the HER2 binding site is a single chain variable fragment (scFv). In some aspects, anti-HER2 antibody VL of the HER2 binding site is fused to the anti-HER2 antibody VH of the HER2 binding site by an scFv linker peptide comprising SEQ ID NO: 25. In some aspects, the HER2 binding site comprises a sequence at least 95% identical to SEQ ID NO: 89.

In some aspects, the anti-HER2 antibody VL of the HER2 binding site has a CDR1, a CDR2, and CDR3 sequence of SEQ ID NO: 112, SEQ ID NO: 113, and SEQ ID NO: 114, respectively, and the anti-HER2 antibody VH of the HER2 binding site has a CDR1, CDR2, and CDR3 sequence of SEQ ID NO: 109, SEQ ID NO: 110, and SEQ ID NO: 111, respectively. In some aspects, the anti-HER2 antibody VL and VH of the HER2 binding site comprise sequences at least 95% identical to SEQ ID NO: 8 and SEQ ID NO: 7, respectively. In some aspects, the HER2 binding site is a single chain variable fragment (scFv). In some aspects, anti-HER2 antibody VL of the HER2 binding site is fused to the anti-HER2 antibody VH of the HER2 binding site by an scFv linker peptide comprising SEQ ID NO: 26. In some aspects, the HER2 binding site comprises a sequence at least 95% identical to SEQ ID NO: 90.

In some aspects, the anti-HER2 antibody VL of the HER2 binding site has a CDR1, a CDR2, and CDR3 sequence of SEQ ID NO: 118, SEQ ID NO: 119, and SEQ ID NO: 120, respectively, and the anti-HER2 antibody VH of the HER2 binding site has a CDR1, CDR2, and CDR3 sequence of SEQ ID NO: 115, SEQ ID NO: 116, and SEQ ID NO: 117, respectively. In some aspects, the anti-HER2 antibody VL and VH of the HER2 binding site comprise sequences at least 95% identical to SEQ ID NO: 10 and SEQ ID NO: 11, respectively. In some aspects, the HER2 binding site is a single chain variable fragment (scFv). In some aspects, the anti-HER2 antibody VL of the HER2 binding site is fused to the anti-HER2 antibody VH of the HER2 binding site by an scFv linker peptide comprising an amino acid sequence of SEQ ID NO: 27. In some aspects, the HER2 binding site comprises a sequence at least 95% identical to SEQ ID NO: 12.

In some aspects, the anti-HER2 antibody VL of the HER2 binding site has a CDR1, a CDR2, and CDR3 sequence of SEQ ID NO: 124, SEQ ID NO: 125, and SEQ ID NO: 126, respectively, and the anti-HER2 antibody VH of the HER2 binding site has a CDR1, CDR2, and CDR3 sequence of SEQ ID NO: 121, SEQ ID NO: 122, and SEQ ID NO: 123, respectively. In some aspects, the anti-HER2 antibody VL and VH of the HER2 binding site comprise sequences at least 95% identical to SEQ ID NO: 13 and SEQ ID NO: 14, respectively. In some aspects, the HER2 binding site is a single chain variable fragment (scFv). In some aspects, the anti-HER2 antibody VL of the HER2 binding site is fused to the anti-HER2 antibody VH of the HER2 binding site by an scFv linker peptide comprising an amino acid sequence of SEQ ID NO: 27. In some aspects, the HER2 binding site comprises a sequence at least 95% identical to SEQ ID NO: 15.

In some aspects, the linker peptide comprises a sequence identical to SEQ ID NO: 29.

In some aspects, the anti-CD3 antibody VH of the CD3 binding site has a CDR1, a CDR2, and CDR3 sequence of SEQ ID NO: 127, SEQ ID NO: 128, and SEQ ID NO: 129, respectively, and the anti-CD3 antibody VL has a CDR1, CDR2, and CDR3 sequence of SEQ ID NO: 130, SEQ ID NO: 131, and SEQ ID NO: 132, respectively.

In some aspects, the anti-CD3 antibody VL and VH of the CD3 binding site comprise sequences at least 95% identical to SEQ ID NO: 16 and SEQ ID NO: 17, respectively. In some aspects, the CD3 binding site is a single chain variable fragment (scFv). In some aspects, the anti-CD3 antibody VL of the CD3 binding site is fused to the anti-CD3 antibody VH of the CD3 binding site by an scFv linker peptide comprising a sequence identical to SEQ ID NO: 28. In some aspects, the CD3 binding site comprises a sequence at least 95% identical to SEQ ID NO: 18.

In some aspects, the bispecific fusion protein comprises a sequence at least 95% identical to SEQ ID NO: 19, SEQ ID NO: 20, SEQ ID NO: 21, SEQ ID NO: 22, or SEQ ID NO: 23.

In some aspects, the bispecific fusion protein has an N-terminal signal peptide comprising a sequence at least 95% identical to SEQ ID NO: 34.

In some aspects, the transgene comprises a sequence at least 95% identical to SEQ ID NO: 59, SEQ ID NO: 60, SEQ ID NO: 61, SEQ ID NO: 58, or SEQ ID NO: 62.

In some aspects, the transgene further has a regulatory element 5′ or 3′ of the sequence encoding the bispecific fusion protein. In some aspects, the regulatory element is 3′ of the sequence encoding the bispecific fusion protein. In some aspects, the regulatory element is derived from a woodchuck hepatitis virus post-transcriptional regulatory element (WPRE) and comprises a sequence at least 95% identical to SEQ ID NO: 80.

In some aspects, the transgene further has a Kozak sequence.

In some aspects, the vector further has a polyadenylation sequence 3′ of the transgene sequence and 5′ of the 3′ AAV ITR. In some aspects, the polyadenylation sequence is a bovine growth hormone (BGH) polyadenylation sequence at least 95% identical to SEQ ID NO: 81.

In some aspects, the vector further has an antibiotic resistance gene sequence. In some aspects, the antibiotic resistance gene is a kanamycin resistance gene.

In some aspects, the vector comprises a sequence at least 95% identical to SEQ ID NO: 83, SEQ ID NO: 84, SEQ ID NO: 85, SEQ ID NO: 82, or SEQ ID NO: 86.

In some aspects, the present disclosure provides an adeno-associated virus (AAV) comprising the recombinant AAV vector according to any one of the above aspects.

In some aspects, the present disclosure provides a pharmaceutical formulation comprising an adeno-associated virus (AAV) of any one of the above aspect, and a pharmaceutically acceptable carrier.

### BRIEF DESCRIPTION OF THE DRAWINGS

FIGS. 1A-1D are vector maps of AAV2 plasmids encoding bispecific fusion proteins that bind HER2 and CD3. FIG. 1A is a vector map encoding a bispecific fusion protein including the VL and VH of pertuzumab and the VH and VL of an anti-CD3 antibody. FIG. 1B is a vector map encoding a bispecific fusion protein including the VL and VH of trastuzumab and the VH and VL of an anti-CD3 antibody. FIG. 1C is a vector map encoding a bispecific fusion protein including the VL and VH of hersintuzumab and the VH and VL of an anti-CD3 antibody. FIG. 1D is a vector map encoding a bispecific fusion protein including the VL and VH of an anti-HER2 antibody (huA21) and the VH and VL of an anti-CD3 antibody.

FIG. 2 depicts construct designs of various constructs encoding bispecific anti-HER2/anti-CD3ε bispecific fusion proteins.

FIGS. 3A-3B depict histograms of flow cytometry results for anti-HER2 staining of BT474 (FIG. 3A) or BT474-Clone 5 (FIG. 3B) cells. Vertical axes depict normalized cell counts; horizontal axes depict fluorescence of anti-HER2 staining.

FIGS. 4A-4B depict graphs of cell viability of BT474 (FIG. 4A) or BT474-Clone 5 (FIG. 4B) cells following incubation with human peripheral blood mononuclear cells (huPBMCs) and indicated volume of supernatant containing an anti-HER2/anti-CD3ε bispecific fusion protein.

FIGS. 5A-5B depict flow cytometry results of a competition assay of indicated anti-HER2/anti-CD3ε bispecific fusion proteins with anti-HER2 antibody H2 MB for BT474 (FIG. 5A) or BT474-Clone 5 (FIG. 5B) cells.

FIGS. 6A-6B depict flow cytometry results of a competition assay of indicated anti-HER2/anti-CD3ε bispecific fusion proteins with anti-CD3ε antibody OKT3 for Jurkat T cells. FIG. 6A depicts histograms of anti-CD3ε staining. FIG. 6B depicts a graph showing mean fluorescence intensity (MFI) values for each protein.

FIG. 7 depicts a graph showing viability of BT474 cells following incubation with huPBMCs and indicated anti-HER2/anti-CD3ε bispecific fusion proteins.

FIG. 8 depicts a graph showing viability of BT474 or BT474-Clone 5 cells following incubation with huPBMCs and indicated anti-HER2/anti-CD3ε bispecific fusion proteins.

FIGS. 9A-9E depict results of a murine tumor model testing the anti-tumor activity of anti-HER2/anti-CD3ε bispecific fusion proteins. FIG. 9A depicts the experimental design. FIG. 9B depicts spectral images of tumor-bearing mice receiving indicated treatments at indicated time points following tumor engraftment. FIG. 9C depicts tumor size as measured by radiance (top panels) or change in weight (bottom panels). FIG. 9D depicts a Kaplan-Meier plot of survival for indicated mice. FIG. 9E depicts serum levels of anti-HER2/anti-CD3ε bispecific fusion proteins at day 31 post-tumor engraftment.

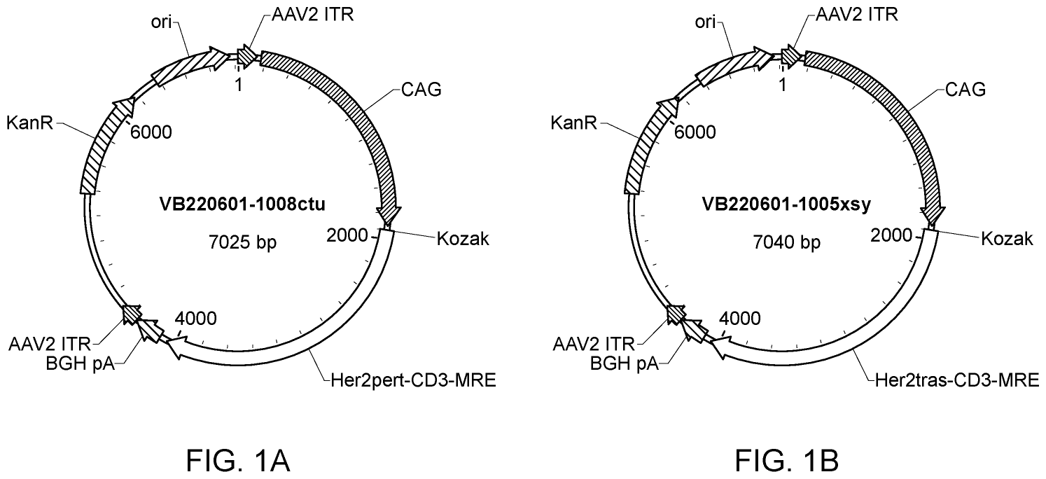

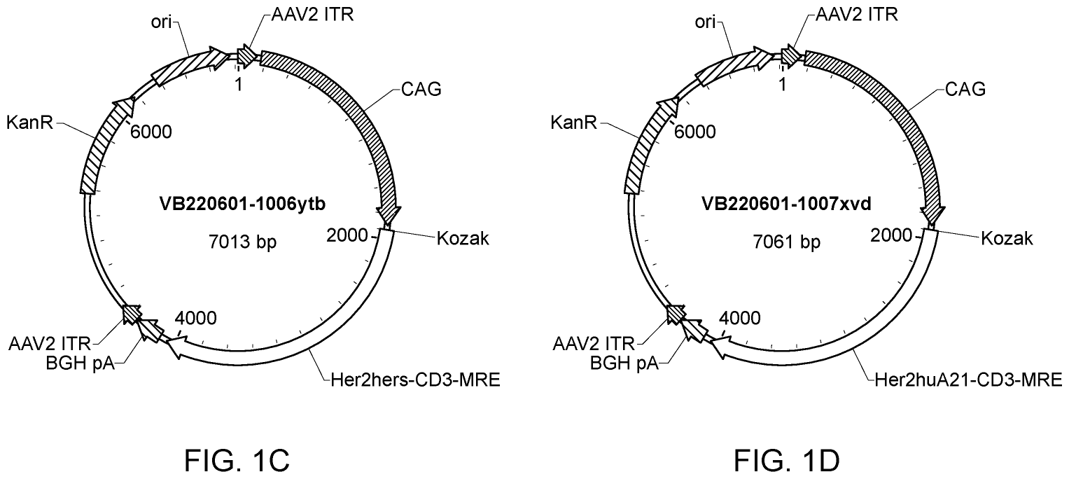

### DETAILED DESCRIPTION

The present disclosure provides recombinant adeno-associated virus (rAAV) vectors comprising a nucleic acid encoding a bispecific fusion protein that includes a heavy chain variable region (VH) and a light chain variable region (VL) of an anti-HER2 antibody, and a VH and a VL of an anti-CD3 antibody.

The present disclosure also provides methods for using rAAVs described herein for reducing the risk of, preventing, or treating cancer and metastasis in a patient.

To facilitate an understanding of the present disclosure, a number of terms and phrases are defined below.

The terms “a” and “an” as used herein mean “one or more” and include the plural unless the context is inappropriate.

The term “nucleic acid,” “nucleotide,” or “oligonucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons).

A “promoter” is defined as one or more nucleic acid control sequence(s) that direct transcription of a nucleic acid. As used herein, a promoter includes nucleic acid sequences near the start site of transcription. A promoter also optionally includes distal enhancer or repressor elements, which can be located as much as several thousand base pairs from the start site of transcription.

A “regulatory element” as used herein refers to a nucleic acid sequence capable of regulating transcription of a gene (e.g., a transgene), and/or regulate the stability or translation of a transcribed mRNA product. In some embodiments, regulatory elements can regulate tissue-specific transcription of a gene. Regulatory elements can comprise at least one transcription factor binding site, for example, a transcription factor binding site for a muscle-specific transcription factor. Regulatory elements as used herein increase or enhance promoter-driven gene expression when compared to the transcription of the gene from the promoter alone in the absence of the regulatory element. Regulatory elements as used herein may occur at any distance (i.e., proximal or distal) to the transgene they regulate. Regulatory elements as used herein may comprise part of a larger sequence involved in transcriptional control, e.g., part of a promoter sequence. However, regulatory elements alone are typically not sufficient to initiate transcription on its own and require the presence of a promoter.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

As used herein, the term “sequence of equivalent coding potential” refers to a nucleic acid sequence having functional equivalence to another reference nucleic acid. A sequence of equivalent coding potential may or may not have the same primary nucleotide sequence. For example, for a reference nucleic acid coding for an expressed polypeptide, a sequence of equivalent coding potential is functionally able to code for the same expressed polypeptide and may comprise an identical primary nucleotide sequence as the reference nucleic acid, or may comprise one or more alternative codon(s) as compared to the reference nucleic acid. For example, an endogenous nucleic acid sequence encoding a polypeptide may be altered via codon optimization to result in a sequence that codes for an identical polypeptide. A codon optimized sequence may be one in which codons in a polynucleotide encoding a polypeptide have been substituted in order to modify the activity, expression, and/or stability of the polynucleotide. For example, codon optimization can be used to vary the degree of sequence similarity of a sequence of equivalent coding potential as compared to an endogenous gene sequence, while preserving the potential to encode the protein product of the endogenous gene.

“Polypeptide,” “peptide,” and “protein” are used interchangeably herein to refer to a polymer of amino acid residues. As used herein, the terms encompass amino acid chains of any length, including full-length proteins, and functional fragments thereof, wherein the amino acid residues are linked by covalent peptide bonds.

The terms “variable domain” (e.g., VH domain or VL domain) and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

As used herein, the term “complementary” or “complementarity” refers to specific base pairing between nucleotides or nucleic acids. Complementary nucleotides are, generally, A and T (or A and U), and G and C.

As used herein, the term “transgene” refers to an exogenous gene artificially introduced into the genome of a cell, or an endogenous gene artificially introduced into a non-natural locus in the genome of a cell. A transgene can refer to a segment of DNA involved in producing or encoding a polypeptide chain. Transgenes may include regions preceding and following the coding region (leader and trailer) as well as intervening sequences (introns) between individual coding segments (exons).

As used herein, the terms “introducing” or “delivering” in the context of nucleic acids, for example, AAV vectors, refers to the translocation of the nucleic acid from outside a cell to inside the cell, for example, a muscle cell. In some cases, introducing refers to translocation of the nucleic acid from outside the cell to inside the nucleus of the cell. Various methods of such translocation are contemplated, including but not limited to, electroporation, contact with nanowires or nanotubes, receptor mediated internalization, translocation via cell penetrating peptides, liposome-mediated translocation, and the like.

As used herein, the terms “packaged” or “encapsidated” refers to the inclusion of a AAV vector in a viral capsid to form an AAV particle.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, 99%, or 100%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “recipient,” “individual,” “subject,” “host,” and “patient,” are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel and/or a cancer diagnosis or current cancer treatment.

As used herein, the term “efficient delivery” or “efficiently delivering” refers to administration of recombinant adeno-associated virus vector encoding a transgene resulting in expression of the transgene in a desired cell or tissue.

As used herein, the term “effective amount” refers to the amount of a substance (e.g., a recombinant adeno-associated virus of the present disclosure) sufficient to effect beneficial or desired results (e.g., expression of a protein, or a desired prophylactic or therapeutic effect). An effective amount can be administered in one or more administration(s), application(s) or dosage(s) and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

### 1. Recombinant Adeno-Associated Viral (AAV) Vectors

As used herein, a “recombinant adeno-associated viral (rAAV) vector” refers to a vector (e.g., nucleic acid vector) comprising a promoter and one or more transgenes, or polynucleotide of interest, that are flanked by AAV inverted terminal repeat (ITR) sequences. rAAV vectors described herein can be replicated, and packaged into viral particles when introduced into a host cell also comprising one or more vectors encoding rep and cap gene products.

### Inverted Terminal Repeats

Inverted terminal repeats (ITR) are palindromic 145 nucleotide sequences that flank a transgene. The 5′ and 3′ ITRs of a recombinant adeno-associated viral (rAAV) vector are necessary for both the integration of the transgene into the host cell genome (e.g., chromosome 19 in humans) and for encapsidation into the AAV particle.

In some embodiments, rAAV vectors of the present disclosure comprise ITR sequences from any one AAV serotype, for example, AAVrh.74, AAV1, AAV2, AAV3, AAV4, AAV5, AAV6, AAV8, AAV9, AAV10, AAV11, AAV12, or AAV13. In some embodiments, the AAV serotype is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the recombinant AAV vectors disclosed herein comprise AAV2 5′ and 3′ ITR sequences. In some embodiments, the recombinant AAV vectors disclosed herein comprise AAV8 5′ and 3′ ITR sequences.

In some embodiments, recombinant AAV vectors described herein comprise a 5′ AAV2 ITR having at least 80%, 85%, 90%, 91%, 92%, 93%, 94%, 95%, 96%, 97%, 98%, 99%, or 100% sequence identity to SEQ ID NO: 75 (see TABLE 1A). In some embodiments, the 5′ AAV2 ITR comprises a sequence having at least about 80% identity to SEQ ID NO: 75. In some embodiments, the 5′ AAV2 ITR comprises a sequence having at least about 85% identity to SEQ ID NO: 75. In some embodiments, the 5′ AAV2 ITR comprises a sequence having at least about 90% identity to SEQ ID NO: 75. In some embodiments, the 5′ AAV2 ITR comprises a sequence having at least about 95% identity to SEQ ID NO: 75. In some embodiments, the 5′ AAV2 ITR comprises a sequence having at least about 96% identity to SEQ ID NO: 75. In some embodiments, the 5′ AAV2 ITR comprises a sequence having at least about 97% identity to SEQ ID NO: 75. In some embodiments, the 5′ AAV2 ITR comprises a sequence having at least about 98% identity to SEQ ID NO: 75. In some embodiments, the 5′ AAV2 ITR comprises a sequence having at least about 99% identity to SEQ ID NO: 75. In some embodiments, the 5′ AAV2 ITR comprises a sequence having 100% identity to SEQ ID NO: 75. In some embodiments, the 5′ AAV2 ITR comprises SEQ ID NO: 75. In some embodiments, the 5′ AAV2 ITR consists of SEQ ID NO: 75.

In some embodiments, recombinant AAV vectors described herein comprises a 3′ AAV2 ITR having at least 80%, 85%, 90%, 91%, 92%, 93%, 94%, 95%, 96%, 97%, 98%, 99%, or 100% sequence identity to SEQ ID NO: 91 (see TABLE 1A). In some embodiments, the 3′ AAV2 ITR comprises a sequence having at least about 80% identity to SEQ ID NO: 91. In some embodiments, the 3′ AAV2 ITR comprises a sequence having at least about 85% identity to SEQ ID NO: 91. In some embodiments, the 3′ AAV2 ITR comprises a sequence having at least about 90% identity to SEQ ID NO: 91. In some embodiments, the 3′ AAV2 ITR comprises a sequence having at least about 95% identity to SEQ ID NO: 91. In some embodiments, the 3′ AAV2 ITR comprises a sequence having at least about 96% identity to SEQ ID NO: 91. In some embodiments, the 3′ AAV2 ITR comprises a sequence having at least about 97% identity to SEQ ID NO: 91. In some embodiments, the 3′ AAV2 ITR comprises a sequence having at least about 98% identity to SEQ ID NO: 91. In some embodiments, the 3′ AAV2 ITR comprises a sequence having at least about 99% identity to SEQ ID NO: 91. In some embodiments, the 3′ AAV2 ITR comprises a sequence having 100% identity to SEQ ID NO: 91. In some embodiments, the 3′ AAV2 ITR comprises SEQ ID NO: 91. In some embodiments, the 3′ AAV2 ITR consists of SEQ ID NO: 91.

TABLE 1A
AAV ITR SEQUENCES

| Element | Nucleotide sequence |
|---|---|
| AAV2 | CCTGCAGGCAGCTGCGCGCTCGCTCGCTCACTGAGGCCGCC |
| ITR | CGGGCAAAGCCCGGGCGTCGGGCGACCTTTGGTCGCCCGGC |
| (SEQ ID | CTCAGTGAGCGAGCGAGCGCGCAGAGAGGGAGTGGCCAACT |
| NO: 75) | CCATCACTAGGGGTTCCT |
| AAV2 | AGGAACCCCTAGTGATGGAGTTGGCCACTCCCTCTCTGCGC |
| ITR | GCTCGCTCGCTCACTGAGGCCGGGCGACCAAAGGTCGCCCG |
| (SEQ ID | ACGCCCGGGCTTTGCCCGGGCGGCCTCAGTGAGCGAGCGAG |
| NO: 91) | CGCGCAGCTGCCTGCAGG |

### Promoters

Promoters drive the expression of the rAAV vector transgene and are typically located upstream (or 5′) of the transgene whose expression they regulate.

In some embodiments, recombinant AAV vectors of the present disclosure comprise a mammalian promoter, for example, human, non-human primate (e.g., cynomolgous macaque), mouse, horse, cow, pig, cat, and dog promoters. In some embodiments, recombinant AAV vectors disclosed herein comprise strong, constitutively active promoters to drive high-level expression of the transgene. For example, in some embodiments the promoter is a CAG promoter (a cytomegalovirus early enhancer fused with a chicken β-actin promoter), a cytomegalovirus (CMV) promoter/enhancer, an elongation factor 1α (EF1α) promoter, a simian virus 40 (SV40) promoter, or a chicken β-actin promoter.

In some embodiments, a promoter described herein comprise a CAG promoter having at least 80%, 85%, 90%, 91%, 92%, 93%, 94%, 95%, 96%, 97%, 98%, 99%, or 100% sequence identity to SEQ ID NO: 87. In some embodiments, the CAG promoter comprises a sequence having at least about 80% identity to SEQ ID NO: 87. In some embodiments, the CAG promoter comprises a sequence having at least about 85% identity to SEQ ID NO: 87. In some embodiments, the CAG promoter comprises a sequence having at least about 90% identity to SEQ ID NO: 87. In some embodiments, the CAG promoter comprises a sequence having at least about 95% identity to SEQ ID NO: 87. In some embodiments, the CAG promoter comprises a sequence having at least about 96% identity to SEQ ID NO: 87. In some embodiments, the CAG promoter comprises a sequence having at least about 97% identity to SEQ ID NO: 87. In some embodiments, the CAG promoter comprises a sequence having at least about 98% identity to SEQ ID NO: 87. In some embodiments, the CAG promoter comprises a sequence having at least about 99% identity to SEQ ID NO: 87. In some embodiments, the CAG promoter comprises a sequence having 100% identity to SEQ ID NO: 87.

CAG PROMOTER SEQUENCE:
(SEQ ID NO: 87)
CTCGACATTGATTATTGACTAGTTATTAATAGTAATCAATTACGGG
GTCATTAGTTCATAGCCCATATATGGAGTTCCGCGTTACATAACTT
ACGGTAAATGGCCCGCCTGGCTGACCGCCCAACGACCCCCGCCCAT
TGACGTCAATAATGACGTATGTTCCCATAGTAACGCCAATAGGGAC
TTTCCATTGACGTCAATGGGTGGAGTATTTACGGTAAACTGCCCAC
TTGGCAGTACATCAAGTGTATCATATGCCAAGTACGCCCCCTATTG
ACGTCAATGACGGTAAATGGCCCGCCTGGCATTATGCCCAGTACAT
GACCTTATGGGACTTTCCTACTTGGCAGTACATCTACGTATTAGTCA
TCGCTATTACCATGGTCGAGGTGAGCCCCACGTTCTGCTTCACTCTC
CCCATCTCCCCCCCCTCCCCACCCCCAATTTTGTATTTATTTATTTTT
TAATTATTTTGTGCAGCGATGGGGGCGGGGGGGGGGGGGGGGCGC
GCGCCAGGCGGGGCGGGGCGGGGCGAGGGGCGGGGCGGGGCGAG
GCGGAGAGGTGCGGCGGCAGCCAATCAGAGCGGCGCGCTCCGAAA
GTTTCCTTTTATGGCGAGGCGGCGGCGGCGGCGGCCCTATAAAAAG
CGAAGCGCGCGGCGGGCGGGAGTCGCTGCGCGCTGCCTTCGCCCC
GTGCCCCGCTCCGCCGCCGCCTCGCGCCGCCCGCCCCGGCTCTGAC
TGACCGCGTTACTCCCACAGGTGAGCGGGCGGGACGGCCCTTCTCC
TCCGGGCTGTAATTAGCGCTTGGTTTAATGACGGCTTGTTTCTTTTC
TGTGGCTGCGTGAAAGCCTTGAGGGGCTCCGGGAGGGCCCTTTGTG
CGGGGGGAGCGGCTCGGGGGGTGCGTGCGTGTGTGTGTGCGTGGG
GAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGG
AGCGCGGCCGGGGGCGGTGCCCCGCGGTGCGGGGGGGGCTGCGAG
GGGAACAAAGGCTGCGTGCGGGGTGTGTGCGTGGGGGGGTGAGCA
GGGGGTGTGGGCGCGTCGGTCGGGCTGCAACCCCCCCTGCACCCCC
CTCCCCGAGTTGCTGAGCACGGCCCGGCTTCGGGTGCGGGGCTCCG
TACGGGGCGTGGCGCGGGGCTCGCCGTGCCGGGCGGGGGGTGGCG
GCAGGTGGGGGTGCCGGGCGGGGCGGGGCCGCCTCGGGCCGGGGA
GGGCTCGGGGGAGGGGCGCGGCGGCCCCCGGAGCGCCGGCGGCTG
TCGAGGCGCGGCGAGCCGCAGCCATTGCCTTTTATGGTAATCGTGC
GAGAGGGCGCAGGGACTTCCTTTGTCCCAAATCTGTGCGGAGCCGA
AATCTGGGAGGCGCCGCCGCACCCCCTCTAGCGGGCGCGGGGCGA
AGCGGTGCGGCGCCGGCAGGAAGGAAATGGGCGGGGAGGGCCTTC
GTGCGTCGCCGCGCCGCCGTCCCCTTCTCCCTCTCCAGCCTCGGGG
CTGTCCGCGGGGGGACGGCTGCCTTCGGGGGGGACGGGGCAGGGC
GGGGTTCGGCTTCTGGCGTGTGACCGGCGGCTCTAGAGCCTCTGCT
AACCATGTTCATGCCTTCTTCTTTTTCCTACAGCTCCTGGGCAACGT
GCTGGTTATTGTGCTGTCTCATCATTTTGGCAAAGAATTG

### SV40 Intron

In some embodiments, recombinant AAV vectors of the present disclosure comprise an SV40 intron. The SV40 intron is a commonly used regulatory element in gene therapy vectors and enhances translation and stability of the expressed RNA transcript.

In certain embodiments, the SV40 intron is downstream (i.e., 3′) of the promoter and upstream (i.e., 5′) of the transgene. In other embodiments, the SV40 intron can be downstream (i.e., 3′) of the transgene.

In some embodiments, the SV40 intron comprises a sequence having at least 80%, 85%, 90%, 91%, 92%, 93%, 94%, 95%, 96%, 97%, 98%, 99%, or 100% sequence identity to SEQ ID NO: 92. In some embodiments, the SV40 intron comprises SEQ ID NO: 92. In some embodiments, the SV40 intron consists of SEQ ID NO: 92. In some embodiments, the SV40 intron comprises a sequence having at least about 80% identity to SEQ ID NO: 92. In some embodiments, the SV40 intron comprises a sequence having at least about 85% identity to SEQ ID NO: 92. In some embodiments, the SV40 intron comprises a sequence having at least about 90% identity to SEQ ID NO: 92. In some embodiments, the SV40 intron comprises a sequence having at least about 95% identity to SEQ ID NO: 92. In some embodiments, the SV40 intron comprises a sequence having at least about 96% identity to SEQ ID NO: 92. In some embodiments, the SV40 intron comprises a sequence having at least about 97% identity to SEQ ID NO: 92. In some embodiments, the SV40 intron comprises a sequence having at least about 98% identity to SEQ ID NO: 92. In some embodiments, the SV40 intron comprises a sequence having at least about 99% identity to SEQ ID NO: 92. In some embodiments, the SV40 intron comprises a sequence having 100% identity to SEQ ID NO: 92.

SV40 INTRON SEQUENCE:
(SEQ ID NO: 92)
GTAAGTTTAGTCTTTTTTGTCTTTTATTTCAGGTCCCGGATCCGGTG
GTGGTGCAAATCAAAGAACTGCTCCTCAGTCGATGTTGCCTTTACT
TCTAG

### Polyadenylation Sequence

In some embodiments, recombinant AAV vectors of the present disclosure comprise a sequence encoding a polyadenylation sequence, such as a bovine growth hormone (BGH) polyadenylation sequence (SEQ ID NO: 81) or an SV40 polyadenylation sequence (SEQ ID NO: 139). Polyadenylation sequences are commonly used nucleic acid elements in gene therapy vectors that assists in RNA export from the nucleus, translation of RNA, and RNA stability.

In some embodiments recombinant AAV vectors of the present disclosure comprise a sequence encoding a BGH poly(A) tail having at least 80%, 85%, 90%, 91%, 92%, 93%, 94%, 95%, 96%, 97%, 98%, 99%, or 100% sequence identity to SEQ ID NO: 81. In some embodiments, the BGH poly(A) tail comprises a sequence having at least about 80% identity to SEQ ID NO: 81. In some embodiments, the BGH poly(A) tail comprises a sequence having at least about 85% identity to SEQ ID NO: 81. In some embodiments, the BGH poly(A) tail comprises a sequence having at least about 90% identity to SEQ ID NO: 81. In some embodiments, the BGH poly(A) tail comprises a sequence having at least about 95% identity to SEQ ID NO: 81. In some embodiments, the BGH poly(A) tail comprises a sequence having at least about 96% identity to SEQ ID NO: 81. In some embodiments, the BGH poly(A) tail comprises a sequence having at least about 97% identity to SEQ ID NO: 81. In some embodiments, the BGH poly(A) tail comprises a sequence having at least about 98% identity to SEQ ID NO: 81. In some embodiments, the BGH poly(A) tail comprises a sequence having at least about 99% identity to SEQ ID NO: 81. In some embodiments, the BGH poly(A) tail comprises a sequence having 100% identity to SEQ ID NO: 81. In some embodiments, the BGH poly(A) tail comprises a sequence according to SEQ ID NO: 81. In some embodiments, the BGH poly(A) tail consists of a sequence according to SEQ ID NO: 81.

In some embodiments recombinant AAV vectors of the present disclosure comprise a sequence encoding a SV40 poly(A) tail having at least 80%, 85%, 90%, 91%, 92%, 93%, 94%, 95%, 96%, 97%, 98%, 99%, or 100% sequence identity to SEQ ID NO: 139. In some embodiments, the SV40 poly(A) tail comprises a sequence having at least about 80% identity to SEQ ID NO: 139. In some embodiments, the SV40 poly(A) tail comprises a sequence having at least about 85% identity to SEQ ID NO: 139. In some embodiments, the SV40 poly(A) tail comprises a sequence having at least about 90% identity to SEQ ID NO: 139. In some embodiments, the SV40 poly(A) tail comprises a sequence having at least about 95% identity to SEQ ID NO: 139. In some embodiments, the SV40 poly(A) tail comprises a sequence having at least about 96% identity to SEQ ID NO: 139. In some embodiments, the SV40 poly(A) tail comprises a sequence having at least about 97% identity to SEQ ID NO: 139. In some embodiments, the SV40 poly(A) tail comprises a sequence having at least about 98% identity to SEQ ID NO: 139. In some embodiments, the SV40 poly(A) tail comprises a sequence having at least about 99% identity to SEQ ID NO: 139. In some embodiments, the SV40 poly(A) tail comprises a sequence having 100% identity to SEQ ID NO: 139. In some embodiments, the SV40 poly(A) tail comprises a sequence according to SEQ ID NO: 139. In some embodiments, the SV40 poly(A) tail consists of a sequence according to SEQ ID NO: 139.

BGH poly(A) tail sequence:
(SEQ ID NO: 81)
CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCAT
TCTATTCTGGGGGGTGGGGGGGGCAGGACAGCAAGGGGGAGG
ATTGGGAAGAGAATAGCAGGCATGCTGGGGA.
SV40 poly(A) tail sequence:
(SEQ ID NO: 139)
AACTTGTTTATTGCAGCTTATAATGGTTACAAATAAAGCAATAG
CATCACAAATTTCACAAATAAAGCATTTTTTTCACTGC

### Enhancers

In some embodiments, recombinant AAV vectors of the present disclosure comprise one or more enhancer sequence(s). Enhancer sequences can increase the level of transcription of the transgene, for example, by serving as binding sites for transcription factors and co-regulators that assist in DNA looping and recruitment of the transcriptional machinery to promoters.

In some embodiments, the enhancer is downstream (i.e., 3′) of the 5′ ITR and upstream (i.e., 5′) of the promoter. In some embodiments, the enhancer is downstream (i.e., 3′) of the promoter and upstream (i.e., 5′) of the transgene. In some embodiments, the enhancer is downstream (i.e., 3′) of the transgene and upstream (i.e., 5′) of the 3′ UTR.

### Antibiotic Resistance Genes

In some embodiments, recombinant AAV vectors of the present disclosure comprise an antibiotic resistance gene. In some embodiments, the antibiotic resistance gene encodes kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the antibiotic resistance gene encodes kanamycin.

In some embodiments, the kanamycin resistance gene comprises a nucleotide sequence having at least 85% (e.g., 85%, 90%, 95%, 97%, 98%, or 99%) sequence identity to the nucleic acid sequence of SEQ ID NO: 150. In some embodiments, the kanamycin resistance gene comprises a nucleotide sequence having at least 85% sequence identity to the nucleic acid sequence of SEQ ID NO: 150. In some embodiments, the kanamycin resistance gene comprises a nucleotide sequence having at least 90% sequence identity to the nucleic acid sequence of SEQ ID NO: 150. In some embodiments, the kanamycin resistance gene comprises a nucleotide sequence having at least 95% sequence identity to the nucleic acid sequence of SEQ ID NO: 150. In some embodiments, the kanamycin resistance gene comprises a nucleotide sequence having at least 97% sequence identity to the nucleic acid sequence of SEQ ID NO: 150. In some embodiments, the kanamycin resistance gene comprises a nucleotide sequence having at least 98% sequence identity to the nucleic acid sequence of SEQ ID NO: 150. In some embodiments, the kanamycin resistance gene comprises a nucleotide sequence having at least 99% sequence identity to the nucleic acid sequence of SEQ ID NO: 150. In some embodiments, the kanamycin resistance gene comprises a nucleotide sequence having the nucleic acid sequence of SEQ ID NO: 150.

In some embodiments, the kanamycin resistance gene comprises a nucleotide sequence having at least 85% (e.g., 85%, 90%, 95%, 97%, 98%, or 99%) sequence identity to the nucleic acid sequence of SEQ ID NO: 177. In some embodiments, the kanamycin resistance gene comprises a nucleotide sequence having at least 85% sequence identity to the nucleic acid sequence of SEQ ID NO: 177. In some embodiments, the kanamycin resistance gene comprises a nucleotide sequence having at least 90% sequence identity to the nucleic acid sequence of SEQ ID NO: 177. In some embodiments, the kanamycin resistance gene comprises a nucleotide sequence having at least 95% sequence identity to the nucleic acid sequence of SEQ ID NO: 177. In some embodiments, the kanamycin resistance gene comprises a nucleotide sequence having at least 97% sequence identity to the nucleic acid sequence of SEQ ID NO: 177. In some embodiments, the kanamycin resistance gene comprises a nucleotide sequence having at least 98% sequence identity to the nucleic acid sequence of SEQ ID NO: 177. In some embodiments, the kanamycin resistance gene comprises a nucleotide sequence having at least 99% sequence identity to the nucleic acid sequence of SEQ ID NO: 177. In some embodiments, the kanamycin resistance gene comprises a nucleotide sequence having the nucleic acid sequence of SEQ ID NO: 177.

Kanamycin Variant 1
(SEQ ID NO: 150)
ATGGCTAAAATGAGAATATCACCGGAATTGAAAAAACTGATCG
AAAAATACCGCTGCGTAAAAGATACGGAAGGAATGTCTCCTGC
TAAGGTATATAAGCTGGTGGGAGAAAATGAAAACCTATATTTA
AAAATGACGGACAGCCGGTATAAAGGGACCACCTATGATGTGG
AACGGGAAAAGGACATGATGCTATGGCTGGAAGGAAAGCTGCC
TGTTCCAAAGGTCCTGCACTTTGAACGGCATGATGGCTGGAGCA
ATCTGCTCATGAGTGAGGCCGATGGCGTCCTTTGCTCGGAAGAG
TATGAAGATGAACAAAGCCCTGAAAAGATTATCGAGCTGTATG
CGGAGTGCATCAGGCTCTTTCACTCCATCGACATATCGGATTGT
CCCTATACGAATAGCTTAGACAGCCGCTTAGCCGAATTGGATTA
CTTACTGAATAACGATCTGGCCGATGTGGATTGCGAAAACTGG
GAAGAAGACACTCCATTTAAAGATCCGCGCGAGCTGTATGATTT
TTTAAAGACGGAAAAGCCCGAAGAGGAACTTGTCTTTTCCCAC
GGCGACCTGGGAGACAGCAACATCTTTGTGAAAGATGGCAAAG
TAAGTGGCTTTATTGATCTTGGGAGAAGCGGCAGGGCGGACAA
GTGGTATGACATTGCCTTCTGCGTCCGGTCGATCAGGGAGGATA
TCGGGGAAGAACAGTATGTCGAGCTATTTTTTGACTTACTGGGG
ATCAAGCCTGATTGGGAGAAAATAAAATATTATATTTTACTGGA
TGAATTGTTTTAG
Kanamycin Variant 2
(SEQ ID NO: 177)
TTAGAAAAACTCATCGAGCATCAAATGAAACTGCAATTTATTCA
TATCAGGATTATCAATACCATATTTTTGAAAAAGCCGTTTCTGT
AATGAAGGAGAAAACTCACCGAGGCAGTTCCATAGGATGGCAA
GATCCTGGTATCGGTCTGCGATTCCGACTCGTCCAACATCAATA
CAACCTATTAATTTCCCCTCGTCAAAAATAAGGTTATCAAGTGA
GAAATCACCATGAGTGACGACTGAATCCGGTGAGAATGGCAAA
AGCTTATGCATTTCTTTCCAGACTTGTTCAACAGGCCAGCCATT
ACGCTCGTCATCAAAATCACTCGCATCAACCAAACCGTTATTCA
TTCGTGATTGCGCCTGAGCGAGACGAAATACGCGATCGCTGTTA
AAAGGACAATTACAAACAGGAATCGAATGCAACCGGCGCAGG
AACACTGCCAGCGCATCAACAATATTTTCACCTGAATCAGGATA
TTCTTCTAATACCTGGAATGCTGTTTTCCCGGGGATCGCAGTGG
TGAGTAACCATGCATCATCAGGAGTACGGATAAAATGCTTGAT
GGTCGGAAGAGGCATAAATTCCGTCAGCCAGTTTAGTCTGACC
ATCTCATCTGTAACATCATTGGCAACGCTACCTTTGCCATGTTTC
AGAAACAACTCTGGCGCATCGGGCTTCCCATACAATCGATAGA
TTGTCGCACCTGATTGCCCGACATTATCGCGAGCCCATTTATAC
CCATATAAATCAGCATCCATGTTGGAATTTAATCGCGGCCTCGA
GCAAGACGTTTCCCGTTGAATATGGCTCAT

### Kozak Sequences

In some embodiments, recombinant AAV vectors of the present disclosure comprise a Kozak sequence. In some embodiments, the Kozak sequence is an AAV2 Kozak sequence. In some embodiments, the Kozak sequence is an AAV8 Kozak sequence. In some embodiments, the Kozak sequence is an AAV-rh74 Kozak sequence. Exemplary Kozak sequences are seen in the TABLE 1B below.

TABLE 1B
Exemplary Kozak Sequences

| SEQ ID NO | Sequence |
|---|---|
| 151 | CAUUGUAUGUC |
| 152 | UCGUUUAUGGA |
| 153 | CAGUUUAUGGU |
| 154 | CAUUGUAUGGU |
| 155 | UAGUGUAUGCU |
| 156 | UCUUUUAUGUC |
| 157 | UGUUUUAUGUC |
| 158 | UAGUUUAUGUC |
| 159 | UAGUGUAUGUC |
| 160 | UAGCGCAUGGC |
| 161 | UGGUAUAUGGC |
| 162 | UAGUUUAUGGC |
| 163 | CAGUGUAUGGC |
| 164 | CAUUGUAUGGC |
| 165 | CCGUUUAUGGG |
| 166 | ACUUGUAUGGG |
| 167 | CAUUUUAUGGG |
| 168 | UAGUGUAUGGG |
| 169 | UAGUUUAUGGG |
| 170 | UGUUUUAUGGG |
| 171 | UCUUUUAUGGG |
| 172 | UAGUGUAUGGC |
| 173 | UGUUUUAUGGC |
| 174 | UCUUUUAUGGC |

In some embodiments, the Kozak sequence comprises a sequence having at least 85% (e.g., 85%, 90%, 95%, 97%, 98%, or 99%) sequence identity to the nucleic acid sequence of any one of SEQ ID NOs: 151-174. In some embodiments, the Kozak sequence comprises a sequence having at least 85% sequence identity to the nucleic acid sequence of any one of SEQ ID NOs: 151-174. In some embodiments, the Kozak sequence comprises a sequence having at least 90% sequence identity to the nucleic acid sequence of any one of SEQ ID NOs: 151-174. In some embodiments, the Kozak sequence comprises a sequence having at least 95% sequence identity to the nucleic acid sequence of any one of SEQ ID NOs: 151-174. In some embodiments, the Kozak sequence comprises a sequence having at least 97% sequence identity to the nucleic acid sequence of any one of SEQ ID NOs: 151-174. In some embodiments, the Kozak sequence comprises a sequence having at least 98% sequence identity to the nucleic acid sequence of any one of SEQ ID NOs: 151-174. In some embodiments, the Kozak sequence comprises a sequence having at least 99% sequence identity to the nucleic acid sequence of any one of SEQ ID NOs: 151-174. In some embodiments, the Kozak sequence comprises a sequence having the nucleic acid sequence of any one of SEQ ID NOs: 151-174.

### αHER2-αCD3 Transgene

In some embodiments, transgenes of the present disclosure are nucleic acid sequences encoding a bispecific fusion protein having a HER2 binding site and a CD3 binding site. In some embodiments, the HER2 binding site comprises a VH and a VL of an anti-HER2 antibody, and the CD3 binding site comprises a VH and a VL of an anti-CD3 antibody.

In some embodiments, the transgene is incorporated into the genome of the cell or is expressed episomally.

### HER2 Binding Site

As used herein, a HER2 binding site comprises a polypeptide or complex of two or more polypeptides that specifically binds a protein comprising a sequence of SEQ ID NO: 93 and, in some embodiments, related isoforms and orthologs. In some embodiments, HER2 binding sites specifically bind one or more than one epitope on the extracellular domain of HER2.

(SEQ ID NO: 93)
MELAALCRWGLLLALLPPGAASTQVCTGTDMKLRLPASPETHLDM
LRHLYQGCQVVQGNLELTYLPTNASLSFLQDIQEVQGYVLIAHNQV
RQVPLQRLRIVRGTQLFEDNYALAVLDNGDPLNNTTPVTGASPGGL
RELQLRSLTEILKGGVLIQRNPQLCYQDTILWKDIFHKNNQLALTLI
DTNRSRACHPCSPMCKGSRCWGESSEDCQSLTRTVCAGGCARCKG
PLPTDCCHEQCAAGCTGPKHSDCLACLHFNHSGICELHCPALVTYN
TDTFESMPNPEGRYTFGASCVTACPYNYLSTDVGSCTLVCPLHNQE
VTAEDGTQRCEKCSKPCARVCYGLGMEHLREVRAVTSANIQEFAG
CKKIFGSLAFLPESFDGDPASNTAPLQPEQLQVFETLEEITGYLYISA
WPDSLPDLSVFQNLQVIRGRILHNGAYSLTLQGLGISWLGLRSLREL
GSGLALIHHNTHLCFVHTVPWDQLFRNPHQALLHTANRPEDECVG
EGLACHQLCARGHCWGPGPTQCVNCSQFLRGQECVEECRVLQGLP
REYVNARHCLPCHPECQPQNGSVTCFGPEADQCVACAHYKDPPFC
VARCPSGVKPDLSYMPIWKFPDEEGACQPCPINCTHSCVDLDDKGC
PAEQRASPLTSIISAVVGILLVVVLGVVFGILIKRRQQKIRKYTMRRL
LQETELVEPLTPSGAMPNQAQMRILKETELRKVKVLGSGAFGTVYK
GIWIPDGENVKIPVAIKVLRENTSPKANKEILDEAYVMAGVGSPYVS
RLLGICLTSTVQLVTQLMPYGCLLDHVRENRGRLGSQDLLNWCMQ
IAKGMSYLEDVRLVHRDLAARNVLVKSPNHVKITDFGLARLLDIDE
TEYHADGGKVPIKWMALESILRRRFTHQSDVWSYGVTVWELMTFG
AKPYDGIPAREIPDLLEKGERLPQPPICTIDVYMIMVKCWMIDSECR
PRFRELVSEFSRMARDPQRFVVIQNEDLGPASPLDSTFYRSLLEDDD
MGDLVDAEEYLVPQQGFFCPDPAPGAGGMVHHRHRSSSTRSGGGD
LTLGLEPSEEEAPRSPLAPSEGAGSDVFDGDLGMGAAKGLQSLPTH
DPSPLQRYSEDPTVPLPSETDGYVAPLTCSPQPEYVNQPDVRPQPPS
PREGPLPAARPAGATLERPKTLSPGKNGVVKDVFAFGGAVENPEYL
TPQGGAAPQPHPPPAFSPAFDNLYYWDQDPPERGAPPSTFKGTPTA
ENPEYLGLDVPV
*Underlined text indicates extracellular domain

In some embodiments, a HER2 binding site comprises a heavy chain variable region (VH) and a light chain variable region (VL). TABLE 2A lists VH and VL domains of anti-HER2 antibodies, and their corresponding complementarity-determining regions (CDRs) that, in combination, can specifically bind to HER2. TABLE 2B lists the corresponding nucleotide sequences of the VH and VL domains of anti-HER2 antibodies.

TABLE 2A
αHER2 VH/VL and CDRs amino acid sequences

| Identifier | VH | VL |
|---|---|---|
| Pertuzumab | EVQLVESGGGLVQPGGSLRLS | DIQMTQSPSSLSASVGDRV |
| | CAASGFTFTDYTMDWVRQAP | TITCKASQDVSIGVAWYQQ |
| | GKGLEWVADVNPNSGGSIYN | KPGKAPKLLIYSASYRYTG |
| | QRFKGRFTLSVDRSKNTLYLQ | VPSRFSGSGSGTDFTLTIS |
| | MNSLRAEDTAVYYCARNLGP | SLQPEDFATYYCQQYYIY |
| | SFYFDYWGQGTLVTVSS | PYTFGQGTKVEIK |
| | (SEQ ID NO: 1) | (SEQ ID NO: 2) |
| | CDR1: GFTFTDYTMD | CDR1: KASQDVSIG |
| | (SEQ ID NO: 97) | (SEQ ID NO: 100) |
| | CDR2: DVNPNSGGSIYNQRFKG | CDR2: SASYRY |
| | (SEQ ID NO: 98) | (SEQ ID NO: 101) |
| | CDR3: NLGPSFYFDY | CDR3: QQYYIYPY |
| | (SEQ ID NO: 99) | (SEQ ID NO: 102) |
| Herceptin | EVQLVESGGGLVQPGGSLRLS | DIQMTQSPSSLSASVGDRV |
| (Trastuzumab) | CAASGFNIKDTYIHWVRQAPG | TITCRASQDVNTAVAWYQQ |
| | KGLEWVARIYPTNGYTRYAD | KPGKAPKLLIYSASFLYSG |
| | SVKGRFTISADTSKNTAYLQM | VPSRFSGSRSGTDFTLTIS |
| | NSLRAEDTAVYYCSRWGGDG | SLQPEDFATYYCQQHY |
| | FYAMDYWGQGTLVTVSS | TTPPTFGQGTKVEIK |
| | (SEQ ID NO: 4) | (SEQ ID NO: 5) |
| | CDR1: GFNIKDTY | CDR1: QDVNTA |
| | (SEQ ID NO: 103) | (SEQ ID NO: 106) |
| | CDR2: IYPTNGYT | CDR2: SAS |
| | (SEQ ID NO: 104) | (SEQ ID NO: 107) |
| | CDR3: SRWGGDGFYAMDY | CDR3: QQHYTTPPT |
| | (SEQ ID NO: 105) | (SEQ ID NO: 108) |
| Hersintuzumab | EVKLVESGGGLVKPGGSLRLS | DIVMTQSPSSLSASVGDRV |
| | CATSGFSFSSYYMYWVRQAP | TITCRASQNVRTAVAWFQQ |
| | GKRLEWVAYISSGSEIYYSDS | KPGKAPKALIYLASNRHTG |
| | VKGRFTISRDSAKNTLYLQMN | VPDRFTGSGSGTEFTLTIS |
| | SLRAEDTAVYYCARLGDDGM | NLQPEDFATYFCLQHN |
| | DWWGQGTTWTVSS | SYPLTFGGGTKVEIK |
| | (SEQ ID NO: 7) | (SEQ ID NO: 8) |
| | CDR1: SYYMY | CDR1: RASQNVRTAVA |
| | (SEQ ID NO: 109) | (SEQ ID NO: 112) |
| | CDR2: ISSGSEIYYSDSVKGR | CDR2: LASNRHT |
| | (SEQ ID NO: 110) | (SEQ ID NO: 113) |
| | CDR3: LGDDGMDW | CDR3: LQHNSYPLT |
| | (SEQ ID NO: 111) | (SEQ ID NO: 114) |
| huA21 | QVQLVQSGAEVVKPGASVKIS | ADIVLTQSPDSLAVSLGER |
| | CKASGYPFTQYFIHWVKQNPG | VTINCKSSQPLEYSNNQWN |
| | QRLEWIGQISSSYATVDYNQK | YLAWYQQKPGQSPKLLISW |
| | FKGKATLTVDTSASIAYMELS | ASTRKSGVPDRFSGSGSGT |
| | SLRSEDTAVYYCVRSGNYEEY | DFTLTISSVQAEDVAV |
| | AMDYWGQGTLVTVS | YYCGQYSDYPNTFGAG |
| | (SEQ ID NO: 11) | (SEQ ID NO: 10) |
| | CDR1: QYFIH | CDR1: KSSQPLEYSNNQWNYLA |
| | (SEQ ID NO: 115) | (SEQ ID NO: 118) |
| | CDR2: QISSSYATVDYNQKFKG | CDR2: WASTRKS |
| | (SEQ ID NO: 116) | (SEQ ID NO: 119) |
| | CDR3: SGNYEEYAMDY | CDR3: GQYSDYPNT |
| | (SEQ ID NO: 117) | (SEQ ID NO: 120) |
| 3H3B chA21 | EVQLQQSGPEVVKTGASVKIS | AAQPADIVLTQTPSSLPVS |
| | CKASGYSFTGYFINWVKKNSG | VGEKVTMTCKSSQTLLYSN |
| | KSPEWIGHISSSYATSTYNQKF | NQKNYLAWYQQKPGQSPKL |
| | KNKAAFTVDTSSSTAFMQLNS | LISWAFTRKSGVPDRFTGS |
| | LTSEDSAVYYCVRSGNYEEYA | GSGTDFTLTIGSVKAEDL |
| | MDYWGQGTSVTVSS | AVYYCQQYSNYPWTFGGG |
| | (SEQ ID NO: 14) | (SEQ ID NO: 13) |
| | CDR1: GYFIN | CDR1: KSSQTLLYSNNQKNYLA |
| | (SEQ ID NO: 121) | (SEQ ID NO: 124) |
| | CDR2: HISSSYATSTYNQKFKN | CDR2: WAFTRKS |
| | (SEQ ID NO: 122) | (SEQ ID NO: 125) |
| | CDR3: SGNYEEYAMDY | CDR3: QQYSNYPWT |
| | (SEQ ID NO: 123) | (SEQ ID NO: 126) |

TABLE 2B
αHER2 VH/VL Nucleotide Sequences

| Identifier | Nucleotide sequence |
|---|---|
| Pertuzamab VH | GAGGTGCAGCTGGTGGAGTCTGGGGGAGGCCTGGTGCAGCC |
| (SEQ ID NO: 40) | AGGGGGCTCACTGAGACTGAGCTGTGCTGCCTCTGGGTTTA |
| | CATTTACAGACTATACCATGGACTGGGTGAGACAGGCCCCA |
| | GGGAAGGGCCTGGAGTGGGTGGCAGATGTGAACCCAAATTC |
| | TGGAGGGTCCATCTACAATCAGAGGTTCAAGGGGAGATTTA |
| | CCCTGTCTGTGGATAGAAGCAAGAACACACTGTACCTTCAG |
| | ATGAACTCCCTGAGAGCTGAGGACACAGCTGTGTACTACTG |
| | TGCCAGAAACCTGGGCCCATCCTTTTACTTTGACTACTGGG |
| | GACAGGGCACACTGGTGACAGTCTCCTCT |
| Pertuzamab VL | GACATTCAGATGACTCAGTCTCCCAGCTCCCTGTCTGCCTC |
| (SEQ ID NO: 39) | TGTGGGAGACAGAGTGACAATCACATGTAAGGCCTCCCAGG |
| | ATGTGTCTATTGGAGTGGCCTGGTACCAGCAGAAACCTGGA |
| | AAGGCCCCAAAACTGCTGATCTACTCTGCCTCCTACAGGTA |
| | TACAGGAGTGCCTTCCAGATTCTCTGGCTCTGGCTCTGGCA |
| | CAGATTTCACTCTTACCATCAGTTCCCTCCAGCCTGAGGAT |
| | TTTGCTACCTACTATTGCCAGCAGTACTACATCTATCCTTA |
| | CACATTTGGCCAGGGA |
| Trastuzumab VH | GACATTCAGATGACACAGTCCCCATCTTCCCTGTCTGCCTC |
| (SEQ ID NO: 43) | TGTGGGAGACAGGGTGACAATCACTTGTAGGGCCAGCCAGG |
| | ATGTGAACACAGCTGTGGCCTGGTATCAGCAGAAACCTGGA |
| | AAGGCCCCAAAGCTGCTGATCTACTCAGCTTCCTTCCTGTA |
| | TTCTGGAGTGCCATCTAGGTTTTCTGGCTCCAGGTCTGGCA |
| | CAGACTTTACCCTGACCATCTCTTCTCTGCAGCCTGAGGAC |
| | TTTGCCACTTACTACTGTCAGCAGCATTACACAACCCCCCC |
| | AACCTTTGGCCAGGGC |
| Trastuzumab VL | GAGGTGCAGCTGGTGGAGTCTGGGGGAGGCCTGGTGCAGCC |
| (SEQ ID NO: 44) | TGGAGGCTCCCTGAGACTCTCCTGTGCTGCCTCTGGATTCA |
| | ATATCAAAGATACTTACATCCACTGGGTGAGACAGGCCCCT |
| | GGCAAAGGCCTGGAATGGGTGGCCAGAATCTATCCTACTAA |
| | TGGCTACACCAGATATGCTGACTCTGTGAAGGGCAGATTTA |
| | CAATCTCTGCTGACACCTCTAAAAATACAGCCTACCTGCAG |
| | ATGAATTCCCTGAGAGCTGAAGACACAGCAGTGTACTACTG |
| | CAGCAGGTGGGGTGGAGATGGCTTCTATGCCATGGACTATT |
| | GGGGCCAGGGCACCCTGGTGACAGTCTCA |
| Hersintuzumab | GAGGTAAAGCTGGTGGAGTCTGGAGGAGGCCTGGTGAAACC |
| VH | AGGGGGCTCTCTGAGACTGAGTTGTGCCACATCTGGCTTTT |
| (SEQ ID NO: 48) | CCTTTAGCAGCTATTATATGTACTGGGTGAGGCAGGCCCCA |
| | GGCAAAAGGCTGGAGTGGGTGGCCTACATCAGCTCTGGCTC |
| | TGAAATTTATTACTCTGACTCTGTCAAGGGCAGATTCACCA |
| | TCAGCAGAGATTCTGCCAAGAACACCCTGTATCTCCAGATG |
| | AACTCTCTGAGAGCTGAGGACACAGCTGTGTATTACTGTGC |
| | TAGACTGGGAGATGATGGAATGGACTGGTGGGGCCAGGGAA |
| | CCACATGGACAGTGTCTTCT |
| Hersintuzumab | GACATTGTGATGACACAGTCCCCCTCTAGCCTGTCTGCTTC |
| VL | TGTGGGAGACAGGGTGACAATCACATGCAGAGCCTCTCAGA |
| (SEQ ID NO: 47) | ATGTGAGAACAGCTGTGGCATGGTTCCAGCAGAAACCAGGA |
| | AAGGCCCCCAAGGCCCTGATCTACCTGGCCTCTAACAGGCA |
| | CACAGGGGTGCCTGATAGGTTCACAGGCTCTGGATCTGGGA |
| | CAGAGTTTACCCTGACCATCAGCAACCTGCAGCCTGAGGAC |
| | TTTGCTACATACTTCTGCCTGCAGCACAACAGCTATCCTCT |
| | GACTTTTGGAGGAGGCACCAAAGTGGAGATCAAG |
| huA21 VH | GCTGACATAGTGCTGACCCAGAGTCCTGATTCCCTGGCTGT |
| (SEQ ID NO: 35) | GTCTCTGGGAGAGAGAGTGACCATCAATTGCAAGTCCAGCC |
| | AGCCCCTGGAATACAGCAATAATCAGTGGAACTACCTGGCT |
| | TGGTACCAGCAGAAACCTGGACAGTCCCCTAAGCTGCTGAT |
| | TAGCTGGGCCAGCACCAGAAAGTCTGGAGTCCCAGACAGAT |
| | TTTCTGGTTCTGGCTCTGGAACTGACTTCACCCTCACAATC |
| | AGCTCTGTGCAGGCTGAAGATGTGGCTGTGTATTACTGTGG |
| | CCAGTACTCTGATTATCCCAACACCTTTGGGGCTGGC |
| huA21 VL | CAGGTGCAGCTGGTGCAGTCTGGAGCTGAGGTGGTGAAACC |
| (SEQ ID NO: 36) | AGGGGCTTCTGTGAAGATTTCCTGTAAGGCCTCTGGCTACC |
| | CTTTCACTCAGTACTTCATTCATTGGGTCAAGCAGAATCCT |
| | GGGCAGAGACTGGAATGGATTGGACAAATCTCCTCCAGCTA |
| | TGCCACAGTGGACTACAATCAGAAGTTCAAGGGCAAGGCAA |
| | CCCTGACTGTGGACACATCTGCCAGCATTGCCTATATGGAA |
| | CTGAGCAGCCTGAGATCTGAAGATACAGCTGTGTATTACTG |
| | TGTGAGATCTGGCAATTATGAGGAGTATGCCATGGACTACT |
| | GGGGACAGGGCACCCTGGTGACTGTGTCTGGAGGAGGAGGT |
| | TCTGACATCAAACTGCAGCAGAGTGGGGCTGAGCTGGCCAG |
| | ACCTGGAGCCTCTGTCAAGATGAGCTGCAAAACCTCAGGCT |
| | ACACATTCACCAGATACACCATGCACTGGGTGAAACAGAGA |
| | CCTGGACAGGGCCTGGAGTGGATTGGCTACATTAACCCATC |
| | AAGAGGCTATACCAACTATAACCAGAAATTCAAGGACAAGG |
| | CCACCCTGACCACAGACAAAAGCAG |
| 3H3B VH | GAAGTGCAGCTGCAGCAGAGCGGCCCGGAAGTGGTGAAAAC |
| (SEQ ID NO: 52) | CGGCGCGAGCGTGAAAATTAGCTGCAAAGCGAGCGGCTATA |
| | GCTTTACCGGCTATTTTATTAACTGGGTGAAAAAAAACAGC |
| | GGCAAAAGCCCGGAATGGATTGGCCATATTAGCAGCAGCTA |
| | TGCGACCAGCACCTATAACCAGAAATTTAAAAACAAAGCGG |
| | CGTTTACCGTGGATACCAGCAGCAGCACCGCGTTTATGCAG |
| | CTGAACAGCCTGACCAGCGAAGATAGCGCGGTGTATTATTG |
| | CGTGCGCAGCGGCAACTATGAAGAATATGCGATGGATTATT |
| | GGGGCCAGGGCACCAGCGTGACCGTGAGCAGC |
| 3H3B VL | GCGGCGCAGCCGGCGGATATTGTGCTGACCCAGACCCCGAG |
| (SEQ ID NO: 51) | CAGCCTGCCGGTGAGCGTGGGCGAAAAAGTGACCATGACCT |
| | GCAAAAGCAGCCAGACCCTGCTGTATAGCAACAACCAGAAA |
| | AACTATCTGGCGTGGTATCAGCAGAAACCGGGCCAGAGCCC |
| | GAAACTGCTGATTAGCTGGGCGTTTACCCGCAAAAGCGGCG |
| | TGCCGGATCGCTTTACCGGCAGCGGCAGCGGCACCGATTTT |
| | ACCCTGACCATTGGCAGCGTGAAAGCGGAAGATCTGGCGGT |
| | GTATTATTGCCAGCAGTATAGCAACTATCCGTGGACCTTTG |
| | GCGGCGGCACCCGCCTGGAAATTAAACGC |

In some embodiments, a HER2 binding site comprises VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A, determined under Kabat (see Kabat et al., (1991) Sequences of Proteins of Immunological Interest, NIH Publication No. 91-3242, Bethesda), IMGT unique numbering scheme, Chothia (see, e.g., Chothia C & Lesk A M, (1987), J. Mol. Biol. 196:901-917), MacCallum (see MacCallum R M et al., (1996) J. Mol. Biol. 262:732-745), or any other CDR determination method known in the art.

Unless indicated otherwise, the CDR sequences provided in TABLE 2A are determined under the Kabat numbering scheme.

In some embodiments, a HER2 binding site comprises: (i) a VL CDR1 comprising an amino acid sequence of SEQ ID NO: 100; (ii) a VL CDR2 comprising an amino acid sequence of SEQ ID NO: 101; (iii) a VL CDR3 comprising an amino acid sequence of SEQ ID NO: 102; (iv) a VH CDR1 comprising an amino acid sequence of SEQ ID NO: 97; (v) a VH CDR2 comprising an amino acid sequence of SEQ ID NO: 98; and (vi) a VH CDR3 comprising an amino acid sequence of SEQ ID NO: 99.

In some embodiments, a HER2 binding site comprises: (i) a VL CDR1 comprising an amino acid sequence of SEQ ID NO: 106; (ii) a VL CDR2 comprising an amino acid sequence of SEQ ID NO: 107; (iii) a VL CDR3 comprising an amino acid sequence of SEQ ID NO: 108; (iv) a VH CDR1 comprising an amino acid sequence of SEQ ID NO: 103; (v) a VH CDR2 comprising an amino acid sequence of SEQ ID NO: 104; and (vi) a VH CDR3 comprising an amino acid sequence of SEQ ID NO: 105.

In some embodiments, a HER2 binding site comprises: (i) a VL CDR1 comprising an amino acid sequence of SEQ ID NO: 112; (ii) a VL CDR2 comprising an amino acid sequence of SEQ ID NO: 113; (iii) a VL CDR3 comprising an amino acid sequence of SEQ ID NO: 114; (iv) a VH CDR1 comprising an amino acid sequence of SEQ ID NO: 109; (v) a VH CDR2 comprising an amino acid sequence of SEQ ID NO: 110; and (vi) a VH CDR3 comprising an amino acid sequence of SEQ ID NO: 111.

In some embodiments, a HER2 binding site comprises: (i) a VL CDR1 comprising an amino acid sequence of SEQ ID NO: 118; (ii) a VL CDR2 comprising an amino acid sequence of SEQ ID NO: 119; (iii) a VL CDR3 comprising an amino acid sequence of SEQ ID NO: 120; (iv) a VH CDR1 comprising an amino acid sequence of SEQ ID NO: 115; (v) a VH CDR2 comprising an amino acid sequence of SEQ ID NO: 116; and (vi) a VH CDR3 comprising an amino acid sequence of SEQ ID NO: 117.

In some embodiments, a HER2 binding site comprises: (i) a VL CDR1 comprising an amino acid sequence of SEQ ID NO: 124; (ii) a VL CDR2 comprising an amino acid sequence of SEQ ID NO: 125; (iii) a VL CDR3 comprising an amino acid sequence of SEQ ID NO: 126; (iv) a VH CDR1 comprising an amino acid sequence of SEQ ID NO: 121; (v) a VH CDR2 comprising an amino acid sequence of SEQ ID NO: 122; and (vi) a VH CDR3 comprising an amino acid sequence of SEQ ID NO: 123.

TABLE 2A additionally lists amino acid sequences of exemplary VL and VH domains that, in combination, can specifically bind to HER2. In some embodiments, HER2 binding sites of the present disclosure comprise VL domain and VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to VH domain and VL domain sequences listed in TABLE 2A. In some embodiments, the HER2 binding site comprises a VH comprising an amino acid sequence having at least 80% sequence identity with an amino acid sequence according to any one of SEQ ID NOs: 1, 4, 7, 11, and 14; and a VL comprising an amino acid sequence having at least 80% sequence identity with an amino acid sequence according to any one of SEQ ID NOs: 2, 5, 8, 10, and 13. In some embodiments, the HER2 binding site comprises a VH comprising an amino acid sequence having at least 85% sequence identity with an amino acid sequence according to any one of SEQ ID NOs: 1, 4, 7, 11, and 14; and a VL comprising an amino acid sequence having at least 85% sequence identity with an amino acid sequence according to any one of SEQ ID NOs: 2, 5, 8, 10, and 13. In some embodiments, the HER2 binding site comprises a VH comprising an amino acid sequence having at least 90% sequence identity with an amino acid sequence according to any one of SEQ ID NOs: 1, 4, 7, 11, and 14; and a VL comprising an amino acid sequence having at least 90% sequence identity with an amino acid sequence according to any one of SEQ ID NOs: 2, 5, 8, 10, and 13. In some embodiments, the HER2 binding site comprises a VH comprising an amino acid sequence having at least 95% sequence identity with an amino acid sequence according to any one of SEQ ID NOs: 1, 4, 7, 11, and 14; and a VL comprising an amino acid sequence having at least 95% sequence identity with an amino acid sequence according to any one of SEQ ID NOs: 2, 5, 8, 10, and 13. In some embodiments, the HER2 binding site comprises a VH comprising an amino acid sequence having at least 96% sequence identity with an amino acid sequence according to any one of SEQ ID NOs: 1, 4, 7, 11, and 14; and a VL comprising an amino acid sequence having at least 96% sequence identity with an amino acid sequence according to any one of SEQ ID NOs: 2, 5, 8, 10, and 13. In some embodiments, the HER2 binding site comprises a VH comprising an amino acid sequence having at least 97% sequence identity with an amino acid sequence according to any one of SEQ ID NOs: 1, 4, 7, 11, and 14; and a VL comprising an amino acid sequence having at least 97% sequence identity with an amino acid sequence according to any one of SEQ ID NOs: 2, 5, 8, 10, and 13. In some embodiments, the HER2 binding site comprises a VH comprising an amino acid sequence having at least 98% sequence identity with an amino acid sequence according to any one of SEQ ID NOs: 1, 4, 7, 11, and 14; and a VL comprising an amino acid sequence having at least 98% sequence identity with an amino acid sequence according to any one of SEQ ID NOs: 2, 5, 8, 10, and 13. In some embodiments, the HER2 binding site comprises a VH comprising an amino acid sequence having at least 99% sequence identity with an amino acid sequence according to any one of SEQ ID NOs: 1, 4, 7, 11, and 14; and a VL comprising an amino acid sequence having at least 99% sequence identity with an amino acid sequence according any one of SEQ ID NOs: 2, 5, 8, 10, and 13. In some embodiments, the HER2 binding site comprises a VH comprising an amino acid sequence according to any one of SEQ ID NOs: 1, 4, 7, 11, and 14; and a VL comprising an amino acid sequence according to any one of SEQ ID NOs: 2, 5, 8, 10, and 13.

In some embodiments, a HER2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 1; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 2. In some embodiments, the HER2 binding site comprises a VH comprising an amino acid sequence having at least 80% sequence identity with an amino acid sequence according to SEQ ID NO: 1; and a VL comprising an amino acid sequence having at least 80% sequence identity with an amino acid sequence according to SEQ ID NO: 2. In some embodiments, the HER2 binding site comprises a VH comprising an amino acid sequence having at least 85% sequence identity with an amino acid sequence according to SEQ ID NO: 1; and a VL comprising an amino acid sequence having at least 85% sequence identity with an amino acid sequence according to SEQ ID NO: 2. In some embodiments, the HER2 binding site comprises a VH comprising an amino acid sequence having at least 90% sequence identity with an amino acid sequence according to SEQ ID NO: 1; and a VL comprising an amino acid sequence having at least 90% sequence identity with an amino acid sequence according to SEQ ID NO: 2. In some embodiments, the HER2 binding site comprises a VH comprising an amino acid sequence having at least 95% sequence identity with an amino acid sequence according to SEQ ID NO: 1; and a VL comprising an amino acid sequence having at least 95% sequence identity with an amino acid sequence according to SEQ ID NO: 2. In some embodiments, the HER2 binding site comprises a VH comprising an amino acid sequence having at least 96% sequence identity with an amino acid sequence according to SEQ ID NO: 1; and a VL comprising an amino acid sequence having at least 96% sequence identity with an amino acid sequence according to SEQ ID NO: 2. In some embodiments, the HER2 binding site comprises a VH comprising an amino acid sequence having at least 97% sequence identity with an amino acid sequence according to SEQ ID NO: 1; and a VL comprising an amino acid sequence having at least 97% sequence identity with an amino acid sequence according to SEQ ID NO: 2. In some embodiments, the HER2 binding site comprises a VH comprising an amino acid sequence having at least 98% sequence identity with an amino acid sequence according to SEQ ID NO: 1; and a VL comprising an amino acid sequence having at least 98% sequence identity with an amino acid sequence according to SEQ ID NO: 2. In some embodiments, the HER2 binding site comprises a VH comprising an amino acid sequence having at least 99% sequence identity with an amino acid sequence according to SEQ ID NO: 1; and a VL comprising an amino acid sequence having at least 99% sequence identity with an amino acid sequence according SEQ ID NO: 2. In some embodiments, the HER2 binding site comprises a VH comprising an amino acid sequence according to SEQ ID NO: 1; and a VL comprising an amino acid sequence according to SEQ ID NO: 2.

In some embodiments, a HER2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 4; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5. In some embodiments, the HER2 binding site comprises a VH comprising an amino acid sequence having at least 80% sequence identity with an amino acid sequence according to SEQ ID NO: 4; and a VL comprising an amino acid sequence having at least 80% sequence identity with an amino acid sequence according to SEQ ID NO: 5. In some embodiments, the HER2 binding site comprises a VH comprising an amino acid sequence having at least 85% sequence identity with an amino acid sequence according to SEQ ID NO: 4; and a VL comprising an amino acid sequence having at least 85% sequence identity with an amino acid sequence according to SEQ ID NO: 5. In some embodiments, the HER2 binding site comprises a VH comprising an amino acid sequence having at least 90% sequence identity with an amino acid sequence according to SEQ ID NO: 4; and a VL comprising an amino acid sequence having at least 90% sequence identity with an amino acid sequence according to SEQ ID NO: 5. In some embodiments, the HER2 binding site comprises a VH comprising an amino acid sequence having at least 95% sequence identity with an amino acid sequence according to SEQ ID NO: 4; and a VL comprising an amino acid sequence having at least 95% sequence identity with an amino acid sequence according to SEQ ID NO: 5. In some embodiments, the HER2 binding site comprises a VH comprising an amino acid sequence having at least 96% sequence identity with an amino acid sequence according to SEQ ID NO: 4; and a VL comprising an amino acid sequence having at least 96% sequence identity with an amino acid sequence according to SEQ ID NO: 5. In some embodiments, the HER2 binding site comprises a VH comprising an amino acid sequence having at least 97% sequence identity with an amino acid sequence according to SEQ ID NO: 4; and a VL comprising an amino acid sequence having at least 97% sequence identity with an amino acid sequence according to SEQ ID NO: 5. In some embodiments, the HER2 binding site comprises a VH comprising an amino acid sequence having at least 98% sequence identity with an amino acid sequence according to SEQ ID NO: 4; and a VL comprising an amino acid sequence having at least 98% sequence identity with an amino acid sequence according to SEQ ID NO: 5. In some embodiments, the HER2 binding site comprises a VH comprising an amino acid sequence having at least 99% sequence identity with an amino acid sequence according to SEQ ID NO: 4; and a VL comprising an amino acid sequence having at least 99% sequence identity with an amino acid sequence according SEQ ID NO: 5. In some embodiments, the HER2 binding site comprises a VH comprising an amino acid sequence according to SEQ ID NO: 4; and a VL comprising an amino acid sequence according to SEQ ID NO: 5.

In some embodiments, a HER2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 8. In some embodiments, the HER2 binding site comprises a VH comprising an amino acid sequence having at least 80% sequence identity with an amino acid sequence according to SEQ ID NO: 7; and a VL comprising an amino acid sequence having at least 80% sequence identity with an amino acid sequence according to SEQ ID NO: 8. In some embodiments, the HER2 binding site comprises a VH comprising an amino acid sequence having at least 85% sequence identity with an amino acid sequence according to SEQ ID NO: 7; and a VL comprising an amino acid sequence having at least 85% sequence identity with an amino acid sequence according to SEQ ID NO: 8. In some embodiments, the HER2 binding site comprises a VH comprising an amino acid sequence having at least 90% sequence identity with an amino acid sequence according to SEQ ID NO: 7; and a VL comprising an amino acid sequence having at least 90% sequence identity with an amino acid sequence according to SEQ ID NO: 8. In some embodiments, the HER2 binding site comprises a VH comprising an amino acid sequence having at least 95% sequence identity with an amino acid sequence according to SEQ ID NO: 7; and a VL comprising an amino acid sequence having at least 95% sequence identity with an amino acid sequence according to SEQ ID NO: 8. In some embodiments, the HER2 binding site comprises a VH comprising an amino acid sequence having at least 96% sequence identity with an amino acid sequence according to SEQ ID NO: 7; and a VL comprising an amino acid sequence having at least 96% sequence identity with an amino acid sequence according to SEQ ID NO: 8. In some embodiments, the HER2 binding site comprises a VH comprising an amino acid sequence having at least 97% sequence identity with an amino acid sequence according to SEQ ID NO: 7; and a VL comprising an amino acid sequence having at least 97% sequence identity with an amino acid sequence according to SEQ ID NO: 8. In some embodiments, the HER2 binding site comprises a VH comprising an amino acid sequence having at least 98% sequence identity with an amino acid sequence according to SEQ ID NO: 7; and a VL comprising an amino acid sequence having at least 98% sequence identity with an amino acid sequence according to SEQ ID NO: 8. In some embodiments, the HER2 binding site comprises a VH comprising an amino acid sequence having at least 99% sequence identity with an amino acid sequence according to SEQ ID NO: 7; and a VL comprising an amino acid sequence having at least 99% sequence identity with an amino acid sequence according SEQ ID NO: 8. In some embodiments, the HER2 binding site comprises a VH comprising an amino acid sequence according to SEQ ID NO: 7; and a VL comprising an amino acid sequence according to SEQ ID NO: 8.

In some embodiments, a HER2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11; (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10. In some embodiments, the HER2 binding site comprises a VH comprising an amino acid sequence having at least 80% sequence identity with an amino acid sequence according to SEQ ID NO: 11; and a VL comprising an amino acid sequence having at least 80% sequence identity with an amino acid sequence according to SEQ ID NO: 10. In some embodiments, the HER2 binding site comprises a VH comprising an amino acid sequence having at least 85% sequence identity with an amino acid sequence according to SEQ ID NO: 11; and a VL comprising an amino acid sequence having at least 85% sequence identity with an amino acid sequence according to SEQ ID NO: 10. In some embodiments, the HER2 binding site comprises a VH comprising an amino acid sequence having at least 90% sequence identity with an amino acid sequence according to SEQ ID NO: 11; and a VL comprising an amino acid sequence having at least 90% sequence identity with an amino acid sequence according to SEQ ID NO: 10. In some embodiments, the HER2 binding site comprises a VH comprising an amino acid sequence having at least 95% sequence identity with an amino acid sequence according to SEQ ID NO: 11; and a VL comprising an amino acid sequence having at least 95% sequence identity with an amino acid sequence according to SEQ ID NO: 10. In some embodiments, the HER2 binding site comprises a VH comprising an amino acid sequence having at least 96% sequence identity with an amino acid sequence according to SEQ ID NO: 11; and a VL comprising an amino acid sequence having at least 96% sequence identity with an amino acid sequence according to SEQ ID NO: 10. In some embodiments, the HER2 binding site comprises a VH comprising an amino acid sequence having at least 97% sequence identity with an amino acid sequence according to SEQ ID NO: 11; and a VL comprising an amino acid sequence having at least 97% sequence identity with an amino acid sequence according to SEQ ID NO: 10. In some embodiments, the HER2 binding site comprises a VH comprising an amino acid sequence having at least 98% sequence identity with an amino acid sequence according to SEQ ID NO: 11; and a VL comprising an amino acid sequence having at least 98% sequence identity with an amino acid sequence according to SEQ ID NO: 10. In some embodiments, the HER2 binding site comprises a VH comprising an amino acid sequence having at least 99% sequence identity with an amino acid sequence according to SEQ ID NO: 11; and a VL comprising an amino acid sequence having at least 99% sequence identity with an amino acid sequence according SEQ ID NO: 10. In some embodiments, the HER2 binding site comprises a VH comprising an amino acid sequence according to SEQ ID NO: 11; and a VL comprising an amino acid sequence according to SEQ ID NO: 10.

In some embodiments, a HER2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 14; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13. In some embodiments, the HER2 binding site comprises a VH comprising an amino acid sequence having at least 80% sequence identity with an amino acid sequence according to SEQ ID NO: 14; and a VL comprising an amino acid sequence having at least 80% sequence identity with an amino acid sequence according to SEQ ID NO: 13. In some embodiments, the HER2 binding site comprises a VH comprising an amino acid sequence having at least 85% sequence identity with an amino acid sequence according to SEQ ID NO: 14; and a VL comprising an amino acid sequence having at least 85% sequence identity with an amino acid sequence according to SEQ ID NO: 13. In some embodiments, the HER2 binding site comprises a VH comprising an amino acid sequence having at least 90% sequence identity with an amino acid sequence according to SEQ ID NO: 14; and a VL comprising an amino acid sequence having at least 90% sequence identity with an amino acid sequence according to SEQ ID NO: 13. In some embodiments, the HER2 binding site comprises a VH comprising an amino acid sequence having at least 95% sequence identity with an amino acid sequence according to SEQ ID NO: 14; and a VL comprising an amino acid sequence having at least 95% sequence identity with an amino acid sequence according to SEQ ID NO: 13. In some embodiments, the HER2 binding site comprises a VH comprising an amino acid sequence having at least 96% sequence identity with an amino acid sequence according to SEQ ID NO: 14; and a VL comprising an amino acid sequence having at least 96% sequence identity with an amino acid sequence according to SEQ ID NO: 13. In some embodiments, the HER2 binding site comprises a VH comprising an amino acid sequence having at least 97% sequence identity with an amino acid sequence according to SEQ ID NO: 14; and a VL comprising an amino acid sequence having at least 97% sequence identity with an amino acid sequence according to SEQ ID NO: 13. In some embodiments, the HER2 binding site comprises a VH comprising an amino acid sequence having at least 98% sequence identity with an amino acid sequence according to SEQ ID NO: 14; and a VL comprising an amino acid sequence having at least 98% sequence identity with an amino acid sequence according to SEQ ID NO: 13. In some embodiments, the HER2 binding site comprises a VH comprising an amino acid sequence having at least 99% sequence identity with an amino acid sequence according to SEQ ID NO: 14; and a VL comprising an amino acid sequence having at least 99% sequence identity with an amino acid sequence according SEQ ID NO: 13. In some embodiments, the HER2 binding site comprises a VH comprising an amino acid sequence according to SEQ ID NO: 14; and a VL comprising an amino acid sequence according to SEQ ID NO: 13.

In some embodiments, a HER2 binding site comprises, but is not limited to, a single-chain variable fragment (scFv), an antibody, a Fab, a Fab′, a F(ab′)₂, a minibody, or a nanobody (VHH). For example, in some embodiments, bispecific fusion proteins of the present disclosure comprises an scFv polypeptide that each specifically binds to HER2.

In some embodiments, a HER2 binding site of the present disclosure is in an scFv format. In some embodiments, a HER2-binding scFv of the present disclosure comprises an scFv linker polypeptide that operably connects a VH domain and a VL domain. For example, in some embodiments, a HER2 binding scFv comprises, from N-terminus to C-terminus, a VL domain of an anti-HER2 antibody, an scFv linker polypeptide, and a VH domain of an anti-HER2 antibody. In other embodiments, a HER2 binding scFv comprises, from N-terminus to C-terminus, a VH domain of an anti-HER2 antibody, an scFv linker polypeptide, and a VL domain of an anti-HER2 antibody.

In some embodiments, an scFv linker polypeptide comprises a sequence selected from the linker sequences in TABLE 3A.

TABLE 3A
scFv Linker Peptide Sequences

| scFv Linker | Sequence |
|---|---|
| Linker 1 | SCGGGSGGGGSGGGGS |
| (SEQ ID NO: 24) | |
| Linker 2 | GGGGSGGGGSGGGGSGGGG |
| (SEQ ID NO: 25) | |
| Linker 3 | GGGSGGGGSGGGGS |
| (SEQ ID NO: 26) | |
| Linker 4 | GGGGSGGGGSGGGGSGGGGS |
| (SEQ ID NO: 27) | |
| Linker 5 | GGSGGSGGSGGS |
| (SEQ ID NO: 28) | |

In some embodiments, a HER2 binding scFv of the present disclosure comprises a spacer peptide fused to the N-terminus of the scFv linker peptide, at the C-terminus of the VH domain, or at the C-terminus of the VL domain. In some embodiments, the spacer peptide comprises a sequence selected from the spacer sequences listed in TABLE 3B.

TABLE 3B
Spacer Peptide sequences

| Spacer | Sequence |
|---|---|
| Spacer 1 | TKVEIK |
| (SEQ ID NO: 30) | |
| Spacer 2 | TKLEIKR |
| (SEQ ID NO: 31) | |
| Spacer 3 | TKLELK |
| (SEQ ID NO: 32) | |

TABLE 4A lists amino acid sequences of exemplary HER2-binding scFvs. In some embodiments, bispecific fusion proteins of the present disclosure comprise a HER2-binding scFv comprising a sequence at least 85% identical (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 4A. TABLE 4B lists the corresponding nucleotide sequences of the exemplary HER2-binding scFvs.

TABLE 4A
HER2-binding scFv Amino Acid Sequences

| Identifier | scFv sequence |
|---|---|
| Pertuzumab Anti- | EVQLVESGGGLVQPGGSLRLSCAASGFTFTDYTMDWVRQAPGK |
| Her2 scFv | GLEWVADVNPNSGGSIYNQRFKGRFTLSVDRSKNTLYLQMNSL |
| (VH-VL) | RAEDTAVYYCARNLGPSFYFDYWGQGTLVTVSSSCGGGSGGGG |
| (SEQ ID NO: 3) | SGGGGSDIQMTQSPSSLSASVGDRVTITCKASQDVSIGVAWYQ |
| | QKPGKAPKLLIYSASYRYTGVPSRFSGSGSGTDFTLTISSLQP |
| | EDFATYYCQQYYIYPYTFGQGTKVEIK |
| Pertuzumab Anti- | DIQMTQSPSSLSASVGDRVTITCKASQDVSIGVAWYQQKPGKA |
| Her2 scFv | PKLLIYSASYRYTGVPSRFSGSGSGTDFTLTISSLQPEDFATY |
| (VL-VH) | YCQQYYIYPYTFGQGTKVEIKSCGGGSGGGGSGGGGSEVQLVE |
| (SEQ ID NO: 88) | SGGGLVQPGGSLRLSCAASGFTFTDYTMDWVRQAPGKGLEWVA |
| | DVNPNSGGSIYNQRFKGRFTLSVDRSKNTLYLQMNSLRAEDTA |
| | VYYCARNLGPSFYFDYWGQGTLVTVSS |
| Herceptin | EVQLVESGGGLVQPGGSLRLSCAASGFNIKDTYIHWVRQAPGK |
| (Trastuzumab) | GLEWVARIYPTNGYTRYADSVKGRFTISADTSKNTAYLQMNSL |
| Anti-Her2 scFv- | RAEDTAVYYCSRWGGDGFYAMDYWGQGTLVTVSSGGGGSGGGG |
| (VH-VL) | SGGGGSGGGGSDIQMTQSPSSLSASVGDRVTITCRASQDVNTA |
| (SEQ ID NO: 6) | VAWYQQKPGKAPKLLIYSASFLYSGVPSRFSGSRSGTDFTLTI |
| | SSLQPEDFATYYCQQHYTTPPTFGQGTKVEIK |
| Herceptin | DIQMTQSPSSLSASVGDRVTITCRASQDVNTAVAWYQQKPGKA |
| (Trastuzumab) | PKLLIYSASFLYSGVPSRFSGSRSGTDFTLTISSLQPEDFATY |
| Anti-Her2 scFv- | YCQQHYTTPPTFGQGTKVEIKGGGGSGGGGSGGGGSGGGGSEV |
| (VL-VH) | QLVESGGGLVQPGGSLRLSCAASGFNIKDTYIHWVRQAPGKGL |
| (SEQ ID NO: 89) | EWVARIYPTNGYTRYADSVKGRFTISADTSKNTAYLQMNSLRA |
| | EDTAVYYCSRWGGDGFYAMDYWGQGTLVTVSS |
| Hersintuzumab | EVKLVESGGGLVKPGGSLRLSCATSGFSFSSYYMYWVRQAPGK |
| Anti-Her2 scFv | RLEWVAYISSGSEIYYSDSVKGRFTISRDSAKNTLYLQMNSLR |
| (VH-VL) | AEDTAVYYCARLGDDGMDWWGQGTTWTVSSGGGSGGGGSGGGG |
| (SEQ ID NO: 9) | SDIVMTQSPSSLSASVGDRVTITCRASQNVRTAVAWFQQKPGK |
| | APKALIYLASNRHTGVPDRFTGSGSGTEFTLTISNLQPEDFAT |
| | YFCLQHNSYPLTFGGGTKVEIK |
| Hersintuzumab | DIVMTQSPSSLSASVGDRVTITCRASQNVRTAVAWFQQKPGKA |
| Anti-Her2 scFv | PKALIYLASNRHTGVPDRFTGSGSGTEFTLTISNLQPEDFATY |
| (VL-VH) | FCLQHNSYPLTFGGGTKVEIKGGGGSGGGGSGGGGSEVKLVES |
| (SEQ ID NO: 90) | GGGLVKPGGSLRLSCATSGFSFSSYYMYWVRQAPGKRLEWVAY |
| | ISSGSEIYYSDSVKGRFTISRDSAKNTLYLQMNSLRAEDTAVY |
| | YCARLGDDGMDWWGQGTTWTVSS |
| huA21 | QVQLVQSGAEVVKPGASVKISCKASGYPFTQYFIHWVKQNPGQ |
| Anti-Her2 scFv | RLEWIGQISSSYATVDYNQKFKGKATLTVDTSASIAYMELSSL |
| (VH-VL) | RSEDTAVYYCVRSGNYEEYAMDYWGQGTLVTVSTKLEIKRGGG |
| (SEQ ID NO: 140) | GSGGGGSGGGGSGGGGSADIVLTQSPDSLAVSLGERVTINCKS |
| | SQPLEYSNNQWNYLAWYQQKPGQSPKLLISWASTRKSGVPDRF |
| | SGSGSGTDFTLTISSVQAEDVAVYYCGQYSDYPNTFGAG |
| huA21 | ADIVLTQSPDSLAVSLGERVTINCKSSQPLEYSNNQWNYLAWY |
| Anti-Her2 scFv | QQKPGQSPKLLISWASTRKSGVPDRFSGSGSGTDFTLTISSVQ |
| (VL-VH) | AEDVAVYYCGQYSDYPNTFGAGTKLEIKRGGGGSGGGGSGGGG |
| (SEQ ID NO: 12) | SGGGGSQVQLVQSGAEVVKPGASVKISCKASGYPFTQYFIHWV |
| | KQNPGQRLEWIGQISSSYATVDYNQKFKGKATLTVDTSASIAY |
| | MELSSLRSEDTAVYYCVRSGNYEEYAMDYWGQGTLVTVS |
| 3H3B chA21 | EVQLQQSGPEVVKTGASVKISCKASGYSFTGYFINWVKKNSGK |
| Anti-Her2 scFv | SPEWIGHISSSYATSTYNQKFKNKAAFTVDTSSSTAFMQLNSL |
| (VH-VL) | TSEDSAVYYCVRSGNYEEYAMDYWGQGTSVTVSSTRLEIKRGG |
| (SEQ ID NO: 141) | GGSGGGGSGGGGSGGGGSAAQPADIVLTQTPSSLPVSVGEKVT |
| | MTCKSSQTLLYSNNQKNYLAWYQQKPGQSPKLLISWAFTRKSG |
| | VPDRFTGSGSGTDFTLTIGSVKAEDLAVYYCQQYSNYPWTFGG |
| | G |
| 3H3B chA21 | AAQPADIVLTQTPSSLPVSVGEKVTMTCKSSQTLLYSNNQKNY |
| Anti-Her2 scFv | LAWYQQKPGQSPKLLISWAFTRKSGVPDRFTGSGSGTDFTLTI |
| (VL-VH) | GSVKAEDLAVYYCQQYSNYPWTFGGGTRLEIKRGGGGSGGGGS |
| (SEQ ID NO: 15) | GGGGSGGGGSEVQLQQSGPEVVKTGASVKISCKASGYSFTGYF |
| | INWVKKNSGKSPEWIGHISSSYATSTYNQKFKNKAAFTVDTSS |
| | STAFMQLNSLTSEDSAVYYCVRSGNYEEYAMDYWGQGTSVTVS |
| | S |

Underlined italicized text indicates scFv linker sequence;
underlined text indicates spacer sequence.

TABLE 4B
HER2-binding scFv Nucleotide Sequences

| Identifier | Nucleotide sequence |
|---|---|
| Pertuzumab | GACATTCAGATGACTCAGTCTCCCAGCTCCCTGTCTGCCTC |
| Anti-Her2 scFv | TGTGGGAGACAGAGTGACAATCACATGTAAGGCCTCCCAGG |
| (VL-VH) | ATGTGTCTATTGGAGTGGCCTGGTACCAGCAGAAACCTGGA |
| (SEQ ID NO: 54) | AAGGCCCCAAAACTGCTGATCTACTCTGCCTCCTACAGGTA |
| | TACAGGAGTGCCTTCCAGATTCTCTGGCTCTGGCTCTGGCA |
| | CAGATTTCACTCTTACCATCAGTTCCCTCCAGCCTGAGGAT |
| | TTTGCTACCTACTATTGCCAGCAGTACTACATCTATCCTTA |
| | CACATTTGGCCAGGGAACAAAAGTGGAGATCAAGTCCTGTG |
| | GAGGGGGATCTGGGGGAGGGGGGTCAGGAGGGGGAGGCTCT |
| | GAGGTGCAGCTGGTGGAGTCTGGGGGAGGCCTGGTGCAGCC |
| | AGGGGGCTCACTGAGACTGAGCTGTGCTGCCTCTGGGTTTA |
| | CATTTACAGACTATACCATGGACTGGGTGAGACAGGCCCCA |
| | GGGAAGGGCCTGGAGTGGGTGGCAGATGTGAACCCAAATTC |
| | TGGAGGGTCCATCTACAATCAGAGGTTCAAGGGGAGATTTA |
| | CCCTGTCTGTGGATAGAAGCAAGAACACACTGTACCTTCAG |
| | ATGAACTCCCTGAGAGCTGAGGACACAGCTGTGTACTACTG |
| | TGCCAGAAACCTGGGCCCATCCTTTTACTTTGACTACTGGG |
| | GACAGGGCACACTGGTGACAGTCTCCTCT |
| Herceptin | GACATTCAGATGACACAGTCCCCATCTTCCCTGTCTGCCTC |
| (Trastuzumab) | TGTGGGAGACAGGGTGACAATCACTTGTAGGGCCAGCCAGG |
| Anti-Her2 scFv | ATGTGAACACAGCTGTGGCCTGGTATCAGCAGAAACCTGGA |
| (VL-VH) | AAGGCCCCAAAGCTGCTGATCTACTCAGCTTCCTTCCTGTA |
| (SEQ ID NO: 55) | TTCTGGAGTGCCATCTAGGTTTTCTGGCTCCAGGTCTGGCA |
| | CAGACTTTACCCTGACCATCTCTTCTCTGCAGCCTGAGGAC |
| | TTTGCCACTTACTACTGTCAGCAGCATTACACAACCCCCCC |
| | AACCTTTGGCCAGGGCACCAAGGTGGAGATCAAGGGAGGAG |
| | GGGGAAGTGGGGGAGGAGGATCTGGAGGAGGGGGATCAGGA |
| | GGGGGAGGCTCAGAGGTGCAGCTGGTGGAGTCTGGGGGAGG |
| | CCTGGTGCAGCCTGGAGGCTCCCTGAGACTCTCCTGTGCTG |
| | CCTCTGGATTCAATATCAAAGATACTTACATCCACTGGGTG |
| | AGACAGGCCCCTGGCAAAGGCCTGGAATGGGTGGCCAGAAT |
| | CTATCCTACTAATGGCTACACCAGATATGCTGACTCTGTGA |
| | AGGGCAGATTTACAATCTCTGCTGACACCTCTAAAAATACA |
| | GCCTACCTGCAGATGAATTCCCTGAGAGCTGAAGACACAGC |
| | AGTGTACTACTGCAGCAGGTGGGGTGGAGATGGCTTCTATG |
| | CCATGGACTATTGGGGCCAGGGCACCCTGGTGACAGTCTCA |
| | TCT |
| Hersintuzumab | GACATTGTGATGACACAGTCCCCCTCTAGCCTGTCTGCTTC |
| Anti-Her2 scFv | TGTGGGAGACAGGGTGACAATCACATGCAGAGCCTCTCAGA |
| (VL-VH) | ATGTGAGAACAGCTGTGGCATGGTTCCAGCAGAAACCAGGA |
| (SEQ ID NO: 56) | AAGGCCCCCAAGGCCCTGATCTACCTGGCCTCTAACAGGCA |
| | CACAGGGGTGCCTGATAGGTTCACAGGCTCTGGATCTGGGA |
| | CAGAGTTTACCCTGACCATCAGCAACCTGCAGCCTGAGGAC |
| | TTTGCTACATACTTCTGCCTGCAGCACAACAGCTATCCTCT |
| | GACTTTTGGAGGAGGCACCAAAGTGGAGATCAAGGGAGGAG |
| | GAGGTTCTGGGGGAGGAGGATCTGGAGGAGGAGGATCTGAG |
| | GTAAAGCTGGTGGAGTCTGGAGGAGGCCTGGTGAAACCAGG |
| | GGGCTCTCTGAGACTGAGTTGTGCCACATCTGGCTTTTCCT |
| | TTAGCAGCTATTATATGTACTGGGTGAGGCAGGCCCCAGGC |
| | AAAAGGCTGGAGTGGGTGGCCTACATCAGCTCTGGCTCTGA |
| | AATTTATTACTCTGACTCTGTCAAGGGCAGATTCACCATCA |
| | GCAGAGATTCTGCCAAGAACACCCTGTATCTCCAGATGAAC |
| | TCTCTGAGAGCTGAGGACACAGCTGTGTATTACTGTGCTAG |
| | ACTGGGAGATGATGGAATGGACTGGTGGGGCCAGGGAACCA |
| | CATGGACAGTGTCTTCT |
| huA21 | GCTGACATAGTGCTGACCCAGAGTCCTGATTCCCTGGCTGT |
| Anti-Her2 scFv | GTCTCTGGGAGAGAGAGTGACCATCAATTGCAAGTCCAGCC |
| (VL-VH) | AGCCCCTGGAATACAGCAATAATCAGTGGAACTACCTGGCT |
| (SEQ ID NO: 53) | TGGTACCAGCAGAAACCTGGACAGTCCCCTAAGCTGCTGAT |
| | TAGCTGGGCCAGCACCAGAAAGTCTGGAGTCCCAGACAGAT |
| | TTTCTGGTTCTGGCTCTGGAACTGACTTCACCCTCACAATC |
| | AGCTCTGTGCAGGCTGAAGATGTGGCTGTGTATTACTGTGG |
| | CCAGTACTCTGATTATCCCAACACCTTTGGGGCTGGCACAA |
| | AGCTGGAAATCAAGAGAGGAGGTGGAGGTTCTGGAGGAGGG |
| | GGATCTGGTGGAGGGGGCTCTGGAGGAGGAGGGTCTCAGGT |
| | GCAGCTGGTGCAGTCTGGAGCTGAGGTGGTGAAACCAGGGG |
| | CTTCTGTGAAGATTTCCTGTAAGGCCTCTGGCTACCCTTTC |
| | ACTCAGTACTTCATTCATTGGGTCAAGCAGAATCCTGGGCA |
| | GAGACTGGAATGGATTGGACAAATCTCCTCCAGCTATGCCA |
| | CAGTGGACTACAATCAGAAGTTCAAGGGCAAGGCAACCCTG |
| | ACTGTGGACACATCTGCCAGCATTGCCTATATGGAACTGAG |
| | CAGCCTGAGATCTGAAGATACAGCTGTGTATTACTGTGTGA |
| | GATCTGGCAATTATGAGGAGTATGCCATGGACTACTGGGGA |
| | CAGGGCACCCTGGTGACTGTGTCT |
| 3H3B chA21 | GCGGCGCAGCCGGCGGATATTGTGCTGACCCAGACCCCGAG |
| Anti-Her2 scFv | CAGCCTGCCGGTGAGCGTGGGCGAAAAAGTGACCATGACCT |
| (VL-VH) | GCAAAAGCAGCCAGACCCTGCTGTATAGCAACAACCAGAAA |
| (SEQ ID NO: 57) | AACTATCTGGCGTGGTATCAGCAGAAACCGGGCCAGAGCCC |
| | GAAACTGCTGATTAGCTGGGCGTTTACCCGCAAAAGCGGCG |
| | TGCCGGATCGCTTTACCGGCAGCGGCAGCGGCACCGATTTT |
| | ACCCTGACCATTGGCAGCGTGAAAGCGGAAGATCTGGCGGT |
| | GTATTATTGCCAGCAGTATAGCAACTATCCGTGGACCTTTG |
| | GCGGCGGCACCCGCCTGGAAATTAAACGCGGCGGCGGCGGC |
| | AGCGGCGGCGGCGGCAGCGGCGGCGGCGGCAGCGGCGGCGG |
| | CGGCAGCGAAGTGCAGCTGCAGCAGAGCGGCCCGGAAGTGG |
| | TGAAAACCGGCGCGAGCGTGAAAATTAGCTGCAAAGCGAGC |
| | GGCTATAGCTTTACCGGCTATTTTATTAACTGGGTGAAAAA |
| | AAACAGCGGCAAAAGCCCGGAATGGATTGGCCATATTAGCA |
| | GCAGCTATGCGACCAGCACCTATAACCAGAAATTTAAAAAC |
| | AAAGCGGCGTTTACCGTGGATACCAGCAGCAGCACCGCGTT |
| | TATGCAGCTGAACAGCCTGACCAGCGAAGATAGCGCGGTGT |
| | ATTATTGCGTGCGCAGCGGCAACTATGAAGAATATGCGATG |
| | GATTATTGGGGCCAGGGCACCAGCGTGACCGTGAGCAGC |

Underlined text indicates scFv linker sequence, or spacer sequence and scFv linker sequence.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 3. In some embodiments, the scFv that specifically binds HER2 comprises a sequence having at least about 85% identity to SEQ ID NO: 3. In some embodiments, the scFv that specifically binds HER2 comprises a sequence having at least about 90% identity to SEQ ID NO: 3. In some embodiments, the scFv that specifically binds HER2 comprises a sequence having at least about 95% identity to SEQ ID NO: 3. In some embodiments, the scFv that specifically binds HER2 comprises a sequence having at least about 96% identity to SEQ ID NO: 3. In some embodiments, the scFv that specifically binds HER2 comprises a sequence having at least about 97% identity to SEQ ID NO: 3. In some embodiments, the scFv that specifically binds HER2 comprises a sequence having at least about 98% identity to SEQ ID NO: 3. In some embodiments, the scFv that specifically binds HER2 comprises a sequence having at least about 99% identity to SEQ ID NO: 3. In some embodiments, the scFv that specifically binds HER2 comprises a sequence having 100% identity to SEQ ID NO: 3.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88. In some embodiments, the scFv that specifically binds HER2 comprises a sequence having at least about 85% identity to SEQ ID NO: 88. In some embodiments, the scFv that specifically binds HER2 comprises a sequence having at least about 90% identity to SEQ ID NO: 88. In some embodiments, the scFv that specifically binds HER2 comprises a sequence having at least about 95% identity to SEQ ID NO: 88. In some embodiments, the scFv that specifically binds HER2 comprises a sequence having at least about 96% identity to SEQ ID NO: 88. In some embodiments, the scFv that specifically binds HER2 comprises a sequence having at least about 97% identity to SEQ ID NO: 88. In some embodiments, the scFv that specifically binds HER2 comprises a sequence having at least about 98% identity to SEQ ID NO: 88. In some embodiments, the scFv that specifically binds HER2 comprises a sequence having at least about 99% identity to SEQ ID NO: 88. In some embodiments, the scFv that specifically binds HER2 comprises a sequence having 100% identity to SEQ ID NO: 88.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 6. In some embodiments, the scFv that specifically binds HER2 comprises a sequence having at least about 85% identity to SEQ ID NO: 6. In some embodiments, the scFv that specifically binds HER2 comprises a sequence having at least about 90% identity to SEQ ID NO: 6. In some embodiments, the scFv that specifically binds HER2 comprises a sequence having at least about 95% identity to SEQ ID NO: 6. In some embodiments, the scFv that specifically binds HER2 comprises a sequence having at least about 96% identity to SEQ ID NO: 6. In some embodiments, the scFv that specifically binds HER2 comprises a sequence having at least about 97% identity to SEQ ID NO: 6. In some embodiments, the scFv that specifically binds HER2 comprises a sequence having at least about 98% identity to SEQ ID NO: 6. In some embodiments, the scFv that specifically binds HER2 comprises a sequence having at least about 99% identity to SEQ ID NO: 6. In some embodiments, the scFv that specifically binds HER2 comprises a sequence having 100% identity to SEQ ID NO: 6.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89. In some embodiments, the scFv that specifically binds HER2 comprises a sequence having at least about 85% identity to SEQ ID NO: 89. In some embodiments, the scFv that specifically binds HER2 comprises a sequence having at least about 90% identity to SEQ ID NO: 89. In some embodiments, the scFv that specifically binds HER2 comprises a sequence having at least about 95% identity to SEQ ID NO: 89. In some embodiments, the scFv that specifically binds HER2 comprises a sequence having at least about 96% identity to SEQ ID NO: 89. In some embodiments, the scFv that specifically binds HER2 comprises a sequence having at least about 97% identity to SEQ ID NO: 89. In some embodiments, the scFv that specifically binds HER2 comprises a sequence having at least about 98% identity to SEQ ID NO: 89. In some embodiments, the scFv that specifically binds HER2 comprises a sequence having at least about 99% identity to SEQ ID NO: 89. In some embodiments, the scFv that specifically binds HER2 comprises a sequence having 100% identity to SEQ ID NO: 89.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9. In some embodiments, the scFv that specifically binds HER2 comprises a sequence having at least about 85% identity to SEQ ID NO: 9. In some embodiments, the scFv that specifically binds HER2 comprises a sequence having at least about 90% identity to SEQ ID NO: 9. In some embodiments, the scFv that specifically binds HER2 comprises a sequence having at least about 95% identity to SEQ ID NO: 9. In some embodiments, the scFv that specifically binds HER2 comprises a sequence having at least about 96% identity to SEQ ID NO: 9. In some embodiments, the scFv that specifically binds HER2 comprises a sequence having at least about 97% identity to SEQ ID NO: 9. In some embodiments, the scFv that specifically binds HER2 comprises a sequence having at least about 98% identity to SEQ ID NO: 9. In some embodiments, the scFv that specifically binds HER2 comprises a sequence having at least about 99% identity to SEQ ID NO: 9. In some embodiments, the scFv that specifically binds HER2 comprises a sequence having 100% identity to SEQ ID NO: 9.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90. In some embodiments, the scFv that specifically binds HER2 comprises a sequence having at least about 85% identity to SEQ ID NO: 90. In some embodiments, the scFv that specifically binds HER2 comprises a sequence having at least about 90% identity to SEQ ID NO: 90. In some embodiments, the scFv that specifically binds HER2 comprises a sequence having at least about 95% identity to SEQ ID NO: 90. In some embodiments, the scFv that specifically binds HER2 comprises a sequence having at least about 96% identity to SEQ ID NO: 90. In some embodiments, the scFv that specifically binds HER2 comprises a sequence having at least about 97% identity to SEQ ID NO: 90. In some embodiments, the scFv that specifically binds HER2 comprises a sequence having at least about 98% identity to SEQ ID NO: 90. In some embodiments, the scFv that specifically binds HER2 comprises a sequence having at least about 99% identity to SEQ ID NO: 90. In some embodiments, the scFv that specifically binds HER2 comprises a sequence having 100% identity to SEQ ID NO: 90.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 140. In some embodiments, the scFv that specifically binds HER2 comprises a sequence having at least about 85% identity to SEQ ID NO: 140. In some embodiments, the scFv that specifically binds HER2 comprises a sequence having at least about 90% identity to SEQ ID NO: 140. In some embodiments, the scFv that specifically binds HER2 comprises a sequence having at least about 95% identity to SEQ ID NO: 140. In some embodiments, the scFv that specifically binds HER2 comprises a sequence having at least about 96% identity to SEQ ID NO: 140. In some embodiments, the scFv that specifically binds HER2 comprises a sequence having at least about 97% identity to SEQ ID NO: 140. In some embodiments, the scFv that specifically binds HER2 comprises a sequence having at least about 98% identity to SEQ ID NO: 140. In some embodiments, the scFv that specifically binds HER2 comprises a sequence having at least about 99% identity to SEQ ID NO: 140. In some embodiments, the scFv that specifically binds HER2 comprises a sequence having 100% identity to SEQ ID NO: 140.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12. In some embodiments, the scFv that specifically binds HER2 comprises a sequence having at least about 85% identity to SEQ ID NO: 12. In some embodiments, the scFv that specifically binds HER2 comprises a sequence having at least about 90% identity to SEQ ID NO: 12. In some embodiments, the scFv that specifically binds HER2 comprises a sequence having at least about 95% identity to SEQ ID NO: 12. In some embodiments, the scFv that specifically binds HER2 comprises a sequence having at least about 96% identity to SEQ ID NO: 12. In some embodiments, the scFv that specifically binds HER2 comprises a sequence having at least about 97% identity to SEQ ID NO: 12. In some embodiments, the scFv that specifically binds HER2 comprises a sequence having at least about 98% identity to SEQ ID NO: 12. In some embodiments, the scFv that specifically binds HER2 comprises a sequence having at least about 99% identity to SEQ ID NO: 12. In some embodiments, the scFv that specifically binds HER2 comprises a sequence having 100% identity to SEQ ID NO: 12.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 141. In some embodiments, the scFv that specifically binds HER2 comprises a sequence having at least about 85% identity to SEQ ID NO: 141. In some embodiments, the scFv that specifically binds HER2 comprises a sequence having at least about 90% identity to SEQ ID NO: 141. In some embodiments, the scFv that specifically binds HER2 comprises a sequence having at least about 95% identity to SEQ ID NO: 141. In some embodiments, the scFv that specifically binds HER2 comprises a sequence having at least about 96% identity to SEQ ID NO: 141. In some embodiments, the scFv that specifically binds HER2 comprises a sequence having at least about 97% identity to SEQ ID NO: 141. In some embodiments, the scFv that specifically binds HER2 comprises a sequence having at least about 98% identity to SEQ ID NO: 141. In some embodiments, the scFv that specifically binds HER2 comprises a sequence having at least about 99% identity to SEQ ID NO: 141. In some embodiments, the scFv that specifically binds HER2 comprises a sequence having 100% identity to SEQ ID NO: 141.

In some embodiments, bispecific fusion proteins of the present disclosure comprise an scFv that specifically binds HER2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15. In some embodiments, the scFv that specifically binds HER2 comprises a sequence having at least about 85% identity to SEQ ID NO: 15. In some embodiments, the scFv that specifically binds HER2 comprises a sequence having at least about 90% identity to SEQ ID NO: 15. In some embodiments, the scFv that specifically binds HER2 comprises a sequence having at least about 95% identity to SEQ ID NO: 15. In some embodiments, the scFv that specifically binds HER2 comprises a sequence having at least about 96% identity to SEQ ID NO: 15. In some embodiments, the scFv that specifically binds HER2 comprises a sequence having at least about 97% identity to SEQ ID NO: 15. In some embodiments, the scFv that specifically binds HER2 comprises a sequence having at least about 98% identity to SEQ ID NO: 15. In some embodiments, the scFv that specifically binds HER2 comprises a sequence having at least about 99% identity to SEQ ID NO: 15. In some embodiments, the scFv that specifically binds HER2 comprises a sequence having 100% identity to SEQ ID NO: 15.

### CD3 Binding Site

Bispecific fusion proteins of the present disclosure can include a polypeptide or complex of two or more polypeptides that specifically bind CD3 on the surface of T cells. In some embodiments, bispecific fusion proteins of the present disclosure bind to CD3 expressed on mature T lymphocytes, for example, αβ T cells, γδ T cells, NK-T cells, mucosal-associated invariant T (MAIT) cells, and their phenotypic subsets. In some embodiments, binding of CD3 induces activation of the T cell when bridged to HER2.

As used herein, a CD3 binding site is a polypeptide or complex of two or more polypeptides that, in some embodiments, specifically binds CD3 (SEQ ID NO: 142). For example, the CD3 binding site binds to the CD3ε chain.

CD3 Sequence (ε chain):
(SEQ ID NO: 142)
MRWNTFWGILCLSLLAVGTCQDDAENIEYKVSISGTSVELTCPLDSDENL
KWEKNGQELPQKHDKHLVLQDFSEVEDSGYYVCYTPASNKNTYLYLKARV
CEYCVEVDLTAVAIIIIVDICITLGLLMVIYYWSKNRKAKAKPVTRGTGA
GSRPRGQNKERPPPVPNPDYEPIRKGQRDLYSGLNQRAV

In some embodiments, a CD3 binding site comprises a heavy chain variable region (VH) and a light chain variable region (VL). TABLE 5A lists VH and VL domains of anti-CD3 antibodies, and their corresponding complementarity-determining regions (CDRs) that, in combination, can specifically bind to CD3. TABLE 5B lists the corresponding nucleotide sequences of the VH and VL regions of anti-CD3 antibodies.

TABLE 5A
αCD3 VH/VL Sequences and CDRs

| Identi- | | |
|---|---|---|
| fier | VH | VL |
| L2K07 | DIKLQQSGAELARPGASVKMS | GGVDDIQLTQSPAIMS |
| | CKTSGYTFTRYTMHWVKQRPG | ASPGEKVTMTCRASSS |
| | QGLEWIGYINPSRGYTNYNQK | VSYMNWYQQKSGTSPK |
| | FKDKATLTTDKSSSTAYMQLS | RWIYDTSKVASGVPYR |
| | SLTSEDSAVYYCARYYDDHYC | FSGSGSGTSYSLTISS |
| | LDYWGQGTTLTVSSVE | MEAEDAATYYCQQWSS |
| | (SEQ ID NO: 16) | NPLTFGAGTKLELK |
| | CDR1: RYTMH | (SEQ ID NO: 17) |
| | (SEQ ID NO: 127) | CDR1: RASSSVSYMN |
| | CDR2: YINPSRGYTNYNQKF | (SEQ ID NO: 130) |
| | KD | CDR2: DTSKVAS |
| | (SEQ ID NO: 128) | (SEQ ID NO: 131) |
| | CDR3: YYDDHYCLDY | CDR3: QQWSSNPLT |
| | (SEQ ID NO: 129) | (SEQ ID NO: 132) |
| huOKT3 | QVQLVQSGGGVVQPGRSLRLS | DIQMTQSPSSLSASVG |
| | CKASGYTFTRYTMHWVRQAPG | DRVTITCASSSSVSYM |
| | KGLEWIGYINPSRGYTNYNQK | NWYQQTPGKAPKRWIY |
| | FKDRFTISRDNSKNTAFLQMD | DTSKLASGVPSRFSGS |
| | SLRPEDTGVYFCARYYDDHYC | GSGTDYTFTISSLQPE |
| | LDYWGQGTPVTVSS | DIATYYCQQWSSNPFT |
| | (SEQ ID NO: 95) | FGQGTKLQITR |
| | CDR1: RYTMH | (SEQ ID NO: 96) |
| | (SEQ ID NO: 133) | CDR1: SASSSVSYMN |
| | CDR2: YINPSRGYTNYNQKF | (SEQ ID NO: 136) |
| | KD | CDR2: DTSKLAS |
| | (SEQ ID NO: 134) | (SEQ ID NO: 137) |
| | CDR3: YYDDHYCLDY | CDR3: QQWSSNPFT |
| | (SEQ ID NO: 135) | (SEQ ID NO: 138) |

TABLE 5B
αCD3 VH/VL nucleotide sequences

| Identi- | |
|---|---|
| fier | Nucleotide sequence |
| L2K07 | GACATCAAACTGCAGCAGAGTGGGGCTGAGCTGGCCAGACCT |
| VH (1) | GGAGCCTCTGTCAAGATGAGCTGCAAAACCTCAGGCTACACA |
| (SEQ ID | TTCACCAGATACACCATGCACTGGGTGAAACAGAGACCTGGA |
| NO: 37) | CAGGGCCTGGAGTGGATTGGCTACATTAACCCATCAAGAGGC |
| | TATACCAACTATAACCAGAAATTCAAGGACAAGGCCACCCTG |
| | ACCACAGACAAAAGCAGCAGTACAGCTTACATGCAGCTGTCC |
| | TCCCTGACCTCTGAGGACTCTGCTGTGTACTATTGTGCCAGA |
| | TACTATGATGACCACTACTGCCTGGATTATTGGGGCCAGGGC |
| | ACAACACTGACAGTGTCTTCTGTGGAG |
| L2K07 | GGTGGAGTGGATGACATCCAGCTCACACAGTCCCCTGCCATC |
| VL (1) | ATGTCTGCCTCCCCTGGAGAGAAGGTGACAATGACCTGCAGA |
| (SEQ | GCAAGCTCATCTGTTTCCTACATGAACTGGTATCAGCAGAAG |
| ID NO: | TCTGGGACAAGCCCCAAAAGGTGGATCTATGACACCAGCAAA |
| 38) | GTGGCCTCTGGAGTGCCTTACAGATTCTCTGGATCTGGATCT |
| | GGCACCAGCTATTCTCTGACCATCTCCAGTATGGAAGCTGAG |
| | GATGCTGCCACCTACTACTGCCAGCAGTGGTCATCTAATCCC |
| | CTGACCTTTGGAGCTGGAACCAAACTGGAGCTGAAG |
| L2K07 | GATATTAAGCTCCAGCAGTCTGGAGCTGAGCTGGCCAGACCT |
| VH (2) | GGAGCCTCTGTGAAGATGAGTTGCAAGACCTCTGGCTACACC |
| (SEQ ID | TTTACCAGATACACAATGCATTGGGTGAAGCAGAGGCCAGGA |
| NO: 41) | CAGGGGCTGGAATGGATTGGCTATATCAACCCATCTAGAGGC |
| | TATACCAACTACAACCAGAAGTTTAAGGATAAAGCCACACTG |
| | ACCACAGACAAGAGCTCCTCCACAGCCTATATGCAGCTGTCT |
| | AGTCTGACCTCTGAGGATTCTGCTGTGTATTATTGTGCCAGG |
| | TATTATGATGACCATTACTGCCTGGATTACTGGGGCCAGGGC |
| | ACCACTCTGACAGTGAGCTCTGTGGAG |
| L2K07 | GGGGGAGTGGATGACATCCAACTGACCCAGTCCCCTGCCATC |
| VL (2) | ATGTCTGCCTCCCCAGGGGAAAAGGTCACCATGACCTGTAGA |
| (SEQ ID | GCCTCTTCCTCTGTGTCCTACATGAACTGGTATCAGCAGAAG |
| NO: 42) | TCTGGCACCTCTCCTAAGAGGTGGATTTATGATACTAGCAAG |
| | GTGGCTTCTGGGGTGCCATACAGGTTTTCTGGATCTGGTTCT |
| | GGAACCTCCTACTCCCTGACAATCTCCTCCATGGAAGCTGAG |
| | GATGCAGCCACTTACTACTGTCAGCAGTGGAGTTCCAATCCA |
| | CTCACTTTTGGGGCAGGCACAAAACTGGAGCTGAA |
| L2K07 | GACATCAAGCTGCAGCAGTCTGGAGCTGAGCTGGCTAGACCT |
| VH (3) | GGAGCCTCTGTGAAGATGTCCTGTAAGACCTCTGGTTACACA |
| (SEQ ID | TTTACCAGATATACTATGCATTGGGTGAAACAGAGACCAGGC |
| NO: 45) | CAGGGACTGGAGTGGATTGGGTACATCAACCCTTCCAGAGGC |
| | TACACCAATTACAATCAGAAGTTTAAGGATAAAGCCACTCTG |
| | ACCACTGACAAGTCCAGCAGCACAGCTTACATGCAGCTGAGC |
| | TCCCTGACATCTGAGGACTCTGCTGTGTATTATTGTGCAAGA |
| | TATTATGATGATCACTATTGCCTGGACTACTGGGGGCAGGGC |
| | ACTACACTGACAGTGTCCTCTGTGGAA |
| L2K07 | GGAGGAGTGGATGATATCCAGCTGACACAGAGCCCTGCAATC |
| VL (3) | ATGTCTGCTTCTCCTGGAGAGAAAGTGACCATGACATGCAGA |
| (SEQ ID | GCCTCATCCTCTGTGAGCTATATGAATTGGTACCAACAGAAG |
| NO: 46) | TCTGGGACATCCCCCAAGAGATGGATCTATGATACAAGCAAA |
| | GTGGCCTCTGGGGTGCCATACAGATTCTCTGGATCTGGCTCT |
| | GGAACATCCTACAGCCTGACTATTAGCAGTATGGAGGCTGAG |
| | GATGCTGCCACCTACTACTGCCAGCAGTGGTCCAGCAACCCA |
| | CTGACCTTTGGAGCTGGAACCAAACTGGAGCTGAAG |
| L2K07 | GACATCAAGCTGCAGCAGTCTGGAGCTGAGCTGGCCAGACCT |
| VH (4) | GGAGCCTCAGTGAAGATGAGCTGCAAAACATCTGGATACACC |
| (SEQ ID | TTCACCAGATACACCATGCACTGGGTCAAGCAGAGACCTGGA |
| NO: 49) | CAGGGACTGGAGTGGATTGGCTATATTAACCCTTCTAGAGGC |
| | TACACCAACTACAACCAAAAGTTCAAGGACAAAGCCACACTG |
| | ACCACAGACAAGTCCTCCAGCACTGCATACATGCAGTTGAGT |
| | AGCCTGACCTCAGAGGATTCTGCTGTGTACTATTGTGCTAGG |
| | TATTATGATGACCATTACTGTCTGGATTATTGGGGACAGGGC |
| | ACCACCCTGACAGTGAGCTCTGTGGAA |
| L2K07 | GGAGGAGTGGATGACATTCAGCTGACCCAGAGCCCTGCCATT |
| VL (4) | ATGTCTGCATCACCAGGAGAGAAGGTGACCATGACATGCAGG |
| (SEQ ID | GCAAGTTCTTCTGTGTCCTACATGAACTGGTATCAGCAGAAG |
| NO: 50 | TCTGGAACCTCCCCTAAAAGATGGATCTATGATACCAGTAAG |
| | GTGGCATCAGGAGTGCCCTACAGATTCTCTGGGTCTGGATCT |
| | GGAACAAGCTACTCCCTGACCATCTCTAGCATGGAGGCTGAG |
| | GATGCTGCCACCTACTACTGCCAGCAGTGGTCCAGCAACCCC |
| | CTGACATTTGGGGCTGGGACCAAGCTGGAACTGAAA |
| OKT3 VH | CAGGTGCAGCTGGTGCAGTCTGGAGGAGGGGTGGTTCAGCCT |
| (SEQ ID | GGCAGAAGCCTGAGACTGTCATGCAAGGCCTCAGGGTATACT |
| NO: | TTCACCAGATATACAATGCACTGGGTGAGACAGGCCCCAGGC |
| 147) | AAGGGACTGGAATGGATTGGCTACATTAACCCATCTAGGGGA |
| | TATACAAATTATAATCAGAAATTCAAGGACAGATTTACAATC |
| | TCCAGGGACAACTCTAAGAATACTGCCTTTCTGCAGATGGAC |
| | TCTCTGAGGCCTGAGGACACTGGAGTGTATTTCTGTGCCAGA |
| | TACTATGATGACCATTATTGTCTGGATTATTGGGGCCAGGGC |
| | ACACCTGTGACAGTGTCCAGT |
| OKT3 VL | GATATCCAGATGACCCAGTCCCCAAGTAGCCTGAGTGCCTCA |
| (SEQ ID | GTGGGAGACAGAGTGACCATCACCTGCTCTGCAAGCAGCTCT |
| NO: | GTGTCCTACATGAACTGGTATCAGCAGACACCAGGCAAGGCC |
| 148) | CCCAAGAGGTGGATTTATGACACCTCCAAGCTGGCTTCTGGG |
| | GTGCCAAGCAGATTCTCAGGATCTGGAAGTGGGACAGACTAC |
| | ACATTTACCATCAGTTCACTGCAGCCTGAGGACATTGCTACC |
| | TACTATTGTCAGCAGTGGTCCTCCAACCCCTTCACCTTTGGC |
| | CAGGGAACCAAGCTGCAGATCACCAGG |

In some embodiments, a CD3 binding site comprises VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196:901-917), MacCallum (see MacCallum R M et al., (1996) J. Mol. Biol. 262:732-745), or any other CDR determination method known in the art.

Unless indicated otherwise, the CDR sequences provided in TABLE 5A are determined under the Kabat numbering scheme.

In some embodiments, a CD3 binding site comprises (i) a VH CDR1 comprising an amino acid sequence of SEQ ID NO: 127; (ii) a VH CDR2 comprising an amino acid sequence of SEQ ID NO: 128; (iii) a VH CDR3 comprising an amino acid sequence of SEQ ID NO: 129; (iv) a VL CDR1 comprising an amino acid sequence of SEQ ID NO: 130; (v) a VL CDR2 comprising an amino acid sequence of SEQ ID NO: 131; and (vi) a VL CDR3 comprising an amino acid sequence of SEQ ID NO: 132.

In some embodiments, a CD3 binding site comprises (i) a VH CDR1 comprising an amino acid sequence of SEQ ID NO: 133; (ii) a VH CDR2 comprising an amino acid sequence of SEQ ID NO: 134; (iii) a VH CDR3 comprising an amino acid sequence of SEQ ID NO: 135; (iv) a VL CDR1 comprising an amino acid sequence of SEQ ID NO: 136; (v) a VL CDR2 comprising an amino acid sequence of SEQ ID NO: 137; and (vi) a VL CDR3 comprising an amino acid sequence of SEQ ID NO: 138.

TABLE 5A additionally lists amino acid sequences of exemplary VH and VL domains that, in combination, can specifically bind to CD3. In some embodiments, CD3 binding sites of the present disclosure comprise VH and VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to VH domain and VL domain sequences listed in TABLE 5A.

In some embodiments, a CD3 binding site of the present disclosure comprises (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17. In some embodiments, the CD3 binding site comprises a VH comprising an amino acid sequence having at least 80% sequence identity with an amino acid sequence according to SEQ ID NO: 16; and a VL comprising an amino acid sequence having at least 80% sequence identity with an amino acid sequence according to SEQ ID NO: 17. In some embodiments, the CD3 binding site comprises a VH comprising an amino acid sequence having at least 85% sequence identity with an amino acid sequence according to SEQ ID NO: 16; and a VL comprising an amino acid sequence having at least 85% sequence identity with an amino acid sequence according to SEQ ID NO: 17. In some embodiments, the CD3 binding site comprises a VH comprising an amino acid sequence having at least 90% sequence identity with an amino acid sequence according to SEQ ID NO: 16; and a VL comprising an amino acid sequence having at least 90% sequence identity with an amino acid sequence according to SEQ ID NO: 17. In some embodiments, the CD3 binding site comprises a VH comprising an amino acid sequence having at least 95% sequence identity with an amino acid sequence according to SEQ ID NO: 16; and a VL comprising an amino acid sequence having at least 95% sequence identity with an amino acid sequence according to SEQ ID NO: 17. In some embodiments, the CD3 binding site comprises a VH comprising an amino acid sequence having at least 96% sequence identity with an amino acid sequence according to SEQ ID NO: 16; and a VL comprising an amino acid sequence having at least 96% sequence identity with an amino acid sequence according to SEQ ID NO: 17. In some embodiments, the CD3 binding site comprises a VH comprising an amino acid sequence having at least 97% sequence identity with an amino acid sequence according to SEQ ID NO: 16; and a VL comprising an amino acid sequence having at least 97% sequence identity with an amino acid sequence according to SEQ ID NO: 17. In some embodiments, the CD3 binding site comprises a VH comprising an amino acid sequence having at least 98% sequence identity with an amino acid sequence according to SEQ ID NO: 16; and a VL comprising an amino acid sequence having at least 98% sequence identity with an amino acid sequence according to SEQ ID NO: 17. In some embodiments, the CD3 binding site comprises a VH comprising an amino acid sequence having at least 99% sequence identity with an amino acid sequence according to SEQ ID NO: 16; and a VL comprising an amino acid sequence having at least 99% sequence identity with an amino acid sequence according SEQ ID NO: 17. In some embodiments, the CD3 binding site comprises a VH comprising an amino acid sequence according to SEQ ID NO: 16; and a VL comprising an amino acid sequence according to SEQ ID NO: 17.

In some embodiments, a CD3 binding site of the present disclosure comprises (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 95; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 96. In some embodiments, the CD3 binding site comprises a VH comprising an amino acid sequence having at least 80% sequence identity with an amino acid sequence according to SEQ ID NO: 95; and a VL comprising an amino acid sequence having at least 80% sequence identity with an amino acid sequence according to SEQ ID NO: 96. In some embodiments, the CD3 binding site comprises a VH comprising an amino acid sequence having at least 85% sequence identity with an amino acid sequence according to SEQ ID NO: 95; and a VL comprising an amino acid sequence having at least 85% sequence identity with an amino acid sequence according to SEQ ID NO: 96. In some embodiments, the CD3 binding site comprises a VH comprising an amino acid sequence having at least 90% sequence identity with an amino acid sequence according to SEQ ID NO: 95; and a VL comprising an amino acid sequence having at least 90% sequence identity with an amino acid sequence according to SEQ ID NO: 96. In some embodiments, the CD3 binding site comprises a VH comprising an amino acid sequence having at least 95% sequence identity with an amino acid sequence according to SEQ ID NO: 95; and a VL comprising an amino acid sequence having at least 95% sequence identity with an amino acid sequence according to SEQ ID NO: 96. In some embodiments, the CD3 binding site comprises a VH comprising an amino acid sequence having at least 96% sequence identity with an amino acid sequence according to SEQ ID NO: 95; and a VL comprising an amino acid sequence having at least 96% sequence identity with an amino acid sequence according to SEQ ID NO: 96. In some embodiments, the CD3 binding site comprises a VH comprising an amino acid sequence having at least 97% sequence identity with an amino acid sequence according to SEQ ID NO: 95; and a VL comprising an amino acid sequence having at least 97% sequence identity with an amino acid sequence according to SEQ ID NO: 96. In some embodiments, the CD3 binding site comprises a VH comprising an amino acid sequence having at least 98% sequence identity with an amino acid sequence according to SEQ ID NO: 95; and a VL comprising an amino acid sequence having at least 98% sequence identity with an amino acid sequence according to SEQ ID NO: 96. In some embodiments, the CD3 binding site comprises a VH comprising an amino acid sequence having at least 99% sequence identity with an amino acid sequence according to SEQ ID NO: 95; and a VL comprising an amino acid sequence having at least 99% sequence identity with an amino acid sequence according SEQ ID NO: 96. In some embodiments, the CD3 binding site comprises a VH comprising an amino acid sequence according to SEQ ID NO: 95; and a VL comprising an amino acid sequence according to SEQ ID NO: 96.

In some embodiments, a CD3 binding site includes, but is not limited to, a single-chain variable fragment (scFv), an antibody, a Fab, a Fab′, a F(ab′)₂, a minibody, or a nanobody (VHH). For example, in some embodiments, bispecific fusion proteins of the present disclosure comprises an scFv polypeptide that each specifically binds to CD3.

In some embodiments, a CD3 binding site of the present disclosure is in an scFv format. In some embodiments, a CD3-binding scFv of the present disclosure comprises an scFv linker polypeptide that operably connects a VH domain and a VL domain. For example, in some embodiments, a CD3 binding scFv comprises, from N-terminus to C-terminus, a VL domain of an anti-CD3 antibody, an scFv linker polypeptide, and a VH domain of an anti-CD3 antibody. In other embodiments, a CD3 binding scFv comprises, from N-terminus to C-terminus, a VH domain of an anti-CD3 antibody, an scFv linker polypeptide, and a VL domain of an anti-CD3 antibody.

In some embodiments, an scFv linker polypeptide comprises a sequence selected from the linker sequences in TABLE 3A.

TABLE 6A lists amino acid sequences of exemplary CD3-binding scFvs. In some embodiments, bispecific fusion proteins of the present disclosure comprise a CD3-binding scFv comprising a sequence at least 85% identical (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 6A. TABLE 6B lists the corresponding nucleotide sequences of the exemplary CD3-binding scFvs.

TABLE 6A
αCD3 scFv Amino Acid Sequences

| Identifier | scFv |
|---|---|
| L2K07 scFv | DIKLQQSGAELARPGASVKMSCKTSGYTFTRYTMHW |
| (VH-VL) | VKQRPGQGLEWIGYINPSRGYTNYNQKFKDKATLTT |
| (SEQ ID NO: | DKSSSTAYMQLSSLTSEDSAVYYCARYYDDHYCLDY |
| 18) | WGQGTTLTVSSVEGGSGGSGGSGGSGGVDDIQLTQS |
| | PAIMSASPGEKVTMTCRASSSVSYMNWYQQKSGTSP |
| | KRWIYDTSKVASGVPYRFSGSGSGTSYSLTISSMEA |
| | EDAATYYCQQWSSNPLTFGAGTKLEL |
| huOKT3 scFv | QVQLVQSGGGVVQPGRSLRLSCKASGYTFTRYTMHW |
| (VH-VL) | VRQAPGKGLEWIGYINPSRGYTNYNQKFKDRFTISR |
| (SEQ ID NO: | DNSKNTAFLQMDSLRPEDTGVYFCARYYDDHYCLDY |
| 94) | WGQGTPVTVSSGGGGSGGGGSGGGGSDIQMTQSPSS |
| | LSASVGDRVTITCSASSSVSYMNWYQQTPGKAPKRW |
| | IYDTSKLASGVPSRFSGSGSGTDYTFTISSLQPEDI |
| | ATYYCQQWSSNPFTFGQGTKLQITR |

Underlined italicized text indicates scFv linker sequence.

TABLE 6B
αCD3 scFv Nucleotide Sequences

| Identifier | scFv |
|---|---|
| L2K07 (1) | GACATCAAACTGCAGCAGAGTGGGGCTGAGCTGGCCAGACC |
| scFv (VH-VL) | TGGAGCCTCTGTCAAGATGAGCTGCAAAACCTCAGGCTACA |
| (SEQ ID NO: 143) | CATTCACCAGATACACCATGCACTGGGTGAAACAGAGACCT |
| | GGACAGGGCCTGGAGTGGATTGGCTACATTAACCCATCAAG |
| | AGGCTATACCAACTATAACCAGAAATTCAAGGACAAGGCCA |
| | CCCTGACCACAGACAAAAGCAGCAGTACAGCTTACATGCAG |
| | CTGTCCTCCCTGACCTCTGAGGACTCTGCTGTGTACTATTG |
| | TGCCAGATACTATGATGACCACTACTGCCTGGATTATTGGG |
| | GCCAGGGCACAACACTGACAGTGTCTTCTGTGGAGGGTGGC |
| | TCAGGTGGGTCTGGGGGCTCTGGAGGCTCTGGTGGAGTGGA |
| | TGACATCCAGCTCACACAGTCCCCTGCCATCATGTCTGCCT |
| | CCCCTGGAGAGAAGGTGACAATGACCTGCAGAGCAAGCTCA |
| | TCTGTTTCCTACATGAACTGGTATCAGCAGAAGTCTGGGAC |
| | AAGCCCCAAAAGGTGGATCTATGACACCAGCAAAGTGGCCT |
| | CTGGAGTGCCTTACAGATTCTCTGGATCTGGATCTGGCACC |
| | AGCTATTCTCTGACCATCTCCAGTATGGAAGCTGAGGATGC |
| | TGCCACCTACTACTGCCAGCAGTGGTCATCTAATCCCCTGA |
| | CCTTTGGAGCTGGAACCAAACTGGAGCT |
| L2K07 (2) | GATATTAAGCTCCAGCAGTCTGGAGCTGAGCTGGCCAGACC |
| scFv (VH-VL) | TGGAGCCTCTGTGAAGATGAGTTGCAAGACCTCTGGCTACA |
| (SEQ ID NO: 144) | CCTTTACCAGATACACAATGCATTGGGTGAAGCAGAGGCCA |
| | GGACAGGGGCTGGAATGGATTGGCTATATCAACCCATCTAG |
| | AGGCTATACCAACTACAACCAGAAGTTTAAGGATAAAGCCA |
| | CACTGACCACAGACAAGAGCTCCTCCACAGCCTATATGCAG |
| | CTGTCTAGTCTGACCTCTGAGGATTCTGCTGTGTATTATTG |
| | TGCCAGGTATTATGATGACCATTACTGCCTGGATTACTGGG |
| | GCCAGGGCACCACTCTGACAGTGAGCTCTGTGGAGGGGGGG |
| | TCTGGGGGCTCTGGAGGCTCTGGGGGCAGTGGGGGAGTGGA |
| | TGACATCCAACTGACCCAGTCCCCTGCCATCATGTCTGCCT |
| | CCCCAGGGGAAAAGGTCACCATGACCTGTAGAGCCTCTTCC |
| | TCTGTGTCCTACATGAACTGGTATCAGCAGAAGTCTGGCAC |
| | CTCTCCTAAGAGGTGGATTTATGATACTAGCAAGGTGGCTT |
| | CTGGGGTGCCATACAGGTTTTCTGGATCTGGTTCTGGAACC |
| | TCCTACTCCCTGACAATCTCCTCCATGGAAGCTGAGGATGC |
| | AGCCACTTACTACTGT |
| L2K07 (3) | GACATCAAGCTGCAGCAGTCTGGAGCTGAGCTGGCTAGACC |
| scFv (VH-VL) | TGGAGCCTCTGTGAAGATGTCCTGTAAGACCTCTGGTTACA |
| (SEQ ID NO: 145) | CATTTACCAGATATACTATGCATTGGGTGAAACAGAGACCA |
| | GGCCAGGGACTGGAGTGGATTGGGTACATCAACCCTTCCAG |
| | AGGCTACACCAATTACAATCAGAAGTTTAAGGATAAAGCCA |
| | CTCTGACCACTGACAAGTCCAGCAGCACAGCTTACATGCAG |
| | CTGAGCTCCCTGACATCTGAGGACTCTGCTGTGTATTATTG |
| | TGCAAGATATTATGATGATCACTATTGCCTGGACTACTGGG |
| | GGCAGGGCACTACACTGACAGTGTCCTCTGTGGAAGGAGGT |
| | TCTGGAGGCTCTGGAGGCTCTGGGGGCTCTGGAGGAGTGGA |
| | TGATATCCAGCTGACACAGAGCCCTGCAATCATGTCTGCTT |
| | CTCCTGGAGAGAAAGTGACCATGACATGCAGAGCCTCATCC |
| | TCTGTGAGCTATATGAATTGGTACCAACAGAAGTCTGGGAC |
| | ATCCCCCAAGAGATGGATCTATGATACAAGCAAAGTGGCCT |
| | CTGGGGTGCCATACAGATTCTCTGGATCTGGCTCTGGAACA |
| | TCCTACAGCCTGACTATTAGCAGTATGGAGGCTGAGGATGC |
| | TGCCACCTACTACTGCCAGCAGTGGTCCAGCAACCCACTGA |
| | CCTTTGGAGCTGGAACCAAACTGGAGCTGAAG |
| L2K07 (4) | GACATCAAGCTGCAGCAGTCTGGAGCTGAGCTGGCCAGACC |
| scFv (VH-VL) | TGGAGCCTCAGTGAAGATGAGCTGCAAAACATCTGGATACA |
| (SEQ ID NO: 146) | CCTTCACCAGATACACCATGCACTGGGTCAAGCAGAGACCT |
| | GGACAGGGACTGGAGTGGATTGGCTATATTAACCCTTCTAG |
| | AGGCTACACCAACTACAACCAAAAGTTCAAGGACAAAGCCA |
| | CACTGACCACAGACAAGTCCTCCAGCACTGCATACATGCAG |
| | TTGAGTAGCCTGACCTCAGAGGATTCTGCTGTGTACTATTG |
| | TGCTAGGTATTATGATGACCATTACTGTCTGGATTATTGGG |
| | GACAGGGCACCACCCTGACAGTGAGCTCTGTGGAAGGAGGC |
| | TCTGGAGGCTCTGGAGGCTCTGGAGGCAGTGGAGGAGTGGA |
| | TGACATTCAGCTGACCCAGAGCCCTGCCATTATGTCTGCAT |
| | CACCAGGAGAGAAGGTGACCATGACATGCAGGGCAAGTTCT |
| | TCTGTGTCCTACATGAACTGGTATCAGCAGAAGTCTGGAAC |
| | CTCCCCTAAAAGATGGATCTATGATACCAGTAAGGTGGCAT |
| | CAGGAGTGCCCTACAGATTCTCTGGGTCTGGATCTGGAACA |
| | AGCTACTCCCTGACCATCTCTAGCATGGAGGCTGAGGATGC |
| | TGCCACCTACTACTGCCAGCAGTGGTCCAGCAACCCCCTGA |
| | CATTTGGGGCTGGGACCAAGCTGGAACTGAAA |
| huOKT3 scFv | CAGGTGCAGCTGGTGCAGTCTGGAGGAGGGGTGGTTCAGCC |
| (VH-VL) | TGGCAGAAGCCTGAGACTGTCATGCAAGGCCTCAGGGTATA |
| (SEQ ID NO: 149) | CTTTCACCAGATATACAATGCACTGGGTGAGACAGGCCCCA |
| | GGCAAGGGACTGGAATGGATTGGCTACATTAACCCATCTAG |
| | GGGATATACAAATTATAATCAGAAATTCAAGGACAGATTTA |
| | CAATCTCCAGGGACAACTCTAAGAATACTGCCTTTCTGCAG |
| | ATGGACTCTCTGAGGCCTGAGGACACTGGAGTGTATTTCTG |
| | TGCCAGATACTATGATGACCATTATTGTCTGGATTATTGGG |
| | GCCAGGGCACACCTGTGACAGTGTCCAGTGGAGGAGGAGGC |
| | TCAGGGGGAGGGGGCTCTGGAGGGGGAGGCTCTGATATCCA |
| | GATGACCCAGTCCCCAAGTAGCCTGAGTGCCTCAGTGGGAG |
| | ACAGAGTGACCATCACCTGCTCTGCAAGCAGCTCTGTGTCC |
| | TACATGAACTGGTATCAGCAGACACCAGGCAAGGCCCCCAA |
| | GAGGTGGATTTATGACACCTCCAAGCTGGCTTCTGGGGTGC |
| | CAAGCAGATTCTCAGGATCTGGAAGTGGGACAGACTACACA |
| | TTTACCATCAGTTCACTGCAGCCTGAGGACATTGCTACCTA |
| | CTATTGTCAGCAGTGGTCCTCCAACCCCTTCACCTTTGGCC |
| | AGGGAACCAAGCTGCAGATCACCAGG |

Underlined text indicates scFv linker sequence.

In some embodiments, bispecific fusion proteins of the present disclosure comprise an scFv that specifically binds CD3 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18. In some embodiments, the scFv that specifically binds CD3 comprises a sequence having at least about 85% identity to SEQ ID NO: 18. In some embodiments, the scFv that specifically binds CD3 comprises a sequence having at least about 90% identity to SEQ ID NO: 18. In some embodiments, the scFv that specifically binds CD3 comprises a sequence having at least about 95% identity to SEQ ID NO: 18. In some embodiments, the scFv that specifically binds CD3 comprises a sequence having at least about 96% identity to SEQ ID NO: 18. In some embodiments, the scFv that specifically binds CD3 comprises a sequence having at least about 97% identity to SEQ ID NO: 18. In some embodiments, the scFv that specifically binds CD3 comprises a sequence having at least about 98% identity to SEQ ID NO: 18. In some embodiments, the scFv that specifically binds CD3 comprises a sequence having at least about 99% identity to SEQ ID NO: 18. In some embodiments, the scFv that specifically binds CD3 comprises a sequence having 100% identity to SEQ ID NO: 18.

In some embodiments, bispecific fusion proteins of the present disclosure comprise an scFv that specifically binds CD3 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 94. In some embodiments, the scFv that specifically binds CD3 comprises a sequence having at least about 85% identity to SEQ ID NO: 94. In some embodiments, the scFv that specifically binds CD3 comprises a sequence having at least about 90% identity to SEQ ID NO: 94. In some embodiments, the scFv that specifically binds CD3 comprises a sequence having at least about 95% identity to SEQ ID NO: 94. In some embodiments, the scFv that specifically binds CD3 comprises a sequence having at least about 96% identity to SEQ ID NO: 94. In some embodiments, the scFv that specifically binds CD3 comprises a sequence having at least about 97% identity to SEQ ID NO: 94. In some embodiments, the scFv that specifically binds CD3 comprises a sequence having at least about 98% identity to SEQ ID NO: 94. In some embodiments, the scFv that specifically binds CD3 comprises a sequence having at least about 99% identity to SEQ ID NO: 94. In some embodiments, the scFv that specifically binds CD3 comprises a sequence having 100% identity to SEQ ID NO: 94.

### Exemplary αHER2-αCD3 Bispecific Fusion Proteins

Listed below are examples of bispecific fusion proteins of the present disclosure comprising a HER2 binding site fused via a linker peptide to a CD3 binding site.

In some embodiments, bispecific fusion proteins of the present disclosure comprise (i) a HER2 binding site comprising VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A; (ii) a linker peptide comprising a sequence selected from TABLE 7; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A. In some embodiments, bispecific fusion proteins of the present disclosure comprise (i) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A; (ii) a linker peptide comprising a sequence selected from TABLE 7; and (iii) a HER2 binding site comprising VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A. The order of HER2 binding site and CD3 binding site is not meant to be limited. For example, the HER2 binding site is at the amino terminus of the bispecific fusion protein and the CD3 binding site is at the carboxy terminus of the bispecific fusion protein. In some embodiments, the CD3 binding site is at the amino terminus of the bispecific fusion protein and the HER2 binding site is at the carboxy terminus of the bispecific fusion protein.

TABLE 7
Linker Peptides

| Linker | Sequence |
|---|---|
| Linker 6 | GGGGS |
| (SEQ ID | |
| NO: 29) | |

In some embodiments, bispecific fusion proteins of the present disclosure comprise (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, bispecific fusion proteins of the present disclosure comprise (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29 and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, bispecific fusion proteins of the present disclosure comprise (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, bispecific fusion proteins of the present disclosure comprise (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, bispecific fusion proteins of the present disclosure comprise (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence listed in TABLE 2A, respectively; (ii) a linker peptide comprising a sequence selected from TABLE 7; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence listed in TABLE 5A.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively.

In some embodiments, bispecific fusion proteins of the present disclosure comprise (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 4A; (ii) a linker peptide comprising a sequence selected from TABLE 7; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 6A.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18.

In some embodiments, bispecific fusion proteins of the present disclosure comprise (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18.

In some embodiments, bispecific fusion proteins of the present disclosure comprise a spacer peptide fused to the N-terminus of the scFv linker peptide, at the C-terminus of the VH domain of the HER2 scFv, at the C-terminus of the VL domain of the HER2 scFv, at the C-terminus of the VH domain of the CD3 scFv, and/or at the C-terminus of the VL domain of the CD3 scFv. In some embodiments, the spacer peptide comprises a sequence selected from the spacer sequences listed in TABLE 3B.

In some embodiments, bispecific fusion proteins of the present disclosure comprise an amino acid sequence corresponding to a sequences listed in TABLE 8A. TABLE 8B lists the corresponding nucleotide sequences of the bispecific fusion proteins.

TABLE 8A
Bispecific Fusion Protein Amino Acid Sequences

| Identifier | Amino Acid Sequence |
|---|---|
| Pertuzumab scFv | DIQMTQSPSSLSASVGDRVTITCKASQDVSIGVAWYQQKPGKA |
| (VL-VH) + L2K07 | PKLLIYSASYRYTGVPSRFSGSGSGTDFTLTISSLQPEDFATY |
| CD3 scFv | YCQQYYIYPYTFGQGTKVEIKSCGGGSGGGGSGGGGSEVQLVE |
| (VH-VL) | SGGGLVQPGGSLRLSCAASGFTFTDYTMDWVRQAPGKGLEWVA |
| (SEQ ID NO: 19) | DVNPNSGGSIYNQRFKGRFTLSVDRSKNTLYLQMNSLRAEDTA |
| | VYYCARNLGPSFYFDYWGQGTLVTVSSGGGGSDIKLQQSGAEL |
| | ARPGASVKMSCKTSGYTFTRYTMHWVKQRPGQGLEWIGYINPS |
| | RGYTNYNQKFKDKATLTTDKSSSTAYMQLSSLTSEDSAVYYCA |
| | RYYDDHYCLDYWGQGTTLTVSSVEGGSGGSGGSGGSGGVDDIQ |
| | LTQSPAIMSASPGEKVTMTCRASSSVSYMNWYQQKSGTSPKRW |
| | IYDTSKVASGVPYRFSGSGSGTSYSLTISSMEAEDAATYYCQQ |
| | WSSNPLTFGAGTKLELK |
| Herceptin | DIQMTQSPSSLSASVGDRVTITCRASQDVNTAVAWYQQKPGKA |
| (Trastuzumab) | PKLLIYSASFLYSGVPSRFSGSRSGTDFTLTISSLQPEDFATY |
| scFv (VL-VH) + | YCQQHYTTPPTFGQGTKVEIKGGGGSGGGGSGGGGSGGGGSEV |
| L2K07 CD3 scFv | QLVESGGGLVQPGGSLRLSCAASGFNIKDTYIHWVRQAPGKGL |
| (VH-VL) | EWVARIYPTNGYTRYADSVKGRFTISADTSKNTAYLQMNSLRA |
| (SEQ ID NO: 20) | EDTAVYYCSRWGGDGFYAMDYWGQGTLVTVSSGGGGSDIKLQQ |
| | SGAELARPGASVKMSCKTSGYTFTRYTMHWVKQRPGQGLEWIG |
| | YINPSRGYTNYNQKFKDKATLTTDKSSSTAYMQLSSLTSEDSA |
| | VYYCARYYDDHYCLDYWGQGTTLTVSSVEGGSGGSGGSGGSGG |
| | VDDIQLTQSPAIMSASPGEKVTMTCRASSSVSYMNWYQQKSGT |
| | SPKRWIYDTSKVASGVPYRFSGSGSGTSYSLTISSMEAEDAAT |
| | YYCQQWSSNPLTFGAGTKLELK |
| Hersintuzumab | DIVMTQSPSSLSASVGDRVTITCRASQNVRTAVAWFQQKPGKA |
| scFv (VL-VH) + | PKALIYLASNRHTGVPDRFTGSGSGTEFTLTISNLQPEDFATY |
| L2K07 CD3 scFv | FCLQHNSYPLTFGGGTKVEIKGGGGSGGGGSGGGGSEVKLVES |
| (VH-VL) | GGGLVKPGGSLRLSCATSGFSFSSYYMYWVRQAPGKRLEWVAY |
| (SEQ ID NO: 21) | ISSGSEIYYSDSVKGRFTISRDSAKNTLYLQMNSLRAEDTAVY |
| | YCARLGDDGMDWWGQGTTWTVSSGGGGSDIKLQQSGAELARPG |
| | ASVKMSCKTSGYTFTRYTMHWVKQRPGQGLEWIGYINPSRGYT |
| | NYNQKFKDKATLTTDKSSSTAYMQLSSLTSEDSAVYYCARYYD |
| | DHYCLDYWGQGTTLTVSSVEGGSGGSGGSGGSGGVDDIQLTQS |
| | PAIMSASPGEKVTMTCRASSSVSYMNWYQQKSGTSPKRWIYDT |
| | SKVASGVPYRFSGSGSGTSYSLTISSMEAEDAATYYCQQWSSN |
| | PLTFGAGTKLELK |
| huA21 scFv | ADIVLTQSPDSLAVSLGERVTINCKSSQPLEYSNNQWNYLAWY |
| (VL-VH) + L2K07 | QQKPGQSPKLLISWASTRKSGVPDRFSGSGSGTDFTLTISSVQ |
| CD3 scFv | AEDVAVYYCGQYSDYPNTFGAGTKLEIKRGGGGSGGGGSGGGG |
| (VH-VL) | SGGGGSQVQLVQSGAEVVKPGASVKISCKASGYPFTQYFIHWV |
| (SEQ ID NO: 22) | KQNPGQRLEWIGQISSSYATVDYNQKFKGKATLTVDTSASIAY |
| | MELSSLRSEDTAVYYCVRSGNYEEYAMDYWGQGTLVTVSGGGG |
| | SDIKLQQSGAELARPGASVKMSCKTSGYTFTRYTMHWVKQRPG |
| | QGLEWIGYINPSRGYTNYNQKFKDKATLTTDKSSSTAYMQLSS |
| | LTSEDSAVYYCARYYDDHYCLDYWGQGTTLTVSSVEGGSGGSG |
| | GSGGSGGVDDIQLTQSPAIMSASPGEKVTMTCRASSSVSYMNW |
| | YQQKSGTSPKRWIYDTSKVASGVPYRFSGSGSGTSYSLTISSM |
| | EAEDAATYYCQQWSSNPLTFGAGTKLELK |
| 3H3B chA21 | AAQPADIVLTQTPSSLPVSVGEKVTMTCKSSQTLLYSNNQKNY |
| (VL-VH) + L2K07 | LAWYQQKPGQSPKLLISWAFTRKSGVPDRFTGSGSGTDFTLTI |
| CD3 scFv | GSVKAEDLAVYYCQQYSNYPWTFGGGTRLEIKRGGGGSGGGGS |
| (VH-VL) | GGGGSGGGGSEVQLQQSGPEVVKTGASVKISCKASGYSFTGYF |
| (SEQ ID NO: 23) | INWVKKNSGKSPEWIGHISSSYATSTYNQKFKNKAAFTVDTSS |
| | STAFMQLNSLTSEDSAVYYCVRSGNYEEYAMDYWGQGTSVTVS |
| | SGGGGSDIKLQQSGAELARPGASVKMSCKTSGYTFTRYTMHWV |
| | KQRPGQGLEWIGYINPSRGYTNYNQKFKDKATLTTDKSSSTAY |
| | MQLSSLTSEDSAVYYCARYYDDHYCLDYWGQGTTLTVSSVEGG |
| | SGGSGGSGGSGGVDDIQLTQSPAIMSASPGEKVTMTCRASSSV |
| | SYMNWYQQKSGTSPKRWIYDTSKVASGVPYRFSGSGSGTSYSL |
| | TISSMEAEDAATYYCQQWSSNPLTFGAGTKLELK |

Underlined italicized text indicates scFv linker sequence;
underlined text indicates spacer sequence; bold text indicates linker sequence.

TABLE 8B
Bispecific Fusion Protein Nucleotide Sequences

| Identifier | Nucleotide Sequence |
|---|---|
| Pertuzumab | GACATTCAGATGACTCAGTCTCCCAGCTCCCTGTCTGCCTCTGT |
| scFv | GGGAGACAGAGTGACAATCACATGTAAGGCCTCCCAGGATGTGT |
| (VL-VH) + | CTATTGGAGTGGCCTGGTACCAGCAGAAACCTGGAAAGGCCCCA |
| L2K07 CD3 | AAACTGCTGATCTACTCTGCCTCCTACAGGTATACAGGAGTGCC |
| scFv (VH-VL) | TTCCAGATTCTCTGGCTCTGGCTCTGGCACAGATTTCACTCTTA |
| (SEQ ID NO: | CCATCAGTTCCCTCCAGCCTGAGGATTTTGCTACCTACTATTGC |
| 59) | CAGCAGTACTACATCTATCCTTACACATTTGGCCAGGGAACAAA |
| | AGTGGAGATCAAGTCCTGTGGAGGGGGATCTGGGGGAGGGGGGT |
| | CAGGAGGGGGAGGCTCTGAGGTGCAGCTGGTGGAGTCTGGGGGA |
| | GGCCTGGTGCAGCCAGGGGGCTCACTGAGACTGAGCTGTGCTGC |
| | CTCTGGGTTTACATTTACAGACTATACCATGGACTGGGTGAGAC |
| | AGGCCCCAGGGAAGGGCCTGGAGTGGGTGGCAGATGTGAACCCA |
| | AATTCTGGAGGGTCCATCTACAATCAGAGGTTCAAGGGGAGATT |
| | TACCCTGTCTGTGGATAGAAGCAAGAACACACTGTACCTTCAGA |
| | TGAACTCCCTGAGAGCTGAGGACACAGCTGTGTACTACTGTGCC |
| | AGAAACCTGGGCCCATCCTTTTACTTTGACTACTGGGGACAGGG |
| | CACACTGGTGACAGTCTCCTCTGGGGGAGGAGGCTCTGATATTA |
| | AGCTCCAGCAGTCTGGAGCTGAGCTGGCCAGACCTGGAGCCTCT |
| | GTGAAGATGAGTTGCAAGACCTCTGGCTACACCTTTACCAGATA |
| | CACAATGCATTGGGTGAAGCAGAGGCCAGGACAGGGGCTGGAAT |
| | GGATTGGCTATATCAACCCATCTAGAGGCTATACCAACTACAAC |
| | CAGAAGTTTAAGGATAAAGCCACACTGACCACAGACAAGAGCTC |
| | CTCCACAGCCTATATGCAGCTGTCTAGTCTGACCTCTGAGGATT |
| | CTGCTGTGTATTATTGTGCCAGGTATTATGATGACCATTACTGC |
| | CTGGATTACTGGGGCCAGGGCACCACTCTGACAGTGAGCTCTGT |
| | GGAGGGGGGGTCTGGGGGCTCTGGAGGCTCTGGGGGCAGTGGGG |
| | GAGTGGATGACATCCAACTGACCCAGTCCCCTGCCATCATGTCT |
| | GCCTCCCCAGGGGAAAAGGTCACCATGACCTGTAGAGCCTCTTC |
| | CTCTGTGTCCTACATGAACTGGTATCAGCAGAAGTCTGGCACCT |
| | CTCCTAAGAGGTGGATTTATGATACTAGCAAGGTGGCTTCTGGG |
| | GTGCCATACAGGTTTTCTGGATCTGGTTCTGGAACCTCCTACTC |
| | CCTGACAATCTCCTCCATGGAAGCTGAGGATGCAGCCACTTACT |
| | ACTGT |
| Herceptin | GACATTCAGATGACACAGTCCCCATCTTCCCTGTCTGCCTCTGT |
| (Trastuzumab) | GGGAGACAGGGTGACAATCACTTGTAGGGCCAGCCAGGATGTGA |
| scFv | ACACAGCTGTGGCCTGGTATCAGCAGAAACCTGGAAAGGCCCCA |
| (VL-VH) + | AAGCTGCTGATCTACTCAGCTTCCTTCCTGTATTCTGGAGTGCC |
| L2K07 CD3 | ATCTAGGTTTTCTGGCTCCAGGTCTGGCACAGACTTTACCCTGA |
| scFv (VH-VL) | CCATCTCTTCTCTGCAGCCTGAGGACTTTGCCACTTACTACTGT |
| (SEQ ID NO: | CAGCAGCATTACACAACCCCCCCAACCTTTGGCCAGGGCACCAA |
| 60) | GGTGGAGATCAAGGGAGGAGGGGGAAGTGGGGGAGGAGGATCTG |
| | GAGGAGGGGGATCAGGAGGGGGAGGCTCAGAGGTGCAGCTGGTG |
| | GAGTCTGGGGGAGGCCTGGTGCAGCCTGGAGGCTCCCTGAGACT |
| | CTCCTGTGCTGCCTCTGGATTCAATATCAAAGATACTTACATCC |
| | ACTGGGTGAGACAGGCCCCTGGCAAAGGCCTGGAATGGGTGGCC |
| | AGAATCTATCCTACTAATGGCTACACCAGATATGCTGACTCTGT |
| | GAAGGGCAGATTTACAATCTCTGCTGACACCTCTAAAAATACAG |
| | CCTACCTGCAGATGAATTCCCTGAGAGCTGAAGACACAGCAGTG |
| | TACTACTGCAGCAGGTGGGGTGGAGATGGCTTCTATGCCATGGA |
| | CTATTGGGGCCAGGGCACCCTGGTGACAGTCTCATCTGGGGGAG |
| | GGGGCAGTGACATCAAGCTGCAGCAGTCTGGAGCTGAGCTGGCT |
| | AGACCTGGAGCCTCTGTGAAGATGTCCTGTAAGACCTCTGGTTA |
| | CACATTTACCAGATATACTATGCATTGGGTGAAACAGAGACCAG |
| | GCCAGGGACTGGAGTGGATTGGGTACATCAACCCTTCCAGAGGC |
| | TACACCAATTACAATCAGAAGTTTAAGGATAAAGCCACTCTGAC |
| | CACTGACAAGTCCAGCAGCACAGCTTACATGCAGCTGAGCTCCC |
| | TGACATCTGAGGACTCTGCTGTGTATTATTGTGCAAGATATTAT |
| | GATGATCACTATTGCCTGGACTACTGGGGGCAGGGCACTACACT |
| | GACAGTGTCCTCTGTGGAAGGAGGTTCTGGAGGCTCTGGAGGCT |
| | CTGGGGGCTCTGGAGGAGTGGATGATATCCAGCTGACACAGAGC |
| | CCTGCAATCATGTCTGCTTCTCCTGGAGAGAAAGTGACCATGAC |
| | ATGCAGAGCCTCATCCTCTGTGAGCTATATGAATTGGTACCAAC |
| | AGAAGTCTGGGACATCCCCCAAGAGATGGATCTATGATACAAGC |
| | AAAGTGGCCTCTGGGGTGCCATACAGATTCTCTGGATCTGGCTC |
| | TGGAACATCCTACAGCCTGACTATTAGCAGTATGGAGGCTGAGG |
| | ATGCTGCCACCTACTACTGCCAGCAGTGGTCCAGCAACCCACTG |
| | ACCTTTGGAGCTGGAACCAAACTGGAGCTGAAG |
| Hersintuzumab | GACATTGTGATGACACAGTCCCCCTCTAGCCTGTCTGCTTCTGT |
| scFv (VL-VH) | GGGAGACAGGGTGACAATCACATGCAGAGCCTCTCAGAATGTGA |
| + L2K07 CD3 | GAACAGCTGTGGCATGGTTCCAGCAGAAACCAGGAAAGGCCCCC |
| scFv (VH-VL) | AAGGCCCTGATCTACCTGGCCTCTAACAGGCACACAGGGGTGCC |
| (SEQ ID NO: | TGATAGGTTCACAGGCTCTGGATCTGGGACAGAGTTTACCCTGA |
| 61) | CCATCAGCAACCTGCAGCCTGAGGACTTTGCTACATACTTCTGC |
| | CTGCAGCACAACAGCTATCCTCTGACTTTTGGAGGAGGCACCAA |
| | AGTGGAGATCAAGGGAGGAGGAGGTTCTGGGGGAGGAGGATCTG |
| | GAGGAGGAGGATCTGAGGTAAAGCTGGTGGAGTCTGGAGGAGGC |
| | CTGGTGAAACCAGGGGGCTCTCTGAGACTGAGTTGTGCCACATC |
| | TGGCTTTTCCTTTAGCAGCTATTATATGTACTGGGTGAGGCAGG |
| | CCCCAGGCAAAAGGCTGGAGTGGGTGGCCTACATCAGCTCTGGC |
| | TCTGAAATTTATTACTCTGACTCTGTCAAGGGCAGATTCACCAT |
| | CAGCAGAGATTCTGCCAAGAACACCCTGTATCTCCAGATGAACT |
| | CTCTGAGAGCTGAGGACACAGCTGTGTATTACTGTGCTAGACTG |
| | GGAGATGATGGAATGGACTGGTGGGGCCAGGGAACCACATGGAC |
| | AGTGTCTTCTGGAGGAGGAGGATCTGACATCAAGCTGCAGCAGT |
| | CTGGAGCTGAGCTGGCCAGACCTGGAGCCTCAGTGAAGATGAGC |
| | TGCAAAACATCTGGATACACCTTCACCAGATACACCATGCACTG |
| | GGTCAAGCAGAGACCTGGACAGGGACTGGAGTGGATTGGCTATA |
| | TTAACCCTTCTAGAGGCTACACCAACTACAACCAAAAGTTCAAG |
| | GACAAAGCCACACTGACCACAGACAAGTCCTCCAGCACTGCATA |
| | CATGCAGTTGAGTAGCCTGACCTCAGAGGATTCTGCTGTGTACT |
| | ATTGTGCTAGGTATTATGATGACCATTACTGTCTGGATTATTGG |
| | GGACAGGGCACCACCCTGACAGTGAGCTCTGTGGAAGGAGGCTC |
| | TGGAGGCTCTGGAGGCTCTGGAGGCAGTGGAGGAGTGGATGACA |
| | TTCAGCTGACCCAGAGCCCTGCCATTATGTCTGCATCACCAGGA |
| | GAGAAGGTGACCATGACATGCAGGGCAAGTTCTTCTGTGTCCTA |
| | CATGAACTGGTATCAGCAGAAGTCTGGAACCTCCCCTAAAAGAT |
| | GGATCTATGATACCAGTAAGGTGGCATCAGGAGTGCCCTACAGA |
| | TTCTCTGGGTCTGGATCTGGAACAAGCTACTCCCTGACCATCTC |
| | TAGCATGGAGGCTGAGGATGCTGCCACCTACTACTGCCAGCAGT |
| | GGTCCAGCAACCCCCTGACATTTGGGGCTGGGACCAAGCTGGAA |
| | CTGAAA |
| huA21 scFv | GCTGACATAGTGCTGACCCAGAGTCCTGATTCCCTGGCTGTGTC |
| (VL-VH) + | TCTGGGAGAGAGAGTGACCATCAATTGCAAGTCCAGCCAGCCCC |
| L2K07 CD3 | TGGAATACAGCAATAATCAGTGGAACTACCTGGCTTGGTACCAG |
| scFv (VH-VL) | CAGAAACCTGGACAGTCCCCTAAGCTGCTGATTAGCTGGGCCAG |
| (SEQ ID NO: | CACCAGAAAGTCTGGAGTCCCAGACAGATTTTCTGGTTCTGGCT |
| 58) | CTGGAACTGACTTCACCCTCACAATCAGCTCTGTGCAGGCTGAA |
| | GATGTGGCTGTGTATTACTGTGGCCAGTACTCTGATTATCCCAA |
| | CACCTTTGGGGCTGGCACAAAGCTGGAAATCAAGAGAGGAGGTG |
| | GAGGTTCTGGAGGAGGGGGATCTGGTGGAGGGGGCTCTGGAGGA |
| | GGAGGGTCTCAGGTGCAGCTGGTGCAGTCTGGAGCTGAGGTGGT |
| | GAAACCAGGGGCTTCTGTGAAGATTTCCTGTAAGGCCTCTGGCT |
| | ACCCTTTCACTCAGTACTTCATTCATTGGGTCAAGCAGAATCCT |
| | GGGCAGAGACTGGAATGGATTGGACAAATCTCCTCCAGCTATGC |
| | CACAGTGGACTACAATCAGAAGTTCAAGGGCAAGGCAACCCTGA |
| | CTGTGGACACATCTGCCAGCATTGCCTATATGGAACTGAGCAGC |
| | CTGAGATCTGAAGATACAGCTGTGTATTACTGTGTGAGATCTGG |
| | CAATTATGAGGAGTATGCCATGGACTACTGGGGACAGGGCACCC |
| | TGGTGACTGTGTCTGGAGGAGGAGGTTCTGACATCAAACTGCAG |
| | CAGAGTGGGGCTGAGCTGGCCAGACCTGGAGCCTCTGTCAAGAT |
| | GAGCTGCAAAACCTCAGGCTACACATTCACCAGATACACCATGC |
| | ACTGGGTGAAACAGAGACCTGGACAGGGCCTGGAGTGGATTGGC |
| | TACATTAACCCATCAAGAGGCTATACCAACTATAACCAGAAATT |
| | CAAGGACAAGGCCACCCTGACCACAGACAAAAGCAGCAGTACAG |
| | CTTACATGCAGCTGTCCTCCCTGACCTCTGAGGACTCTGCTGTG |
| | TACTATTGTGCCAGATACTATGATGACCACTACTGCCTGGATTA |
| | TTGGGGCCAGGGCACAACACTGACAGTGTCTTCTGTGGAGGGTG |
| | GCTCAGGTGGGTCTGGGGGCTCTGGAGGCTCTGGTGGAGTGGAT |
| | GACATCCAGCTCACACAGTCCCCTGCCATCATGTCTGCCTCCCC |
| | TGGAGAGAAGGTGACAATGACCTGCAGAGCAAGCTCATCTGTTT |
| | CCTACATGAACTGGTATCAGCAGAAGTCTGGGACAAGCCCCAAA |
| | AGGTGGATCTATGACACCAGCAAAGTGGCCTCTGGAGTGCCTTA |
| | CAGATTCTCTGGATCTGGATCTGGCACCAGCTATTCTCTGACCA |
| | TCTCCAGTATGGAAGCTGAGGATGCTGCCACCTACTACTGCCAG |
| | CAGTGGTCATCTAATCCCCTGACCTTTGGAGCTGGAACCAAACT |
| | GGAGCT |
| 3H3B chA21 | GCGGCGCAGCCGGCGGATATTGTGCTGACCCAGACCCCGAGCAG |
| (VL-VH) + | CCTGCCGGTGAGCGTGGGCGAAAAAGTGACCATGACCTGCAAAA |
| L2K07 CD3 | GCAGCCAGACCCTGCTGTATAGCAACAACCAGAAAAACTATCTG |
| scFv (VH-VL) | GCGTGGTATCAGCAGAAACCGGGCCAGAGCCCGAAACTGCTGAT |
| (SEQ ID NO: | TAGCTGGGCGTTTACCCGCAAAAGCGGCGTGCCGGATCGCTTTA |
| 62) | CCGGCAGCGGCAGCGGCACCGATTTTACCCTGACCATTGGCAGC |
| | GTGAAAGCGGAAGATCTGGCGGTGTATTATTGCCAGCAGTATAG |
| | CAACTATCCGTGGACCTTTGGCGGCGGCACCCGCCTGGAAATTA |
| | AACGCGGCGGCGGCGGCAGCGGCGGCGGCGGCAGCGGCGGCGGC |
| | GGCAGCGGCGGCGGCGGCAGCGAAGTGCAGCTGCAGCAGAGCGG |
| | CCCGGAAGTGGTGAAAACCGGCGCGAGCGTGAAAATTAGCTGCA |
| | AAGCGAGCGGCTATAGCTTTACCGGCTATTTTATTAACTGGGTG |
| | AAAAAAAACAGCGGCAAAAGCCCGGAATGGATTGGCCATATTAG |
| | CAGCAGCTATGCGACCAGCACCTATAACCAGAAATTTAAAAACA |
| | AAGCGGCGTTTACCGTGGATACCAGCAGCAGCACCGCGTTTATG |
| | CAGCTGAACAGCCTGACCAGCGAAGATAGCGCGGTGTATTATTG |
| | CGTGCGCAGCGGCAACTATGAAGAATATGCGATGGATTATTGGG |
| | GCCAGGGCACCAGCGTGACCGTGAGCAGCGGCGGCGGCGGCAGC |
| | GATATTAAACTGCAGCAGAGCGGCGCGGAACTGGCGCGCCCGGG |
| | CGCGAGCGTGAAAATGAGCTGCAAAACCAGCGGCTATACCTTTA |
| | CCCGCTATACCATGCATTGGGTGAAACAGCGCCCGGGCCAGGGC |
| | CTGGAATGGATTGGCTATATTAACCCGAGCCGCGGCTATACCAA |
| | CTATAACCAGAAATTTAAAGATAAAGCGACCCTGACCACCGATA |
| | AAAGCAGCAGCACCGCGTATATGCAGCTGAGCAGCCTGACCAGC |
| | GAAGATAGCGCGGTGTATTATTGCGCGCGCTATTATGATGATCA |
| | TTATTGCCTGGATTATTGGGGCCAGGGCACCACCCTGACCGTGA |
| | GCAGCGTGGAAGGCGGCAGCGGCGGCAGCGGCGGCAGCGGCGGC |
| | AGCGGCGGCGTGGATGATATTCAGCTGACCCAGAGCCCGGCGAT |
| | TATGAGCGCGAGCCCGGGCGAAAAAGTGACCATGACCTGCCGCG |
| | CGAGCAGCAGCGTGAGCTATATGAACTGGTATCAGCAGAAAAGC |
| | GGCACCAGCCCGAAACGCTGGATTTATGATACCAGCAAAGTGGC |
| | GAGCGGCGTGCCGTATCGCTTTAGCGGCAGCGGCAGCGGCACCA |
| | GCTATAGCCTGACCATTAGCAGCATGGAAGCGGAAGATGCGGCG |
| | ACCTATTATTGCCAGCAGTGGAGCAGCAACCCGCTGACCTTTGG |
| | CGCGGGCACCAAACTGGAACTGAAA |

In addition to the sequences presented in TABLE 8A bispecific fusion proteins of the present disclosure may further comprise signal peptides fused to the N-terminus. It is understood that mature versions of the proteins are expressed from which the signal peptide has been cleaved.

For example, bispecific fusion proteins of the present disclosure may further comprise a signal peptide comprising an amino acid sequence having at least 85% identity (e.g., at least 85%, at least 90%, at least 91%, at least 92%, at least 93%, at least 94%, at least 95%, at least 96%, at least 97%, at least 98%, at least 99%, or 100%) to SEQ ID NO: 34.

| | Signal Peptide Sequence: |
|---|---|
| (SEQ ID NO: 34) | |
| | MWWRLWWLLLLLLLLWPMVWAA |

In some embodiments, bispecific fusion proteins of the present disclosure comprise an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19. In some embodiments, the bispecific fusion protein comprises an amino acid sequence having at least about 85% identity to SEQ ID NO: 19. In some embodiments, the bispecific fusion protein comprises an amino acid sequence having at least about 90% identity to SEQ ID NO: 19. In some embodiments, the bispecific fusion protein comprises an amino acid sequence having at least about 95% identity to SEQ ID NO: 19. In some embodiments, the bispecific fusion protein comprises an amino acid sequence having at least about 96% identity to SEQ ID NO: 19. In some embodiments, the bispecific fusion protein comprises an amino acid sequence having at least about 97% identity to SEQ ID NO: 19. In some embodiments, the bispecific fusion protein comprises an amino acid sequence having at least about 98% identity to SEQ ID NO: 19. In some embodiments, the bispecific fusion protein comprises an amino acid sequence having at least about 99% identity to SEQ ID NO: 19. In some embodiments, the bispecific fusion protein comprises an amino acid sequence having 100% identity to SEQ ID NO: 19.

In some embodiments, bispecific fusion proteins of the present disclosure comprise an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20. In some embodiments, the bispecific fusion protein comprises an amino acid sequence having at least about 85% identity to SEQ ID NO: 20. In some embodiments, the bispecific fusion protein comprises an amino acid sequence having at least about 90% identity to SEQ ID NO: 20. In some embodiments, the bispecific fusion protein comprises an amino acid sequence having at least about 95% identity to SEQ ID NO: 20. In some embodiments, the bispecific fusion protein comprises an amino acid sequence having at least about 96% identity to SEQ ID NO: 20. In some embodiments, the bispecific fusion protein comprises an amino acid sequence having at least about 97% identity to SEQ ID NO: 20. In some embodiments, the bispecific fusion protein comprises an amino acid sequence having at least about 98% identity to SEQ ID NO: 20. In some embodiments, the bispecific fusion protein comprises an amino acid sequence having at least about 99% identity to SEQ ID NO: 20. In some embodiments, the bispecific fusion protein comprises an amino acid sequence having 100% identity to SEQ ID NO: 20.

In some embodiments, bispecific fusion proteins of the present disclosure comprise an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21. In some embodiments, the bispecific fusion protein comprises an amino acid sequence having at least about 85% identity to SEQ ID NO: 21. In some embodiments, the bispecific fusion protein comprises an amino acid sequence having at least about 90% identity to SEQ ID NO: 21. In some embodiments, the bispecific fusion protein comprises an amino acid sequence having at least about 95% identity to SEQ ID NO: 21. In some embodiments, the bispecific fusion protein comprises an amino acid sequence having at least about 96% identity to SEQ ID NO: 21. In some embodiments, the bispecific fusion protein comprises an amino acid sequence having at least about 97% identity to SEQ ID NO: 21. In some embodiments, the bispecific fusion protein comprises an amino acid sequence having at least about 98% identity to SEQ ID NO: 21. In some embodiments, the bispecific fusion protein comprises an amino acid sequence having at least about 99% identity to SEQ ID NO: 21. In some embodiments, the bispecific fusion protein comprises an amino acid sequence having 100% identity to SEQ ID NO: 21.

In some embodiments, bispecific fusion proteins of the present disclosure comprise an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22. In some embodiments, the bispecific fusion protein comprises an amino acid sequence having at least about 85% identity to SEQ ID NO: 22. In some embodiments, the bispecific fusion protein comprises an amino acid sequence having at least about 90% identity to SEQ ID NO: 22. In some embodiments, the bispecific fusion protein comprises an amino acid sequence having at least about 95% identity to SEQ ID NO: 22. In some embodiments, the bispecific fusion protein comprises an amino acid sequence having at least about 96% identity to SEQ ID NO: 22. In some embodiments, the bispecific fusion protein comprises an amino acid sequence having at least about 97% identity to SEQ ID NO: 22. In some embodiments, the bispecific fusion protein comprises an amino acid sequence having at least about 98% identity to SEQ ID NO: 22. In some embodiments, the bispecific fusion protein comprises an amino acid sequence having at least about 99% identity to SEQ ID NO: 22. In some embodiments, the bispecific fusion protein comprises an amino acid sequence having 100% identity to SEQ ID NO: 22.

In some embodiments, bispecific fusion proteins of the present disclosure comprise an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23. In some embodiments, the bispecific fusion protein comprises an amino acid sequence having at least about 85% identity to SEQ ID NO: 23. In some embodiments, the bispecific fusion protein comprises an amino acid sequence having at least about 90% identity to SEQ ID NO: 23. In some embodiments, the bispecific fusion protein comprises an amino acid sequence having at least about 95% identity to SEQ ID NO: 23. In some embodiments, the bispecific fusion protein comprises an amino acid sequence having at least about 96% identity to SEQ ID NO: 23. In some embodiments, the bispecific fusion protein comprises an amino acid sequence having at least about 97% identity to SEQ ID NO: 23. In some embodiments, the bispecific fusion protein comprises an amino acid sequence having at least about 98% identity to SEQ ID NO: 23. In some embodiments, the bispecific fusion protein comprises an amino acid sequence having at least about 99% identity to SEQ ID NO: 23. In some embodiments, the bispecific fusion protein comprises an amino acid sequence having 100% identity to SEQ ID NO: 23.

Exemplary rAAV Vectors

In some embodiments, recombinant adeno-associated viral (rAAV) vectors of the present disclosure comprise a transgene nucleotide sequence corresponding to any one of the sequences listed in TABLE 8B. In some embodiments, the transgene comprises a sequence having at least about 85% identity to any one of SEQ ID NOs: 58-62. In some embodiments, the transgene comprises a sequence having at least about 90% identity to any one of SEQ ID NOs: 58-62. In some embodiments, the transgene comprises a sequence having at least about 95% identity to any one of SEQ ID NOs: 58-62. In some embodiments, the transgene comprises a sequence having at least about 96% identity to any one of SEQ ID NOs: 58-62. In some embodiments, the transgene comprises a sequence having at least about 97% identity to any one of SEQ ID NOs: 58-62. In some embodiments, the transgene comprises a sequence having at least about 98% identity to any one of SEQ ID NOs: 58-62. In some embodiments, the transgene comprises a sequence having at least about 99% identity to any one of SEQ ID NOs: 58-62. In some embodiments, the transgene comprises a sequence having 100% identity to any one of SEQ ID NOs: 58-62.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62.

In some embodiments, rAAV vectors of the present disclosure comprise one or more than one regulatory element. In some embodiments, the one or more than one regulatory element is 5′ of the sequence encoding the bispecific fusion protein. In some embodiments, the one or more than one regulatory element is 3′ of the sequence encoding the bispecific fusion protein. For example, in some embodiments, the regulatory element is 3′ of the sequence encoding the bispecific fusion protein and derived from a woodchuck hepatitis virus post-transcriptional regulatory element (WPRE). In some embodiments, the regulatory element is at least 85% identical (e.g., at least 85%, at least 90%, at least 91%, at least 92%, at least 93%, at least 94%, at least 95%, at least 96%, at least 97%, at least 98%, at least 99%, or 100%) to SEQ ID NO: 80. In some embodiments, the WPRE comprises a sequence having at least about 85% identity to SEQ ID NO: 80. In some embodiments, the WPRE comprises a sequence having at least about 90% identity to SEQ ID NO: 80. In some embodiments, the WPRE comprises a sequence having at least about 95% identity to SEQ ID NO: 80. In some embodiments, the WPRE comprises a sequence having at least about 96% identity to SEQ ID NO: 80. In some embodiments, the WPRE comprises a sequence having at least about 97% identity to SEQ ID NO: 80. In some embodiments, the WPRE comprises a sequence having at least about 98% identity to SEQ ID NO: 80. In some embodiments, the WPRE comprises a sequence having at least about 99% identity to SEQ ID NO: 80. In some embodiments, the WPRE comprises a sequence having 100% identity to SEQ ID NO: 80.

WPRE-Derived Regulatory Element:
(SEQ ID NO: 80)
TAACGATAATCAACCTCTGGATTACAAAATTTGTGAAAGATTGACTGGTA
TTCTTAACTATGTTGCTCCTTTTACGCTATGTGGATACGCTGCTTTAATG
CCTTTGTATCATGCTATTGCTTCCCGTATGGCTTTCATTTTCTCCTCCTT
GTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCC
CCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGG
AAATCATCGTCCTTTCCTTGGCTGCTCGCCTGTGTTGCCACCTGGATTCT
GCGCGGGACGTCCTTCTGCTACGTCCCTTCGGCCCTCAATCCAGCGGACC
TTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCATCG
G

In some embodiments, the regulatory element is 3′ of the sequence encoding the bispecific fusion protein and is a modified RNA stability regulatory element (MRE). In some embodiments, the regulatory element is at least 85% identical (e.g., at least 85%, at least 90%, at least 91%, at least 92%, at least 93%, at least 94%, at least 95%, at least 96%, at least 97%, at least 98%, at least 99%, or 100%) to SEQ ID NO: 175. In some embodiments, the MRE comprises a sequence having at least about 85% identity to SEQ ID NO: 175. In some embodiments, the MRE comprises a sequence having at least about 90% identity to SEQ ID NO: 175. In some embodiments, the MRE comprises a sequence having at least about 95% identity to SEQ ID NO: 175. In some embodiments, the MRE comprises a sequence having at least about 96% identity to SEQ ID NO: 175. In some embodiments, the MRE comprises a sequence having at least about 97% identity to SEQ ID NO: 175. In some embodiments, the MRE comprises a sequence having at least about 98% identity to SEQ ID NO: 175. In some embodiments, the MRE comprises a sequence having at least about 99% identity to SEQ ID NO: 175. In some embodiments, the MRE comprises a sequence having 100% identity to SEQ ID NO: 175.

In some embodiments, the MRE comprises a sequence having at least about 85% identity to SEQ ID NO: 176. In some embodiments, the MRE comprises a sequence having at least about 90% identity to SEQ ID NO: 176. In some embodiments, the MRE comprises a sequence having at least about 95% identity to SEQ ID NO: 176. In some embodiments, the MRE comprises a sequence having at least about 96% identity to SEQ ID NO: 176. In some embodiments, the MRE comprises a sequence having at least about 97% identity to SEQ ID NO: 176. In some embodiments, the MRE comprises a sequence having at least about 98% identity to SEQ ID NO: 176. In some embodiments, the MRE comprises a sequence having at least about 99% identity to SEQ ID NO: 176. In some embodiments, the MRE comprises a sequence having 100% identity to SEQ ID NO: 176.

Modified RNA stability regulatory element (MRE)
Variant 1
(SEQ ID NO: 175)
CGATAATCAACCTCTGGATTACAAAATTTGTGAAAGATTGACTGGT
ATTCTTAACTATGTTGCTCCTTTTACGCTATGTGGATACGCTGCTTT
AATGCCTTTGTATCATGCTATTGCTTCCCGTATGGCTTTCATTTTCTC
CTCCTTGTATAAATCCTGGTTGCTGTCTCTTTATGAGGAGTTGTGGC
CCGTTGTCAGGCAACGTGGCGTGGTGTGCACTGTGTTTGCTGACGC
AACCCCCACTGGTTGGGGCATTGCCACCACCTGTCAGCTCCTTTCC
GGGACTTTCGCTTTCCCCCTCCCTATTGCCACGGCGGAACTCATCGC
CGCCTGCCTTGCCCGCTGCTGGACAGGGGCTCGGCTGTTGGGCACT
GACAATTCCGTGGTGTTGTCGGGGAAATCATCGTCCTTTCCTTGGCT
GCTCGCCTGTGTTGCCACCTGGATTCTGCGCGGGACGTCCTTCTGCT
ACGTCCCTTCGGCCCTCAATCCAGCGGACCTTCCTTCCCGCGGCCT
GCTGCCGGCTCTGCGGCCTCTTCCGCGTCTTCGCCTTCGCCCTCAGA
CGAGTCGGATCTCCCTTTGGGCCGCCTCCCCGCATCGG
Modified RNA stability regulatory element (MRE)
Variant 2
(SEQ ID NO: 176)
GAGCATCTTACCGCCATTTATACCCATATTTGTTCTGTTTTTCTTGAT
TTGGGTATACATTTAAATGTTAATAAAACAAAATGGTGGGGCAATC
ATTTACATTTTTAGGGATATGTAATTACTAGTTCAGGTGTATTGCCA
CAAGACAAACATGTTAAGAAACTTTCCCGTTATTTACGCTCTGTTC
CTGTTAATCAACCTCTGGATTACAAAATTTGTGAAAGATTGACTGA
TATTCTTAACTATGTTGCTCCTTTTACGCTGTGTGGATATGCTGCTTT
ATAGCCTCTGTATCTAGCTATTGCTTCCCGTACGGCTTTCGTTTTCT
CCTCCTTGTATAAATCCTGGTTGCTGTCTCTTTTAGAGGAGTTGTGG
CCCGTTGTCCGTCAACGTGGCGTGGTGTGCTCTGTGTTTGCTGACGC
AACCCCCACTGGCTGGGGCATTGCCACCACCTGTCAACTCCTTTCT
GGGACTTTCGCTTTCCCCCTCCCGATCGCCACGGCAGAACTCATCG
CCGCCTGCCTTGCCCGCTGCTGGACAGGGGCTAGGTTGCTGGGCAC
TGATAATTCCGTGGTGTTGTC

In some embodiments, rAAV vectors of the present disclosure have nucleotide sequences at least 85% sequence identical (e.g., at least 85%, at least 90%, at least 91%, at least 92%, at least 93%, at least 94%, at least 95%, at least 96%, at least 97%, at least 98%, at least 99%, or 100%) to a nucleotide sequence listed in TABLE 9.

TABLE 9
Exemplary AAV2 Vector Sequences

| Construct | Nucleotide Sequence |
|---|---|
| AAV2- | CCTGCAGGCAGCTGCGCGCTCGCTCGCTCACTGAGGCCGCCCG |
| Pertuzamab - | GGCAAAGCCCGGGCGTCGGGCGACCTTTGGTCGCCCGGCCTCA |
| CD3 | GTGAGCGAGCGAGCGCGCAGAGAGGGAGTGGCCAACTCCATC |
| (SEQ ID NO: | ACTAGGGGTTCCTTCTAGCAACTTTGTATAGAAAAGTTGCTCGA |
| 83) | CATTGATTATTGACTAGTTATTAATAGTAATCAATTACGGGGTC |
| | ATTAGTTCATAGCCCATATATGGAGTTCCGCGTTACATAACTTA |
| | CGGTAAATGGCCCGCCTGGCTGACCGCCCAACGACCCCCGCCC |
| | ATTGACGTCAATAATGACGTATGTTCCCATAGTAACGCCAATA |
| | GGGACTTTCCATTGACGTCAATGGGTGGAGTATTTACGGTAAA |
| | CTGCCCACTTGGCAGTACATCAAGTGTATCATATGCCAAGTAC |
| | GCCCCCTATTGACGTCAATGACGGTAAATGGCCCGCCTGGCAT |
| | TATGCCCAGTACATGACCTTATGGGACTTTCCTACTTGGCAGTA |
| | CATCTACGTATTAGTCATCGCTATTACCATGGTCGAGGTGAGCC |
| | CCACGTTCTGCTTCACTCTCCCCATCTCCCCCCCCTCCCCACCCC |
| | CAATTTTGTATTTATTTATTTTTTAATTATTTTGTGCAGCGATGG |
| | GGGCGGGGGGGGGGGGGGGGCGCGCGCCAGGCGGGGCGGGGC |
| | GGGGCGAGGGGCGGGGGGGGCGAGGCGGAGAGGTGCGGCGG |
| | CAGCCAATCAGAGCGGCGCGCTCCGAAAGTTTCCTTTTATGGC |
| | GAGGCGGCGGCGGCGGCGGCCCTATAAAAAGCGAAGCGCGCG |
| | GCGGGCGGGAGTCGCTGCGCGCTGCCTTCGCCCCGTGCCCCGC |
| | TCCGCCGCCGCCTCGCGCCGCCCGCCCCGGCTCTGACTGACCGC |
| | GTTACTCCCACAGGTGAGCGGGCGGGACGGCCCTTCTCCTCCG |
| | GGCTGTAATTAGCGCTTGGTTTAATGACGGCTTGTTTCTTTTCT |
| | GTGGCTGCGTGAAAGCCTTGAGGGGCTCCGGGAGGGCCCTTTG |
| | TGCGGGGGGAGCGGCTCGGGGGGTGCGTGCGTGTGTGTGTGCG |
| | TGGGGAGCGCCGCGTGCGGCTCCGCGCTGCCCGGCGGCTGTGA |
| | GCGCTGCGGGCGCGGCGCGGGGCTTTGTGCGCTCCGCAGTGTG |
| | CGCGAGGGGAGCGCGGCCGGGGGCGGTGCCCCGCGGTGCGGG |
| | GGGGGCTGCGAGGGGAACAAAGGCTGCGTGCGGGGTGTGTGC |
| | GTGGGGGGGTGAGCAGGGGGTGTGGGCGCGTCGGTCGGGCTG |
| | CAACCCCCCCTGCACCCCCCTCCCCGAGTTGCTGAGCACGGCCC |
| | GGCTTCGGGTGCGGGGCTCCGTACGGGGCGTGGCGCGGGGCTC |
| | GCCGTGCCGGGCGGGGGGTGGCGGCAGGTGGGGGTGCCGGGC |
| | GGGGCGGGGCCGCCTCGGGCCGGGGAGGGCTCGGGGGAGGGG |
| | CGCGGCGGCCCCCGGAGCGCCGGCGGCTGTCGAGGCGCGGCG |
| | AGCCGCAGCCATTGCCTTTTATGGTAATCGTGCGAGAGGGCGC |
| | AGGGACTTCCTTTGTCCCAAATCTGTGCGGAGCCGAAATCTGG |
| | GAGGCGCCGCCGCACCCCCTCTAGCGGGCGCGGGGCGAAGCG |
| | GTGCGGCGCCGGCAGGAAGGAAATGGGCGGGGAGGGCCTTCG |
| | TGCGTCGCCGCGCCGCCGTCCCCTTCTCCCTCTCCAGCCTCGGG |
| | GCTGTCCGCGGGGGGACGGCTGCCTTCGGGGGGGACGGGGCA |
| | GGGCGGGGTTCGGCTTCTGGCGTGTGACCGGCGGCTCTAGAGC |
| | CTCTGCTAACCATGTTCATGCCTTCTTCTTTTTCCTACAGCTCCT |
| | GGGCAACGTGCTGGTTATTGTGCTGTCTCATCATTTTGGCAAAG |
| | AATTGCAAGTTTGTACAAAAAAGCAGGCTGCCACCATGTGGTG |
| | GAGACTGTGGTGGCTGCTGCTCCTGCTGCTCCTGCTGTGGCCAA |
| | TGGTGTGGGCTGCTGACATTCAGATGACTCAGTCTCCCAGCTCC |
| | CTGTCTGCCTCTGTGGGAGACAGAGTGACAATCACATGTAAGG |
| | CCTCCCAGGATGTGTCTATTGGAGTGGCCTGGTACCAGCAGAA |
| | ACCTGGAAAGGCCCCAAAACTGCTGATCTACTCTGCCTCCTAC |
| | AGGTATACAGGAGTGCCTTCCAGATTCTCTGGCTCTGGCTCTGG |
| | CACAGATTTCACTCTTACCATCAGTTCCCTCCAGCCTGAGGATT |
| | TTGCTACCTACTATTGCCAGCAGTACTACATCTATCCTTACACA |
| | TTTGGCCAGGGAACAAAAGTGGAGATCAAGTCCTGTGGAGGGG |
| | GATCTGGGGGAGGGGGGTCAGGAGGGGGAGGCTCTGAGGTGC |
| | AGCTGGTGGAGTCTGGGGGAGGCCTGGTGCAGCCAGGGGGCTC |
| | ACTGAGACTGAGCTGTGCTGCCTCTGGGTTTACATTTACAGACT |
| | ATACCATGGACTGGGTGAGACAGGCCCCAGGGAAGGGCCTGG |
| | AGTGGGTGGCAGATGTGAACCCAAATTCTGGAGGGTCCATCTA |
| | CAATCAGAGGTTCAAGGGGAGATTTACCCTGTCTGTGGATAGA |
| | AGCAAGAACACACTGTACCTTCAGATGAACTCCCTGAGAGCTG |
| | AGGACACAGCTGTGTACTACTGTGCCAGAAACCTGGGCCCATC |
| | CTTTTACTTTGACTACTGGGGACAGGGCACACTGGTGACAGTCT |
| | CCTCTGGGGGAGGAGGCTCTGATATTAAGCTCCAGCAGTCTGG |
| | AGCTGAGCTGGCCAGACCTGGAGCCTCTGTGAAGATGAGTTGC |
| | AAGACCTCTGGCTACACCTTTACCAGATACACAATGCATTGGG |
| | TGAAGCAGAGGCCAGGACAGGGGCTGGAATGGATTGGCTATAT |
| | CAACCCATCTAGAGGCTATACCAACTACAACCAGAAGTTTAAG |
| | GATAAAGCCACACTGACCACAGACAAGAGCTCCTCCACAGCCT |
| | ATATGCAGCTGTCTAGTCTGACCTCTGAGGATTCTGCTGTGTAT |
| | TATTGTGCCAGGTATTATGATGACCATTACTGCCTGGATTACTG |
| | GGGCCAGGGCACCACTCTGACAGTGAGCTCTGTGGAGGGGGGG |
| | TCTGGGGGCTCTGGAGGCTCTGGGGGCAGTGGGGGAGTGGATG |
| | ACATCCAACTGACCCAGTCCCCTGCCATCATGTCTGCCTCCCCA |
| | GGGGAAAAGGTCACCATGACCTGTAGAGCCTCTTCCTCTGTGT |
| | CCTACATGAACTGGTATCAGCAGAAGTCTGGCACCTCTCCTAA |
| | GAGGTGGATTTATGATACTAGCAAGGTGGCTTCTGGGGTGCCA |
| | TACAGGTTTTCTGGATCTGGTTCTGGAACCTCCTACTCCCTGAC |
| | AATCTCCTCCATGGAAGCTGAGGATGCAGCCACTTACTACTGTC |
| | AGCAGTGGAGTTCCAATCCACTCACTTTTGGGGCAGGCACAAA |
| | ACTGGAGCTGAAGTAACGATAATCAACCTCTGGATTACAAAAT |
| | TTGTGAAAGATTGACTGGTATTCTTAACTATGTTGCTCCTTTTA |
| | CGCTATGTGGATACGCTGCTTTAATGCCTTTGTATCATGCTATT |
| | GCTTCCCGTATGGCTTTCATTTTCTCCTCCTTGTATAAATCCTGG |
| | TTGCTGTCTCTTTATGAGGAGTTGTGGCCCGTTGTCAGGCAACG |
| | TGGCGTGGTGTGCACTGTGTTTGCTGACGCAACCCCCACTGGTT |
| | GGGGCATTGCCACCACCTGTCAGCTCCTTTCCGGGACTTTCGCT |
| | TTCCCCCTCCCTATTGCCACGGCGGAACTCATCGCCGCCTGCCT |
| | TGCCCGCTGCTGGACAGGGGCTCGGCTGTTGGGCACTGACAAT |
| | TCCGTGGTGTTGTCGGGGAAATCATCGTCCTTTCCTTGGCTGCT |
| | CGCCTGTGTTGCCACCTGGATTCTGCGCGGGACGTCCTTCTGCT |
| | ACGTCCCTTCGGCCCTCAATCCAGCGGACCTTCCTTCCCGCGGC |
| | CTGCTGCCGGCTCTGCGGCCTCTTCCGCGTCTTCGCCTTCGCCC |
| | TCAGACGAGTCGGATCTCCCTTTGGGCCGCCTCCCCGCATCGGA |
| | CCCAGCTTTCTTGTACAAAGTGGGAATTCCTAGAGCTCGCTGAT |
| | CAGCCTCGACTGTGCCTTCTAGTTGCCAGCCATCTGTTGTTTGC |
| | CCCTCCCCCGTGCCTTCCTTGACCCTGGAAGGTGCCACTCCCAC |
| | TGTCCTTTCCTAATAAAATGAGGAAATTGCATCGCATTGTCTGA |
| | GTAGGTGTCATTCTATTCTGGGGGGTGGGGTGGGGCAGGACAG |
| | CAAGGGGGAGGATTGGGAAGAGAATAGCAGGCATGCTGGGGA |
| | GGGCCGCAGGAACCCCTAGTGATGGAGTTGGCCACTCCCTCTC |
| | TGCGCGCTCGCTCGCTCACTGAGGCCGGGCGACCAAAGGTCGC |
| | CCGACGCCCGGGCTTTGCCCGGGCGGCCTCAGTGAGCGAGCGA |
| | GCGCGCAGCTGCCTGCAGGGGCGCCTGATGCGGTATTTTCTCCT |
| | TACGCATCTGTGCGGTATTTCACACCGCATACGTCAAAGCAAC |
| | CATAGTACGCGCCCTGTAGCGGCGCATTAAGCGCGGCGGGTGT |
| | GGTGGTTACGCGCAGCGTGACCGCTACACTTGCCAGCGCCTTA |
| | GCGCCCGCTCCTTTCGCTTTCTTCCCTTCCTTTCTCGCCACGTTC |
| | GCCGGCTTTCCCCGTCAAGCTCTAAATCGGGGGCTCCCTTTAGG |
| | GTTCCGATTTAGTGCTTTACGGCACCTCGACCCCAAAAAACTTG |
| | ATTTGGGTGATGGTTCACGTAGTGGGCCATCGCCCTGATAGAC |
| | GGTTTTTCGCCCTTTGACGTTGGAGTCCACGTTCTTTAATAGTG |
| | GACTCTTGTTCCAAACTGGAACAACACTCAACTCTATCTCGGGC |
| | TATTCTTTTGATTTATAAGGGATTTTGCCGATTTCGGTCTATTGG |
| | TTAAAAAATGAGCTGATTTAACAAAAATTTAACGCGAATTTTA |
| | ACAAAATATTAACGTTTACAATTTTATGGTGCACTCTCAGTACA |
| | ATCTGCTCTGATGCCGCATAGTTAAGCCAGCCCCGACACCCGC |
| | CAACACCCGCTGACGCGCCCTGACGGGCTTGTCTGCTCCCGGC |
| | ATCCGCTTACAGACAAGCTGTGACCGTCTCCGGGAGCTGCATG |
| | TGTCAGAGGTTTTCACCGTCATCACCGAAACGCGCGAGACGAA |
| | AGGGCCTCGTGATACGCCTATTTTTATAGGTTAATGTCATGATA |
| | ATAATGGTTTCTTAGACGTCCTGGCCCGTGTCTCAAAATCTCTG |
| | ATGTTACATTGCACAAGATAAAAATATATCATCATGAACAATA |
| | AAACTGTCTGCTTACATAAACAGTAATACAAGGGGTGTTATGA |
| | GCCATATTCAACGGGAAACGTCGAGGCCGCGATTAAATTCCAA |
| | CATGGATGCTGATTTATATGGGTATAAATGGGCTCGCGATAAT |
| | GTCGGGCAATCAGGTGCGACAATCTATCGCTTGTATGGGAAGC |
| | CCGATGCGCCAGAGTTGTTTCTGAAACATGGCAAAGGTAGCGT |
| | TGCCAATGATGTTACAGATGAGATGGTCAGACTAAACTGGCTG |
| | ACGGAATTTATGCCTCTTCCGACCATCAAGCATTTTATCCGTAC |
| | TCCTGATGATGCATGGTTACTCACCACTGCGATCCCCGGAAAA |
| | ACAGCATTCCAGGTATTAGAAGAATATCCTGATTCAGGTGAAA |
| | ATATTGTTGATGCGCTGGCAGTGTTCCTGCGCCGGTTGCATTCG |
| | ATTCCTGTTTGTAATTGTCCTTTTAACAGCGATCGCGTATTTCGT |
| | CTCGCTCAGGCGCAATCACGAATGAATAACGGTTTGGTTGATG |
| | CGAGTGATTTTGATGACGAGCGTAATGGCTGGCCTGTTGAACA |
| | AGTCTGGAAAGAAATGCATAAACTTTTGCCATTCTCACCGGATT |
| | CAGTCGTCACTCATGGTGATTTCTCACTTGATAACCTTATTTTTG |
| | ACGAGGGGAAATTAATAGGTTGTATTGATGTTGGACGAGTCGG |
| | AATCGCAGACCGATACCAGGATCTTGCCATCCTATGGAACTGC |
| | CTCGGTGAGTTTTCTCCTTCATTACAGAAACGGCTTTTTCAAAA |
| | ATATGGTATTGATAATCCTGATATGAATAAATTGCAGTTTCATT |
| | TGATGCTCGATGAGTTTTTCTAATCAGAATTGGTTAATTGGTTG |
| | TAACACTGGCAGAGCATTACGCTGACTTGACGGGACGGCGCAA |
| | GCTCATGACCAAAATCCCTTAACGTGAGTTACGCGTGAAGATC |
| | CTTTTTGATAATCTCATGACCAAAATCCCTTAACGTGAGTTTTC |
| | GTTCCACTGAGCGTCAGACCCCGTAGAAAAGATCAAAGGATCT |
| | TCTTGAGATCCTTTTTTTCTGCGCGTAATCTGCTGCTTGCAAAC |
| | AAAAAAACCACCGCTACCAGCGGTGGTTTGTTTGCCGGATCAA |
| | GAGCTACCAACTCTTTTTCCGAAGGTAACTGGCTTCAGCAGAG |
| | CGCAGATACCAAATACTGTTCTTCTAGTGTAGCCGTAGTTAGGC |
| | CACCACTTCAAGAACTCTGTAGCACCGCCTACATACCTCGCTCT |
| | GCTAATCCTGTTACCAGTGGCTGCTGCCAGTGGCGATAAGTCGT |
| | GTCTTACCGGGTTGGACTCAAGACGATAGTTACCGGATAAGGC |
| | GCAGCGGTCGGGCTGAACGGGGGGTTCGTGCACACAGCCCAGC |
| | TTGGAGCGAACGACCTACACCGAACTGAGATACCTACAGCGTG |
| | AGCTATGAGAAAGCGCCACGCTTCCCGAAGGGAGAAAGGCGG |
| | ACAGGTATCCGGTAAGCGGCAGGGTCGGAACAGGAGAGCGCA |
| | CGAGGGAGCTTCCAGGGGGAAACGCCTGGTATCTTTATAGTCC |
| | TGTCGGGTTTCGCCACCTCTGACTTGAGCGTCGATTTTTGTGAT |
| | GCTCGTCAGGGGGGCGGAGCCTATGGAAAAACGCCAGCAACG |
| | CGGCCTTTTTACGGTTCCTGGCCTTTTGCTGGCCTTTTGCTCACA |
| | TGT |
| AAV2- | CCTGCAGGCAGCTGCGCGCTCGCTCGCTCACTGAGGCCGCCCG |
| Herceptin | GGCAAAGCCCGGGCGTCGGGCGACCTTTGGTCGCCCGGCCTCA |
| (Trastuzumab)- | GTGAGCGAGCGAGCGCGCAGAGAGGGAGTGGCCAACTCCATC |
| CD3-MRE | ACTAGGGGTTCCTTCTAGCAACTTTGTATAGAAAAGTTGCTCGA |
| (SEQ ID NO: | CATTGATTATTGACTAGTTATTAATAGTAATCAATTACGGGGTC |
| 84) | ATTAGTTCATAGCCCATATATGGAGTTCCGCGTTACATAACTTA |
| | CGGTAAATGGCCCGCCTGGCTGACCGCCCAACGACCCCCGCCC |
| | ATTGACGTCAATAATGACGTATGTTCCCATAGTAACGCCAATA |
| | GGGACTTTCCATTGACGTCAATGGGTGGAGTATTTACGGTAAA |
| | CTGCCCACTTGGCAGTACATCAAGTGTATCATATGCCAAGTAC |
| | GCCCCCTATTGACGTCAATGACGGTAAATGGCCCGCCTGGCAT |
| | TATGCCCAGTACATGACCTTATGGGACTTTCCTACTTGGCAGTA |
| | CATCTACGTATTAGTCATCGCTATTACCATGGTCGAGGTGAGCC |
| | CCACGTTCTGCTTCACTCTCCCCATCTCCCCCCCCTCCCCACCCC |
| | CAATTTTGTATTTATTTATTTTTTAATTATTTTGTGCAGCGATGG |
| | GGGCGGGGGGGGGGGGGGGGCGCGCGCCAGGCGGGGGGGGC |
| | GGGGCGAGGGGGGGGCGGGGCGAGGCGGAGAGGTGCGGCGG |
| | CAGCCAATCAGAGCGGCGCGCTCCGAAAGTTTCCTTTTATGGC |
| | GAGGCGGCGGCGGCGGCGGCCCTATAAAAAGCGAAGCGCGCG |
| | GCGGGGGGAGTCGCTGCGCGCTGCCTTCGCCCCGTGCCCCGC |
| | TCCGCCGCCGCCTCGCGCCGCCCGCCCCGGCTCTGACTGACCGC |
| | GTTACTCCCACAGGTGAGCGGGCGGGACGGCCCTTCTCCTCCG |
| | GGCTGTAATTAGCGCTTGGTTTAATGACGGCTTGTTTCTTTTCT |
| | GTGGCTGCGTGAAAGCCTTGAGGGGCTCCGGGAGGGCCCTTTG |
| | TGCGGGGGGAGCGGCTCGGGGGGTGCGTGCGTGTGTGTGTGCG |
| | TGGGGAGCGCCGCGTGCGGCTCCGCGCTGCCCGGCGGCTGTGA |
| | GCGCTGCGGGCGCGGCGCGGGGCTTTGTGCGCTCCGCAGTGTG |
| | CGCGAGGGGAGCGCGGCCGGGGGCGGTGCCCCGCGGTGCGGG |
| | GGGGGCTGCGAGGGGAACAAAGGCTGCGTGCGGGGTGTGTGC |
| | GTGGGGGGGTGAGCAGGGGGTGTGGGCGCGTCGGTCGGGCTG |
| | CAACCCCCCCTGCACCCCCCTCCCCGAGTTGCTGAGCACGGCCC |
| | GGCTTCGGGTGCGGGGCTCCGTACGGGGCGTGGCGCGGGGCTC |
| | GCCGTGCCGGGCGGGGGGTGGCGGCAGGTGGGGGTGCCGGGC |
| | GGGGCGGGGCCGCCTCGGGCCGGGGAGGGCTCGGGGGAGGGG |
| | CGCGGCGGCCCCCGGAGCGCCGGCGGCTGTCGAGGCGCGGCG |
| | AGCCGCAGCCATTGCCTTTTATGGTAATCGTGCGAGAGGGCGC |
| | AGGGACTTCCTTTGTCCCAAATCTGTGCGGAGCCGAAATCTGG |
| | GAGGCGCCGCCGCACCCCCTCTAGCGGGCGCGGGGCGAAGCG |
| | GTGCGGCGCCGGCAGGAAGGAAATGGGCGGGGAGGGCCTTCG |
| | TGCGTCGCCGCGCCGCCGTCCCCTTCTCCCTCTCCAGCCTCGGG |
| | GCTGTCCGCGGGGGGACGGCTGCCTTCGGGGGGGACGGGGCA |
| | GGGCGGGGTTCGGCTTCTGGCGTGTGACCGGCGGCTCTAGAGC |
| | CTCTGCTAACCATGTTCATGCCTTCTTCTTTTTCCTACAGCTCCT |
| | GGGCAACGTGCTGGTTATTGTGCTGTCTCATCATTTTGGCAAAG |
| | AATTGCAAGTTTGTACAAAAAAGCAGGCTGCCACCATGTGGTG |
| | GAGGCTCTGGTGGCTCCTCCTGCTGCTGCTGCTGCTGTGGCCAA |
| | TGGTGTGGGCTGCTGACATTCAGATGACACAGTCCCCATCTTCC |
| | CTGTCTGCCTCTGTGGGAGACAGGGTGACAATCACTTGTAGGG |
| | CCAGCCAGGATGTGAACACAGCTGTGGCCTGGTATCAGCAGAA |
| | ACCTGGAAAGGCCCCAAAGCTGCTGATCTACTCAGCTTCCTTCC |
| | TGTATTCTGGAGTGCCATCTAGGTTTTCTGGCTCCAGGTCTGGC |
| | ACAGACTTTACCCTGACCATCTCTTCTCTGCAGCCTGAGGACTT |
| | TGCCACTTACTACTGTCAGCAGCATTACACAACCCCCCCAACCT |
| | TTGGCCAGGGCACCAAGGTGGAGATCAAGGGAGGAGGGGGAA |
| | GTGGGGGAGGAGGATCTGGAGGAGGGGGATCAGGAGGGGGAG |
| | GCTCAGAGGTGCAGCTGGTGGAGTCTGGGGGAGGCCTGGTGCA |
| | GCCTGGAGGCTCCCTGAGACTCTCCTGTGCTGCCTCTGGATTCA |
| | ATATCAAAGATACTTACATCCACTGGGTGAGACAGGCCCCTGG |
| | CAAAGGCCTGGAATGGGTGGCCAGAATCTATCCTACTAATGGC |
| | TACACCAGATATGCTGACTCTGTGAAGGGCAGATTTACAATCT |
| | CTGCTGACACCTCTAAAAATACAGCCTACCTGCAGATGAATTC |
| | CCTGAGAGCTGAAGACACAGCAGTGTACTACTGCAGCAGGTGG |
| | GGTGGAGATGGCTTCTATGCCATGGACTATTGGGGCCAGGGCA |
| | CCCTGGTGACAGTCTCATCTGGGGGAGGGGGCAGTGACATCAA |
| | GCTGCAGCAGTCTGGAGCTGAGCTGGCTAGACCTGGAGCCTCT |
| | GTGAAGATGTCCTGTAAGACCTCTGGTTACACATTTACCAGATA |
| | TACTATGCATTGGGTGAAACAGAGACCAGGCCAGGGACTGGAG |
| | TGGATTGGGTACATCAACCCTTCCAGAGGCTACACCAATTACA |
| | ATCAGAAGTTTAAGGATAAAGCCACTCTGACCACTGACAAGTC |
| | CAGCAGCACAGCTTACATGCAGCTGAGCTCCCTGACATCTGAG |
| | GACTCTGCTGTGTATTATTGTGCAAGATATTATGATGATCACTA |
| | TTGCCTGGACTACTGGGGGCAGGGCACTACACTGACAGTGTCC |
| | TCTGTGGAAGGAGGTTCTGGAGGCTCTGGAGGCTCTGGGGGCT |
| | CTGGAGGAGTGGATGATATCCAGCTGACACAGAGCCCTGCAAT |
| | CATGTCTGCTTCTCCTGGAGAGAAAGTGACCATGACATGCAGA |
| | GCCTCATCCTCTGTGAGCTATATGAATTGGTACCAACAGAAGTC |
| | TGGGACATCCCCCAAGAGATGGATCTATGATACAAGCAAAGTG |
| | GCCTCTGGGGTGCCATACAGATTCTCTGGATCTGGCTCTGGAAC |
| | ATCCTACAGCCTGACTATTAGCAGTATGGAGGCTGAGGATGCT |
| | GCCACCTACTACTGCCAGCAGTGGTCCAGCAACCCACTGACCT |
| | TTGGAGCTGGAACCAAACTGGAGCTGAAGTGACGATAATCAAC |
| | CTCTGGATTACAAAATTTGTGAAAGATTGACTGGTATTCTTAAC |
| | TATGTTGCTCCTTTTACGCTATGTGGATACGCTGCTTTAATGCCT |
| | TTGTATCATGCTATTGCTTCCCGTATGGCTTTCATTTTCTCCTCC |
| | TTGTATAAATCCTGGTTGCTGTCTCTTTATGAGGAGTTGTGGCC |
| | CGTTGTCAGGCAACGTGGCGTGGTGTGCACTGTGTTTGCTGACG |
| | CAACCCCCACTGGTTGGGGCATTGCCACCACCTGTCAGCTCCTT |
| | TCCGGGACTTTCGCTTTCCCCCTCCCTATTGCCACGGCGGAACT |
| | CATCGCCGCCTGCCTTGCCCGCTGCTGGACAGGGGCTCGGCTGT |
| | TGGGCACTGACAATTCCGTGGTGTTGTCGGGGAAATCATCGTC |
| | CTTTCCTTGGCTGCTCGCCTGTGTTGCCACCTGGATTCTGCGCG |
| | GGACGTCCTTCTGCTACGTCCCTTCGGCCCTCAATCCAGCGGAC |
| | CTTCCTTCCCGCGGCCTGCTGCCGGCTCTGCGGCCTCTTCCGCG |
| | TCTTCGCCTTCGCCCTCAGACGAGTCGGATCTCCCTTTGGGCCG |
| | CCTCCCCGCATCGGACCCAGCTTTCTTGTACAAAGTGGGAATTC |
| | CTAGAGCTCGCTGATCAGCCTCGACTGTGCCTTCTAGTTGCCAG |
| | CCATCTGTTGTTTGCCCCTCCCCCGTGCCTTCCTTGACCCTGGA |
| | AGGTGCCACTCCCACTGTCCTTTCCTAATAAAATGAGGAAATTG |
| | CATCGCATTGTCTGAGTAGGTGTCATTCTATTCTGGGGGGTGGG |
| | GTGGGGCAGGACAGCAAGGGGGAGGATTGGGAAGAGAATAGC |
| | AGGCATGCTGGGGAGGGCCGCAGGAACCCCTAGTGATGGAGTT |
| | GGCCACTCCCTCTCTGCGCGCTCGCTCGCTCACTGAGGCCGGGC |
| | GACCAAAGGTCGCCCGACGCCCGGGCTTTGCCCGGGCGGCCTC |
| | AGTGAGCGAGCGAGCGCGCAGCTGCCTGCAGGGGCGCCTGATG |
| | CGGTATTTTCTCCTTACGCATCTGTGCGGTATTTCACACCGCAT |
| | ACGTCAAAGCAACCATAGTACGCGCCCTGTAGCGGCGCATTAA |
| | GCGCGGCGGGTGTGGTGGTTACGCGCAGCGTGACCGCTACACT |
| | TGCCAGCGCCTTAGCGCCCGCTCCTTTCGCTTTCTTCCCTTCCTT |
| | TCTCGCCACGTTCGCCGGCTTTCCCCGTCAAGCTCTAAATCGGG |
| | GGCTCCCTTTAGGGTTCCGATTTAGTGCTTTACGGCACCTCGAC |
| | CCCAAAAAACTTGATTTGGGTGATGGTTCACGTAGTGGGCCAT |
| | CGCCCTGATAGACGGTTTTTCGCCCTTTGACGTTGGAGTCCACG |
| | TTCTTTAATAGTGGACTCTTGTTCCAAACTGGAACAACACTCAA |
| | CTCTATCTCGGGCTATTCTTTTGATTTATAAGGGATTTTGCCGAT |
| | TTCGGTCTATTGGTTAAAAAATGAGCTGATTTAACAAAAATTTA |
| | ACGCGAATTTTAACAAAATATTAACGTTTACAATTTTATGGTGC |
| | ACTCTCAGTACAATCTGCTCTGATGCCGCATAGTTAAGCCAGCC |
| | CCGACACCCGCCAACACCCGCTGACGCGCCCTGACGGGCTTGT |
| | CTGCTCCCGGCATCCGCTTACAGACAAGCTGTGACCGTCTCCGG |
| | GAGCTGCATGTGTCAGAGGTTTTCACCGTCATCACCGAAACGC |
| | GCGAGACGAAAGGGCCTCGTGATACGCCTATTTTTATAGGTTA |
| | ATGTCATGATAATAATGGTTTCTTAGACGTCCTGGCCCGTGTCT |
| | CAAAATCTCTGATGTTACATTGCACAAGATAAAAATATATCAT |
| | CATGAACAATAAAACTGTCTGCTTACATAAACAGTAATACAAG |
| | GGGTGTTATGAGCCATATTCAACGGGAAACGTCGAGGCCGCGA |
| | TTAAATTCCAACATGGATGCTGATTTATATGGGTATAAATGGGC |
| | TCGCGATAATGTCGGGCAATCAGGTGCGACAATCTATCGCTTG |
| | TATGGGAAGCCCGATGCGCCAGAGTTGTTTCTGAAACATGGCA |
| | AAGGTAGCGTTGCCAATGATGTTACAGATGAGATGGTCAGACT |
| | AAACTGGCTGACGGAATTTATGCCTCTTCCGACCATCAAGCATT |
| | TTATCCGTACTCCTGATGATGCATGGTTACTCACCACTGCGATC |
| | CCCGGAAAAACAGCATTCCAGGTATTAGAAGAATATCCTGATT |
| | CAGGTGAAAATATTGTTGATGCGCTGGCAGTGTTCCTGCGCCG |
| | GTTGCATTCGATTCCTGTTTGTAATTGTCCTTTTAACAGCGATC |
| | GCGTATTTCGTCTCGCTCAGGCGCAATCACGAATGAATAACGG |
| | TTTGGTTGATGCGAGTGATTTTGATGACGAGCGTAATGGCTGGC |
| | CTGTTGAACAAGTCTGGAAAGAAATGCATAAACTTTTGCCATT |
| | CTCACCGGATTCAGTCGTCACTCATGGTGATTTCTCACTTGATA |
| | ACCTTATTTTTGACGAGGGGAAATTAATAGGTTGTATTGATGTT |
| | GGACGAGTCGGAATCGCAGACCGATACCAGGATCTTGCCATCC |
| | TATGGAACTGCCTCGGTGAGTTTTCTCCTTCATTACAGAAACGG |
| | CTTTTTCAAAAATATGGTATTGATAATCCTGATATGAATAAATT |
| | GCAGTTTCATTTGATGCTCGATGAGTTTTTCTAATCAGAATTGG |
| | TTAATTGGTTGTAACACTGGCAGAGCATTACGCTGACTTGACG |
| | GGACGGCGCAAGCTCATGACCAAAATCCCTTAACGTGAGTTAC |
| | GCGTGAAGATCCTTTTTGATAATCTCATGACCAAAATCCCTTAA |
| | CGTGAGTTTTCGTTCCACTGAGCGTCAGACCCCGTAGAAAAGA |
| | TCAAAGGATCTTCTTGAGATCCTTTTTTTCTGCGCGTAATCTGCT |
| | GCTTGCAAACAAAAAAACCACCGCTACCAGCGGTGGTTTGTTT |
| | GCCGGATCAAGAGCTACCAACTCTTTTTCCGAAGGTAACTGGC |
| | TTCAGCAGAGCGCAGATACCAAATACTGTTCTTCTAGTGTAGCC |
| | GTAGTTAGGCCACCACTTCAAGAACTCTGTAGCACCGCCTACA |
| | TACCTCGCTCTGCTAATCCTGTTACCAGTGGCTGCTGCCAGTGG |
| | CGATAAGTCGTGTCTTACCGGGTTGGACTCAAGACGATAGTTA |
| | CCGGATAAGGCGCAGCGGTCGGGCTGAACGGGGGGTTCGTGCA |
| | CACAGCCCAGCTTGGAGCGAACGACCTACACCGAACTGAGATA |
| | CCTACAGCGTGAGCTATGAGAAAGCGCCACGCTTCCCGAAGGG |
| | AGAAAGGCGGACAGGTATCCGGTAAGCGGCAGGGTCGGAACA |
| | GGAGAGCGCACGAGGGAGCTTCCAGGGGGAAACGCCTGGTAT |
| | CTTTATAGTCCTGTCGGGTTTCGCCACCTCTGACTTGAGCGTCG |
| | ATTTTTGTGATGCTCGTCAGGGGGGCGGAGCCTATGGAAAAAC |
| | GCCAGCAACGCGGCCTTTTTACGGTTCCTGGCCTTTTGCTGGCC |
| | TTTTGCTCACATGT |
| AAV2- | CCTGCAGGCAGCTGCGCGCTCGCTCGCTCACTGAGGCCGCCCG |
| Hersintuzumab- | GGCAAAGCCCGGGCGTCGGGCGACCTTTGGTCGCCCGGCCTCA |
| CD3 | GTGAGCGAGCGAGCGCGCAGAGAGGGAGTGGCCAACTCCATC |
| (SEQ ID NO: | ACTAGGGGTTCCTTCTAGCAACTTTGTATAGAAAAGTTGCTCGA |
| 85) | CATTGATTATTGACTAGTTATTAATAGTAATCAATTACGGGGTC |
| | ATTAGTTCATAGCCCATATATGGAGTTCCGCGTTACATAACTTA |
| | CGGTAAATGGCCCGCCTGGCTGACCGCCCAACGACCCCCGCCC |
| | ATTGACGTCAATAATGACGTATGTTCCCATAGTAACGCCAATA |
| | GGGACTTTCCATTGACGTCAATGGGTGGAGTATTTACGGTAAA |
| | CTGCCCACTTGGCAGTACATCAAGTGTATCATATGCCAAGTAC |
| | GCCCCCTATTGACGTCAATGACGGTAAATGGCCCGCCTGGCAT |
| | TATGCCCAGTACATGACCTTATGGGACTTTCCTACTTGGCAGTA |
| | CATCTACGTATTAGTCATCGCTATTACCATGGTCGAGGTGAGCC |
| | CCACGTTCTGCTTCACTCTCCCCATCTCCCCCCCCTCCCCACCCC |
| | CAATTTTGTATTTATTTATTTTTTAATTATTTTGTGCAGCGATGG |
| | GGGCGGGGGGGGGGGGGGGGCGCGCGCCAGGCGGGGCGGGGC |
| | GGGGCGAGGGGCGGGGCGGGGCGAGGCGGAGAGGTGCGGCGG |
| | CAGCCAATCAGAGCGGCGCGCTCCGAAAGTTTCCTTTTATGGC |
| | GAGGCGGCGGCGGCGGCGGCCCTATAAAAAGCGAAGCGCGCG |
| | GCGGGCGGGAGTCGCTGCGCGCTGCCTTCGCCCCGTGCCCCGC |
| | TCCGCCGCCGCCTCGCGCCGCCCGCCCCGGCTCTGACTGACCGC |
| | GTTACTCCCACAGGTGAGCGGGCGGGACGGCCCTTCTCCTCCG |
| | GGCTGTAATTAGCGCTTGGTTTAATGACGGCTTGTTTCTTTTCT |
| | GTGGCTGCGTGAAAGCCTTGAGGGGCTCCGGGAGGGCCCTTTG |
| | TGCGGGGGGAGCGGCTCGGGGGGTGCGTGCGTGTGTGTGTGCG |
| | TGGGGAGCGCCGCGTGCGGCTCCGCGCTGCCCGGCGGCTGTGA |
| | GCGCTGCGGGCGCGGCGCGGGGCTTTGTGCGCTCCGCAGTGTG |
| | CGCGAGGGGAGCGCGGCCGGGGGCGGTGCCCCGCGGTGCGGG |
| | GGGGGCTGCGAGGGGAACAAAGGCTGCGTGCGGGGTGTGTGC |
| | GTGGGGGGGTGAGCAGGGGGTGTGGGCGCGTCGGTCGGGCTG |
| | CAACCCCCCCTGCACCCCCCTCCCCGAGTTGCTGAGCACGGCCC |
| | GGCTTCGGGTGCGGGGCTCCGTACGGGGCGTGGCGCGGGGCTC |
| | GCCGTGCCGGGCGGGGGGTGGCGGCAGGTGGGGGTGCCGGGC |
| | GGGGCGGGGCCGCCTCGGGCCGGGGAGGGCTCGGGGGAGGGG |
| | CGCGGCGGCCCCCGGAGCGCCGGCGGCTGTCGAGGCGCGGCG |
| | AGCCGCAGCCATTGCCTTTTATGGTAATCGTGCGAGAGGGCGC |
| | AGGGACTTCCTTTGTCCCAAATCTGTGCGGAGCCGAAATCTGG |
| | GAGGCGCCGCCGCACCCCCTCTAGCGGGCGCGGGGCGAAGCG |
| | GTGCGGCGCCGGCAGGAAGGAAATGGGCGGGGAGGGCCTTCG |
| | TGCGTCGCCGCGCCGCCGTCCCCTTCTCCCTCTCCAGCCTCGGG |
| | GCTGTCCGCGGGGGGACGGCTGCCTTCGGGGGGGACGGGGCA |
| | GGGCGGGGTTCGGCTTCTGGCGTGTGACCGGCGGCTCTAGAGC |
| | CTCTGCTAACCATGTTCATGCCTTCTTCTTTTTCCTACAGCTCCT |
| | GGGCAACGTGCTGGTTATTGTGCTGTCTCATCATTTTGGCAAAG |
| | AATTGCAAGTTTGTACAAAAAAGCAGGCTGCCACCATGTGGTG |
| | GAGACTGTGGTGGCTGCTGCTGTTGCTGCTGCTGCTCTGGCCCA |
| | TGGTGTGGGCTGCTGACATTGTGATGACACAGTCCCCCTCTAGC |
| | CTGTCTGCTTCTGTGGGAGACAGGGTGACAATCACATGCAGAG |
| | CCTCTCAGAATGTGAGAACAGCTGTGGCATGGTTCCAGCAGAA |
| | ACCAGGAAAGGCCCCCAAGGCCCTGATCTACCTGGCCTCTAAC |
| | AGGCACACAGGGGTGCCTGATAGGTTCACAGGCTCTGGATCTG |
| | GGACAGAGTTTACCCTGACCATCAGCAACCTGCAGCCTGAGGA |
| | CTTTGCTACATACTTCTGCCTGCAGCACAACAGCTATCCTCTGA |
| | CTTTTGGAGGAGGCACCAAAGTGGAGATCAAGGGAGGAGGAG |
| | GTTCTGGGGGAGGAGGATCTGGAGGAGGAGGATCTGAGGTAA |
| | AGCTGGTGGAGTCTGGAGGAGGCCTGGTGAAACCAGGGGGCTC |
| | TCTGAGACTGAGTTGTGCCACATCTGGCTTTTCCTTTAGCAGCT |
| | ATTATATGTACTGGGTGAGGCAGGCCCCAGGCAAAAGGCTGGA |
| | GTGGGTGGCCTACATCAGCTCTGGCTCTGAAATTTATTACTCTG |
| | ACTCTGTCAAGGGCAGATTCACCATCAGCAGAGATTCTGCCAA |
| | GAACACCCTGTATCTCCAGATGAACTCTCTGAGAGCTGAGGAC |
| | ACAGCTGTGTATTACTGTGCTAGACTGGGAGATGATGGAATGG |
| | ACTGGTGGGGCCAGGGAACCACATGGACAGTGTCTTCTGGAGG |
| | AGGAGGATCTGACATCAAGCTGCAGCAGTCTGGAGCTGAGCTG |
| | GCCAGACCTGGAGCCTCAGTGAAGATGAGCTGCAAAACATCTG |
| | GATACACCTTCACCAGATACACCATGCACTGGGTCAAGCAGAG |
| | ACCTGGACAGGGACTGGAGTGGATTGGCTATATTAACCCTTCT |
| | AGAGGCTACACCAACTACAACCAAAAGTTCAAGGACAAAGCC |
| | ACACTGACCACAGACAAGTCCTCCAGCACTGCATACATGCAGT |
| | TGAGTAGCCTGACCTCAGAGGATTCTGCTGTGTACTATTGTGCT |
| | AGGTATTATGATGACCATTACTGTCTGGATTATTGGGGACAGG |
| | GCACCACCCTGACAGTGAGCTCTGTGGAAGGAGGCTCTGGAGG |
| | CTCTGGAGGCTCTGGAGGCAGTGGAGGAGTGGATGACATTCAG |
| | CTGACCCAGAGCCCTGCCATTATGTCTGCATCACCAGGAGAGA |
| | AGGTGACCATGACATGCAGGGCAAGTTCTTCTGTGTCCTACAT |
| | GAACTGGTATCAGCAGAAGTCTGGAACCTCCCCTAAAAGATGG |
| | ATCTATGATACCAGTAAGGTGGCATCAGGAGTGCCCTACAGAT |
| | TCTCTGGGTCTGGATCTGGAACAAGCTACTCCCTGACCATCTCT |
| | AGCATGGAGGCTGAGGATGCTGCCACCTACTACTGCCAGCAGT |
| | GGTCCAGCAACCCCCTGACATTTGGGGCTGGGACCAAGCTGGA |
| | ACTGAAATAACGATAATCAACCTCTGGATTACAAAATTTGTGA |
| | AAGATTGACTGGTATTCTTAACTATGTTGCTCCTTTTACGCTAT |
| | GTGGATACGCTGCTTTAATGCCTTTGTATCATGCTATTGCTTCC |
| | CGTATGGCTTTCATTTTCTCCTCCTTGTATAAATCCTGGTTGCTG |
| | TCTCTTTATGAGGAGTTGTGGCCCGTTGTCAGGCAACGTGGCGT |
| | GGTGTGCACTGTGTTTGCTGACGCAACCCCCACTGGTTGGGGC |
| | ATTGCCACCACCTGTCAGCTCCTTTCCGGGACTTTCGCTTTCCC |
| | CCTCCCTATTGCCACGGCGGAACTCATCGCCGCCTGCCTTGCCC |
| | GCTGCTGGACAGGGGCTCGGCTGTTGGGCACTGACAATTCCGT |
| | GGTGTTGTCGGGGAAATCATCGTCCTTTCCTTGGCTGCTCGCCT |
| | GTGTTGCCACCTGGATTCTGCGCGGGACGTCCTTCTGCTACGTC |
| | CCTTCGGCCCTCAATCCAGCGGACCTTCCTTCCCGCGGCCTGCT |
| | GCCGGCTCTGCGGCCTCTTCCGCGTCTTCGCCTTCGCCCTCAGA |
| | CGAGTCGGATCTCCCTTTGGGCCGCCTCCCCGCATCGGACCCAG |
| | CTTTCTTGTACAAAGTGGGAATTCCTAGAGCTCGCTGATCAGCC |
| | TCGACTGTGCCTTCTAGTTGCCAGCCATCTGTTGTTTGCCCCTCC |
| | CCCGTGCCTTCCTTGACCCTGGAAGGTGCCACTCCCACTGTCCT |
| | TTCCTAATAAAATGAGGAAATTGCATCGCATTGTCTGAGTAGG |
| | TGTCATTCTATTCTGGGGGGGGGGTGGGGCAGGACAGCAAGG |
| | GGGAGGATTGGGAAGAGAATAGCAGGCATGCTGGGGAGGGCC |
| | GCAGGAACCCCTAGTGATGGAGTTGGCCACTCCCTCTCTGCGC |
| | GCTCGCTCGCTCACTGAGGCCGGGCGACCAAAGGTCGCCCGAC |
| | GCCCGGGCTTTGCCCGGGCGGCCTCAGTGAGCGAGCGAGCGCG |
| | CAGCTGCCTGCAGGGGCGCCTGATGCGGTATTTTCTCCTTACGC |
| | ATCTGTGCGGTATTTCACACCGCATACGTCAAAGCAACCATAG |
| | TACGCGCCCTGTAGCGGCGCATTAAGCGCGGCGGGTGTGGTGG |
| | TTACGCGCAGCGTGACCGCTACACTTGCCAGCGCCTTAGCGCC |
| | CGCTCCTTTCGCTTTCTTCCCTTCCTTTCTCGCCACGTTCGCCGG |
| | CTTTCCCCGTCAAGCTCTAAATCGGGGGCTCCCTTTAGGGTTCC |
| | GATTTAGTGCTTTACGGCACCTCGACCCCAAAAAACTTGATTTG |
| | GGTGATGGTTCACGTAGTGGGCCATCGCCCTGATAGACGGTTTT |
| | TCGCCCTTTGACGTTGGAGTCCACGTTCTTTAATAGTGGACTCT |
| | TGTTCCAAACTGGAACAACACTCAACTCTATCTCGGGCTATTCT |
| | TTTGATTTATAAGGGATTTTGCCGATTTCGGTCTATTGGTTAAA |
| | AAATGAGCTGATTTAACAAAAATTTAACGCGAATTTTAACAAA |
| | ATATTAACGTTTACAATTTTATGGTGCACTCTCAGTACAATCTG |
| | CTCTGATGCCGCATAGTTAAGCCAGCCCCGACACCCGCCAACA |
| | CCCGCTGACGCGCCCTGACGGGCTTGTCTGCTCCCGGCATCCGC |
| | TTACAGACAAGCTGTGACCGTCTCCGGGAGCTGCATGTGTCAG |
| | AGGTTTTCACCGTCATCACCGAAACGCGCGAGACGAAAGGGCC |
| | TCGTGATACGCCTATTTTTATAGGTTAATGTCATGATAATAATG |
| | GTTTCTTAGACGTCCTGGCCCGTGTCTCAAAATCTCTGATGTTA |
| | CATTGCACAAGATAAAAATATATCATCATGAACAATAAAACTG |
| | TCTGCTTACATAAACAGTAATACAAGGGGTGTTATGAGCCATA |
| | TTCAACGGGAAACGTCGAGGCCGCGATTAAATTCCAACATGGA |
| | TGCTGATTTATATGGGTATAAATGGGCTCGCGATAATGTCGGG |
| | CAATCAGGTGCGACAATCTATCGCTTGTATGGGAAGCCCGATG |
| | CGCCAGAGTTGTTTCTGAAACATGGCAAAGGTAGCGTTGCCAA |
| | TGATGTTACAGATGAGATGGTCAGACTAAACTGGCTGACGGAA |
| | TTTATGCCTCTTCCGACCATCAAGCATTTTATCCGTACTCCTGAT |
| | GATGCATGGTTACTCACCACTGCGATCCCCGGAAAAACAGCAT |
| | TCCAGGTATTAGAAGAATATCCTGATTCAGGTGAAAATATTGTT |
| | GATGCGCTGGCAGTGTTCCTGCGCCGGTTGCATTCGATTCCTGT |
| | TTGTAATTGTCCTTTTAACAGCGATCGCGTATTTCGTCTCGCTC |
| | AGGCGCAATCACGAATGAATAACGGTTTGGTTGATGCGAGTGA |
| | TTTTGATGACGAGCGTAATGGCTGGCCTGTTGAACAAGTCTGG |
| | AAAGAAATGCATAAACTTTTGCCATTCTCACCGGATTCAGTCGT |
| | CACTCATGGTGATTTCTCACTTGATAACCTTATTTTTGACGAGG |
| | GGAAATTAATAGGTTGTATTGATGTTGGACGAGTCGGAATCGC |
| | AGACCGATACCAGGATCTTGCCATCCTATGGAACTGCCTCGGT |
| | GAGTTTTCTCCTTCATTACAGAAACGGCTTTTTCAAAAATATGG |
| | TATTGATAATCCTGATATGAATAAATTGCAGTTTCATTTGATGC |
| | TCGATGAGTTTTTCTAATCAGAATTGGTTAATTGGTTGTAACAC |
| | TGGCAGAGCATTACGCTGACTTGACGGGACGGCGCAAGCTCAT |
| | GACCAAAATCCCTTAACGTGAGTTACGCGTGAAGATCCTTTTTG |
| | ATAATCTCATGACCAAAATCCCTTAACGTGAGTTTTCGTTCCAC |
| | TGAGCGTCAGACCCCGTAGAAAAGATCAAAGGATCTTCTTGAG |
| | ATCCTTTTTTTCTGCGCGTAATCTGCTGCTTGCAAACAAAAAAA |
| | CCACCGCTACCAGCGGTGGTTTGTTTGCCGGATCAAGAGCTAC |
| | CAACTCTTTTTCCGAAGGTAACTGGCTTCAGCAGAGCGCAGAT |
| | ACCAAATACTGTTCTTCTAGTGTAGCCGTAGTTAGGCCACCACT |
| | TCAAGAACTCTGTAGCACCGCCTACATACCTCGCTCTGCTAATC |
| | CTGTTACCAGTGGCTGCTGCCAGTGGCGATAAGTCGTGTCTTAC |
| | CGGGTTGGACTCAAGACGATAGTTACCGGATAAGGCGCAGCGG |
| | TCGGGCTGAACGGGGGGTTCGTGCACACAGCCCAGCTTGGAGC |
| | GAACGACCTACACCGAACTGAGATACCTACAGCGTGAGCTATG |
| | AGAAAGCGCCACGCTTCCCGAAGGGAGAAAGGCGGACAGGTA |
| | TCCGGTAAGCGGCAGGGTCGGAACAGGAGAGCGCACGAGGGA |
| | GCTTCCAGGGGGAAACGCCTGGTATCTTTATAGTCCTGTCGGGT |
| | TTCGCCACCTCTGACTTGAGCGTCGATTTTTGTGATGCTCGTCA |
| | GGGGGGCGGAGCCTATGGAAAAACGCCAGCAACGCGGCCTTTT |
| | TACGGTTCCTGGCCTTTTGCTGGCCTTTTGCTCACATGT |
| AAV2-huA21- | CCTGCAGGCAGCTGCGCGCTCGCTCGCTCACTGAGGCCGCCCG |
| CD3 | GGCAAAGCCCGGGCGTCGGGCGACCTTTGGTCGCCCGGCCTCA |
| (SEQ ID NO: | GTGAGCGAGCGAGCGCGCAGAGAGGGAGTGGCCAACTCCATC |
| 82) | ACTAGGGGTTCCTTCTAGCAACTTTGTATAGAAAAGTTGCTCGA |
| | CATTGATTATTGACTAGTTATTAATAGTAATCAATTACGGGGTC |
| | ATTAGTTCATAGCCCATATATGGAGTTCCGCGTTACATAACTTA |
| | CGGTAAATGGCCCGCCTGGCTGACCGCCCAACGACCCCCGCCC |
| | ATTGACGTCAATAATGACGTATGTTCCCATAGTAACGCCAATA |
| | GGGACTTTCCATTGACGTCAATGGGTGGAGTATTTACGGTAAA |
| | CTGCCCACTTGGCAGTACATCAAGTGTATCATATGCCAAGTAC |
| | GCCCCCTATTGACGTCAATGACGGTAAATGGCCCGCCTGGCAT |
| | TATGCCCAGTACATGACCTTATGGGACTTTCCTACTTGGCAGTA |
| | CATCTACGTATTAGTCATCGCTATTACCATGGTCGAGGTGAGCC |
| | CCACGTTCTGCTTCACTCTCCCCATCTCCCCCCCCTCCCCACCCC |
| | CAATTTTGTATTTATTTATTTTTTAATTATTTTGTGCAGCGATGG |
| | GGGCGGGGGGGGGGGGGGGGCGCGCGCCAGGCGGGGCGGGGC |
| | GGGGCGAGGGGCGGGGCGGGGCGAGGCGGAGAGGTGCGGCGG |
| | CAGCCAATCAGAGCGGCGCGCTCCGAAAGTTTCCTTTTATGGC |
| | GAGGCGGCGGCGGCGGCGGCCCTATAAAAAGCGAAGCGCGCG |
| | GCGGGCGGGAGTCGCTGCGCGCTGCCTTCGCCCCGTGCCCCGC |
| | TCCGCCGCCGCCTCGCGCCGCCCGCCCCGGCTCTGACTGACCGC |
| | GTTACTCCCACAGGTGAGCGGGCGGGACGGCCCTTCTCCTCCG |
| | GGCTGTAATTAGCGCTTGGTTTAATGACGGCTTGTTTCTTTTCT |
| | GTGGCTGCGTGAAAGCCTTGAGGGGCTCCGGGAGGGCCCTTTG |
| | TGCGGGGGGAGCGGCTCGGGGGGTGCGTGCGTGTGTGTGTGCG |
| | TGGGGAGCGCCGCGTGCGGCTCCGCGCTGCCCGGCGGCTGTGA |
| | GCGCTGCGGGCGCGGCGCGGGGCTTTGTGCGCTCCGCAGTGTG |
| | CGCGAGGGGAGCGCGGCCGGGGGCGGTGCCCCGCGGTGCGGG |
| | GGGGGCTGCGAGGGGAACAAAGGCTGCGTGCGGGGTGTGTGC |
| | GTGGGGGGGTGAGCAGGGGGTGTGGGCGCGTCGGTCGGGCTG |
| | CAACCCCCCCTGCACCCCCCTCCCCGAGTTGCTGAGCACGGCCC |
| | GGCTTCGGGTGCGGGGCTCCGTACGGGGCGTGGCGCGGGGCTC |
| | GCCGTGCCGGGCGGGGGGTGGCGGCAGGTGGGGGTGCCGGGC |
| | GGGGCGGGGCCGCCTCGGGCCGGGGAGGGCTCGGGGGAGGGG |
| | CGCGGCGGCCCCCGGAGCGCCGGCGGCTGTCGAGGCGCGGCG |
| | AGCCGCAGCCATTGCCTTTTATGGTAATCGTGCGAGAGGGCGC |
| | AGGGACTTCCTTTGTCCCAAATCTGTGCGGAGCCGAAATCTGG |
| | GAGGCGCCGCCGCACCCCCTCTAGCGGGCGCGGGGCGAAGCG |
| | GTGCGGCGCCGGCAGGAAGGAAATGGGCGGGGAGGGCCTTCG |
| | TGCGTCGCCGCGCCGCCGTCCCCTTCTCCCTCTCCAGCCTCGGG |
| | GCTGTCCGCGGGGGGACGGCTGCCTTCGGGGGGGACGGGGCA |
| | GGGCGGGGTTCGGCTTCTGGCGTGTGACCGGCGGCTCTAGAGC |
| | CTCTGCTAACCATGTTCATGCCTTCTTCTTTTTCCTACAGCTCCT |
| | GGGCAACGTGCTGGTTATTGTGCTGTCTCATCATTTTGGCAAAG |
| | AATTGCAAGTTTGTACAAAAAAGCAGGCTGCCACCATGTGGTG |
| | GAGACTGTGGTGGCTGCTGCTGCTGCTGCTGCTCCTGTGGCCTA |
| | TGGTGTGGGCTGCTGCTGACATAGTGCTGACCCAGAGTCCTGA |
| | TTCCCTGGCTGTGTCTCTGGGAGAGAGAGTGACCATCAATTGC |
| | AAGTCCAGCCAGCCCCTGGAATACAGCAATAATCAGTGGAACT |
| | ACCTGGCTTGGTACCAGCAGAAACCTGGACAGTCCCCTAAGCT |
| | GCTGATTAGCTGGGCCAGCACCAGAAAGTCTGGAGTCCCAGAC |
| | AGATTTTCTGGTTCTGGCTCTGGAACTGACTTCACCCTCACAAT |
| | CAGCTCTGTGCAGGCTGAAGATGTGGCTGTGTATTACTGTGGCC |
| | AGTACTCTGATTATCCCAACACCTTTGGGGCTGGCACAAAGCT |
| | GGAAATCAAGAGAGGAGGTGGAGGTTCTGGAGGAGGGGGATC |
| | TGGTGGAGGGGGCTCTGGAGGAGGAGGGTCTCAGGTGCAGCTG |
| | GTGCAGTCTGGAGCTGAGGTGGTGAAACCAGGGGCTTCTGTGA |
| | AGATTTCCTGTAAGGCCTCTGGCTACCCTTTCACTCAGTACTTC |
| | ATTCATTGGGTCAAGCAGAATCCTGGGCAGAGACTGGAATGGA |
| | TTGGACAAATCTCCTCCAGCTATGCCACAGTGGACTACAATCA |
| | GAAGTTCAAGGGCAAGGCAACCCTGACTGTGGACACATCTGCC |
| | AGCATTGCCTATATGGAACTGAGCAGCCTGAGATCTGAAGATA |
| | CAGCTGTGTATTACTGTGTGAGATCTGGCAATTATGAGGAGTAT |
| | GCCATGGACTACTGGGGACAGGGCACCCTGGTGACTGTGTCTG |
| | GAGGAGGAGGTTCTGACATCAAACTGCAGCAGAGTGGGGCTG |
| | AGCTGGCCAGACCTGGAGCCTCTGTCAAGATGAGCTGCAAAAC |
| | CTCAGGCTACACATTCACCAGATACACCATGCACTGGGTGAAA |
| | CAGAGACCTGGACAGGGCCTGGAGTGGATTGGCTACATTAACC |
| | CATCAAGAGGCTATACCAACTATAACCAGAAATTCAAGGACAA |
| | GGCCACCCTGACCACAGACAAAAGCAGCAGTACAGCTTACATG |
| | CAGCTGTCCTCCCTGACCTCTGAGGACTCTGCTGTGTACTATTG |
| | TGCCAGATACTATGATGACCACTACTGCCTGGATTATTGGGGCC |
| | AGGGCACAACACTGACAGTGTCTTCTGTGGAGGGTGGCTCAGG |
| | TGGGTCTGGGGGCTCTGGAGGCTCTGGTGGAGTGGATGACATC |
| | CAGCTCACACAGTCCCCTGCCATCATGTCTGCCTCCCCTGGAGA |
| | GAAGGTGACAATGACCTGCAGAGCAAGCTCATCTGTTTCCTAC |
| | ATGAACTGGTATCAGCAGAAGTCTGGGACAAGCCCCAAAAGGT |
| | GGATCTATGACACCAGCAAAGTGGCCTCTGGAGTGCCTTACAG |
| | ATTCTCTGGATCTGGATCTGGCACCAGCTATTCTCTGACCATCT |
| | CCAGTATGGAAGCTGAGGATGCTGCCACCTACTACTGCCAGCA |
| | GTGGTCATCTAATCCCCTGACCTTTGGAGCTGGAACCAAACTG |
| | GAGCTGAAGTGACGATAATCAACCTCTGGATTACAAAATTTGT |
| | GAAAGATTGACTGGTATTCTTAACTATGTTGCTCCTTTTACGCT |
| | ATGTGGATACGCTGCTTTAATGCCTTTGTATCATGCTATTGCTT |
| | CCCGTATGGCTTTCATTTTCTCCTCCTTGTATAAATCCTGGTTGC |
| | TGTCTCTTTATGAGGAGTTGTGGCCCGTTGTCAGGCAACGTGGC |
| | GTGGTGTGCACTGTGTTTGCTGACGCAACCCCCACTGGTTGGGG |
| | CATTGCCACCACCTGTCAGCTCCTTTCCGGGACTTTCGCTTTCC |
| | CCCTCCCTATTGCCACGGCGGAACTCATCGCCGCCTGCCTTGCC |
| | CGCTGCTGGACAGGGGCTCGGCTGTTGGGCACTGACAATTCCG |
| | TGGTGTTGTCGGGGAAATCATCGTCCTTTCCTTGGCTGCTCGCC |
| | TGTGTTGCCACCTGGATTCTGCGCGGGACGTCCTTCTGCTACGT |
| | CCCTTCGGCCCTCAATCCAGCGGACCTTCCTTCCCGCGGCCTGC |
| | TGCCGGCTCTGCGGCCTCTTCCGCGTCTTCGCCTTCGCCCTCAG |
| | ACGAGTCGGATCTCCCTTTGGGCCGCCTCCCCGCATCGGACCCA |
| | GCTTTCTTGTACAAAGTGGGAATTCCTAGAGCTCGCTGATCAGC |
| | CTCGACTGTGCCTTCTAGTTGCCAGCCATCTGTTGTTTGCCCCTC |
| | CCCCGTGCCTTCCTTGACCCTGGAAGGTGCCACTCCCACTGTCC |
| | TTTCCTAATAAAATGAGGAAATTGCATCGCATTGTCTGAGTAG |
| | GTGTCATTCTATTCTGGGGGGTGGGGTGGGGCAGGACAGCAAG |
| | GGGGAGGATTGGGAAGAGAATAGCAGGCATGCTGGGGAGGGC |
| | CGCAGGAACCCCTAGTGATGGAGTTGGCCACTCCCTCTCTGCG |
| | CGCTCGCTCGCTCACTGAGGCCGGGCGACCAAAGGTCGCCCGA |
| | CGCCCGGGCTTTGCCCGGGCGGCCTCAGTGAGCGAGCGAGCGC |
| | GCAGCTGCCTGCAGGGGCGCCTGATGCGGTATTTTCTCCTTACG |
| | CATCTGTGCGGTATTTCACACCGCATACGTCAAAGCAACCATA |
| | GTACGCGCCCTGTAGCGGCGCATTAAGCGCGGCGGGTGTGGTG |
| | GTTACGCGCAGCGTGACCGCTACACTTGCCAGCGCCTTAGCGC |
| | CCGCTCCTTTCGCTTTCTTCCCTTCCTTTCTCGCCACGTTCGCCG |
| | GCTTTCCCCGTCAAGCTCTAAATCGGGGGCTCCCTTTAGGGTTC |
| | CGATTTAGTGCTTTACGGCACCTCGACCCCAAAAAACTTGATTT |
| | GGGTGATGGTTCACGTAGTGGGCCATCGCCCTGATAGACGGTT |
| | TTTCGCCCTTTGACGTTGGAGTCCACGTTCTTTAATAGTGGACT |
| | CTTGTTCCAAACTGGAACAACACTCAACTCTATCTCGGGCTATT |
| | CTTTTGATTTATAAGGGATTTTGCCGATTTCGGTCTATTGGTTA |
| | AAAAATGAGCTGATTTAACAAAAATTTAACGCGAATTTTAACA |
| | AAATATTAACGTTTACAATTTTATGGTGCACTCTCAGTACAATC |
| | TGCTCTGATGCCGCATAGTTAAGCCAGCCCCGACACCCGCCAA |
| | CACCCGCTGACGCGCCCTGACGGGCTTGTCTGCTCCCGGCATCC |
| | GCTTACAGACAAGCTGTGACCGTCTCCGGGAGCTGCATGTGTC |
| | AGAGGTTTTCACCGTCATCACCGAAACGCGCGAGACGAAAGGG |
| | CCTCGTGATACGCCTATTTTTATAGGTTAATGTCATGATAATAA |
| | TGGTTTCTTAGACGTCCTGGCCCGTGTCTCAAAATCTCTGATGT |
| | TACATTGCACAAGATAAAAATATATCATCATGAACAATAAAAC |
| | TGTCTGCTTACATAAACAGTAATACAAGGGGTGTTATGAGCCA |
| | TATTCAACGGGAAACGTCGAGGCCGCGATTAAATTCCAACATG |
| | GATGCTGATTTATATGGGTATAAATGGGCTCGCGATAATGTCG |
| | GGCAATCAGGTGCGACAATCTATCGCTTGTATGGGAAGCCCGA |
| | TGCGCCAGAGTTGTTTCTGAAACATGGCAAAGGTAGCGTTGCC |
| | AATGATGTTACAGATGAGATGGTCAGACTAAACTGGCTGACGG |
| | AATTTATGCCTCTTCCGACCATCAAGCATTTTATCCGTACTCCT |
| | GATGATGCATGGTTACTCACCACTGCGATCCCCGGAAAAACAG |
| | CATTCCAGGTATTAGAAGAATATCCTGATTCAGGTGAAAATAT |
| | TGTTGATGCGCTGGCAGTGTTCCTGCGCCGGTTGCATTCGATTC |
| | CTGTTTGTAATTGTCCTTTTAACAGCGATCGCGTATTTCGTCTCG |
| | CTCAGGCGCAATCACGAATGAATAACGGTTTGGTTGATGCGAG |
| | TGATTTTGATGACGAGCGTAATGGCTGGCCTGTTGAACAAGTCT |
| | GGAAAGAAATGCATAAACTTTTGCCATTCTCACCGGATTCAGT |
| | CGTCACTCATGGTGATTTCTCACTTGATAACCTTATTTTTGACG |
| | AGGGGAAATTAATAGGTTGTATTGATGTTGGACGAGTCGGAAT |
| | CGCAGACCGATACCAGGATCTTGCCATCCTATGGAACTGCCTC |
| | GGTGAGTTTTCTCCTTCATTACAGAAACGGCTTTTTCAAAAATA |
| | TGGTATTGATAATCCTGATATGAATAAATTGCAGTTTCATTTGA |
| | TGCTCGATGAGTTTTTCTAATCAGAATTGGTTAATTGGTTGTAA |
| | CACTGGCAGAGCATTACGCTGACTTGACGGGACGGCGCAAGCT |
| | CATGACCAAAATCCCTTAACGTGAGTTACGCGTGAAGATCCTTT |
| | TTGATAATCTCATGACCAAAATCCCTTAACGTGAGTTTTCGTTC |
| | CACTGAGCGTCAGACCCCGTAGAAAAGATCAAAGGATCTTCTT |
| | GAGATCCTTTTTTTCTGCGCGTAATCTGCTGCTTGCAAACAAAA |
| | AAACCACCGCTACCAGCGGTGGTTTGTTTGCCGGATCAAGAGC |
| | TACCAACTCTTTTTCCGAAGGTAACTGGCTTCAGCAGAGCGCA |
| | GATACCAAATACTGTTCTTCTAGTGTAGCCGTAGTTAGGCCACC |
| | ACTTCAAGAACTCTGTAGCACCGCCTACATACCTCGCTCTGCTA |
| | ATCCTGTTACCAGTGGCTGCTGCCAGTGGCGATAAGTCGTGTCT |
| | TACCGGGTTGGACTCAAGACGATAGTTACCGGATAAGGCGCAG |
| | CGGTCGGGCTGAACGGGGGGTTCGTGCACACAGCCCAGCTTGG |
| | AGCGAACGACCTACACCGAACTGAGATACCTACAGCGTGAGCT |
| | ATGAGAAAGCGCCACGCTTCCCGAAGGGAGAAAGGCGGACAG |
| | GTATCCGGTAAGCGGCAGGGTCGGAACAGGAGAGCGCACGAG |
| | GGAGCTTCCAGGGGGAAACGCCTGGTATCTTTATAGTCCTGTC |
| | GGGTTTCGCCACCTCTGACTTGAGCGTCGATTTTTGTGATGCTC |
| | GTCAGGGGGGCGGAGCCTATGGAAAAACGCCAGCAACGCGGC |
| | CTTTTTACGGTTCCTGGCCTTTTGCTGGCCTTTTGCTCACATGT |
| AAV2- 3H3B | CCTGCAGGCAGCTGCGCGCTCGCTCGCTCACTGAGGCCGCCCG |
| chA21 - CD3 | GGCAAAGCCCGGGCGTCGGGCGACCTTTGGTCGCCCGGCCTCA |
| (SEQ ID NO: | GTGAGCGAGCGAGCGCGCAGAGAGGGAGTGGCCAACTCCATC |
| 86) | ACTAGGGGTTCCTTCTAGCAACTTTGTATAGAAAAGTTGCTCGA |
| | CATTGATTATTGACTAGTTATTAATAGTAATCAATTACGGGGTC |
| | ATTAGTTCATAGCCCATATATGGAGTTCCGCGTTACATAACTTA |
| | CGGTAAATGGCCCGCCTGGCTGACCGCCCAACGACCCCCGCCC |
| | ATTGACGTCAATAATGACGTATGTTCCCATAGTAACGCCAATA |
| | GGGACTTTCCATTGACGTCAATGGGTGGAGTATTTACGGTAAA |
| | CTGCCCACTTGGCAGTACATCAAGTGTATCATATGCCAAGTAC |
| | GCCCCCTATTGACGTCAATGACGGTAAATGGCCCGCCTGGCAT |
| | TATGCCCAGTACATGACCTTATGGGACTTTCCTACTTGGCAGTA |
| | CATCTACGTATTAGTCATCGCTATTACCATGGTCGAGGTGAGCC |
| | CCACGTTCTGCTTCACTCTCCCCATCTCCCCCCCCTCCCCACCCC |
| | CAATTTTGTATTTATTTATTTTTTAATTATTTTGTGCAGCGATGG |
| | GGGCGGGGGGGGGGGGGGGGCGCGCGCCAGGCGGGGCGGGGC |
| | GGGGCGAGGGGCGGGGCGGGGCGAGGCGGAGAGGTGCGGCGG |
| | CAGCCAATCAGAGCGGCGCGCTCCGAAAGTTTCCTTTTATGGC |
| | GAGGCGGCGGCGGCGGCGGCCCTATAAAAAGCGAAGCGCGCG |
| | GCGGGCGGGAGTCGCTGCGCGCTGCCTTCGCCCCGTGCCCCGC |
| | TCCGCCGCCGCCTCGCGCCGCCCGCCCCGGCTCTGACTGACCGC |
| | GTTACTCCCACAGGTGAGCGGGCGGGACGGCCCTTCTCCTCCG |
| | GGCTGTAATTAGCGCTTGGTTTAATGACGGCTTGTTTCTTTTCT |
| | GTGGCTGCGTGAAAGCCTTGAGGGGCTCCGGGAGGGCCCTTTG |
| | TGCGGGGGGAGCGGCTCGGGGGGTGCGTGCGTGTGTGTGTGCG |
| | TGGGGAGCGCCGCGTGCGGCTCCGCGCTGCCCGGCGGCTGTGA |
| | GCGCTGCGGGCGCGGCGCGGGGCTTTGTGCGCTCCGCAGTGTG |
| | CGCGAGGGGAGCGCGGCCGGGGGCGGTGCCCCGCGGTGCGGG |
| | GGGGGCTGCGAGGGGAACAAAGGCTGCGTGCGGGGTGTGTGC |
| | GTGGGGGGGTGAGCAGGGGGTGTGGGCGCGTCGGTCGGGCTG |
| | CAACCCCCCCTGCACCCCCCTCCCCGAGTTGCTGAGCACGGCCC |
| | GGCTTCGGGTGCGGGGCTCCGTACGGGGCGTGGCGCGGGGCTC |
| | GCCGTGCCGGGCGGGGGGTGGCGGCAGGTGGGGGTGCCGGGC |
| | GGGGCGGGGCCGCCTCGGGCCGGGGAGGGCTCGGGGGAGGGG |
| | CGCGGCGGCCCCCGGAGCGCCGGCGGCTGTCGAGGCGCGGCG |
| | AGCCGCAGCCATTGCCTTTTATGGTAATCGTGCGAGAGGGCGC |
| | AGGGACTTCCTTTGTCCCAAATCTGTGCGGAGCCGAAATCTGG |
| | GAGGCGCCGCCGCACCCCCTCTAGCGGGCGCGGGGCGAAGCG |
| | GTGCGGCGCCGGCAGGAAGGAAATGGGCGGGGAGGGCCTTCG |
| | TGCGTCGCCGCGCCGCCGTCCCCTTCTCCCTCTCCAGCCTCGGG |
| | GCTGTCCGCGGGGGGACGGCTGCCTTCGGGGGGGACGGGGCA |
| | GGGCGGGGTTCGGCTTCTGGCGTGTGACCGGCGGCTCTAGAGC |
| | CTCTGCTAACCATGTTCATGCCTTCTTCTTTTTCCTACAGCTCCT |
| | GGGCAACGTGCTGGTTATTGTGCTGTCTCATCATTTTGGCAAAG |
| | AATTGCAAGTTTGTACAAAAAAGCAGGCTGCCACCGCGGCGCA |
| | GCCGGCGGATATTGTGCTGACCCAGACCCCGAGCAGCCTGCCG |
| | GTGAGCGTGGGCGAAAAAGTGACCATGACCTGCAAAAGCAGC |
| | CAGACCCTGCTGTATAGCAACAACCAGAAAAACTATCTGGCGT |
| | GGTATCAGCAGAAACCGGGCCAGAGCCCGAAACTGCTGATTAG |
| | CTGGGCGTTTACCCGCAAAAGCGGCGTGCCGGATCGCTTTACC |
| | GGCAGCGGCAGCGGCACCGATTTTACCCTGACCATTGGCAGCG |
| | TGAAAGCGGAAGATCTGGCGGTGTATTATTGCCAGCAGTATAG |
| | CAACTATCCGTGGACCTTTGGCGGCGGCACCCGCCTGGAAATT |
| | AAACGCGGCGGCGGCGGCAGCGGCGGCGGCGGCAGCGGCGGC |
| | GGCGGCAGCGGCGGCGGCGGCAGCGAAGTGCAGCTGCAGCAG |
| | AGCGGCCCGGAAGTGGTGAAAACCGGCGCGAGCGTGAAAATT |
| | AGCTGCAAAGCGAGCGGCTATAGCTTTACCGGCTATTTTATTAA |
| | CTGGGTGAAAAAAAACAGCGGCAAAAGCCCGGAATGGATTGG |
| | CCATATTAGCAGCAGCTATGCGACCAGCACCTATAACCAGAAA |
| | TTTAAAAACAAAGCGGCGTTTACCGTGGATACCAGCAGCAGCA |
| | CCGCGTTTATGCAGCTGAACAGCCTGACCAGCGAAGATAGCGC |
| | GGTGTATTATTGCGTGCGCAGCGGCAACTATGAAGAATATGCG |
| | ATGGATTATTGGGGCCAGGGCACCAGCGTGACCGTGAGCAGCG |
| | GCGGCGGCGGCAGCGATATTAAACTGCAGCAGAGCGGCGCGG |
| | AACTGGCGCGCCCGGGCGCGAGCGTGAAAATGAGCTGCAAAA |
| | CCAGCGGCTATACCTTTACCCGCTATACCATGCATTGGGTGAAA |
| | CAGCGCCCGGGCCAGGGCCTGGAATGGATTGGCTATATTAACC |
| | CGAGCCGCGGCTATACCAACTATAACCAGAAATTTAAAGATAA |
| | AGCGACCCTGACCACCGATAAAAGCAGCAGCACCGCGTATATG |
| | CAGCTGAGCAGCCTGACCAGCGAAGATAGCGCGGTGTATTATT |
| | GCGCGCGCTATTATGATGATCATTATTGCCTGGATTATTGGGGC |
| | CAGGGCACCACCCTGACCGTGAGCAGCGTGGAAGGCGGCAGC |
| | GGCGGCAGCGGCGGCAGCGGCGGCAGCGGCGGCGTGGATGAT |
| | ATTCAGCTGACCCAGAGCCCGGCGATTATGAGCGCGAGCCCGG |
| | GCGAAAAAGTGACCATGACCTGCCGCGCGAGCAGCAGCGTGA |
| | GCTATATGAACTGGTATCAGCAGAAAAGCGGCACCAGCCCGAA |
| | ACGCTGGATTTATGATACCAGCAAAGTGGCGAGCGGCGTGCCG |
| | TATCGCTTTAGCGGCAGCGGCAGCGGCACCAGCTATAGCCTGA |
| | CCATTAGCAGCATGGAAGCGGAAGATGCGGCGACCTATTATTG |
| | CCAGCAGTGGAGCAGCAACCCGCTGACCTTTGGCGCGGGCACC |
| | AAACTGGAACTGAAATAACGATAATCAACCTCTGGATTACAAA |
| | ATTTGTGAAAGATTGACTGGTATTCTTAACTATGTTGCTCCTTTT |
| | ACGCTATGTGGATACGCTGCTTTAATGCCTTTGTATCATGCTAT |
| | TGCTTCCCGTATGGCTTTCATTTTCTCCTCCTTGTATAAATCCTG |
| | GTTGCTGTCTCTTTATGAGGAGTTGTGGCCCGTTGTCAGGCAAC |
| | GTGGCGTGGTGTGCACTGTGTTTGCTGACGCAACCCCCACTGGT |
| | TGGGGCATTGCCACCACCTGTCAGCTCCTTTCCGGGACTTTCGC |
| | TTTCCCCCTCCCTATTGCCACGGCGGAACTCATCGCCGCCTGCC |
| | TTGCCCGCTGCTGGACAGGGGCTCGGCTGTTGGGCACTGACAA |
| | TTCCGTGGTGTTGTCGGGGAAATCATCGTCCTTTCCTTGGCTGC |
| | TCGCCTGTGTTGCCACCTGGATTCTGCGCGGGACGTCCTTCTGC |
| | TACGTCCCTTCGGCCCTCAATCCAGCGGACCTTCCTTCCCGCGG |
| | CCTGCTGCCGGCTCTGCGGCCTCTTCCGCGTCTTCGCCTTCGCC |
| | CTCAGACGAGTCGGATCTCCCTTTGGGCCGCCTCCCCGCATCGG |
| | ACCCAGCTTTCTTGTACAAAGTGGGAATTCCTAGAGCTCGCTGA |
| | TCAGCCTCGACTGTGCCTTCTAGTTGCCAGCCATCTGTTGTTTG |
| | CCCCTCCCCCGTGCCTTCCTTGACCCTGGAAGGTGCCACTCCCA |
| | CTGTCCTTTCCTAATAAAATGAGGAAATTGCATCGCATTGTCTG |
| | AGTAGGTGTCATTCTATTCTGGGGGGTGGGGTGGGGCAGGACA |
| | GCAAGGGGGAGGATTGGGAAGAGAATAGCAGGCATGCTGGGG |
| | AGGGCCGCAGGAACCCCTAGTGATGGAGTTGGCCACTCCCTCT |
| | CTGCGCGCTCGCTCGCTCACTGAGGCCGGGCGACCAAAGGTCG |
| | CCCGACGCCCGGGCTTTGCCCGGGCGGCCTCAGTGAGCGAGCG |
| | AGCGCGCAGCTGCCTGCAGGGGCGCCTGATGCGGTATTTTCTC |
| | CTTACGCATCTGTGCGGTATTTCACACCGCATACGTCAAAGCAA |
| | CCATAGTACGCGCCCTGTAGCGGCGCATTAAGCGCGGCGGGTG |
| | TGGTGGTTACGCGCAGCGTGACCGCTACACTTGCCAGCGCCTT |
| | AGCGCCCGCTCCTTTCGCTTTCTTCCCTTCCTTTCTCGCCACGTT |
| | CGCCGGCTTTCCCCGTCAAGCTCTAAATCGGGGGCTCCCTTTAG |
| | GGTTCCGATTTAGTGCTTTACGGCACCTCGACCCCAAAAAACTT |
| | GATTTGGGTGATGGTTCACGTAGTGGGCCATCGCCCTGATAGA |
| | CGGTTTTTCGCCCTTTGACGTTGGAGTCCACGTTCTTTAATAGT |
| | GGACTCTTGTTCCAAACTGGAACAACACTCAACTCTATCTCGGG |
| | CTATTCTTTTGATTTATAAGGGATTTTGCCGATTTCGGTCTATTG |
| | GTTAAAAAATGAGCTGATTTAACAAAAATTTAACGCGAATTTT |
| | AACAAAATATTAACGTTTACAATTTTATGGTGCACTCTCAGTAC |
| | AATCTGCTCTGATGCCGCATAGTTAAGCCAGCCCCGACACCCG |
| | CCAACACCCGCTGACGCGCCCTGACGGGCTTGTCTGCTCCCGG |
| | CATCCGCTTACAGACAAGCTGTGACCGTCTCCGGGAGCTGCAT |
| | GTGTCAGAGGTTTTCACCGTCATCACCGAAACGCGCGAGACGA |
| | AAGGGCCTCGTGATACGCCTATTTTTATAGGTTAATGTCATGAT |
| | AATAATGGTTTCTTAGACGTCCTGGCCCGTGTCTCAAAATCTCT |
| | GATGTTACATTGCACAAGATAAAAATATATCATCATGAACAAT |
| | AAAACTGTCTGCTTACATAAACAGTAATACAAGGGGTGTTATG |
| | AGCCATATTCAACGGGAAACGTCGAGGCCGCGATTAAATTCCA |
| | ACATGGATGCTGATTTATATGGGTATAAATGGGCTCGCGATAA |
| | TGTCGGGCAATCAGGTGCGACAATCTATCGCTTGTATGGGAAG |
| | CCCGATGCGCCAGAGTTGTTTCTGAAACATGGCAAAGGTAGCG |
| | TTGCCAATGATGTTACAGATGAGATGGTCAGACTAAACTGGCT |
| | GACGGAATTTATGCCTCTTCCGACCATCAAGCATTTTATCCGTA |
| | CTCCTGATGATGCATGGTTACTCACCACTGCGATCCCCGGAAA |
| | AACAGCATTCCAGGTATTAGAAGAATATCCTGATTCAGGTGAA |
| | AATATTGTTGATGCGCTGGCAGTGTTCCTGCGCCGGTTGCATTC |
| | GATTCCTGTTTGTAATTGTCCTTTTAACAGCGATCGCGTATTTC |
| | GTCTCGCTCAGGCGCAATCACGAATGAATAACGGTTTGGTTGA |
| | TGCGAGTGATTTTGATGACGAGCGTAATGGCTGGCCTGTTGAA |
| | CAAGTCTGGAAAGAAATGCATAAACTTTTGCCATTCTCACCGG |
| | ATTCAGTCGTCACTCATGGTGATTTCTCACTTGATAACCTTATTT |
| | TTGACGAGGGGAAATTAATAGGTTGTATTGATGTTGGACGAGT |
| | CGGAATCGCAGACCGATACCAGGATCTTGCCATCCTATGGAAC |
| | TGCCTCGGTGAGTTTTCTCCTTCATTACAGAAACGGCTTTTTCA |
| | AAAATATGGTATTGATAATCCTGATATGAATAAATTGCAGTTTC |
| | ATTTGATGCTCGATGAGTTTTTCTAATCAGAATTGGTTAATTGG |
| | TTGTAACACTGGCAGAGCATTACGCTGACTTGACGGGACGGCG |
| | CAAGCTCATGACCAAAATCCCTTAACGTGAGTTACGCGTGAAG |
| | ATCCTTTTTGATAATCTCATGACCAAAATCCCTTAACGTGAGTT |
| | TTCGTTCCACTGAGCGTCAGACCCCGTAGAAAAGATCAAAGGA |
| | TCTTCTTGAGATCCTTTTTTTCTGCGCGTAATCTGCTGCTTGCAA |
| | ACAAAAAAACCACCGCTACCAGCGGTGGTTTGTTTGCCGGATC |
| | AAGAGCTACCAACTCTTTTTCCGAAGGTAACTGGCTTCAGCAG |
| | AGCGCAGATACCAAATACTGTTCTTCTAGTGTAGCCGTAGTTAG |
| | GCCACCACTTCAAGAACTCTGTAGCACCGCCTACATACCTCGCT |
| | CTGCTAATCCTGTTACCAGTGGCTGCTGCCAGTGGCGATAAGTC |
| | GTGTCTTACCGGGTTGGACTCAAGACGATAGTTACCGGATAAG |
| | GCGCAGCGGTCGGGCTGAACGGGGGGTTCGTGCACACAGCCCA |
| | GCTTGGAGCGAACGACCTACACCGAACTGAGATACCTACAGCG |
| | TGAGCTATGAGAAAGCGCCACGCTTCCCGAAGGGAGAAAGGC |
| | GGACAGGTATCCGGTAAGCGGCAGGGTCGGAACAGGAGAGCG |
| | CACGAGGGAGCTTCCAGGGGGAAACGCCTGGTATCTTTATAGT |
| | CCTGTCGGGTTTCGCCACCTCTGACTTGAGCGTCGATTTTTGTG |
| | ATGCTCGTCAGGGGGGCGGAGCCTATGGAAAAACGCCAGCAA |
| | CGCGGCCTTTTTACGGTTCCTGGCCTTTTGCTGGCCTTTTGCTCA |
| | CATGT |

In some embodiments, the rAAV vector comprises a sequence having at least about 85% identity to any one of SEQ ID NOs: 82-86. In some embodiments, the rAAV vector comprises a sequence having at least about 90% identity to any one of SEQ ID NOs: 82-86. In some embodiments, the rAAV vector comprises a sequence having at least about 95% identity to any one of SEQ ID NOs: 82-86. In some embodiments, the rAAV vector comprises a sequence having at least about 96% identity to any one of SEQ ID NOs: 82-86. In some embodiments, the rAAV vector comprises a sequence having at least about 97% identity to any one of SEQ ID NOs: 82-86. In some embodiments, the rAAV vector comprises a sequence having at least about 98% identity to any one of SEQ ID NOs: 82-86. In some embodiments, the rAAV vector comprises a sequence having at least about 99% identity to any one of SEQ ID NOs: 82-86. In some embodiments, the rAAV vector comprises a sequence having 100% identity to any one of SEQ ID NOs: 82-86.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 83.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 84.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 85.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 82.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 86.

In some embodiments, rAAV vectors of the present disclosure comprise one or more components (e.g., regulatory elements, transgene) comprising reduced CpG dinucleotides and/or increased methylation of CpG dinucleotides as compared to a parental equivalent. In some embodiments, CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides are reduced in a range of about 5% to about 90%, about 10% to about 80%, about 15% to about 75%, about 20% to about 70%, about 25% to about 65%, or about 30% to about 60%. In some embodiments, methylation of CpG dinucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, methylation of CpG dinucleotides is increased in a range of about 5% to about 90%, about 10% to about 80%, about 15% to about 75%, about 20% to about 70%, about 25% to about 65%, or about 30% to about 60%.

### 2. Recombinant Adeno-Associated Viral (AAV) Vector Production

Recombinant AAV particles can be produced by any standard method (see, for example, WO 2001/083692; Masic et al. 2014. Molecular Therapy, 22 (11): 1900-1909; Carter, 1992, Current Opinions in Biotechnology, 1533-539; Muzyczka, 1992, Curr. Topics in Microbial, and Immunol., 158:97-129); Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al, J. Virol, 62:1963 (1988); and Lebkowski et al, Mol. Cell. Biol, 7:349 (1988). Samulski et al, J. Virol., 63:3822-3828 (1989); U.S. Pat. No. 5,173,414; WO 95/13365; U.S. Pat. No. 5,658,776; WO95/13392; WO 96/17947; PCT/US98/18600; WO 97/09441 (PCT/US 96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and U.S. Pat. No. 6,258,595, herein incorporated by reference in their entirety). For example, in some embodiments, AAV vectors described herein can be transformed into Escherichia coli to scale-up DNA production, purified using any standard method (for example, a Maxi-Prep K), and verified by restriction digest or sequencing. Purified AAV vectors can then be transfected using a standard method (e.g., calcium phosphate transfection, liposomal, polyethyleneimine, electroporation, and the like) into an appropriate packaging cell line (e.g., HEK293, HeLa, Sf9, PerC.6, MRC-5, WI-38, Vera, or FRhL-2 cells) in combination with a plasmid comprising AAV rep and AAV cap genes, and an AAV helper plasmid. The AAV rep and cap genes may be from any AAV serotype and may be the same or different from that of the recombinant AAV vector ITRs including, but not limited to, AAV1, AAV2, AAV3, AAV4, AAV5, AAV6, AAV7, AAVrh. 74, AAV8, AAV9, AAV10, AAV11, AAV12, and AAV13. In certain embodiments, recombinant AAV described herein comprise AAV rep and cap genes derived from AAV2 and AAV9, respectively. The AAV helper plasmid may be from any AAV serotype and may be the same or different from that of the recombinant AAV vector ITRs including, but not limited to, AAV1, AAV2, AAV3, AAV4, AAV5, AAV6, AAV7, AAVrh.74, AAV8, AAV9, AAV10, AAV11, AAV12, and AAV13. In certain embodiments, recombinant AAV described herein comprise plasmids with helper genes derived from AAV2.

In some embodiments, the AAV rep and cap genes are from AAVrh.74. In some embodiments, the rep and cap genes comprise a nucleotide sequence having at least 85% (e.g., 85%, 90%, 95%, 97%, 98%, or 99%) sequence identity to the nucleic acid sequence of SEQ ID NO: 178. In some embodiments, the rep and cap genes comprise a nucleotide sequence having at least 85% sequence identity to the nucleic acid sequence of SEQ ID NO: 178. In some embodiments, the rep and cap genes comprise a nucleotide sequence having at least 90% sequence identity to the nucleic acid sequence of SEQ ID NO: 178. In some embodiments, the rep and cap genes comprise a nucleotide sequence having at least 95% sequence identity to the nucleic acid sequence of SEQ ID NO: 178. In some embodiments, the rep and cap genes comprise a nucleotide sequence having at least 97% sequence identity to the nucleic acid sequence of SEQ ID NO: 178. In some embodiments, the rep and cap genes comprise a nucleotide sequence having at least 98% sequence identity to the nucleic acid sequence of SEQ ID NO: 178. In some embodiments, the rep and cap genes comprise a nucleotide sequence having at least 99% sequence identity to the nucleic acid sequence of SEQ ID NO: 178. In some embodiments, the rep and cap genes comprise a nucleotide sequence having the nucleic acid sequence of SEQ ID NO: 178.

TABLE 10
Rep and Cap Sequences

| | SEQ ID | |
|---|---|---|
| Description | NO | Sequence |
| Rep and | 178 | CGGGCCCCCCCTCGAGGTCGACGGTATCGGGGGAGCT |
| Cap | | CGCAGGGTCTCCATTTTGAAGCGGGAGGTTTGAACGC |
| | | GCAGCCGCCATGCCGGGGTTTTACGAGATTGTGATTA |
| | | AGGTCCCCAGCGACCTTGACGAGCATCTGCCCGGCAT |
| | | TTCTGACAGCTTTGTGAACTGGGTGGCCGAGAAGGAA |
| | | TGGGAGTTGCCGCCAGATTCTGACATGGATCTGAATC |
| | | TGATTGAGCAGGCACCCCTGACCGTGGCCGAGAAGCT |
| | | GCAGCGCGACTTTCTGACGGAATGGCGCCGTGTGAGT |
| | | AAGGCCCCGGAGGCTCTTTTCTTTGTGCAATTTGAGAA |
| | | GGGAGAGAGCTACTTCCACATGCACGTGCTCGTGGAA |
| | | ACCACCGGGGTGAAATCCATGGTTTTGGGACGTTTCCT |
| | | GAGTCAGATTCGCGAAAAACTGATTCAGAGAATTTAC |
| | | CGCGGGATCGAGCCGACTTTGCCAAACTGGTTCGCGG |
| | | TCACAAAGACCAGAAATGGCGCCGGAGGCGGGAACA |
| | | AGGTGGTGGATGAGTGCTACATCCCCAATTACTTGCTC |
| | | CCCAAAACCCAGCCTGAGCTCCAGTGGGCGTGGACTA |
| | | ATATGGAACAGTATTTAAGCGCCTGTTTGAATCTCACG |
| | | GAGCGTAAACGGTTGGTGGCGCAGCATCTGACGCACG |
| | | TGTCGCAGACGCAGGAGCAGAACAAAGAGAATCAGA |
| | | ATCCCAATTCTGATGCGCCGGTGATCAGATCAAAAAC |
| | | TTCAGCCAGGTACATGGAGCTGGTCGGGTGGCTCGTG |
| | | GACAAGGGGATTACCTCGGAGAAGCAGTGGATCCAG |
| | | GTGAGTAATTGACAAAGCCAAACACCACCATTTGCCG |
| | | AGCACTTTAGAGTTTACAGGTTTGTTTCTCTTGACCCT |
| | | CAAAACAAACCTGTGAGGCATAGGGAGTATTGCTATC |
| | | CCTTAAGAATTCACCCCCAGTGTGCCCATCAAAACCTC |
| | | CCAGGCTGAGTCTGCACAGTTGAAGGAGGAAGGATAG |
| | | GAATGGGAGGGTCGATGGGTGAAAGCATGATTCTCTT |
| | | AACCAGTCCAGATTATCAGGTAATCCCTTCAACAACC |
| | | ACCACCCACTCCCTGGGCAATCCAGCTGGAGTTTACA |
| | | GACAGACTTAGCTGGCTATAGCACCACCGTGCTACTC |
| | | TCTGTTCTTCCTGGTTGCTCAAATGCCCTAGAAAAGTG |
| | | GAACAGGTGAGCATCAACTCACAGGGCTCTATGCTGG |
| | | CTGCTGCTGCGAGGGATGTTATGCTATAGTACCAGGG |
| | | GCCACCATTCCATAGGCACTTCCTGTGTTTAATACCCT |
| | | ATATGCTTTACTTCATCTCATCTTCCTCCATATCCTGAG |
| | | AGGTGGTTCTATTCTTCTCCCCATTTTACGGATGAAAA |
| | | AACCGAGACACAGAAAGGTGAAATAGCTTAAGATAA |
| | | ATGGTGCCTTGCAGCCTTAGACTCTGGTGGCCTCTAGT |
| | | TAATGTGGGAAATTAAGGGTGAGGGGATTGGCAGCTG |
| | | ATGGAGGGTGCAGGGTGCCAGACAGAGGCGTTTAGCT |
| | | CTGATCCCTTAGCAATAGAGAGTCCTTGTAGGCACTTG |
| | | GTCAGGCGAGTGATGCGATGAAAGCTGTGTTTAAGAA |
| | | AGATTATGCTTTCTGCTGATTTCATACCCCCAACACCC |
| | | AAGCTCTGAGGCCCCTCCTCACAGGTCCTTGCAGGGC |
| | | TGGCCAAAATAAAGCAGCTTCACTCCGTTGTGCTGCTT |
| | | TCCAGCTAATGTGTCTGTTTGGCAGAAGTTTCCCTCAA |
| | | AGGCAGATCAGTGAAATAAGCAGAAGCCTCGACCCCC |
| | | CTTTGTCAGCCAGAGCTGCTGAAGTGCCTTGCCCCAG |
| | | GGTCACTTTGTGTGAGGGGATTAGAGAGCACTGGGGC |
| | | TGCCAAGAAACACTGCCGTTTCTACAGATTAGCAGGA |
| | | CGCTGGCTTGTGGCCTTCTAGCGAGGCTCAGAGCTGC |
| | | GGTGGCCCTAGTCTGCATGGGCTAAAGACAAGCTCCA |
| | | TCTCCTGTCCTTGTTCCCTCCTTCCTGGGCACAGCCGC |
| | | CCTGCTTCTTGGTTCTCTCTGTTGGTTCCTGTCCGCACG |
| | | GTAGTTAGGCTGGCAGCGTGTGTAGGATTTGGCTTAG |
| | | AAGATTGACAACATTGCCTTTGAGCCCTTCTTTGCTAC |
| | | TCCTCCCTCTCCCCTCCCATCAGACTCCTCTCTGGAGT |
| | | CTGCTCTGCGAGGCCTCTGCTCTGTGGTATCCCAGCAG |
| | | CCTTCTCAGCCTTGACTTCCAGAAGGGGGCTGTGCAGT |
| | | GTCCGGGGTGTGCAGGCCCCAGACACGGGGTAGGCTC |
| | | ATGGAGATCCAAGTGCTGATCTAGTGTCAAGGCTGGC |
| | | CTGGAGACTGGGCTGGGTTGGTGTCTGCCTGCTGTGGT |
| | | CATGTGCCCTCCCTTGGGCCTGTATCCTCTCTCCAGAC |
| | | TTGCTGCAGGGAGAGGTGGCAGATGTCAGCCTAGTTC |
| | | TGGCCTCTCAGAGCAGCATGGCAGCTCCCTTTCACTCA |
| | | GGCCCAGGCTGGGCCCTCCTGCTGGCTGACCCCTGGG |
| | | GAGAGGGTGCTCCAGAGCTCCCCAAGGAACAGCTTCC |
| | | CGAAGCAGCCAGGCCAGCCCAGAGGGGCTGTGGCCA |
| | | ATCCTGAAGCTTTATGTTCCTGCTGACATTTTTTCTAA |
| | | GTTTTCTCTTGCTTTCCTCTTAAATGCCAATCTGGAGA |
| | | GTCTCCGTTAGGAGAAATGGACCCCAGCCAGGAAGAA |
| | | GAGTTGAGTTGTATTTAAAACACGAGCTCCCCCTAAA |
| | | GCATCCTTCTTTAGCTTCTAAGGAGAGGCAGAGACTG |
| | | ACAGGCAGGACTCAGCAGGAAAAGGTACCCCCCTGAC |
| | | CTGCTCAGTCAGGCCCTAGGCCCAGCTCCACCCAGCC |
| | | TGTGGCCCCCAGAGTTTCGGTAAAGAGTTCCCTGGGC |
| | | CTTAAGGAACCTTGAGAGAGCATTTGAGGGGTGCCAC |
| | | CACAAACTTGGCAGAAAAAACCCTCCCCCTCCAAGTC |
| | | CAGTCCTAGAGAAGGAGCTGGCAACCTTGCCTTGCTT |
| | | TGTAAGCAAAAGCCTCTTAGGGCTTGAGCTCAGATGT |
| | | AGTGTTTGAGCTGTGGCTGGTGCCCTGCCCCATCAGG |
| | | GAGCCAATGGTAGACATCCTATGGGCATCTTTGTTTTC |
| | | CGTAAGAGCAGGCTGTCTGGGGATGGGCCAGAGGAA |
| | | GAGGCGACCTGGAGTCAACCAAGAGGAGGCCTTAACC |
| | | AAGCCTTAACCACAGAGGTTAACCAAGCCTTGAAAGC |
| | | GCTTCCCCCTGAGCAGGCAGGAAGCACTGAGTCCACA |
| | | TGGTTGCCTCGCTGTTTCATTTCCTTACACTCAATTCTC |
| | | TCAGTCTTTAAATGATCACTTGGCCTTGAAGTTACGGA |
| | | TATTTGGGGTCTGAACTGAAGTTGAAGAAAAGAGGAA |
| | | ATGATTTAAGCTTTGTTTAAGATTAGGGGCCAGGTGC |
| | | GGTGGCTCACGCCTGTAATCCCAGCACCTTGGGAGCC |
| | | TGAGGCGGGTGGATCACCTGAGGTCAGGAGTTCCAGA |
| | | CCAGCCTGGCCAACATAGCAAAACCCAGTCTCTACTA |
| | | AAAATAACAATAAAAAAATTAGCCAGGTGTGGTGACA |
| | | CATGCCTGTAATCCCAGTTACTCAGGAGGCTGAGGCA |
| | | GAATTGCTTGAACTTGAGAGGTGGAGGTTGTAGTGAG |
| | | CCAAGACCGCACCACTGCACTCCAGCCTGGCGACAGA |
| | | GCCAGACTCCGTCTCAAAAACAACAACAAAAAAGATT |
| | | AGAAGAAGCCCATTACTGCCTTCTGGCCACCCACTCG |
| | | CACAGACACCAAAACTGCAGCCCACACCTCGCCATCC |
| | | TCGTGCTCTGCCCTGGGACACCCCAGGCACAGTGTGT |
| | | CCTTCGTTTTCTGTAAGGGTGGGCTGGGAGCAGGGAC |
| | | GGACAGGGCCTGTGGGCACCTCTCATGGTCACTTCCTT |
| | | CTTGCTCACAGGAGGACCAGGCCTCATACATCTCCTTC |
| | | AATGCGGCCTCCAACTCGCGGTCCCAAATCAAGGCTG |
| | | CCTTGGACAATGCGGGAAAGATTATGAGCCTGACTAA |
| | | AACCGCCCCCGACTACCTGGTGGGCCAGCAGCCCGTG |
| | | GAGGACATTTCCAGCAATCGGATTTATAAAATTTTGG |
| | | AACTAAACGGGTACGATCCCCAATATGCGGCTTCCGT |
| | | CTTTCTGGGATGGGCCACGAAAAAGTTCGGCAAGAGG |
| | | AACACCATCTGGCTGTTTGGGCCTGCAACTACCGGGA |
| | | AGACCAACATCGCGGAGGCCATAGCCCACACTGTGCC |
| | | CTTCTACGGGTGCGTAAACTGGACCAATGAGAACTTT |
| | | CCCTTCAACGACTGTGTCGACAAGATGGTGATCTGGT |
| | | GGGAGGAGGGGAAGATGACCGCCAAGGTCGTGGAGT |
| | | CGGCCAAAGCCATTCTCGGAGGAAGCAAGGTGCGCGT |
| | | GGACCAGAAATGCAAGTCCTCGGCCCAGATAGACCCG |
| | | ACTCCCGTGATCGTCACCTCCAACACCAACATGTGCG |
| | | CCGTGATTGACGGGAACTCAACGACCTTCGAACACCA |
| | | GCAGCCGTTGCAAGACCGGATGTTCAAATTTGAACTC |
| | | ACCCGCCGTCTGGATCATGACTTTGGGAAGGTCACCA |
| | | AGCAGGAAGTCAAAGACTTTTTCCGGTGGGCAAAGGA |
| | | TCACGTGGTTGAGGTGGAGCATGAATTCTACGTCAAA |
| | | AAGGGTGGAGCCAAGAAAAGACCCGCCCCCAGTGAC |
| | | GCAGATATAAGTGAGCCCAAACGGGTGCGCGAGTCAG |
| | | TTGCGCAGCCATCGACGTCAGACGCGGAAGCTTCGAT |
| | | CAACTACGCAGACAGGTACCAAAACAAATGTTCTCGT |
| | | CACGTGGGCATGAATCTGATGCTGTTTCCCTGCAGAC |
| | | AATGCGAGAGAATGAATCAGAATTCAAATATCTGCTT |
| | | CACTCACGGACAGAAAGACTGTTTAGAGTGCTTTCCC |
| | | GTGTCAGAATCTCAACCCGTTTCTGTCGTCAAAAAGG |
| | | CGTATCAGAAACTGTGCTACATTCATCATATCATGGG |
| | | AAAGGTGCCAGACGCTTGCACTGCCTGCGATCTGGTC |
| | | AATGTGGATTTGGATGACTGCATCTTTGAACAATAAA |
| | | TGATTTAAATCAGGTATGGCTGCCGATGGTTATCTTCC |
| | | AGATTGGCTCGAGGACAACCTCTCTGAGGGCATTCGC |
| | | GAGTGGTGGGACCTGAAACCTGGAGCCCCGAAACCCA |
| | | AAGCCAACCAGCAAAAGCAGGACAACGGCCGGGGTC |
| | | TGGTGCTTCCTGGCTACAAGTACCTCGGACCCTTCAAC |
| | | GGACTCGACAAGGGGGAGCCCGTCAACGCGGCGGAC |
| | | GCAGCGGCCCTCGAGCACGACAAGGCCTACGACCAGC |
| | | AGCTCCAAGCGGGTGACAATCCGTACCTGCGGTATAA |
| | | TCACGCCGACGCCGAGTTTCAGGAGCGTCTGCAAGAA |
| | | GATACGTCTTTTGGGGGCAACCTCGGGCGCGCAGTCT |
| | | TCCAGGCCAAAAAGCGGGTTCTCGAACCTCTGGGCCT |
| | | GGTTGAATCGCCGGTTAAGACGGCTCCTGGAAAGAAG |
| | | AGACCGGTAGAGCCATCACCCCAGCGCTCTCCAGACT |
| | | CCTCTACGGGCATCGGCAAGAAAGGCCAGCAGCCCGC |
| | | AAAAAAGAGACTCAATTTTGGGCAGACTGGCGACTCA |
| | | GAGTCAGTCCCCGACCCTCAACCAATCGGAGAACCAC |
| | | CAGCAGGCCCCTCTGGTCTGGGATCTGGTACAATGGC |
| | | TGCAGGCGGTGGCGCTCCAATGGCAGACAATAACGAA |
| | | GGCGCCGACGGAGTGGGTAGTTCCTCAGGAAATTGGC |
| | | ATTGCGATTCCACATGGCTGGGCGACAGAGTCATCAC |
| | | CACCAGCACCCGCACCTGGGCCCTGCCCACCTACAAC |
| | | AACCACCTCTACAAGCAAATCTCCAACGGGACCTCGG |
| | | GAGGAAGCACCAACGACAACACCTACTTCGGCTACAG |
| | | CACCCCCTGGGGGTATTTTGACTTCAACAGATTCCACT |
| | | GCCACTTTTCACCACGTGACTGGCAGCGACTCATCAA |
| | | CAACAACTGGGGATTCCGGCCCAAGAGGCTCAACTTC |
| | | AAGCTCTTCAACATCCAAGTCAAGGAGGTCACGCAGA |
| | | ATGAAGGCACCAAGACCATCGCCAATAACCTTACCAG |
| | | CACGATTCAGGTCTTTACGGACTCGGAATACCAGCTC |
| | | CCGTACGTGCTCGGCTCGGCGCACCAGGGCTGCCTGC |
| | | CTCCGTTCCCGGCGGACGTCTTCATGATTCCTCAGTAC |
| | | GGGTACCTGACTCTGAACAATGGCAGTCAGGCTGTGG |
| | | GCCGGTCGTCCTTCTACTGCCTGGAGTACTTTCCTTCT |
| | | CAAATGCTGAGAACGGGCAACAACTTTGAATTCAGCT |
| | | ACAACTTCGAGGACGTGCCCTTCCACAGCAGCTACGC |
| | | GCACAGCCAGAGCCTGGACCGGCTGATGAACCCTCTC |
| | | ATCGACCAGTACTTGTACTACCTGTCCCGGACTCAAA |
| | | GCACGGGCGGTACTGCAGGAACTCAGCAGTTGCTATT |
| | | TTCTCAGGCCGGGCCTAACAACATGTCGGCTCAGGCC |
| | | AAGAACTGGCTACCCGGTCCCTGCTACCGGCAGCAAC |
| | | GCGTCTCCACGACACTGTCGCAGAACAACAACAGCAA |
| | | CTTTGCCTGGACGGGTGCCACCAAGTATCATCTGAAT |
| | | GGCAGAGACTCTCTGGTGAATCCTGGCGTTGCCATGG |
| | | CTACCCACAAGGACGACGAAGAGCGATTTTTTCCATC |
| | | CAGCGGAGTCTTAATGTTTGGGAAACAGGGAGCTGGA |
| | | AAAGACAACGTGGACTATAGCAGCGTGATGCTAACCA |
| | | GCGAGGAAGAAATAAAGACCACCAACCCAGTGGCCA |
| | | CAGAACAGTACGGCGTGGTGGCCGATAACCTGCAACA |
| | | GCAAAACGCCGCTCCTATTGTAGGGGCCGTCAATAGT |
| | | CAAGGAGCCTTACCTGGCATGGTGTGGCAGAACCGGG |
| | | ACGTGTACCTGCAGGGTCCCATCTGGGCCAAGATTCC |
| | | TCATACGGACGGCAACTTTCATCCCTCGCCGCTGATGG |
| | | GAGGCTTTGGACTGAAGCATCCGCCTCCTCAGATCCT |
| | | GATTAAAAACACACCTGTTCCCGCGGATCCTCCGACC |
| | | ACCTTCAATCAGGCCAAGCTGGCTTCTTTCATCACGCA |
| | | GTACAGTACCGGCCAGGTCAGCGTGGAGATCGAGTGG |
| | | GAGCTGCAGAAGGAGAACAGCAAACGCTGGAACCCA |
| | | GAGATTCAGTACACTTCCAACTACTACAAATCTACAA |
| | | ATGTGGACTTTGCTGTCAATACTGAGGGTACTTATTCC |
| | | GAGCCTCGCCCCATTGGCACCCGTTACCTCACCCGTA |
| | | ATCTGTAATTACATGTTAATCAATAAACCGGTTAATTC |
| | | GTTTCAGTTGAACTTTGGTCTCCTGTCCTTCTTATCTTA |
| | | TCGGTTACCATAGAAACTGGTTACTTATTAACTGCTTG |
| | | GTGCGCTTCGCGATAAAAGACTTACGTCATCGGGTTA |
| | | CCCCTAGTGATGGAGCGGCCGCTTTCAGTTGAACTTTG |
| | | GTCTCTGCGTATTTCTTTCTTATCTAGTTTCCATGCTCT |
| | | AGAGGTCCTGTATTAGAGGTCACGTGAGTGTTTTGCG |
| | | ACATTTTGCGACACCATGTGGTCACGCTGGGTATTTAA |
| | | GCCCGAGTGAGCACGCAGGGTCTCCATTTTGAAGCGG |
| | | GAGGTTTGAACGCGCAGCCGCCAAGCCGAATTCTGCA |
| | | GATAATTCCGTGTATTCTATAGTGTCACCTAAATCGTA |
| | | TGTGTATGATACATAAGGTTATGTATTAATTGTAGCCG |
| | | CGTTCTAACGACAATATGTACAAGCCTAATTGTGTAG |
| | | CATCTGGCTTACTGAAGCAGACCCTATCATCTCTCTCG |
| | | TAAACTGCCGTCAGAGTCGGTTTGGTTGGACGAACCT |
| | | TCTGAGTTTCTGGTAACGCCGTCCCGCACCCGGAAAT |
| | | GGTCAGCGAACCAATCAGCAGGGTCATCGCTAGCCAG |
| | | ATCCTCTACGCCGGACGCATCGTGGCCGGCATCACCG |
| | | GCGCCACAGGTGCGGTTGCTGGCGCCTATATCGCCGA |
| | | CATCACCGATGGGGAAGATCGGGCTCGCCACTTCGGG |
| | | CTCATGAGCGCTTGTTTCGGCGTGGGTATGGTGGCAG |
| | | GCCGCCCTTAGAAAAACTCATCGAGCATCAAATGAAA |
| | | CTGCAATTTATTCATATCAGGATTATCAATACCATATT |
| | | TTTGAAAAAGCCGTTTCTGTAATGAAGGAGAAAACTC |
| | | ACCGAGGCAGTTCCATAGGATGGCAAGATCCTGGTAT |
| | | CGGTCTGCGATTCCGACTCGTCCAACATCAATACAAC |
| | | CTATTAATTTCCCCTCGTCAAAAATAAGGTTATCAAGT |
| | | GAGAAATCACCATGAGTGACGACTGAATCCGGTGAGA |
| | | ATGGCAAAAGCTTATGCATTTCTTTCCAGACTTGTTCA |
| | | ACAGGCCAGCCATTACGCTCGTCATCAAAATCACTCG |
| | | CATCAACCAAACCGTTATTCATTCGTGATTGCGCCTGA |
| | | GCGAGACGAAATACGCGATCGCTGTTAAAAGGACAAT |
| | | TACAAACAGGAATCGAATGCAACCGGCGCAGGAACA |
| | | CTGCCAGCGCATCAACAATATTTTCACCTGAATCAGG |
| | | ATATTCTTCTAATACCTGGAATGCTGTTTTCCCGGGGA |
| | | TCGCAGTGGTGAGTAACCATGCATCATCAGGAGTACG |
| | | GATAAAATGCTTGATGGTCGGAAGAGGCATAAATTCC |
| | | GTCAGCCAGTTTAGTCTGACCATCTCATCTGTAACATC |
| | | ATTGGCAACGCTACCTTTGCCATGTTTCAGAAACAACT |
| | | CTGGCGCATCGGGCTTCCCATACAATCGATAGATTGTC |
| | | GCACCTGATTGCCCGACATTATCGCGAGCCCATTTATA |
| | | CCCATATAAATCAGCATCCATGTTGGAATTTAATCGCG |
| | | GCCTCGAGCAAGACGTTTCCCGTTGAATATGGCTCAT |
| | | AACACCCCTTGTATTACTGTTTATGTAAGCAGACAGTT |
| | | TTATTGTTCATGATGATATATTTTTATCTTGTGCAATGT |
| | | AACATCAGAGATTTTGAGACACAACGTGGTTTGCAGG |
| | | AGTCAGGCAACTATGGATGAACGAAATAGACAGATCG |
| | | CTGAGATAGGTGCCTCACTGATTAAGCATTGGTAACT |
| | | GTCAGACCAAGTTTACTCATATATACTTTAGATTGATT |
| | | TAAAACTTCATTTTTAATTTAAAAGGATCTAGGTGAAG |
| | | ATCCTTTTTGATAATCTCATGACCAAAATCCCTTAACG |
| | | TGAGTTTTCGTTCCACTGAGCGTCAGACCCCGTAGAA |
| | | AAGATCAAAGGATCTTCTTGAGATCCTTTTTTTCTGCG |
| | | CGTAATCTGCTGCTTGCAAACAAAAAAACCACCGCTA |
| | | CCAGCGGTGGTTTGTTTGCCGGATCAAGAGCTACCAA |
| | | CTCTTTTTCCGAAGGTAACTGGCTTCAGCAGAGCGCA |
| | | GATACCAAATACTGTTCTTCTAGTGTAGCCGTAGTTAG |
| | | GCCACCACTTCAAGAACTCTGTAGCACCGCCTACATA |
| | | CCTCGCTCTGCTAATCCTGTTACCAGTGGCTGCTGCCA |
| | | GTGGCGATAAGTCGTGTCTTACCGGGTTGGACTCAAG |
| | | ACGATAGTTACCGGATAAGGCGCAGCGGTCGGGCTGA |
| | | ACGGGGGGTTCGTGCACACAGCCCAGCTTGGAGCGAA |
| | | CGACCTACACCGAACTGAGATACCTACAGCGTGAGCT |
| | | ATGAGAAAGCGCCACGCTTCCCGAAGGGAGAAAGGC |
| | | GGACAGGTATCCGGTAAGCGGCAGGGTCGGAACAGG |
| | | AGAGCGCACGAGGGAGCTTCCAGGGGGAAACGCCTG |
| | | GTATCTTTATAGTCCTGTCGGGTTTCGCCACCTCTGAC |
| | | TTGAGCGTCGATTTTTGTGATGCTCGTCAGGGGGGCG |
| | | GAGCCTATGGAAAAACGCCAGCAACGCGGCCTTTTTA |
| | | CGGTTCCTGGCCTTTTGCTGGCCTTTTGCTCACATGTTC |
| | | TTTCCTGCGTTATCCCCTGATTCTGTGGATAACCGTAT |
| | | TACCGCCTTTGAGTGAGCTGATACCGCTCGCCGCAGC |
| | | CGAACGACCGAGCGCAGCGAGTCAGTGAGCGAGGAA |
| | | GCGGAAGAGCGCCCAATACGCAAACCGCCTCTCCCCG |
| | | CGCGTTGGCCGATTCATTAATGCAGCTGTGGAATGTGT |
| | | GTCAGTTAGGGTGTGGAAAGTCCCCAGGCTCCCCAGC |
| | | AGGCAGAAGTATGCAAAGCATGCATCTCAATTAGTCA |
| | | GCAACCAGGTGTGGAAAGTCCCCAGGCTCCCCAGCAG |
| | | GCAGAAGTATGCAAAGCATGCATCTCAATTAGTCAGC |
| | | AACCATAGTCCCGCCCCTAACTCCGCCCATCCCGCCCC |
| | | TAACTCCGCCCAGTTCCGCCCATTCTCCGCCCCATGGC |
| | | TGACTAATTTTTTTTATTTATGCAGAGGCCGAGGCCGC |
| | | CTCGGCCTCTGAGCTATTCCAGAAGTAGTGAGGAGGC |
| | | TTTTTTGGAGGAACAAACAGCTTTTTTGGGGTGAACAT |
| | | ATTGACTGAATTGGCGAACGTGGCGAGAAAGGAAGG |
| | | GAAGAAAGCGAAAGGAGCGGGCGCTAGGGCGCTGGC |
| | | AAGTGTAGCGGTCACGCTGCGCGTAACCACCACACCC |
| | | GCCGCGCTTAATGCGCCGCTACAGGGCGCGTCCCATT |
| | | CGCCATTCAGGCTGCGCAACTGTTGGGAAGGGCGATC |
| | | GGTGCGGGCCTCTTCGCTATTACGCCAGCTGGCGAAA |
| | | GGGGGATGTGCTGCAAGGCGATTAAGTTGGGTAACGC |
| | | CAGGGTTTTCCCAGTCACGACGTTGTAAAACGACGGC |
| | | CAGTGAGCGCGCGTAATACGACTCACTATAGGGCGAA |
| | | TTGGGTAC |

In some embodiments, recombinant AAV described herein is harvested from packaging cells and purified by methods standard in the art (e.g. Clark et al, Hum. Gene Ther., 10(6): 1031-1039 (1999); Schenpp and Clark, Methods Mol. Med., 69 427-443 (2002); U.S. Pat. No. 6,566,118 and WO 98/09657, incorporated herein in their entirety by reference) such as by cesium chloride ultracentrifugation gradient or column chromatography.

In some embodiments, rAAVs of the present disclosure comprise a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to a nucleotide sequence listed in TABLE 9.

In some embodiments, the rAAVs of the present disclosure is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the rAAV is AAV2, or a derivative thereof. In some embodiments, the rAAV is AAV8, or a derivative thereof. In some embodiments, the rAAV is AAV-rh74, or a derivative thereof.

### 3. Pharmaceutical Compositions

Recombinant AAV vectors described herein can be used in the manufacture of pharmaceutical compositions. In some embodiments, pharmaceutical compositions disclosed herein comprise recombinant AAV vectors of the present disclosure and a pharmaceutically acceptable carrier and, optionally, other medicinal agents, pharmaceutical agents, stabilizing agents, buffers, carriers, adjuvants, diluents, etc. By “pharmaceutically acceptable” it is meant a material that is not toxic or otherwise undesirable, i.e., the material may be administered to a subject without causing any undesirable biological effects.

In some embodiments, pharmaceutical compositions can comprise sterile aqueous and non-aqueous injection solutions, which are optionally isotonic with the blood of the subject to whom the pharmaceutical composition is to be delivered. Pharmaceutical compositions can contain anti-oxidants, buffers, bacteriostats and solutes, which render the composition isotonic with the blood of the intended subject to be administered. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments pharmaceutical compositions comprise pharmaceutically acceptable vehicles and can include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases and the like.

In some embodiments, pharmaceutical compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

In some embodiments, pharmaceutical compositions disclosed herein can be formulated for intravenous, intramuscular, intrathecal, or intracerebroventricular administration.

### 4. Methods of Treatment

Recombinant AAV of the present disclosure or pharmaceutical compositions comprising the same, can be administered to a subject in need thereof by any mode of delivery including, but not limited to, intravenous, intraperitoneal, and intramuscular administration.

In some embodiments, recombinant AAV of the present disclosure or pharmaceutical compositions comprising the same, can be administered in one, two, three, four, five, or more doses. In some embodiments, when multiple doses are administered, doses can be administered to a subject in need thereof simultaneously or at intervals.

The present application provides methods for reducing the risk of, preventing, and treating cancer and metastasis by administering rAAV vectors as described herein, or pharmaceutical formulations thereof, to a patient.

In other embodiments, recombinant AAV vectors of the present disclosure or pharmaceutical compositions comprising the same, can be administered as a single intravenous dose or divided intravenous doses. In some embodiments, doses for intravenous delivery can be 1×10¹⁰ to 1×10¹³ vg/kg, 2×10¹⁰ to 1×10¹³ vg/kg, 3×10¹⁰ to 1×10¹³ vg/kg, 4×10¹⁰ to 1×10¹³ vg/kg, 5×10¹⁰ to 1×10¹³ vg/kg, 6×10¹⁰ to 1×10¹³ vg/kg, 7×10¹⁰ to 1×10¹³ vg/kg, 8×10¹⁰ to 1×10¹³ vg/kg, 9×10¹⁰ to 1×10¹³ vg/kg, 1×10¹¹ to 1×10¹³ vg/kg, 2×10¹¹ to 1×10¹³ vg/kg, 3×10¹¹ to 1×10¹³ vg/kg, 4×10¹¹ to 1×10¹³ vg/kg, 5×10¹¹ to 1×10¹³ vg/kg, 6×10¹¹ to 1×10¹³ vg/kg, 7×10¹¹ to 1×10¹³ vg/kg, 8×10¹¹ to 1×10¹³ vg/kg, 9×10¹¹ to 1×10¹³ vg/kg, 1×10¹² to 1×10¹³ vg/kg, 2×10¹² to 1×10¹³ vg/kg, 3×10¹² to 1×10¹³ vg/kg, 4×10¹² to 1×10¹³ vg/kg, 5×10¹² to 1×10¹³ vg/kg, 6×10¹² to 1×10¹³ vg/kg, 7×10¹² to 1×10¹³ vg/kg, 8×10¹² to 1×10¹³ vg/kg, 9×10¹² to 1×10¹³ vg/kg, 1×10¹⁰ to 1×10¹² vg/kg, 2×10¹⁰ to 1×10¹² vg/kg, 3×10¹⁰ to 1×10¹² vg/kg, 4×10¹⁰ to 1×10¹² vg/kg, 5×10¹⁰ to 1×10¹² vg/kg, 6×10¹⁰ to 1×10¹² vg/kg, 7×10¹⁰ to 1×10¹² vg/kg, 8×10¹⁰ to 1×10¹² vg/kg, 9×10¹⁰ to 1×10¹² vg/kg, 1×10¹¹ to 1×10¹² vg/kg, 2×10¹¹ to 1×10¹² vg/kg, 3×10¹¹ to 1×10¹² vg/kg, 4×10¹¹ to 1×10¹² vg/kg, 5×10¹¹ to 1×10¹² vg/kg, 6×10¹¹ to 1×10¹² vg/kg, 7×10¹¹ to 1×10¹² vg/kg, 8×10¹¹ to 1×10¹² vg/kg, 9×10¹¹ to 1×10¹² vg/kg, 1×10¹⁰ to 1×10¹¹ vg/kg, 2×10¹⁰ to 1×10¹¹ vg/kg, 3×10¹⁰ to 1×10¹¹ vg/kg, 4×10¹⁰ to 1×10¹¹ vg/kg, 5×10¹⁰ to 1×10¹¹ vg/kg, 6×10¹⁰ to 1×10¹¹ vg/kg, 7×10¹⁰ to 1×10¹¹ vg/kg, 8×10¹⁰ to 1×10¹¹ vg/kg, or 9×10¹⁰ to 1×10¹¹ (viral genome (vg) per kilogram (kg) (vg/kg)). For example, recombinant AAV vectors of the present disclosure or pharmaceutical compositions comprising the same, are administered as a single intravenous dose of 1×10¹¹ vg/kg. In some embodiments, recombinant AAV vectors of the present disclosure or pharmaceutical compositions comprising the same, are administered as a single intravenous dose of 3×10¹¹ vg/kg. In some embodiments, recombinant AAV vectors of the present disclosure or pharmaceutical compositions comprising the same, are administered as a single intravenous dose of 1×10¹² vg/kg. In some embodiments, recombinant AAV vectors of the present disclosure or pharmaceutical compositions comprising the same, are administered as a single intravenous dose of 3×10¹² vg/kg. In some embodiments, recombinant AAV vectors of the present disclosure or pharmaceutical compositions comprising the same, are administered as a single intravenous dose of 5×10¹² vg/kg.

In some embodiments, the present application provides methods for treating a cancer in a patient by administering an effective amount of a recombinant AAV vector as described herein, or pharmaceutical formulation thereof to the patient. In some embodiments the cancer is a breast cancer, an ovarian cancer, an esophageal cancer, a bladder cancer, a gastric cancer, a salivary duct carcinoma, an adenocarcinoma, a uterine cancer, a lung cancer, a glioma, a head and neck cancer, a urothelial cancer, a cervical cancer, a bronchial cancer, a colon cancer, a rectal cancer, a melanoma, a renal cell cancer, a pancreatic cancer, a prostate cancer, a pharyngeal cancer, a liver cancer, an intrahepatic bile duct cancer, or a thyroid cancer. For example, in some embodiments, the cancer is a breast cancer.

### Methods of Reducing Risk of Metastasis

In some embodiments, the present application provides a method for reducing the risk of metastasis by administering to a patient an effective amount of a recombinant adeno-associated viral (rAAV) vector, or pharmaceutical formulation thereof, comprising a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively. In some embodiments, the patient has not been diagnosed with cancer. In some embodiments, the patient has not received a cancer treatment.

In some embodiments, the present application provides a method for reducing the risk of metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for reducing the risk of metastasis by administering to a patient an effective amount of a recombinant adeno-associated viral (rAAV) vector, or pharmaceutical formulation thereof, comprising a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively.

In some embodiments, the present application provides a method for reducing the risk of metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for reducing the risk of metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for reducing the risk of metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method for reducing the risk of metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method for reducing the risk of metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method for reducing the risk of metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method for reducing the risk of metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method for reducing the risk of metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method for reducing the risk of metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method for reducing the risk of metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method for reducing the risk of metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method for reducing the risk of metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Methods of Preventing Metastasis

In some embodiments, the present application provides a method for preventing metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof. In some embodiments, the patient has not been diagnosed with cancer (e.g., a HER2 cancer). In some embodiments, the patient has not received a cancer treatment.

In some embodiments, the present application provides a method for preventing metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, 132, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for preventing metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for preventing metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for preventing metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for preventing metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for preventing metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for preventing metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method for preventing metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method for preventing metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method for preventing metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method for preventing metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method for preventing metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method for preventing metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method for preventing metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method for preventing metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method for preventing metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Methods of Treating Metastasis

In some embodiments, the present application provides a method for treating metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, 132, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, 132, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for treating metastasis by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof. In some embodiments, the present application provides a method for treating

metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for treating metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for treating metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for treating metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for treating metastasis by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for treating metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method for treating metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method for treating metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method for treating metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method for treating metastasis by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method for treating metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method for treating metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method for treating metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method for treating metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method for treating metastasis by administering to a patient an effective amount a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Methods of Promoting T Cell-Mediated Killing of Circulating Tumor Cells

In some embodiments, the present application provides a method for promoting T cell-mediated killing of circulating tumor cells by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising VL CDR1, VL CDR2, and VL CDR3, VH CDR1, VH CDR2, VH CDR3, sequences corresponding to SEQ ID NO: 124, SEQ ID NO: 125, SEQ ID NO: 126, SEQ ID NO: 121, SEQ ID NO: 122, SEQ ID and NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method for promoting T cell-mediated killing of circulating tumor cells by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method for promoting T cell-mediated killing of circulating tumor cells by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method for promoting T cell-mediated killing of circulating tumor cells by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method for promoting T cell-mediated killing of circulating tumor cells by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method for promoting T cell-mediated killing of circulating tumor cells by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Methods of Preventing Cancer in Patients Predisposed to Developing Tumors

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and

SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method of preventing cancer in a patient predisposed to developing tumors (e.g., HER2+ tumors) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method of preventing cancer in a patient predisposed to developing tumors (e.g., HER2+ tumors) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Methods of Preventing Cancer Relapse

In some embodiments, the present application provides a method of preventing cancer relapse in a patient in remission for a cancer (e.g., HER2+ cancer) by administering to a patient an effective amount of a rAAV vector encoding a bispecific fusion protein having (i) a HER2 binding site comprising VL CDR1, VL CDR2, VL CDR3, VH CDR1, VH CDR2, and VH CDR3 sequences corresponding to SEQ ID NO: 100, SEQ ID NO: 101, SEQ ID NO: 102, SEQ ID NO: 97, SEQ ID NO: 98, and SEQ ID NO: 99, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 106, SEQ ID NO: 107, SEQ ID NO: 108, SEQ ID NO: 103, SEQ ID NO: 104, and SEQ ID NO: 105, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 112, SEQ ID NO: 113, SEQ ID NO: 114, SEQ ID NO: 109, SEQ ID NO: 110, and SEQ ID NO: 111 respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 118, SEQ ID NO: 119, SEQ ID NO: 120, SEQ ID NO: 115, SEQ ID NO: 116, and SEQ ID NO: 117, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising VL CDR1, VL CDR2, VL CDR3, VH CDR1, VH CDR2, and VH CDR3 sequences corresponding to SEQ ID NO: 124, SEQ ID NO: 125, SEQ ID NO: 126, SEQ ID NO: 121, SEQ ID NO: 122, and SEQ ID NO: 123, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 127, SEQ ID NO: 128, SEQ ID NO: 129, SEQ ID NO: 130, SEQ ID NO: 131, and SEQ ID NO: 132, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 5 and SEQ ID NO: 4, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 8 and SEQ ID NO: 7, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 10 and SEQ ID NO: 11, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 13 and SEQ ID NO: 14, respectively; (ii) a linker peptide comprising a sequence corresponding to SEQ ID NO: 29; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 16 and SEQ ID NO: 17, respectively, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 88; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 89; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein having: (i) a HER2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15; (ii) a linker peptide comprising a sequence of SEQ ID NO: 29; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 20, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 21, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 22, or a pharmaceutical formulation thereof.

In some embodiments, the present application provides a method of preventing cancer relapse in a patient in remission for a cancer (e.g., HER2+ cancer) by administering to a patient an effective amount of a rAAV vector encoding a bispecific fusion protein comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 23, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 59, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 60, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 61, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 58, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 62, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 83, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 84, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 85, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 82, or a pharmaceutical formulation thereof.

In some embodiments, the present application provides a method of preventing cancer relapse in a patient in remission for a cancer (e.g., HER2+ cancer) by administering to a patient an effective amount of a rAAV vector comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 86, or a pharmaceutical formulation thereof.

### Combination Treatments

In another aspect of the present disclosure, rAAVs, or pharmaceutical formulations thereof, are administered concurrently with treatment of a primary tumor.

In some embodiments of the present disclosure, rAAVs, or pharmaceutical formulations thereof, are administered concurrently with surgical resection, radiation therapy, chemotherapy, or immunotherapy, of a primary tumor.

In some embodiments, the rAAVs as described herein, or pharmaceutical formulations thereof, are administered in combination with a checkpoint inhibitor selected from a CTLA-4 inhibitor, a PD-1 inhibitor, and a PD-L1 inhibitor.

In some embodiments, rAAVs as described herein, or pharmaceutical formulations thereof, are administered in combination with a CTLA-4 inhibitor selected from ipilimumab and tremelimumab. For example, in some embodiments, rAAVs as described herein, or pharmaceutical formulations thereof, are administered in combination with ipilimumab.

In some embodiments, the rAAVs as described herein, or pharmaceutical formulations thereof, are administered in combination with a PD-1 inhibitor selected from pembrolizumab, nivolumab, cemiplimab, dostarlimab, JTZ-4014, spartalizumab, camrelizumab, sintilimab, tislelizumab, toripalimab, INCMGA00012, AMP-224, and AMP-514. For example, in some embodiments, rAAVs as described herein, or pharmaceutical formulations thereof, are administered in combination with pembrolizumab or nivolumab.

In some embodiments, rAAVs as described herein, or pharmaceutical formulations thereof, are administered in combination with a PD-L1 inhibitor selected from atezolizumab, avelumab, durvalumab, KN035, CK-301, AUNP12, CA-170, or BMS-986189. For example, in some embodiments, rAAVs as described herein, or pharmaceutical formulations thereof, are administered in combination with atezolizumab.

The present application provides methods for reducing the risk of, preventing, or treating metastasis where rAAVs as described herein, or pharmaceutical formulations thereof, are administered concurrently with treatment of a primary tumor. In some embodiments the primary tumor is a breast tumor, an ovarian tumor, an esophageal tumor, a bladder tumor, a gastric tumor, a salivary duct carcinoma, an adenocarcinoma, a uterine tumor, a lung tumor, a glioma, a head and neck tumor, a urothelial tumor, a cervical tumor, a bronchial tumor, a colon tumor, a rectal tumor, a melanoma, a renal cell tumor, a pancreatic tumor, a prostate tumor, a pharyngeal tumor, a liver tumor, an intrahepatic bile duct tumor, or a thyroid tumor. For example, in some embodiments, the primary tumor is a breast tumor.

In some embodiments, rAAVs as described herein, or pharmaceutical formulations thereof, are administered in combination with one or more than one rAAV encoding a different bispecific fusion protein that targets a different tumor-associated antigen.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1: Molecular Cloning of rAAV Transgene Constructs

A bispecific fusion protein transgene was synthesized by operably joining from 5′ to 3′, (i) a codon-optimized nucleotide sequence encoding an anti-HER2 antibody VL domain; (ii) a nucleotide sequence encoding a first scFv linker peptide; (iii) a codon-optimized nucleotide sequence encoding an anti-HER2 antibody VH domain; (iv) a nucleotide sequence encoding a linker peptide; (v) a codon-optimized nucleotide sequence encoding an anti-CD3 antibody VH domain; (vi) a nucleotide sequence encoding a second scFv linker peptide; and (vii) a codon-optimized nucleotide sequence encoding an anti-CD3 antibody VL domain.

A transgene cassette was synthesized by operably joining a CAG promoter sequence, the bispecific fusion protein transgene, and a bovine growth hormone (BGH) polyadenylation sequence. The transgene cassette was cloned into an appropriate cloning vector (e.g., pUC) and confirmed by DNA sequencing. Confirmed constructs were restriction digested and gel-purified for subsequent cloning into an appropriate AAV8 backbone vector containing AAV2 ITR sites and a kanamycin resistance gene.

Following ligation, DNA was transformed into E. coli (e.g., VB UltraStable™, Vector Builder, Chicago, IL), grown on Kanamycin-selective media and purified. Gene constructs that have undergone successful ligation were identified by restriction digest.

Clones were then scaled-up by bacterial transformation in E. coli. Correct plasmid sequences were re-confirmed by restriction digest.

### Transient Transfection and Viral Packaging

The confirmed AAV vectors were transiently transfected into HEK293 cells in combination with adenoviral helper plasmid and a packaging construct that delivers the AAV rep gene together with the AAV cap gene, using a standard calcium phosphate transfection method (for example, as described in Vandendriessche et al. (2007. J Thromb Haemost 5:16-24), incorporated by reference herein). Two days post transfection, AAV particles were harvested and purified using two successive rounds of cesium chloride density gradient ultracentrifugation and titered.

### Example 2: Binding of Bispecific Fusion Proteins to HER2 and CD3

Purified AAV particles as described in Example 1, were used to transduce production cells (e.g. HEK293 cells) to express the bispecific fusion proteins. Bispecific fusion proteins were collected and purified from culture supernatants and/or cell lysates and assayed for HER2 and CD3 binding.

Human cancer cell lines expressing HER2 (e.g., cell lines with high levels of HER2 expression or cell lines engineered to express exogenous HER2) were used to assay binding of bispecific fusion proteins to HER2. Bispecific fusion proteins at various concentrations were incubated with HER2-expressing cells and binding is detected using a fluorophore-conjugated secondary antibody. Cells were analyzed by flow cytometry and compared to binding of cell incubated with HER2-binding control antibodies (e.g., pertuzumab, or trastuzumab).

Similarly, T cell lines or peripheral blood mononuclear cells (PBMCs) were used to assay binding of bispecific fusion proteins to CD3. Bispecific fusion proteins at various concentrations were incubated with T cell lines or PBMCs and binding was detected using a fluorophore-conjugated secondary antibody. Cells were analyzed by flow cytometry and compared to binding of cell incubated with anti-CD3 control antibodies.

Standard co-culture cytotoxicity assays were also performed using dilution titrations of the purified bispecific fusion protein in culture media alone as well as in 100% mouse or pooled human serum. The concentration of bispecific fusion protein needed to kill 10% (EC10), 50% (EC50) and 90% (EC90) of the target cells under standard conditions after 48 hours was determined. ED10/50/90 for 10 different cell lines that vary in their expression of HER2 were determined (e.g., SKBR3, HCC159, MDAMB134VI, BT483, LY2, MCF7 cells).

### Example 3: Bispecific Fusion Proteins Induce T Cell-Mediated Cytotoxicity

PBMCs were isolated from human peripheral blood buffy coats using density gradient centrifugation. PBMCs were then co-cultured with human cancer cells expressing HER2 in the presence of various concentrations of bispecific fusion protein, or control antibody (e.g., pertuzumab or trastuzumab). After co-culture, cells were lysed and analyzed using a commercially available cytotoxicity assay in accordance with the manufacturer's instructions (e.g., CytoTox96® Non-Radioactive Cytotoxicity Assay, Promega, Madison, WI).

### Example 4: Dose Determination

To determine vector dose and serum levels for administration in human patients, mice were administered different doses of AAV in half-log increments (1×10¹¹, 3×10¹¹, 1×10¹², and 3×10¹² vg/kg) serum levels were analyzed from retro-orbital blood draws at a timepoint predicted to be well after steady state (e.g., 28 days). Vector doses in animal studies were chosen that achieve serum levels above EC90 for most cell lines as determined in Example 2.

### Example 5: AAV8 Delivery of Bispecific Fusion Proteins Prevents Metastasis

To determine the efficacy of AAV8-delivered bispecific fusion proteins in preventing metastasis, mouse treatment groups consist of: (1) no local control, (2) oncolytic HSV1, (3) surgical resection, and (4) radiation therapy. Each treatment group includes its own vehicle control (i.e. no bispecific fusion protein) group for purpose of comparison.

HER2+ human cancer cells (e.g. GFP+, HER2+ cells) are administered to test groups either in the flank of the mice or orthotopically in the mammary fat pad at various concentrations, then mice are administered with controls or purified AAV8 for in vivo expression of bispecific fusion proteins. Primary tumor sizes and the presence of metastatic lesions are monitored by measuring the bioluminescence of the engrafted tumors and satellite metastases (if any) at various time points. Metastases are confirmed and/or counted on necroscopy. Metastatic tissues (e.g., lung and liver) are analyzed by immunohistochemistry and/or flow cytometry for T cell infiltration, activation, and/or exhaustion. Peripheral T cells are also analyzed by flow cytometry for activation and checkpoint marker expression every 2 weeks. Flow cytometry is also used to monitor circulating tumor cells every 2 weeks.

Alternatively, any one of a number of breast cancer xenograft models is used (e.g., HCC 1954, JIMT-1, MDA-MB-361 (D YT2) and 144580 (PDX)). For example, mice (e.g., Balb/c) are subcutaneously engrafted with HER2+ human cancer cells expressing luciferase or green fluorescent protein (GFP). Tumors are allowed to grow to an appropriate size, then mice are intravenously administered with controls or purified AAV8 particles for in vivo expression of bispecific fusion proteins. Primary tumor sizes and the presence of metastatic lesions are monitored by measuring the bioluminescence of the engrafted tumors and satellite metastases (if any) at various time points. Metastases are confirmed and/or counted on necroscopy. Metastatic tissues (e.g., lung and liver) are analyzed by immunohistochemistry and/or flow cytometry for T cell infiltration, activation, and/or exhaustion. Peripheral T cells are also analyzed by flow cytometry for activation and checkpoint marker expression every 2 weeks. Flow cytometry is also used to monitor circulating tumor cells every 2 weeks.

To determine the efficacy of AAV8-delivered bispecific fusion proteins in preventing metastasis, any one of a number of breast cancer xenograft models is used (e.g., HCC 1954, JIMT-1, MDA-MB-361 (D YT2) and 144580 (PDX)). For example, mice (e.g., Balb/c) are subcutaneously engrafted with HER2+ human cancer cells expressing luciferase. Tumors are allowed to grow to an appropriate size, then mice are intravenously administered with purified AAV8 particles for in vivo expression of bispecific fusion proteins.

### Example 6: Determining Immunogenicity of AAV8-Delivered Bispecific Fusion Protein and Loss of Expression Over Time

To determine if AAV8-delivered bispecific fusion proteins elicit an immune reaction, AAV were intravenously administered to immunocompetent C57Bl/6 mice. Mice were monitored with weekly blood tests for measuring levels of bispecific fusion protein over the first 3 months, then monthly over the next 9 months. Weights were monitored at each blood draw as a readout of safety/toxicity.

### Example 7: Evaluation of Effects of Anti-HER2/Anti-CD3ε Fusion Proteins on HER2-Expressing Cell Lines

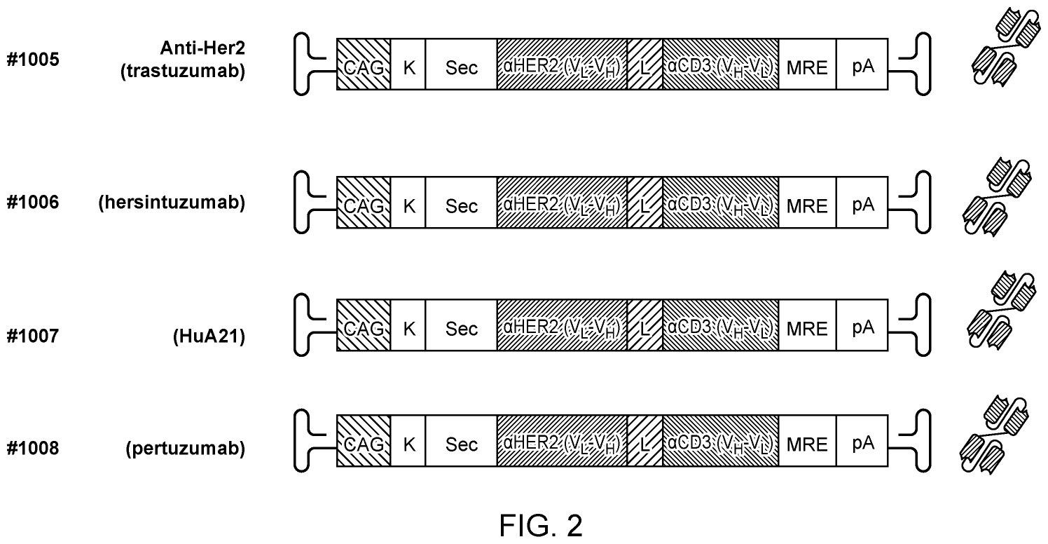

Four anti-HER2/anti-CD3ε bispecific fusion proteins containing one of four anti-HER2 scFvs (trastuzumab, hersintuzumab, HuA21, and pertuzumab) and an anti-CD3 scFv connected by a linker were prepared. AAV construct designs are detailed in FIG. 2.

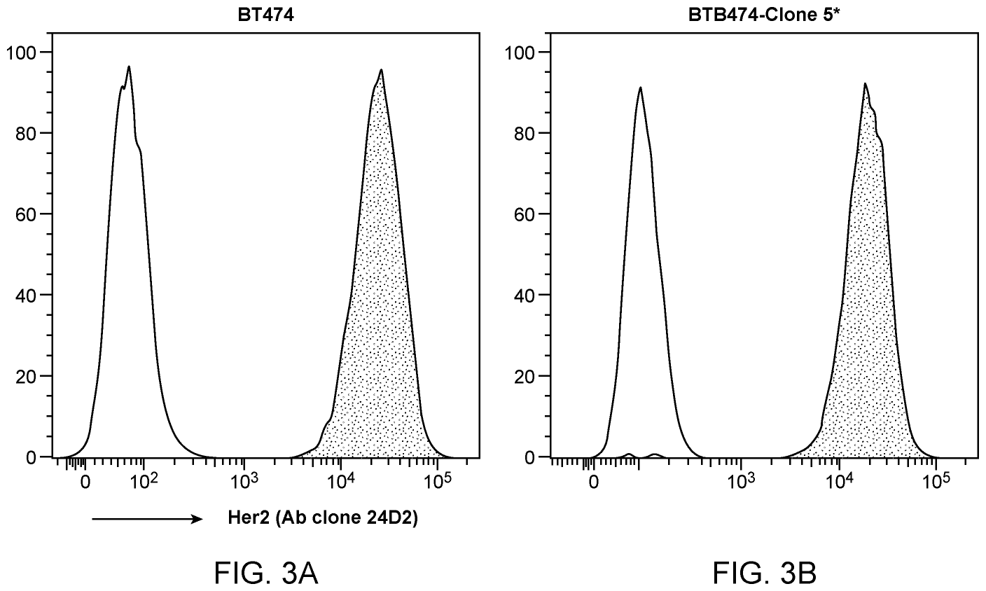

BT474 and “Clone 5” (in vitro-selected Herceptin-resistant line of BT474 cells that retain HER2 expression, can be bound by anti-HER2 monoclonal antibodies, and can respond to antibody-dependent cellular cytotoxicity) were isolated. 5×10⁵ cells of each line were stained with an anti-HER2 antibody, and staining was assessed by flow cytometry (FIGS. 3A-3B). Results show that both normal BT474 cells and Clone 5 cells expressed high levels of HER2.

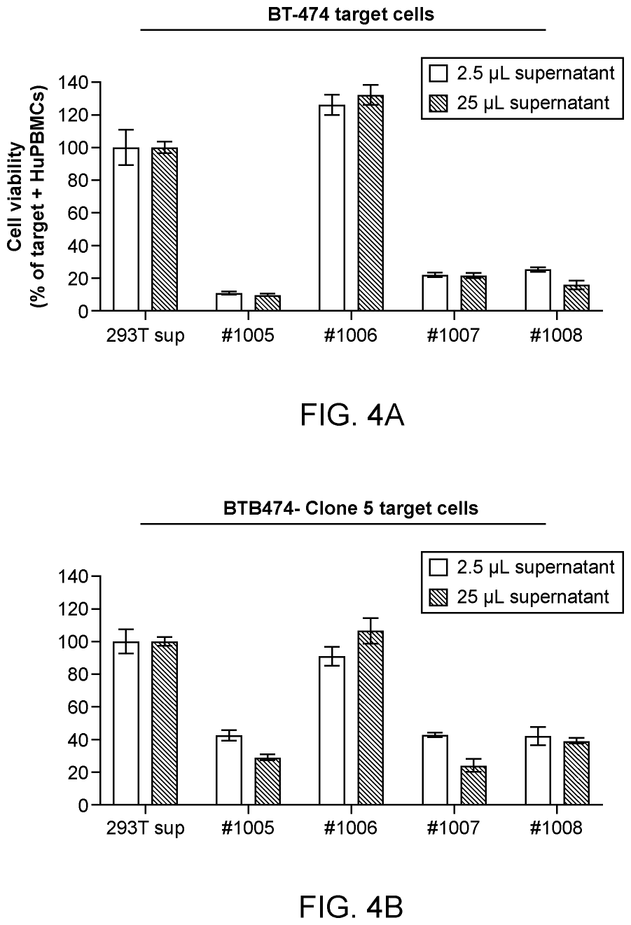

To assess the effects of anti-HER2/anti-CD3E bispecific fusion proteins on the HER2-expressing cells, supernatant was collected from 293T cells engineered to produce control or one of the anti-HER2/anti-CD3ε bispecific fusion proteins (1005, 1006, 1007, or 1008), detailed above, via AAV transduction. BT474 and Clone 5 cells were each incubated with human peripheral blood mononuclear cells (huPBMCs) and 2.5 μL or 25 μL of the supernatant from the 293T cells at an effector:target (E:T) ratio of 10:1. After 48 hours of incubation, target cell viability was measured (FIGS. 4A-4B). Results showed that co-treatment of HER2-expressing target cells with huPBMCs and bispecific fusion protein 1005, 1007, or 1008 resulted in increased target cell killing, with reduced results shown in Clone 5 cells. No target cell killing was observed in either cell line after co-treatment with huPBMCs and protein 1006.

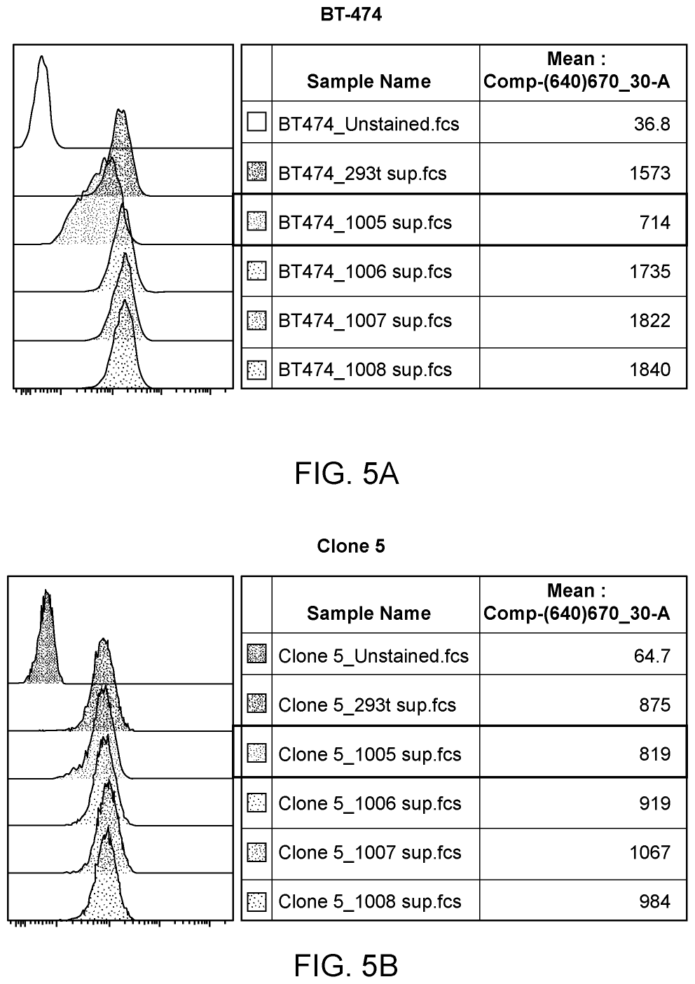

To assess binding of the anti-HER2/anti-CD3ε bispecific fusion proteins to HER2-expressing cells, BT474 and Clone 5 cells were each incubated with 293T supernatant containing an anti-HER2/anti-CD3ε bispecific fusion protein. Cells were then stained for flow cytometry with anti-HER2 antibody H2M5B (a trastuzumab biosimilar) (FIGS. 5A-5B). Results demonstrated that only protein 1005, which contains a trastuzumab-derived scFv, was able to compete with H2M5B for HER2 binding, suggesting that protein 1005 binds to HER2-expressing cells. The other anti-HER2/anti-CD3ε bispecific fusion proteins did not compete with H2M5B for HER2 binding because they bind epitopes different from trastuzumab.

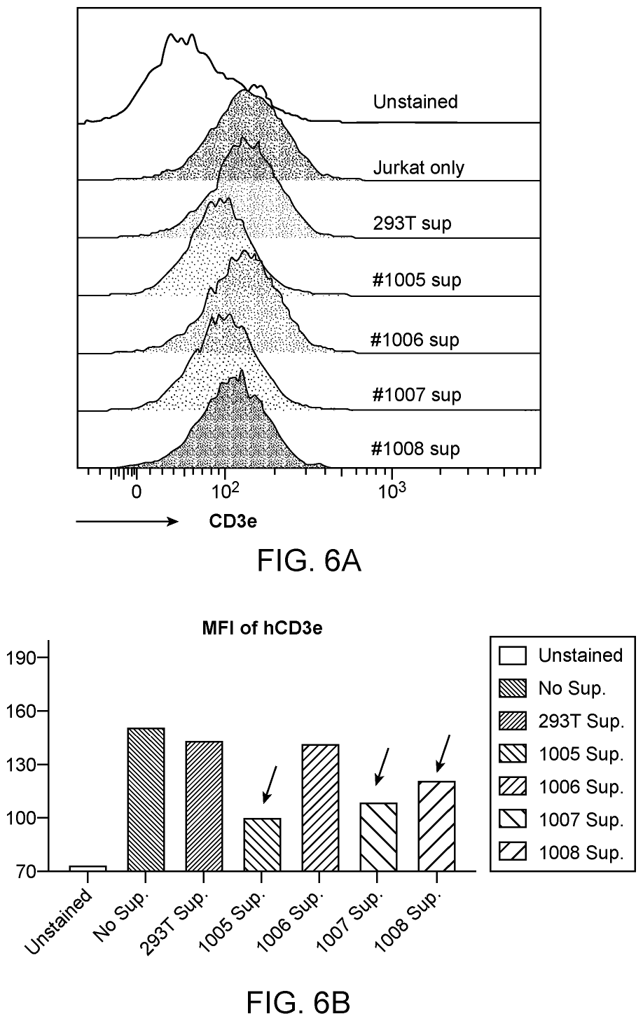

To assess binding of the anti-HER2/anti-CD3ε bispecific fusion proteins to CD3ε-expressing cells, Jurkat T cells were incubated with 293T supernatant containing an anti-HER2/anti-CD3 bispecific fusion protein. Cells were then stained for flow cytometry with anti-CD3ε antibody OKT3 (FIGS. 6A-6B). Results demonstrated that proteins 1005, 1007, and 1008, but not 1006, were able to compete with OKT3 for CD3ε binding, suggesting that these proteins bind to CD3ε-expressing cells. These results were consistent with the ability of these proteins to induce target cell killing shown above.

To further assess the effects of anti-HER2/anti-CD3ε bispecific fusion proteins on HER2-expressing target cells, BT474 cells were incubated with 293T supernatant containing an anti-HER2/anti-CD3ε bispecific fusion protein or a control supernatant containing an anti-EGFR/anti-CD3ε bispecific fusion protein. Cells were imaged every hour with IncuCyte for 4 days and viability was measured. Results demonstrated that proteins 1005, 1007, and 1008, but not 1006, were able to induce target cell killing.

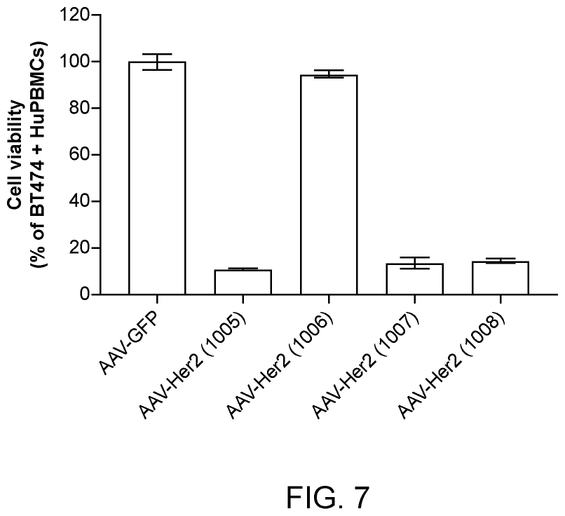

To assess the effects of rAAV8-derived anti-HER2/anti-CD3ε bispecific fusion proteins on HER2-expressing target cells, BT474 cells were incubated with huPBMCs and 293T supernatant containing an anti-HER2/anti-CD3ε bispecific fusion protein or an anti-GFP control. After 72 hours of incubation, viability was measured (FIG. 7). Results demonstrated that proteins 1005, 1007, and 1008, but not 1006, were able to induce target cell killing.

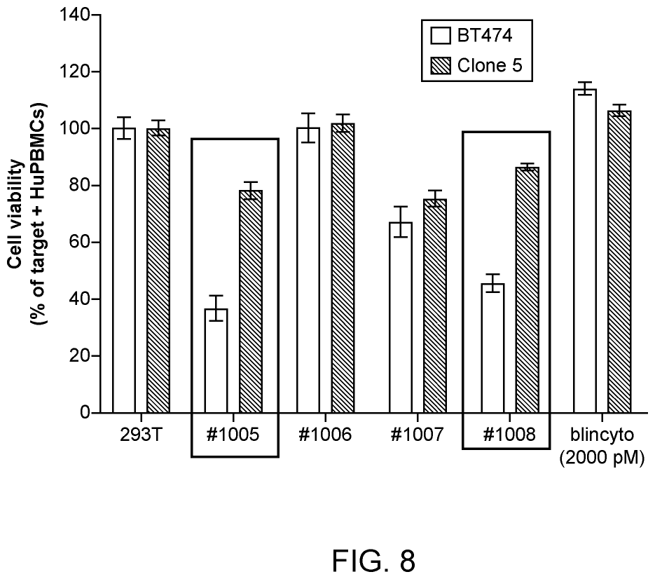

To further assess the effects of rAAV8-derived anti-HER2/anti-CD3ε bispecific fusion proteins on HER2-expressing target cells, BT474 and Clone 5 cells were each incubated with huPBMCs and 293T supernatant containing an anti-HER2/anti-CD3ε bispecific fusion protein or an anti-GFP control. After 72 hours of incubation viability was measured (FIG. 8). Results demonstrated that proteins 1005, 1007, and 1008, but not 1006, were able to induce target cell killing.

### Example 8: In Vivo Evaluation of Effects of Anti-HER2/Anti-CD3ε Fusion Proteins

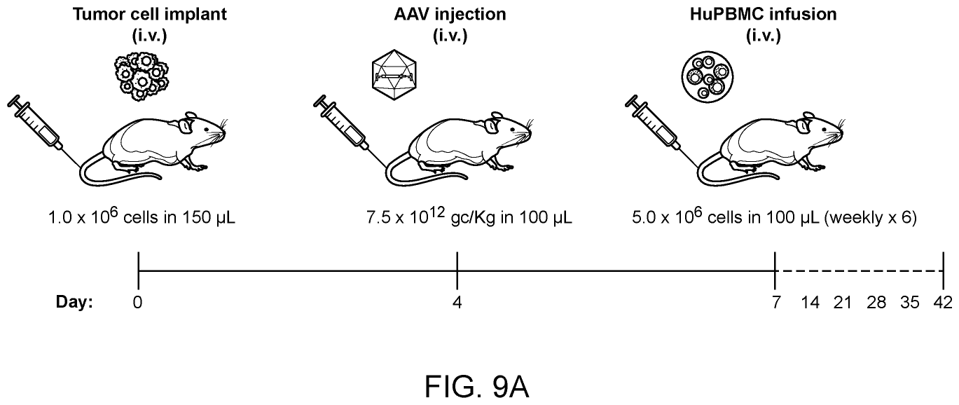

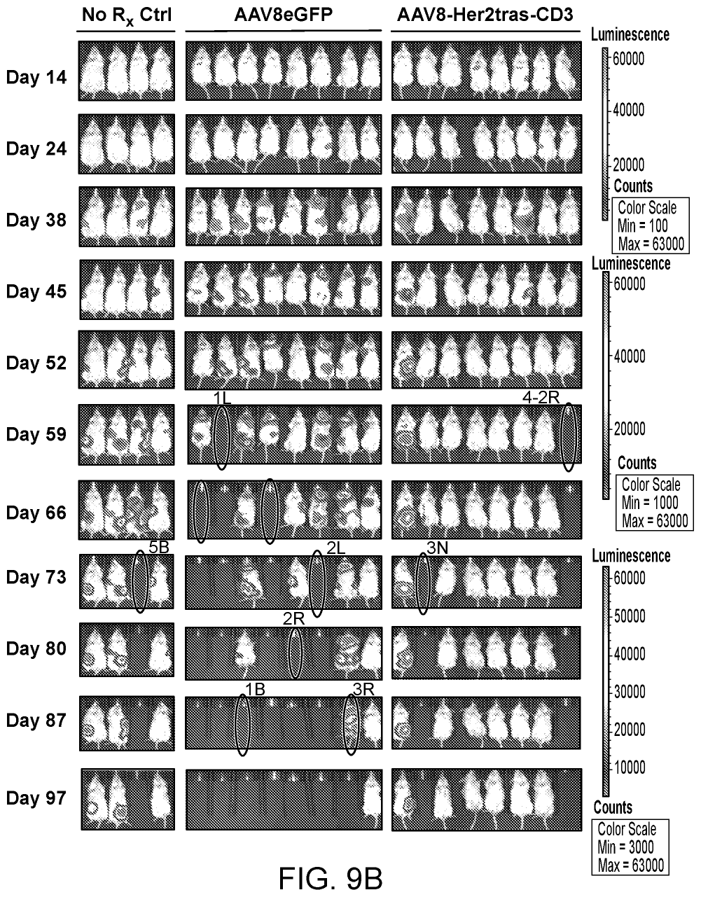

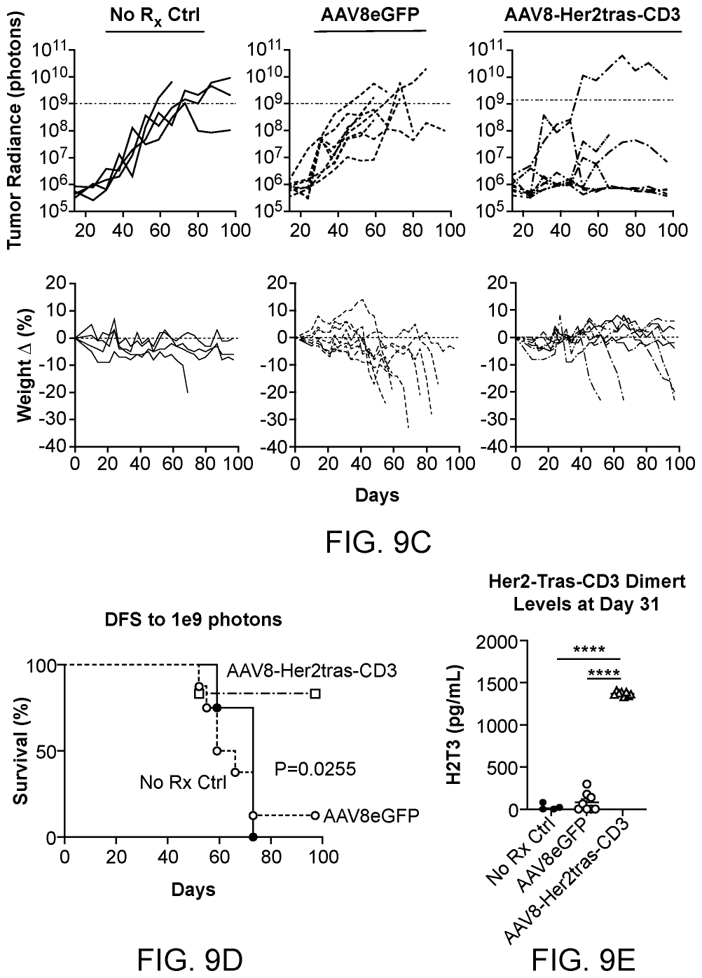

A metastatic BT474 Clone5 tumor model in hu-PBMC-NSG-SGM3 mice was used to assess the anti-tumor potential of AAV8-derived anti-HER2/anti-CD3ε bispecific fusion protein therapy. FIG. 9A outlines the experimental overview. Mice were divided into three groups: untreated, AAV8-GFP control+huPBMC, and AAV8-anti-HER2/anti-CD3ε+huPBMC. 1.0×10⁶ BT474Clone5EGFP-Luc tumor cells were injected into the tail veins of each mouse. 4 days after tumor inoculation, mice were weighed and treated with AAV agents. 7 days after tumor inoculation, mice were treated with AAV agents and huPBMCs. AAV and huPBMC treatment was repeated on days 14, 21, 28, 35 and 42. Mice were tracked by measuring weight, in vivo imaging (IVIS), and survival. Images of mouse tumors on indicated days are shown in FIG. 9B. Tumor growth as measured by radiance (top panels) and change in weight (bottom panels) are shown in FIG. 9C. Survival for each group are shown in FIG. 9D. Overall levels of anti-HER2/anti-CD3ε bispecific fusion protein are shown in FIG. 9E. These results indicate that a single AAV injection was effective preventing metastatic disease as measured by in vivo bioluminescence and animal survival. Serum anti-HER2/anti-CD3ε bispecific fusion protein levels were consistent at day 32 and within the expected therapeutic range previously demonstrated for blinatumomab for all mice at the intravenous dose of 7.5×10¹² gc/kg. Some surviving animals without tumor burden began to lose weight after 5 weeks due to graft vs host disease.

### Example 9: In Vivo Evaluation of Effects of Anti-HER2/Anti-CD3ε Fusion Proteins on Preventing Cancer Relapse

A metastatic BT474 Clone5 tumor model in hu-PBMC-NSG-SGM3 mice is used to assess the anti-tumor potential of AAV8-derived anti-HER2/anti-CD3ε bispecific fusion protein therapy in preventing cancer relapse.

Briefly, mice are divided into three groups: untreated, AAV8-GFP control+huPBMC, and AAV8-anti-HER2/anti-CD38+huPBMC. 1.0×106 BT474Clone5EGFP-Luc tumor cells are injected into the tail veins of each mouse. 4 days after tumor inoculation, mice are weighed and treated with a chemotherapeutic agent (e.g., germcitabine). Chemotherapy treatment occurs on days 14, 21, 28, 35 and 42. Chemotherapy treatment is then stopped and mice are treated with AAV agents followed by AAV agents and huPBMCs. AAV and huPBMC treatment is repeated on days 14, 21, 28, 35 and 42. Mice are tracked by measuring weight, in vivo imaging (IVIS), and survival.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

What is claimed is:

1. A recombinant adeno-associated viral (rAAV) vector, comprising from 5′ to 3′:
(a) a 5′ AAV inverted terminal repeat (ITR);
(b) a promoter;
(c) a transgene encoding a bispecific fusion protein comprising:
(i) a HER2 binding site comprising a light chain variable region (VL) and a heavy chain variable region (VH) of an anti-HER2 antibody,
(ii) a linker peptide, and
(iii) a CD3 binding site comprising a VH and a VL of an anti-CD3 antibody;
(d) a modified RNA stability regulatory element (MRE) and
(e) a 3′ AAV ITR.

2. The rAAV vector of claim 1 wherein the promoter is selected from the group consisting of a chicken β-actin promoter, an elongation factor 1α (EF1α) promoter, a simian virus 40 (SV40) promoter, or a CAG promoter.

3. The rAAV vector of any one of claims 1-2, wherein the promoter is a CAG promoter.

4. The rAAV vector of any one of claims 1-3, wherein the promoter comprises a sequence at least 95% identical to SEQ ID NO: 87.

5. The rAAV vector of any one of claims 1-4, wherein the anti-HER2 antibody VL comprises a CDR1, CDR2, and CDR3 sequence of SEQ ID NO: 106, SEQ ID NO: 107, and SEQ ID NO: 108, respectively, and the anti-HER2 antibody VH comprises a CDR1, CDR2, and CDR3 sequence of SEQ ID NO: 103, SEQ ID NO: 104, and SEQ ID NO: 105, respectively.

6. The rAAV vector of claim 5, wherein the anti-HER2 antibody VL and VH comprise sequences at least 95% identical to SEQ ID NO: 5 and SEQ ID NO: 4, respectively.

7. The rAAV vector of claim 5 or 6, wherein the HER2 binding site is a single chain variable fragment (scFv).

8. The rAAV vector of claim 7, wherein anti-HER2 antibody VL is fused to the anti-HER2 antibody VH using an scFv linker peptide comprising of SEQ ID NO: 25.

9. The rAAV vector of any one of claims 5-8, wherein the HER2 binding site comprises a sequence at least 95% identical to SEQ ID NO: 89.

10. The rAAV vector of any one of claims 1-4, wherein the anti-HER2 antibody VL comprises a complementarity determining region 1 (CDR1), complementarity determining region 2 (CDR2), and complementarity determining region 3 (CDR3) sequence of SEQ ID NO: 100, SEQ ID NO: 101, and SEQ ID NO: 102, respectively, and the anti-HER2 antibody VH comprises a CDR1, CDR2, and CDR3 sequence of SEQ ID NO: 97, SEQ ID NO: 98, and SEQ ID NO: 99, respectively.

11. The rAAV vector of claim 10, wherein the anti-HER2 antibody VL and VH comprise sequences at least 95% identical to SEQ ID NO: 2 and SEQ ID NO: 1, respectively.

12. The rAAV vector of claim 10 or 11, wherein the HER2 binding site is a single chain variable fragment (scFv).

13. The rAAV vector of claim 12, wherein the anti-HER2 antibody VL is fused to the anti-HER2 antibody VH using an scFv linker peptide comprising a sequence of SEQ ID NO: 24.

14. The rAAV vector of claim 13, wherein the scFv comprises a sequence at least 95% identical to SEQ ID NO: 88.

15. The rAAV vector of any one of claims 1-4, wherein the anti-HER2 antibody VL comprises a CDR1, CDR2, and CDR3 sequence of SEQ ID NO: 112, SEQ ID NO: 113, and SEQ ID NO: 114, respectively, and the anti-HER2 antibody VH comprises a CDR1, CDR2, and CDR3 sequence of SEQ ID NO: 109, SEQ ID NO: 110, and SEQ ID NO: 111, respectively.

16. The rAAV vector of claim 15, wherein the anti-HER2 antibody VL and VH comprise sequences at least 95% identical to SEQ ID NO: 8 and SEQ ID NO: 7, respectively.

17. The rAAV vector of claim 15 or 16, wherein the HER2 binding site is a single chain variable fragment (scFv).

18. The rAAV vector of claim 17, wherein anti-HER2 antibody VL is fused to the anti-HER2 antibody VH using an scFv linker peptide comprising of SEQ ID NO: 26.

19. The rAAV vector of any one of claims 15-18, wherein the HER2 binding site comprises a sequence at least 95% identical to SEQ ID NO: 90.

20. The rAAV vector of any one of claims 1-4, wherein the anti-HER2 antibody VL comprises a CDR1, CDR2, and CDR3 sequence of SEQ ID NO: 118, SEQ ID NO: 119, and SEQ ID NO: 120, respectively, and the anti-HER2 antibody VH comprises a CDR1, CDR2, and CDR3 sequence of SEQ ID NO: 115, SEQ ID NO: 116, and SEQ ID NO: 117, respectively.

21. The rAAV vector of claim 20, wherein the anti-HER2 antibody VL and VH comprise sequences at least 95% identical to SEQ ID NO: 10 and SEQ ID NO: 11, respectively.

22. The rAAV vector of claim 20 or 21, wherein the HER2 binding site is a single chain variable fragment (scFv).

23. The rAAV vector of claim 22, wherein the anti-HER2 antibody VL is fused to the anti-HER2 antibody VH by an scFv linker peptide comprising an amino acid sequence of SEQ ID NO: 27.

24. The rAAV vector of any one of claims 20-23, wherein the HER2 binding site comprises a sequence at least 95% identical to SEQ ID NO: 12.

25. The rAAV vector of any one of claims 1-4, wherein the anti-HER2 antibody VL comprises a CDR1, CDR2, and CDR3 sequence of SEQ ID NO: 124, SEQ ID NO: 125, and SEQ ID NO: 126, respectively, and the anti-HER2 antibody VH comprises a CDR1, CDR2, and CDR3 sequence of SEQ ID NO: 121, SEQ ID NO: 122, and SEQ ID NO: 123, respectively.

26. The rAAV vector of claim 25, wherein the anti-HER2 antibody VL and VH comprise sequences at least 95% identical to SEQ ID NO: 13 and SEQ ID NO: 14, respectively.

27. The rAAV vector of claim 25 or 26, wherein the HER2 binding site is a single chain variable fragment (scFv).

28. The rAAV vector of claim 27, wherein anti-HER2 antibody VL is fused to the anti-HER2 antibody VH by an scFv linker peptide comprising an amino acid sequence of SEQ ID NO: 27.

29. The rAAV vector of any one of claims 25-28, wherein the HER2 binding site comprises a sequence at least 95% identical to SEQ ID NO: 15.

30. The rAAV vector of any one of claims 1-29, wherein the linker peptide comprises a sequence identical to SEQ ID NO: 29.

31. The rAAV vector of any one of claims 1-30, wherein the anti-CD3 antibody VH comprises a CDR1, CDR2, and CDR3 sequence of SEQ ID NO: 127, SEQ ID NO: 128, and SEQ ID NO: 129, respectively, and the anti-CD3 antibody VL comprises a CDR1, CDR2, and CDR3 sequence of SEQ ID NO: 130, SEQ ID NO: 131, and SEQ ID NO: 132, respectively.

32. The rAAV vector of claim 31, wherein the anti-CD3 antibody VH and VL comprise sequences at least 95% identical to SEQ ID NO: 16 and SEQ ID NO: 17, respectively.

33. The rAAV vector of claim 31 or 32, wherein the CD3 binding site is a single chain variable fragment (scFv).

34. The rAAV vector of claim 33, wherein the anti-CD3 antibody VH is fused to the anti-CD3 antibody VL using an scFv linker peptide comprising a sequence identical to SEQ ID NO: 28.

35. The rAAV vector of any one of claims 31-34, wherein the CD3 binding site comprises a sequence at least 95% identical to SEQ ID NO: 18.

36. The rAAV vector of any one of claims 1-35, wherein the rAAV vector further comprises a Kozak sequence.

37. The rAAV vector of any one of claims 1-36, wherein the rAAV vector further comprises a polyadenylation sequence 3′ of the transgene sequence and 5′ of the 3′ AAV ITR.

38. The rAAV vector of claim 37, wherein the polyadenylation sequence is a bovine growth hormone (BGH) polyadenylation sequence at least 95% identical to SEQ ID NO: 81.

39. The rAAV vector of any one of claims 1-38, wherein the vector further comprises an antibiotic resistance gene sequence.

40. The rAAV vector of claim 39, wherein the antibiotic resistance gene is a kanamycin resistance gene.

41. The rAAV vector of any one of claims 1-40, wherein the AAV is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

42. The rAAV vector of any one of claims 1-41, wherein the bispecific fusion protein comprises an amino acid sequence at least 90% identical to SEQ ID NO: 19, SEQ ID NO: 20, SEQ ID NO: 21, SEQ ID NO: 22, or SEQ ID NO: 23.

43. The rAAV vector of any one of claims 1-42, wherein the transgene comprises a nucleotide sequence at least 95% identical to SEQ ID NO: 59, SEQ ID NO: 60, SEQ ID NO: 61, SEQ ID NO: 58, or SEQ ID NO: 62.

44. The rAAV vector of any one of claims 1-43, wherein the transgene comprises reduced CpG dinucleotides and/or increased methylation of CpG dinucleotides as compared to a parental equivalent.

45. The rAAV vector of any one of claims 1-44, wherein the rAAV vector comprises a sequence at least 90% identical to SEQ ID NO: 83, SEQ ID NO: 84, SEQ ID NO: 85, SEQ ID NO: 82, or SEQ ID NO: 86.

46. A recombinant adeno-associated viral (rAAV) vector, comprising from 5′ to 3′:
(a) a 5′ AAV inverted terminal repeat (ITR) comprising a sequence at least 90% identical to SEQ ID NO: 75 or SEQ ID NO: 91;
(b) a promoter;
(c) a transgene comprising a sequence encoding a bispecific fusion protein comprising,
(i) a HER2 binding site comprising a light chain variable region (VL) comprising a CDR1, CDR2, and CDR3 sequence of SEQ ID NO: 106, SEQ ID NO: 107, and SEQ ID NO: 108, respectively, and a heavy chain variable region (VH) comprising a CDR1, CDR2, and CDR3 sequence of SEQ ID NO: 103, SEQ ID NO: 104, and SEQ ID NO: 105, respectively of an anti-HER2 antibody;
(ii) a linker peptide comprising a sequence according to SEQ ID NO: 29, and
(iii) a CD3 binding site comprising a VH comprising a CDR1, CDR2, and CDR3 sequence of SEQ ID NO: 127, SEQ ID NO: 128, and SEQ ID NO: 129, respectively, and a VL comprising a CDR1, CDR2, and CDR3 sequence of SEQ ID NO: 130, SEQ ID NO: 131, and SEQ ID NO: 132, respectively of an anti-CD3 antibody;
(d) modified RNA stability regulatory element (MRE) and
(e) a 3′ AAV ITR comprising a sequence at least 90% identical to SEQ ID NO: 75 or SEQ ID NO: 91.

47. A recombinant adeno-associated viral (rAAV) vector, comprising from 5′ to 3′:
(a) a 5′ AAV inverted terminal repeat (ITR) comprising a sequence at least 90% identical SEQ ID NO: 75 or SEQ ID NO: 91;
(b) a promoter;
(c) a transgene encoding a bispecific fusion protein comprising a sequence at least 90% identical to SEQ ID NO: 19, SEQ ID NO: 20, SEQ ID NO: 21, SEQ ID NO: 22, or SEQ ID NO: 23;
(d) modified RNA stability regulatory element (MRE) and
(e) a 3′ AAV ITR comprising a sequence at least 90% identical to SEQ ID NO: 75 or SEQ ID NO: 91.

48. A recombinant adeno-associated viral (rAAV) vector comprising a sequence at least 90% identical to SEQ ID NO: 20.

49. A method of reducing the risk of metastatic disease in a patient comprising administering to the patient an effective amount of a recombinant adeno-associated viral (rAAV) vector of any one of claims 1-48 or pharmaceutical formulation thereof.

50. A method of delaying the onset of metastatic disease in a patient comprising administering to the patient an effective amount of a recombinant adeno-associated viral (rAAV) vector of any one of claims 1-48 or pharmaceutical formulation thereof.

51. A method of preventing metastatic disease in a patient comprising administering to the patient an effective amount of a recombinant adeno-associated viral (rAAV) vector of any one of claims 1-48 or pharmaceutical formulation thereof.

52. A method of promoting T cell-mediated killing of circulating tumor cells in a patient comprising administering to the patient an effective amount of a recombinant adeno-associated viral (rAAV) vector of any one of claims 1-48 or pharmaceutical formulation thereof.

53. The method according to any one of claims 49-52, wherein the rAAV or pharmaceutical formulation thereof is administered concurrently with treatment of a primary tumor.

54. The method of claim 53, wherein the primary tumor is a breast tumor.

55. The methods of claim 53 or 54, wherein treatment of the primary tumor includes surgical resection, radiation therapy, chemotherapy, or immunotherapy.

56. A method of preventing cancer in a patient predisposed to developing HER2+ tumors comprising administering to the patient an effective amount of a recombinant adeno-associated viral (rAAV) vector of any one of claims 1-48 or pharmaceutical formulation thereof.

57. A method of preventing cancer relapse in a patient in remission for a HER2+ cancer comprising administering to the patient an effective amount of a recombinant adeno-associated viral (rAAV) vector of any one of claims 1-48 or pharmaceutical formulation thereof.

58. The method according to any one of claims 49-57, wherein the rAAV or pharmaceutical formulation thereof is administered with a checkpoint inhibitor selected from the group consisting of: a CTLA-4 inhibitor, a PD-1 inhibitor, and a PD-L1 inhibitor.

59. The method of claim 58, wherein the checkpoint inhibitor is selected from the group consisting of: pembrolizumab, ipilimumab, nivolumab, and atezolizumab.

60. A pharmaceutical formulation comprising a recombinant adeno-associated viral (rAAV) vector of any one of claims 1-48, and a pharmaceutically acceptable carrier.
